(19) United States
(12) Patent Application Publication
(10) Pub. No.: US 2003/0220241 A1
(43) Pub. Date: Nov. 27, 2003

(54) Method of treating cancer

(76) Inventors: Deborah Defeo-Jones, Rahway, NJ (US); Raymond E. Jones, Lansdale, PA (US); Allen I. Oliff, Gwynedd Valley, PA (US)

Correspondence Address: MERCK AND CO INC, P O BOX 2000, RAHWAY, NJ, 070650907

(21) Appl. No.: 10/244,215

(22) Filed: Sep. 16, 2002

Related U.S. Application Data
(63) Continuation of application No. 09/542,769, filed on Apr. 4, 2000.
(60) Provisional application No. 60/127,746, filed on Apr. 5, 1999.

(51) Int. Cl. A61K 39/00

(52) U.S. Cl. 514/12; 424/185.1

(57) ABSTRACT

The present invention relates to methods of treating cancer using a combination of a compound which is a PSA conjugate and a compound which is a inhibitor of prenyl-protein transferase, which methods comprise administering to said mammal, either sequentially in any order or simultaneously, amounts of at least two therapeutic agents selected from a group consisting of a compound which is a PSA conjugate and a compound which is a inhibitor of prenyl-protein transferase. The invention also relates to methods of preparing such compositions.

### RELATED APPLICATION

The present patent application is a continuation-in-part application of copending provisional application Serial No. 60/127,746, filed Apr. 5, 1999.

### BACKGROUND OF THE INVENTION

The present invention relates to methods of treating cancer, and more particularly cancer associated with cells that produce prostate specific antigen (PSA), which comprise administering to a patient in need thereof at least one inhibitor of a prenyl-protein transferase and at least one conjugate, which comprises an oligopeptide that is selectively cleaved by PSA and a cytotoxic agent.

In 1996 cancer of the prostate gland was expected to be diagnosed in 317,000 men in the U.S. and 42,000 American males die from this disease (Garnick, M. B. (1994). The Dilemmas of Prostate Cancer. Scientific American, April:72-81). Thus, prostate cancer is the most frequently diagnosed malignancy (other than that of the skin) in U.S. men and the second leading cause of cancer-related deaths (behind lung cancer) in that group.

Prostate specific Antigen (PSA) is a single chain 33 kDa glycoprotein that is produced almost exclusively by the human prostate epithelium and occurs at levels of 0.5 to 2.0 mg/ml in human seminal fluid (Nadji, M., Taber, S. Z., Castro, A., et al. (1981) Cancer 48:1229; Papsidero, L., Kuriyama, M., Wang, M., et al. (1981). JNCI 66:37; Qui, S. D., Young, C. Y. F., Bihartz, D. L., et al. (1990), J. Urol. 144:1550; Wang, M. C., Valenzuela, L. A., Murphy, G. P., et al. (1979). Invest. Urol. 17:159). The single carbohydrate unit is attached at asparagine residue number 45 and accounts for 2 to 3 kDa of the total molecular mass. PSA is a protease with chymotrypsin-like specificity (Christensson, A., Laurell, C. B., Lilja, H. (1990). Eur. J. Biochem. 194:755-763). It has been shown that PSA is mainly responsible for dissolution of the gel structure formed at ejaculation by proteolysis of the major proteins in the sperm entrapping gel, Semenogelin I and Semenogelin II, and fibronectin (Lilja, H. (1985). J. Clin. Invest. 76:1899; Lilja, H., Oldbring, J., Rannevik, G., et al. (1987). J. Clin. Invest. 80:281; McGee, R. S., Herr, J. C. (1988). Biol. Reprod. 39:499). The PSA mediated proteolysis of the gel-forming proteins generates several soluble Semenogelin I and Semenogelin II fragments and soluble fibronectin fragments with liquefaction of the ejaculate and release of progressively motile spermatoza (Lilja, H., Laurell, C. B. (1984). Scand. J. Clin. Lab. Invest. 44:447; McGee, R. S., Herr, J. C. (1987). Biol. Reprod. 37:431). Furthermore, PSA may proteolytically degrade IGFBP-3 (insulin-like growth factor binding protein 3) allowing IGF to stimulate specifically the growth of PSA secreting cells (Cohen et al., (1992) J. Clin. Endo. & Meta. 75:1046-1053).

PSA complexed to alpha 1-antichymotrypsin is the predominant molecular form of serum PSA and may account for up to 95% of the detected serum PSA (Christensson, A., Björk, T., Nilsson, O., et al. (1993). J. Urol. 150:100-105; Lilja, H., Christensson, A., Dahlén, U. (1991). Clin. Chem. 37:1618-1625; Stenman, U. H., Leinoven, J., Alfthan, H., et al. (1991). Cancer Res. 51:222-226). The prostatic tissue (normal, benign hyperplastic, or malignant tissue) is implicated to predominantly release the mature, enzymatically active form of PSA, as this form is required for complex formation with alpha 1-antichymotrypsin (Mast, A. E., Enghild, J. J., Pizzo, S. V., et al. (1991). Biochemistry 30:1723-1730; Perlmutter, D. H., Glover, G. I., Rivetna, M., et al. (1990). Proc. Natl. Acad. Sci. USA 87:3753-3757). Therefore, in the microenvironment of prostatic PSA secreting cells the PSA is believed to be processed and secreted in its mature enzymatically active form not complexed to any inhibitory molecule. PSA also forms stable complexes with alpha 2-macroglobulin, but as this results in encapsulation of PSA and complete loss of the PSA epitopes, the in vivo significance of this complex formation is unclear. A free, noncomplexed form of PSA constitutes a minor fraction of the serum PSA (Christensson, A., Björk, T., Nilsson, O., et al. (1993). J. Urol. 150:100-105; Lilja, H., Christensson, A., Dahlén, U. (1991). Clin. Chem. 37:1618-1625). The size of this form of serum PSA is similar to that of PSA in seminal fluid (Lilja, H., Christensson, A., Dahlén, U. (1991). Clin. Chem. 37:1618-1625) but it is yet unknown as to whether the free form of serum PSA may be a zymogen; an internally cleaved, inactive form of mature PSA; or PSA manifesting enzyme activity. However, it seems unlikely that the free form of serum PSA manifests enzyme activity, since there is considerable (100 to 1000 fold) molar excess of both unreacted alpha 1-antichymotrypsin and alpha 2-macroglobulin in serum as compared with the detected serum levels of the free 33 kDa form of PSA (Christensson, A., Björk, T., Nilsson, O., et al. (1993). J. Urol. 150:100-105; Lilja, H., Christensson, A., Dahlén, U. (1991). Clin. Chem. 37:1618-1625).

Serum measurements of PSA are useful for monitoring the treatment of adenocarcinoma of the prostate (Duffy, M. S. (1989). Ann. Clin. Biochem. 26:379-387; Brawer, M. K. and Lange, P. H. (1989). Urol. Suppl. 5:11-16; Hara, M. and Kimura, H. (1989). J. Lab. Clin. Med. 113:541-548), although above normal serum concentrations of PSA have also been reported in benign prostatic hyperplasia and subsequent to surgical trauma of the prostate (Lilja, H., Christensson, A., Dahlén, U. (1991). Clin. Chem. 37:1618-1625). Prostate metastases are also known to secrete immunologically reactive PSA since serum PSA is detectable at high levels in prostatectomized patients showing widespread metatstatic prostate cancer (Ford, T. F., Butcher, D. N., Masters, R. W., et al. (1985). Brit. J. Urology 57:50-55). Therefore, a cytotoxic compound that could be activated by the proteolytic activity of PSA should be prostate cell specific as well as specific for PSA secreting prostate metastases.

Conjugates which comprise an oligopeptide which can be selectively cleaved by enzymatically active PSA attached, either directly or via a linker to a cytotoxic agent and which are useful in the treatment of prostate cancer and benign prostatic hyperplasia have been previously described (U.S. Pat. No. 5,599,686 and 5,866,679).

Prenylation of proteins by intermediates of the isoprenoid biosynthetic pathway represents a class of post-translational modification (Glomset, J. A., Gelb, M. H., and Farnsworth, C. C. (1990). Trends Biochem. Sci. 15, 139-142; Maltese, W. A. (1990). FASEB J. 4, 3319-3328). This modification typically is required for the membrane localization and function of these proteins. Prenylated proteins share characteristic C-terminal sequences including CaaX (C, Cys; a, usually aliphatic amino acid; X, another amino acid), XXCC, or XCXC. Three post-translational processing steps have been described for proteins having a C-terminal CaaX sequence: addition of either a 15 carbon (farnesyl) or 20 carbon (geranylgeranyl) isoprenoid to the Cys residue, proteolytic cleavage of the last 3 amino acids, and methylation of the new C-terminal carboxylate (Cox, A. D. and Der, C. J. (1992a). Critical Rev. Oncogenesis 3:365-400; Newman, C. M. H. and Magee, A. I. (1993). Biochim. Biophys. Acta 1155:79-96). Some proteins may also have a fourth modification: palmitoylation of one or two Cys residues N-terminal to the farnesylated Cys. While some mammalian cell proteins terminating in XCXC are carboxymethylated, it is not clear whether carboxy methylation follows prenylation of proteins terminating with a XXCC motif (Clarke, S. (1992). Annu. Rev. Biochem. 61, 355-386). For all of the prenylated proteins, addition of the isoprenoid is the first step and is required for the subsequent steps (Cox, A. D. and Der, C. J. (1992a). Critical Rev. Oncogenesis 3:365-400; Cox, A. D. and Der, C. J. (1992b) Current Opinion Cell Biol. 4:1008-1016).

Three enzymes have been described that catalyze protein prenylation: farnesyl-protein transferase (FPTase), geranylgeranyl-protein transferase type I (GGPTase-I), and geranylgeranyl-protein transferase type-II (GGPTase-II, also called Rab GGPTase). These enzymes are found in both yeast and mammalian cells (Clarke, 1992; Schafer, W. R. and Rine, J. (1992) Annu. Rev. Genet. 30:209-237). Each of these enzymes selectively uses farnesyl diphosphate (FPP) or geranyl-geranyl diphosphate as the isoprenoid donor and selectively recognizes the protein substrate. FPTase farnesylates CaaX-containing proteins that end with Ser, Met, Cys, Gln or Ala. For FPTase, CaaX tetra-peptides comprise the minimum region required for interaction of the protein substrate with the enzyme. The enzymological characterization of these three enzymes has demonstrated that it is possible to selectively inhibit one with little inhibitory effect on the others (Moores, S. L., Schaber, M. D., Mosser, S. D., Rands, E., O'Hara, M. B., Garsky, V. M., Marshall, M. S., Pompliano, D. L., and Gibbs, J. B., J. Biol. Chem., 266:17438 (1991), U.S. Pat. No. 5,470,832).

The Ras protein is part of a signalling pathway that links cell surface growth factor receptors to nuclear signals initiating cellular proliferation. Biological and biochemical studies of Ras action indicate that Ras functions like a G-regulatory protein. In the inactive state, Ras is bound to GDP. Upon growth factor receptor activation, Ras is induced to exchange GDP for GTP and undergoes a conformational change. The GTP-bound form of Ras propagates the growth stimulatory signal until the signal is terminated by the intrinsic GTPase activity of Ras, which returns the protein to its inactive GDP bound form (D. R. Lowy and D. M. Willumsen, Ann. Rev. Biochem. 62:851-891 (1993)). Activation of Ras leads to activation of multiple intracellular signal transduction pathways, including the MAP Kinase pathway and the Rho/Rac pathway (Joneson et al., Science 271:810-812).

Inhibitors of farnesyl-protein transferase have been described in two general classes. The first class includes analogs of FPP, while the second is related to protein substrates (e.g., Ras) for the enzyme. The peptide derived inhibitors that have been described are generally cysteine containing molecules that are related to the CAAX motif that is the signal for protein prenylation. (Schaber et al., ibid; Reiss et. al., ibid; Reiss et al., PNAS, 88:732-736 (1991)). Such inhibitors may inhibit protein prenylation while serving as alternate substrates for the farnesyl-protein transferase enzyme, or may be purely competitive inhibitors (U.S. Pat. No. 5,141,851, University of Texas; N. E. Kohl et al., Science, 260:1934-1937 (1993); Graham, et al., J. Med. Chem., 37, 725 (1994)).

Numerous other classes of compounds have been described as inhibitors of a prenyl-protein transferase or in particular of farnesyl-protein transferase.

It is the object of the instant invention to provide a method for treating cancer, and more particularly cancer associated with cells that produce prostate specific antigen (PSA), which offers advantages over previously disclosed methods of treatment.

### SUMMARY OF THE INVENTION

A method of treating cancer, and more particularly cancer associated with cells that produce prostate specific antigen (PSA), is disclosed which is comprised of administering to a patient in need of such treatment amounts of at least one inhibitor of a prenyl-protein transferase and at least one conjugate, which comprises an oligopeptide that is selectively cleaved by PSA and a cytotoxic agent.

### BRIEF DESCRIPTION OF THE FIGURES

FIG. 1: PSA Levels after Administration of a Combination of a PSA Conjugate and a Prenyl Protein Transferase Inhibitor

Terminal plasma levels of PSA in nude mice having LNCaP.FGC cells xenographs following administration of: Column 1: vehicle only; Column 2: administration of Compound A via the ALZET® micro-osmotic pump alone; Column 3: administration of Compound B alone; Column 4: administration of Compound A via the ALZET® micro-osmotic pump and administration of Compound B. Each dot represents a PSA level for an individual mouse. Details of the experimental protocol are found in Example 47.

FIG. 2: Tumor Weights after Administration of a Combination of a PSA Conjugate and a Prenyl Protein Transferase Inhibitor

Terminal tumor weights at the sites of the xenographs in nude mice having LNCaP.FGC cells xenographs following administration of: Column 1: vehicle only; Column 2: administration of Compound A via the ALZET® micro-osmotic pump alone; Column 3: administration of Compound B alone; Column 4: administration of Compound A via the ALZET® micro-osmotic pump and administration of Compound B. Each dot represents a tumor weight for an individual mouse. Details of the experimental protocol are found in Example 47.

FIG. 3: Animal Weights after Administration of a Combination of a PSA Conjugate and a Prenyl Protein Transferase Inhibitor

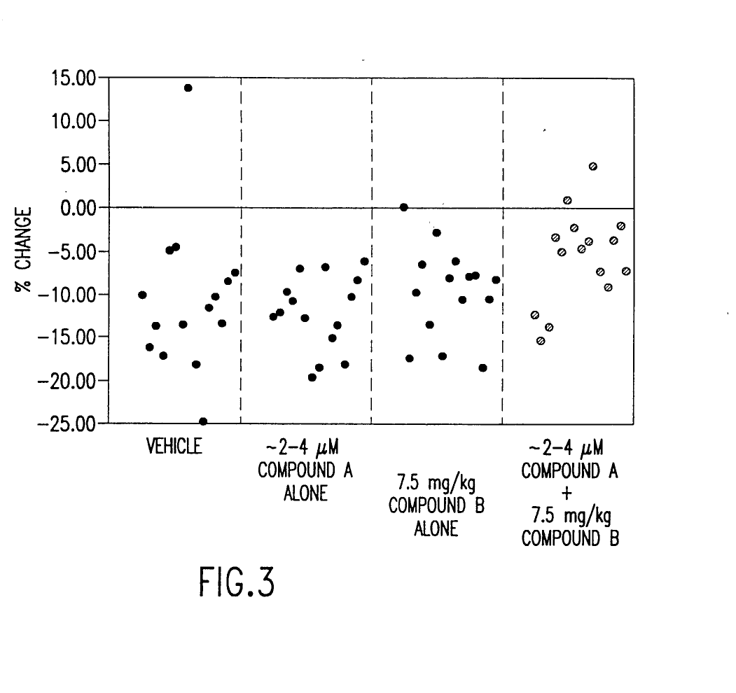

Terminal animal weights of nude mice having LNCaP.FGC cells xenographs following administration of: Column 1: vehicle only; Column 2: administration of Compound A via the ALZET® micro-osmotic pump alone; Column 3: administration of Compound B alone; Column 4: administration of Compound A via the ALZET® micro-osmotic pump and administration of Compound B. Each dot represents a animal weight for an individual mouse. Details of the experimental protocol are found in Example 47.

### DETAILED DESCRIPTION OF THE INVENTION

The present invention relates to a method of treating cancer, and more particularly cancer associated with cells that produce prostate specific antigen (PSA), which is comprised of administering to a patient in need of such treatment amounts of at least one inhibitor of a prenyl-protein transferase and at least one conjugate (hereinafter refered to as a PSA conjugate), which comprises an oligopeptide that is selectively cleaved by PSA and a cytotoxic agent.

In practicing the instant method of treatment, it is understood that the inhibitor(s) of a prenyl protein transferase and the PSA conjugate(s) may be administered either simultaneously in a single pharmaceutical composition or individually in separate pharmaceutical compositions. If the inhibitor(s) of a prenyl protein transferase and the PSA conjugate(s) are administered in separate compositions, such compositions may be administered simultaneously or consecutively.

The term “consecutively” when used in the context of administration of two or more separate pharmaceutical compositions means that administrations of the separate pharmaceutical compositions are at separate times. The term “consecutively” also includes administration of two or more separate pharmaceutical compositions wherein administration of one or more pharmaceutical compositions is a continuous administration over a prolonged period of time and wherein administration of another of the compositions occur at a discrete time during the prolonged period.

The terms prenyl-protein transferase inhibitor and inhibitor of prenyl-protein transferase refer to compounds which antagonize, inhibit or counteract the expression of the gene coding a prenyl-protein transferase or the activity of the protein product thereof. Prenyl-protein transferases include farnesyl-protein transferase and geranylgeranyl-protein transferase type I.

The terms farnesyl-protein transferase inhibitor and inhibitor of farnesyl-protein transferase likewise refer to compounds which antagonize, inhibit or counteract the expression of the gene coding farnesyl-protein transferase or the activity of the protein product thereof.

The present invention is not limited in any way by the specific prenyl-protein transferase inhibitor. Either a protein substrate-competitive inhibitor and/or a prenyl pyrophosphate-competitive inhibitor now known or subsequently discovered or developed may be utilized. Prenyl-protein transferase inhibitors useful in the instant invention are described hereinbelow.

The term selective as used herein with respect to the inhibitors of a prenyl-protein transferase or farnesyl protein transferase refers to the inhibitory activity of the particular compound against prenyl-protein transferase activity. For example, a selective inhibitor of farnesyl-protein transferase exhibits at least 20 times greater activity against farnesyl-protein transferase when comparing its activity against another receptor or enzymatic activity, respectively. Preferably, if a selective inhibitor of farnesyl-protein transferase is desired, the selectivity is at least 100 times or more.

In an embodiment of the invention, the component of the instant composition which is the inhibitor of a prenyl-protein transferase is a selective inhibitor of farnesyl-protein transferase and is characterized by:

a) an IC₅₀ (a measurement of in vitro inhibitory activity) of less than about 500 nM against transfer of a farnesyl residue to a protein or peptide substrate comprising a CAAX^{F} motif by farnesyl-protein transferase.

It is more preferred that the selective inhibitor of farnesyl-protein transferase is characterized by:

a) an IC₅₀ (a measurement of in vitro inhibitory activity) of less than about 100 nM against transfer of a farnesyl residue to a protein or peptide substrate comprising a CAAX^{F} motif by farnesyl-protein transferase.

As used herein, the term “CAAX^{F}” is used to designate a protein or peptide substrate that incorporates four amino acid C-terminus motif that is farnesylated by farnesyl-protein transferase. In particular, such “CAAX^{F}” motifs include (the corresponding human protein is in parentheses): CVLS (H-ras) (SEQ.ID.: 11), CVIM (K4B-Ras) (SEQ.ID.: 1), CVVM (N-Ras) (SEQ.ID.: 3), CKVL (RhoB) (SEQ.ID.: 9), CLIM (PFX) (SEQ.ID.: 10) and CNIQ (Rap2A) (SEQ.ID.: 13). It is understood that certain of the “CAAX^{F}” containing protein or peptide substrates may also be geranylgeranylated by GGTase-I.

A method for measuring the activity of the inhibitors of prenyl-protein transferase utilized in the instant methods against transfer of a farnesyl residue to a protein or peptide substrate comprising a CAAX^{F} motif by farnesyl-protein transferase is described in Example 35.

It is also preferred that the selective inhibitor of farnesyl-protein transferase is further characterized by:

b) an IC₅₀ (a measure of in vitro inhibitory activity) for inhibition of the prenylation of newly synthesized K-Ras protein more than about 100-fold higher than the IC₅₀ for the inhibition of the farnesylation of hDJ protein.

When measuring such IC₅₀s the assays described in Examples 40 and 41 may be utilized.

It is also preferred that the selective inhibitor of farnesyl-protein transferase is further characterized by:

c) an IC₅₀ (a measurement of in vitro inhibitory activity) for inhibition of K4B-Ras dependent activation of MAP kinases in cells at least 100-fold greater than the IC₅₀ for inhibition of the farnesylation of the protein hDJ in cells.

It is also preferred that the selective inhibitor of farnesyl-protein transferase is further characterized by:

d) an IC₅₀ (a measurement of in vitro inhibitory activity) against H-Ras dependent activation of MAP kinases in cells at least 1000 fold lower than the inhibitory activity (IC₅₀) against H-ras-CVLL (SEQ.ID.NO.: 1) dependent activation of MAP kinases in cells.

When measuring Ras dependent activation of MAP kinases in cells the assays described in Example 39 may be utilized.

In another embodiment, the component of the instant composition which is an inhibitor of a prenyl-protein transferase utilized in the instant invention is efficacious in vivo as an inhibitor of both farnesyl-protein transferase and geranylgeranyl-protein transferase type I (GGTase-I). Preferably, such a dual inhibitor of farnesyl-protein transferase and geranylgeranyl-protein transferase type I, which may be termed a Class II prenyl-protein transferase inhibitor, is characterized by:

a) an IC₅₀ (a measurement of in vitro inhibitory activity) of less than about 1 μM for inhibiting the transfer of a geranylgeranyl residue to a protein or peptide substrate comprising a CAAX^{G} motif by geranylgeranyl-protein transferase type I in the presence of a modulating anion; and

b) an IC₅₀ (a measurement of in vitro inhibitory activity) of less than about 500 nM against transfer of a farnesyl residue to a protein or peptide substrate comprising a CAAX^{F} motif by farnesyl-protein transferase.

Preferably, such a Class II prenyl-protein transferase inhibitor is also characterized by:

c) inhibition of the cellular prenylation of greater than (>) about 50% of the newly synthesized K4B-Ras protein after incubation of assay cells with the dual inhibitor of farnesyl-protein transferase and geranylgeranyl-protein transferase type I at a concentration of less than (<)10 μM.

More preferably, such a Class II prenyl-protein transferase inhibitor is also characterized by:

c) inhibition of the cellular prenylation of greater than (>) about 50% of the newly synthesized K4B-Ras protein after incubation of assay cells with the dual inhibitor of farnesyl-protein transferase and geranylgeranyl-protein transferase type I at a concentration of less than (<)₅ μM.

The term “CAAX^{G}” will refer to such motifs that may be geranylgeranylated by GGTase-I. In particular, such “CAAX^{G}” motifs include (the corresponding human protein is in parentheses): CVIM (K4B-Ras) (SEQ.ID.: 1), CVLL (mutated H-Ras) (SEQ.ID.: 2), CVVM (N-Ras) (SEQ.ID.: 3), CVIM (K4A-Ras) (SEQ.ID.: 4), CLLL (Rap-IA) (SEQ. ID.: 5), CQLL (Rap-IB) (SEQ.ID.: 6), CSIM (SEQ.ID.: 7), CAIM (SEQ.ID.: 8), CKVL (RhoB) (SEQ.ID.: 9), CLIM (PFX) (SEQ.ID.: 10) and CVIL (Rap2B) (SEQ.ID.: 12). Preferably, the CAAX motif is CVIM (SEQ.ID.: 1). It is understood that some of the “CAAX^{G} containing protein or peptide substrates may also be farnesylated by farnesyl-protein transferase.

The modulating anion may be selected from any type of molecule containing an anion moiety. Preferably the modulating anion is selected from a phosphate or sulfate containing anion. Particular examples of modulating anions useful in the instant GGTase-I inhibition assay include adenosine 5′-triphosphate (ATP), 2′-deoxyadenosine 5′-triphosphate (dATP), 2′-deoxycytosine 5′-triphosphate (dCTP), b-glycerol phosphate, pyrophosphate, guanosine 5′-triphosphate (GTP), 2′-deoxyguanosine 5′-triphosphate (dGTP), uridine 5′-triphosphate, dithiophosphate, 3′-deoxythymidine 5′-triphosphate, tripolyphosphate, D-myo-inositol 1,4,5-triphosphate, chloride, guanosine 5′-monophosphate, 2′-deoxyguanosine 5′-monophosphate, orthophosphate, formycin A, inosine diphosphate, trimetaphosphate, sulfate and the like. Preferably, the modulating anion is selected from adenosine 5′-triphosphate, 2′-deoxyadenosine 5′-triphosphate, 2′-deoxycytosine 5′-triphosphate, b-glycerol phosphate, pyrophosphate, guanosine 5′-triphosphate, 2′-deoxyguanosine 5′-triphosphate, uridine 5′-triphosphate, dithiophosphate, 3′-deoxythymidine 5′-triphosphate, tripolyphosphate, D-myo-inositol 1,4,5-triphosphate and sulfate. Most preferably, the modulating anion is selected from adenosine 5′-triphosphate, b-glycerol phosphate, pyrophosphate, dithiophosphate and sulfate.

A method for measuring the activity of the inhibitors of prenyl-protein transferase utilized in the instant methods against transfer of a geranylgeranyl residue to protein or peptide substrate comprising a CAAX^{G} motif by geranylgeranyl-protein transferase type I in the presence of a modulating anion is described in Example 36.

Examples of assay cells that may be utilized to determine inhibition of cellular processing of newly synthesized protein that is a substrate of an enzyme that can modify the K4B-Ras protein C-terminus include 3T3, C33a, PSN-1 (a human pancreatic carcinoma cell line) and K-ras-transformed Rat-1 cells. Preferred assay cell line has been found to be PSN-1. The preferred newly synthesized protein, whose percentage of processing is assessed in this assay, is selected from K4B-Ras and Rap1.

A method for measuring the activity of the inhibitors of prenyl-protein transferase, as well as the instant combination compositions, utilized in the instant methods against the cellular processing of newly synthesized protein that is a substrate of an enzyme that can modify the K4B-Ras protein C-terminus after incubation of assay cells with the compound of the invention transferase is described in Example 40 and 41.

A Class II prenyl-protein transferase inhibitor may also be characterized by:

a) an IC₅₀ (a measurement of in vitro inhibitory activity) for inhibiting K4B-Ras dependent activation of MAP kinases in cells of less than 5 μM.

A Class II prenyl-protein transferase inhibitor may also be characterized by:

a) an IC₅₀ (a measurement of in vitro inhibitory activity) for inhibiting K4B-Ras dependent activation of MAP kinases in cells between 0.1 and 100 times the IC₅₀ for inhibiting the farnesylation of the protein hDJ in cells; and

b) an IC₅₀ (a measurement of in vitro inhibitory activity) for inhibiting K4B-Ras dependent activation of MAP kinases in cells greater than 5-fold lower than the inhibitory activity (IC₅₀) against expression of the SEAP protein in cells transfected with the pCMV-SEAP plasmid that constitutively expresses the SEAP protein.

A Class II prenyl-protein transferase inhibitor may also be characterized by:

a) an IC₅₀ (a measurement of in vitro inhibitory activity) against H-Ras dependent activation of MAP kinases in cells greater than 2 fold lower but less than 20,000 fold lower than the inhibitory activity (IC₅₀) against H-ras-CVLL (SEQ.ID.NO.: 1) dependent activation of MAP kinases in cells; and

b) an IC₅₀ (a measurement of in vitro inhibitory activity) against H-ras-CVLL dependent activation of MAP kinases in cells greater than 5-fold lower than the inhibitory activity (IC₅₀) against expression of the SEAP protein in cells transfected with the pCMV-SEAP plasmid that constitutively expresses the SEAP protein.

A Class II prenyl-protein transferase inhibitor may also be characterized by:

a) an IC₅₀ (a measurement of in vitro inhibitory activity) against H-Ras dependent activation of MAP kinases in cells greater than 10-fold lower but less than 2,500 fold lower than the inhibitory activity (IC₅₀) against H-ras-CVLL (SEQ.ID.NO.: 1) dependent activation of MAP kinases in cells; and

b) an IC₅₀ (a measurement of in vitro inhibitory activity) against H-ras-CVLL dependent activation of MAP kinases in cells greater than 5 fold lower than the inhibitory activity (IC₅₀) against expression of the SEAP protein in cells transfected with the pCMV-SEAP plasmid that constitutively expresses the SEAP protein.

In a third embodiment, the prenyl-protein transferase inhibitors useful in the instantly claimed methods are dual inhibitors of farnesyl-protein transferase and geranylgeranyl-protein transferase type I (GGTase-I) and are further characterized in that the inhibitory activity of the compounds against GGTase-I is greater than the inhibitory activity against FPTase. Such dual inhibitor compounds having greater activity against GGTase type I may be termed Class III prenyl protein transferase inhibitors. Preferably, such Class III prenyl protein transferase inhibitors inhibit FPTase in vitro (Example 35) at an IC₅₀ of less than 1 μM and inhibit GGTase-I in vitro (Example 36) at an IC₅₀ of less than 50 nM. Also preferably, the compounds of this embodiment of the instant invention inhibit the cellular processing of the Rap1 protein (Example 42) at an EC₅₀ of less than 50 nM. Also more preferably, the ratio of the IC₅₀ of the compounds of this embodiment of the instant invention for in vitro inhibition of FPTase to the IC₅₀ of the compounds of the instant invention for in vitro inhibition of GGTase type I is greater than 25.

The composition useful in the instant method of treatment also comprises a PSA conjugate. The PSA conjugate comprises an oligopeptide, which is specifically recognized by the free prostate specific antigen (PSA) and are capable of being proteolytically cleaved by the enzymatic activity of the free prostate specific antigen, covalently bonded directly, or through a chemical linker, to a cytotoxic agent. Ideally, the cytotoxic activity of the cytotoxic agent is greatly reduced or absent when the oligopeptide containing the PSA proteolytic cleavage site is bonded directly, or through a chemical linker, to the cytotoxic agent and is intact. Also ideally, the cytotoxic activity of the cytotoxic agent increases significantly or returns to the activity of the unmodified cytotoxic agent upon proteolytic cleavage of the attached oligopeptide at the cleavage site. While it is not necessary for practicing this aspect of the invention, a preferred embodiment of this aspect of the invention is a conjugate wherein the oligopeptide, and the chemical linker if present, are detached from the cytotoxic agent by the proteolytic activity of the free PSA and any other native proteolytic enzymes present in the tissue proximity, thereby releasing unmodified cytotoxic agent into the physiological environment at the place of proteolytic cleavage. Pharmaceutically acceptable salts of the conjugates are also included.

Oligopeptides that are selectively cleaved by enzymatically active PSA can be identified by a number of assays, in particularly the assays described in Examples 43-46 and 48.

In one embodiment of the instant invention, the oligopeptide component of the PSA conjugate incorporates a cyclic amino acid having a hydrophilic substituent as part of the oligopeptides, said cyclic amino acid which contributes to the aqueous solubility of the conjugate. Examples of such hydrophilic cyclic amino acids include but are not limited to hydroxylated, polyhydroxylated and alkoxylated proline and pipecolic acid moieties.

In a prefered embodiment of the invention the oligopeptide component of the PSA conjugate is characterized by having a protecting group on the terminus amino acid moiety that is not attached to the cytotoxic agent. Such protection of the terminal amino acid reduces or eliminates the enzymatic degradation of such peptidyl therapeutic agents by the action of exogenous aminopeptidases and carboxypeptidases which are present in the blood plasma of warm blooded animals. Examples of protecting groups that may be attached to the amino moiety of an N-terminus oligopeptide include, but are not limited to acetyl, benzoyl, pivaloyl, succinyl, glutaryl, hydoxyalkanoyl, polyhydroxyalkanoyl, polyethylene glycol (PEG) containing alkanoyl and the like. Examples of protecting groups that may be attached to the carboxylic acid of a C-terminus oligopeptide include, but are not limited to, formation of an organic or inorganic ester of the carboxylic acid, such as an alkyl, aralkyl, aryl, polyether ester, phosphoryl and sulfuryl, or conversion of the carboxylic acid moiety to a substituted or unsubstituted amide moiety. The N-terminus or C-terminus of the oligopeptide may also be substituted with a unnatural amino acid, such as β-alanine, or a D-amino acid, such as a D-valyl or D-alanyl group.

It is understood that the oligopeptide which is conjugated to the cytotoxic agent, whether through a direct covalent bond or through a chemical linker, does not need to be the oligopeptide that has the greatest recognition by free PSA and is most readily proteolytically cleaved by free PSA. Thus, the oligopeptide that is selected for incorporation in such conjugate will be chosen both for its selective, proteolytic cleavage by free PSA and for the cytotoxic activity of the cytotoxic agent-proteolytic residue conjugate (or, in what is felt to be an ideal situation, the unmodified cytotoxic agent) which results from such a cleavage.

Because the PSA conjugates useful in the instant compositions can be used for modifying a given biological response, cytotoxic agent component of the PSA conjugate is not to be construed as limited to classical chemical therapeutic agents. For example, the cytotoxic agent may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, b-interferon, nerve growth factor, platelet derived growth factor, tissue plasminogen activator- or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”) or other growth factors.

The preferred cytotoxic agents include, in general, alkylating agents, antiproliferative agents, tubulin binding agents and the like. Preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, doxorubicin, carminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. Other useful cytotoxic agents include estramustine, cisplatin and cyclophosphamide. One skilled in the art may make chemical modifications to the desired cytotoxic agent in order to make reactions of that compound more convenient for purposes of preparing PSA conjugates of the invention.

Preferably the cytotoxic agent component of the PSA conjugate is selected from a member of a class of cytotoxic agents selected from the vinca alkaloid drugs and the anthracyclines.

A pharmaceutical composition which is useful for the treatments of the instant invention may comprise one or more inhibitors of prenyl-protein transferase, one or more PSA conjugates, or a combination thereof, preferably, in combination with pharmaceutically acceptable carriers, excipients or diluents, according to standard pharmaceutical practice. The composition may be administered to mammals, preferably humans. The composition can be administered orally or parenterally, including the intravenous, intramuscular, intraperitoneal, subcutaneous, rectal and topical routes of administration.

The pharmaceutical compositions containing the active ingredients may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinylpyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethylcellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions useful in the instant methods of treatment may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The instant compositions may also be administered in the form of a suppositories for rectal administration of the drug. These compositions can be prepared by mixing the instant composition with a suitable non-irritating excipient which is solid at ordinary temperatures but liquid at the rectal temperature and will therefore melt in the rectum to release the composition. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the combination of inhibitor(s) of prenyl-protein transferase and PSA conjugate(s) are employed. (For purposes of this application, topical application shall include mouth washes and gargles.)

The compositions useful in the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

The composition of a prenyl-protein transferase inhibitor(s), a PSA conjugate(s), or a combination thereof useful in the instant methods of treatment may also be co-administered with other well known therapeutic agents that are selected for their particular usefulness against the condition that is being treated.

The instant method of treatment may also be combined with surgical treatment (such as surgical removal of tumor and/or prostatic tissue) where appropriate.

If formulated as a fixed dose, the compositions useful in the instant invention employ the prenyl-protein transferase inhibitor(s) and the PSA conjugate(s) within within the dosage ranges described below.

When compositions according to this invention are administered into a human subject, the daily dosage will normally be determined by the prescribing physician with the dosage generally varying according to the age, weight, and response of the individual patient, as well as the severity of the patient's symptoms.

In one exemplary application, a suitable amount of an inhibitor of prenyl-protein transferase and a suitable amount of a PSA conjugate are administered to a mammal undergoing treatment for prostate cancer. Administration occurs in an amount of prenyl-protein transferase inhibitor of between about 2 mg/m² of body surface area to about 2 g/m² of body surface area per day, preferably between about 12 mg/m² of body surface area to about 1200 mg/m² of body surface area per day, if the prenyl-protein transferase inhibitor is administered continuously over a 7 day period. A particular daily therapeutic dosage that comprises the instant composition includes from about 10 mg to about 3000 mg of an inhibitor of prenyl-protein transferase. Preferably, the daily dosage comprises from about 20 mg to about 2000 mg of an inhibitor of prenyl-protein transferase. A higher dosage of the prenyl-protein transferase inhibitor may be administered if the inhibitor is administered in a single dose once a week. Administration of the PSA conjugate occurs in an amount between about 10 mg/m² of body surface area to about 5 g/m² of body surface area per day, preferably between about 50 mg/m² of body surface area to about 3 g/m² of body surface area per day.

The instant method of treatment may also be combined with administration of a compound which inhibits HMG-CoA reductase in the methods of treatment of the instant invention. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938; 4,294,926; 4,319,039), simvastatin (ZOCOR®; see U.S. Pat. Nos. 4,444,784; 4,820,850; 4,916,239), pravastatin (PRAVACHOL®; see U.S. Pat. Nos. 4,346,227; 4,537,859; 4,410,629; 5,030,447 and 5,180,589), fluvastatin (LESCOL®; see U.S. Pat. Nos. 5,354,772; 4,911,165; 4,929,437; 5,189,164; 5,118,853; 5,290,946; 5,356,896), atorvastatin (LIPITOR®; see U.S. Pat. Nos. 5,273,995; 4,681,893; 5,489,691; 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (Feb. 5, 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

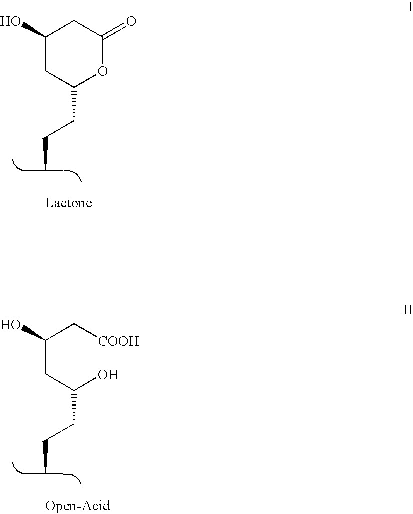

In HMG-CoA reductase inhibitor's where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

The instant method of treatment may be combined with administration of a compound which inhibits a fibroblast growth factor (FGF) receptor function in the methods of treatment of the instant invention. The instant method of treatment may be combined with administration of a compound which inhibits a urokinase in the methods of treatment of the instant invention.

The instant method of treatment may be combined with administration of a compound which inhibits angiogenesis, and thereby inhibit the growth and invasiveness of tumorous cells expressing enzymatically active PSA, in the methods of treatment of the instant invention. Such inhibitors of angiogenesis include, but are not limited to angiostatin and endostatin.

The instant method of treatment may be combined with administration of a compound which inhibits a matrix metalloproteinase in the methods of treatment of the instant invention. Compounds which have inhibitory activity for a matrix metalloproteinase can be readily identified by using assays well-known in the art. For example, see the assays described or cited in PCT Pat. Publ. WO 98/34915 in particular on pp. 24-26.

Prenyl-protein transferase inhibitor compounds that are useful in the methods of the instant invention and are identified by the properties described hereinabove include:

(a) a compound represented by formula (I-a) through (I-c):

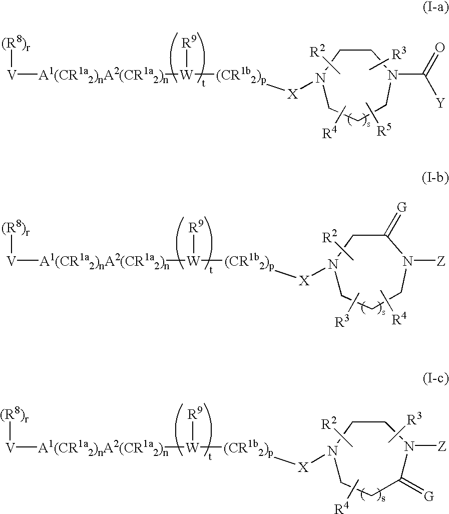

wherein with respect to formula (I-a):

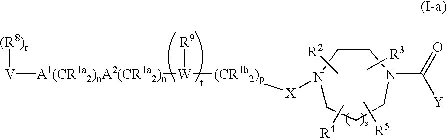

or a pharmaceutically acceptable salt thereof,

R^{1a} and R^{1b} are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰—,

c) C₁-C₆ alkyl unsubstituted or substituted by aryl, heterocyclyl, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, CN, (R¹⁰)₂N—C(NR¹⁰)—, R¹⁰C(O)—, R¹⁰OC(O)—, N₃, —N(R¹⁰)₂, or R¹¹OC(O)—NR¹⁰—;

R² and R³ are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle,

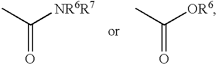

wherein the substituted group is substituted with one or more of:

1) aryl or heterocycle, unsubstituted or substituted with:

a) C₁₋₄ alkyl,

b) (CH₂)ₚOR⁶,

c) (CH₂)ₚNR⁶R⁷,

d) halogen,

2) C₃₋₆ cycloalkyl,

3) OR⁶,

4) SR⁶, S(O)R⁶, SO₂R⁶,

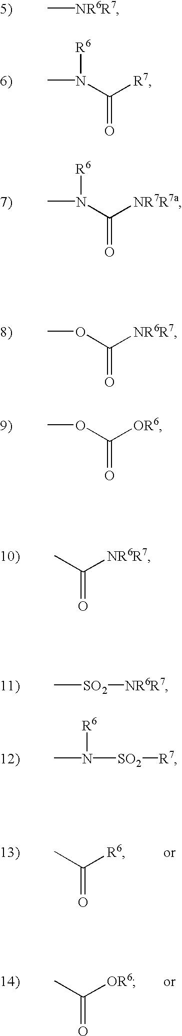

R² and R³ are attached to the same C atom and are combined to form —(CH₂)_{u—} wherein one of the carbon atoms is optionally replaced by a moiety selected from: O, S(O)ₘ, —NC(O)—, and —N(COR¹⁰)—;

R⁴ and R⁵ are independently selected from H and CH₃; and any two of R², R³, R⁴ and R⁵ are optionally attached to the same carbon atom; R⁶, R⁷ and R^{7a} are independently selected from: H; C₁₋₄ alkyl, C₃₋₆ cycloalkyl, heterocycle, aryl, aroyl, heteroaroyl, arylsulfonyl, heteroarylsulfonyl, unsubstituted or substituted with:

a) C₁₋₄ alkoxy,

b) aryl or heterocycle,

c) halogen,

d) HO,

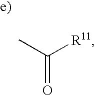

f) —SO₂R¹¹, or

g) N(R¹⁰)₂; or

R⁶ and R⁷ may be joined in a ring;

R⁷ and R^{7a} may be joined in a ring;

R⁸ is independently selected from:

a) hydrogen,

b) aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, CN, NO₂, R¹⁰₂N—C(NR¹⁰)—, R¹⁰C(O)—, R¹⁰OC(O)—, N₃, —N(R¹⁰)₂, or R¹¹OC(O)NR¹⁰—, and

c) C₁-C₆ alkyl unsubstituted or substituted by aryl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, CN, H₂N—C(NH)—, R¹⁰C(O)—, R¹⁰OC(O)—, N₃, —N(R¹⁰)₂, or R¹⁰OC(O)NH—;

R⁹ is selected from:

a) hydrogen,

b) C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, CN, NO₂, (R¹⁰)₂N—C—(NR¹⁰)—, R¹⁰C(O)—, R¹⁰OC(O)—, N₃, —N(R¹⁰)₂, or R¹¹OC(O)NR¹⁰—, and

c) C₁-C₆ alkyl unsubstituted or substituted by perfluoroalkyl, F, Cl, Br, R¹⁰O—, R¹¹S(O)ₘ—, R¹⁰C(O)NR¹⁰—, CN, (R¹⁰)₂N—C(NR¹⁰)—, R¹⁰C(O)—, R¹⁰OC(O)—, N₃, —N(R¹⁰)₂, or R¹¹OC(O)NR¹⁰—;

R¹⁰ is independently selected from hydrogen, C₁-C₆ alkyl, benzyl and aryl;

R¹¹ is independently selected from C₁-C₆ alkyl and aryl;

A¹ and A² are independently selected from: a bond, —CH═CH—, —C≡C—, —C(O)—, —C(O)NR¹⁰—, —NR¹⁰C(O)—, O, —N(R¹⁰)—, —S(O)₂N(R¹⁰)—, —N(R¹⁰)S(O)₂—, or S(O)ₘ;

V is selected from:

a) hydrogen,

b) heterocycle,

c) aryl,

d) C₁-C₂₀ alkyl wherein from 0 to 4 carbon atoms are replaced with a a heteroatom selected from O, S, and N, and

e) C₂-C₂₀ alkenyl,

provided that V is not hydrogen if A¹ is S(O)ₘ and V is not hydrogen if A¹ is a bond, n is 0 and A² is S(O)ₘ;

W is a heterocycle;

X is —CH₂—, —C(═O)—, or —S(═O)ₘ—;

Y is aryl, heterocycle, unsubstituted or substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁶, or

g) —C(O)NR⁶R⁷,

2) aryl or heterocycle,

3) halogen,

4) OR⁶,

5) NR⁶R⁷,

6) CN,

7) NO₂,

8) CF₃;

9) —S(O)ₘR⁶,

10) —C(O)NR⁶R⁷, or

11) C₃-C₆ cycloalkyl;

m is 0, 1 or 2;

n is 0, 1, 2, 3 or 4;

p is 0, 1, 2, 3 or 4;

r is 0 to 5, provided that r is 0 when V is hydrogen;

s is 0 or 1;

t is 0 or 1; and

u is 4 or 5;

with respect to formula (I-b):

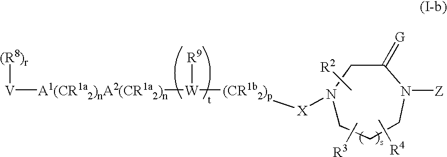

or a pharmaceutically acceptable salt thereof,

R^{1a}, R^{1b}, R¹⁰, R¹¹, m, R², R³, R⁶, R⁷, p, R^{7a}, u, R⁸, A¹, A², V, W, X, n, p, r, s, t and u are as defined above with respect to formula (I-a);

R⁴ is selected from H and CH₁₃;

and any two of R², R³ and R⁴ are optionally attached to the same carbon atom;

R⁹ is selected from:

a) hydrogen,

b) alkenyl, alkynyl, perfluoroalkyl, F, Cl, Br, R¹⁰O—, R¹¹S(O)ₘ—, R¹⁰C(O)NR¹⁰—, CN, NO₂, (R¹⁰)₂N—C—(NR¹⁰)—, R¹⁰C(O)—, R¹⁰OC(O)—, N₃, —N(R¹⁰)₂, or R¹¹OC(O)NR¹⁰—, and

c) C₁-C₆ alkyl unsubstituted or substituted by perfluoroalkyl, F, Cl, Br, R¹⁰O—, R¹¹S(O)ₘ—, R¹⁰C(O)NR¹⁰—, CN, (R¹⁰)₂N—C(NR¹⁰)—, R¹⁰C(O)—, R¹⁰OC(O)—, N₃, —N(R¹⁰)₂, or R¹¹OC(O)NR¹⁰—;

G is H₂ or O;

Z is aryl, heteroaryl, arylmethyl, heteroarylmethyl, arylsulfonyl, heteroarylsulfonyl, unsubstituted or substituted with one or more of the following:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁶, or

g) —C(O)NR⁶R⁷,

2) aryl or heterocycle,

3) halogen,

4) OR⁶,

5) NR⁶R⁷,

6) CN,

7) NO₂,

8) CF₃;

9) —S(O)ₘR⁶,

10) —C(O)NR⁶R⁷, or

11) C₃-C₆ cycloalkyl;

with respect to formula (I-c):

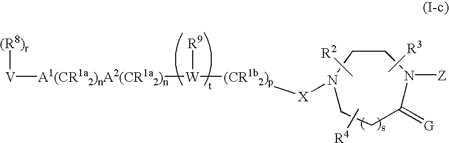

or a pharmaceutically acceptable salt thereof,

R^{1a}, R^{1b}, R¹⁰, R¹¹, m, R², R³, R⁶, R⁷, p, u, R^{7a}, R⁸, A¹, A², V, W, X, n, r and t are as defined above with respect to formula (I-a);

R⁴ is selected from H and CH₃; and any two of R², R³ and R⁴ are optionally attached to the same carbon atom;

G is O;

Z is aryl, heteroaryl, arylmethyl, heteroarylmethyl, arylsulfonyl, heteroarylsulfonyl, unsubstituted or substituted with one or more of the following:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁶, or

g) —C(O)NR⁶R⁷,

2) aryl or heterocycle,

3) halogen,

4) OR⁶,

5) NR⁶R⁷,

6) CN,

7) NO₂,

8) CF₃;

9) —S(O)ₘR⁶,

10) —C(O)NR⁶R⁷, or

11) C₃-C₆ cycloalkyl; and

s is 1;

(b) a compound represented by formula (II):

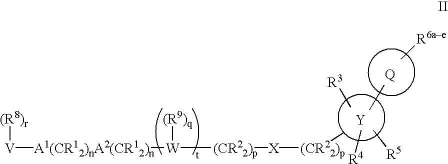

wherein:

Q is a 4, 5, 6 or 7 membered heterocyclic ring which comprises a nitrogen atom through which Q is attached to Y and 0-2 additional heteroatoms selected from N, S and O, and which also comprises a carbonyl, thiocarbonyl, —C(═NR¹³)— or sulfonyl moiety adjacent to the nitrogen atom attached to Y;

Y is a 5, 6 or 7 membered carbocyclic ring wherein from 0 to 3 carbon atoms are replaced by a heteroatom selected from N, S and O, and wherein Y is attached to Q through a carbon atom;

R¹ and R² are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, R¹¹C(O)O—, (R¹⁰)₂NC(O)—, R¹⁰₂N—C(NR¹⁰)—, CN, NO₂, R¹⁰C(O)—, N₃, —N(R¹⁰)₂, or R¹¹OC(O)NR¹⁰—,

c) unsubstituted or substituted C₁-C₆ alkyl wherein the substituent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂, and R¹⁰C(O)—NR¹⁰—;

R³, R⁴ and R⁵ are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R¹²O—, R¹¹S(O)ₘ—, R¹⁰C(O)NR¹⁰—, (R¹⁰)₂NC(O)—, R¹¹C(O)O—, R¹⁰₂N—C(NR¹⁰)—, CN, NO₂, R¹⁰C(O)—, N₃, —N(R¹⁰)₂, or R¹¹OC(O)NR¹⁰—,

c) unsubstituted C₁-C₆ alkyl,

d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R¹²O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂, and R¹¹OC(O)—NR¹⁰—;

R^{6a}, R^{6b}, R^{6c}, R^{6d} and R^{6e} are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, halogen, C₁-C₆ perfluoroalkyl, R¹²O—, R¹¹S(O)ₘ—, R¹⁰C(O)NR¹⁰—, (R¹⁰)₂NC(O)—, R¹¹S(O)₂NR¹⁰—, (R¹⁰)₂NS(O)₂—, R¹¹C(O)O—, R¹⁰₂N—C(NR¹⁰)—, CN, NO₂, R¹⁰C(O)—, N₃, —N(R¹⁰)₂, or R¹¹OC(O)NR¹⁰—,

c) unsubstituted C₁-C₆ alkyl,

d) substituted C₁-C₆ alkyl wherein the substituent on the substituted C₁-C₆ alkyl is selected from unsubstituted or substituted aryl, unsubstituted or substituted heterocyclic, C₃-C₁₀ cycloalkyl, C₂-C₆ alkenyl, C₂-C₆ alkynyl, R¹²O—, R¹¹S(O)ₘ—, R¹⁰C(O)NR¹⁰—, (R¹⁰)₂NC(O)—, R¹¹S(O)₂NR¹⁰—, (R¹⁰)₂NS(O)₂—, R¹⁰₂N—C(NR¹⁰)—, CN, R¹⁰C(O)—, N₃, —N(R¹⁰)₂, and R¹¹OC(O)—NR¹⁰—; or

any two of R^{6a}, R^{6b}, R^{6c}, R^{6d} and R^{6e} on adjacent carbon atoms are combined to form a diradical selected from —CH═CH—CH═CH—, —CH═CH—CH₂—, —(CH₂)₄— and —(CH₂)₃—;

R⁷ is selected from: H; C₁₋₄ alkyl, C₃₋₆ cycloalkyl, heterocycle, aryl, aroyl, heteroaroyl, arylsulfonyl, heteroarylsulfonyl, unsubstituted or substituted with:

a) C₁₋₄ alkoxy,

b) aryl or heterocycle,

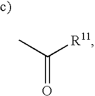

d) —SO₂R¹¹

e) N(R¹⁰)₂ or

f) C₁₋₄ perfluoroalkyl;

R⁸ is independently selected from:

a) hydrogen,

b) aryl, substituted aryl, heterocycle, substituted heterocycle, C₃-C₁₀ cycloalkyl, C₂-C₆ alkenyl, C₂-C₆ alkynyl, perfluoroalkyl, F, Cl, Br, R¹⁰O—, R¹S(O)ₘ—, R¹⁰C(O)NR¹⁰—, (R¹⁰)₂NC(O)—, R¹¹S(O)₂NR¹⁰—, (R¹⁰)₂NS(O)₂—, R¹⁰₂N—C(NR¹⁰)—, CN, NO₂, R¹⁰C(O)—, N₃, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, C₂-C₆ alkenyl, C₂-C₆ alkynyl, perfluoroalkyl, F, Cl, Br, R¹⁰O—, R¹S(O)ₘ—, R¹⁰C(O)NR¹⁰—, (R¹⁰)₂NC(O)—, R¹¹S(O)₂NR¹⁰—, (R¹⁰)₂NS(O)₂—, R¹⁰₂N—C(NR¹⁰)—, CN, R¹⁰C(O)—, N₃, —N(R¹⁰)₂, or R¹⁰OC(O)NH—;

R⁹ is independently selected from:

a) hydrogen,

b) alkenyl, alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, N₃, —N(R¹⁰)₂, or R¹¹OC(O)NR¹⁰—, and

c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂, or R¹⁰C(O)NR¹⁰—;

R¹⁰ is independently selected from hydrogen, C₁-C₆ alkyl, benzyl, 2,2,2-trifluoroethyl and aryl;

R¹¹ is independently selected from C₁-C₆ alkyl and aryl;

R¹² is independently selected from hydrogen, C₁-C₆ alkyl, C₁-C₆ aralkyl, C₁-C₆ substituted aralkyl, C₁-C₆ heteroaralkyl, C₁-C₆ substituted heteroaralkyl, aryl, substituted aryl, heteroaryl, substituted heteraryl, C₁-C₆ perfluoroalkyl, 2-aminoethyl and 2,2,2-trifluoroethyl;

R¹³ is selected from hydrogen, C₁-C₆ alkyl, cyano, C₁-C₆ alkylsulfonyl and C₁-C₆ acyl;

A¹ and A² are independently selected from: a bond, —CH═CH—, —C≡C—, —C(O)—, —C(O)NR¹⁰—, —NR¹⁰C(O)—, O, —N(R¹⁰)—, —S(O)₂N(R¹⁰)—, —N(R¹⁰)S(O)₂—, or S(O)ₘ;

V is selected from:

a) hydrogen,

b) heterocycle,

c) aryl,

d) C₁-C₂₀ alkyl wherein from 0 to 4 carbon atoms are replaced with a heteroatom selected from O, S, and N, and

e) C₂-C₂₀ alkenyl,

provided that V is not hydrogen if A¹ is S(O)ₘ and V is not hydrogen if A¹ is a bond, n is 0 and A² is S(O)ₘ;

W is a heterocycle;

X is a bond, —CH═CH—, O, —C(═O)—, —C(O)NR⁷—, —NR⁷C(O)—, —C(O)O—, —OC(O)—, —C(O)NR⁷C(O)—, —NR⁷—, —S(O)₂N(R¹⁰)—, —N(R¹⁰)S(O)₂— or —S(═O)ₘ—;

m is 0, 1 or 2;

n is independently 0, 1, 2, 3 or 4;

p is independently 0, 1, 2, 3 or 4;

q is 0, 1, 2 or 3;

r is 0 to 5, provided that r is 0 when V is hydrogen; and

t is 0 or 1;

(c) a compound represented by formula (III):

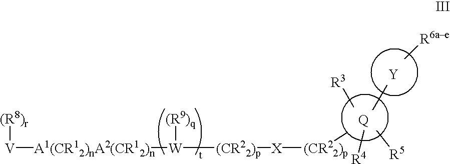

wherein:

R¹, R², R³, R⁴, R⁵, R^{6a-e}, R⁷, R⁸, R⁹, R¹⁰, R¹¹, R¹², R¹³, A¹, A², V, W, m, n, p, q, r and t are as previously defined with respect to formula (II);

Q is a 4, 5, 6 or 7 membered heterocyclic ring which comprises a nitrogen atom through which Q is attached to Y and 0-2 additional heteroatoms selected from N, S and O, and which also comprises a carbonyl, thiocarbonyl, —C(═NR¹³)— or sulfonyl moiety adjacent to the nitrogen atom attached to Y,

provided that Q is not

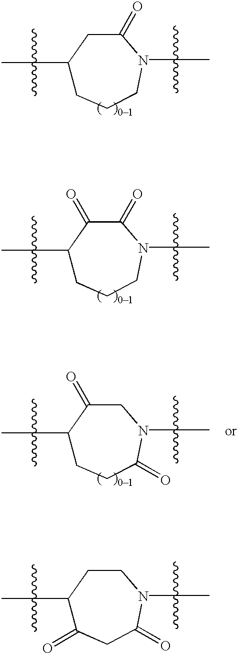

Y is a 5, 6 or 7 membered carbocyclic ring wherein from 0 to 3 carbon atoms are replaced by a heteroatom selected from N, S and O, and wherein Y is attached to Q through a carbon atom;

(d) a compound represented by formula (IV):

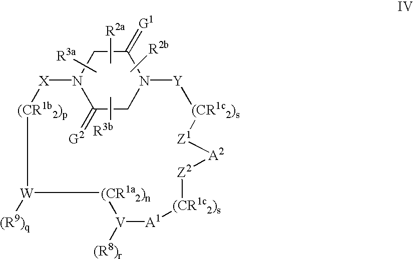

wherein:

R^{1a}, R^{1b}, R^{1c} and R^{1d} are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰—,

c) unsubstituted or substituted C₁-C₆ alkyl wherein the substitutent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, (R¹⁰)₂N—C(NR¹⁰)—, R¹⁰C(O)—, R¹⁰OC(O)—, N₃, —N(R¹⁰)₂, and R¹¹OC(O)—NR¹⁰—;

R^{2a}, R^{2b}, R^{3a} and R^{3b} are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle,

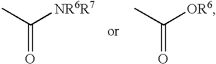

wherein the substituted group is substituted with one or more of:

1) aryl or heterocycle, unsubstituted or substituted with:

a) C₁₋₄ alkyl,

b) (CH₂)ₚOR⁶,

c) (CH₂)ₚNR⁶R⁷,

d) halogen,

e) CN,

2) C₃₋₆ cycloalkyl,

3) OR⁶,

4) SR⁴, S(O)R⁴, SO₂R⁴,

5) —NR⁶R⁷,

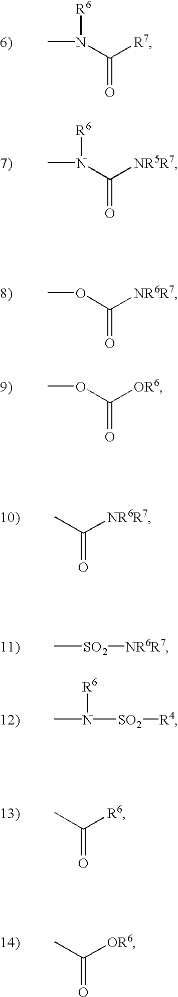

15) N₃, or

16) F; or

R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;

and R¹² and R³ are optionally attached to the same carbon atom;

R⁴ is selected from: C₁₋₄ alkyl, C₃₋₆ cycloalkyl, heterocycle, aryl, unsubstituted or substituted with:

a) C₁₋₄ alkoxy,

b) aryl or heterocycle,

c) halogen,

d) HO,

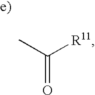

f) —SO₂R¹¹, or

g) N(R¹⁰)₂;

R⁵, R⁶ and R⁷ are independently selected from: H; C₁₋₄ alkyl, C₃₋₆ cycloalkyl, heterocycle, aryl, aroyl, heteroaroyl, arylsulfonyl, heteroarylsulfonyl, unsubstituted or substituted with:

a) C₁₋₄ alkoxy,

b) aryl or heterocycle,

c) halogen,

d) HO,

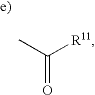

f) —SO₂R¹¹, or

g) N(R¹⁰)₂; or

R⁶ and R⁷ may be joined in a ring; and independently, R⁵ and R⁷ may be joined in a ring;

R⁸ is independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, perfluoroalkyl, F, Cl, Br, R¹⁰O—, R¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and

c) C₁-C₆ alkyl unsubstituted or substituted by unsubstituted or substituted aryl, unsubstituted or substituted heterocycle, C₃-C₁₀ cycloalkyl, C₂-C₆ alkenyl, C₂-C₆ alkynyl, perfluoroalkyl, F, Cl, Br, R¹⁰O—, R¹S(O)ₘ—, R¹⁰C(O)NH—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹⁰OC(O)NH—;

R⁹ is selected from:

a) hydrogen,

b) C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and

c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹¹OC(O)NR¹⁰—;

R¹⁰ is independently selected from hydrogen, C₁-C₆ alkyl, benzyl, unsubstituted or substituted aryl and unsubstituted or substituted heterocycle;

R¹¹ is independently selected from C₁-C₆ alkyl unsubstituted or substituted aryl and unsubstituted or substituted heterocycle;

A¹ is selected from: a bond, —C(O)—, —C(O)NR¹⁰—, —NR¹⁰C(O)—, O, —N(R¹⁰)—, —S(O)₂N(R¹⁰)—, —N(R¹⁰)S(O)₂—, and S(O)ₘ;

A² is selected from: a bond, —C(O)—, —C(O)NR¹⁰—, —NR¹⁰C(O)—, O, —N(R¹⁰)—, —S(O)₂N(R¹⁰)—, —N(R¹⁰)S(O)₂—, S(O)ₘ and —C(R^{1d})₂—;

G¹ G² and G³ are independently selected from: H₂ and O;

W is heterocycle;

V is selected from:

a) heterocycle, and

b) aryl;

X and Y are independently selected from: a bond, —C(═O)— or —S(═O)ₘ—;

Z¹ is selected from: unsubstituted or substituted aryl and unsubstituted or substituted heterocycle, wherein the substituted aryl or substituted heterocycle is substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁴, or

g) —C(O)NR⁶R⁷,

2) aryl or heterocycle,

3) halogen,

4) OR⁶,

5) NR⁶R⁷,

6) CN,

7) NO₂,

8) CF₃,

9) —S(O)ₘR⁴,

10) —C(O)NR⁶R⁷, or

11) C₃-C₆ cycloalkyl;

Z² is selected from: a bond, unsubstituted or substituted aryl and unsubstituted or substituted heterocycle, wherein the substituted aryl or substituted heterocycle is substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁴, or

g) —C(O)NR⁶R⁷,

2) aryl or heterocycle,

3) halogen,

4) OR⁶,

5) NR⁶R⁷,

6) CN,

7) NO₂,

8) CF₃,

9) —S(O)ₘR⁴,

10) —C(O)NR⁶R⁷, or

11) C₃-C₆ cycloalkyl;

m is 0, 1 or 2;

n is 0, 1, 2, 3 or 4;

p is 0, 1, 2, 3 or 4;

q is 1 or 2;

r is 0 to 5;

s is independently 0, 1, 2 or 3; and

u is 4 or 5;

(e) a compound represented by formula (V):

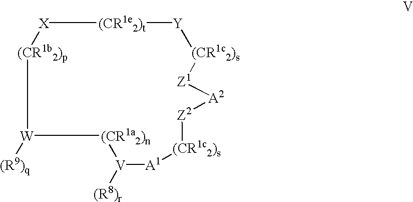

wherein:

R^{1a}, R^{1b}, R^{1c}, R^{1d} and R^{1e} are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰—,

c) unsubstituted or substituted C₁-C₆ alkyl wherein the substitutent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, (R¹⁰)₂N—C(NR¹⁰)—, R¹⁰C(O)—, R¹⁰OC(O)—, N₃, —N(R¹⁰)₂, and R¹¹OC(O)—NR¹⁰—;

R^{2a}, R^{2b}, R^{3a} and R^{3b} are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle,

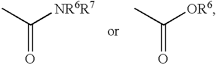

wherein the substituted group is substituted with one or more of:

1) aryl or heterocycle, unsubstituted or substituted with:

a) C₁₋₄ alkyl,

b) (CH₂)ₚOR⁶,

c) (CH₂)ₚNR⁶R⁷,

d) halogen,

e) CN,

2) C₃₋₆ cycloalkyl,

3) OR⁶,

4) SR⁴, S(O)R⁴, SO₂R⁴,

5) —NR⁶R⁷,

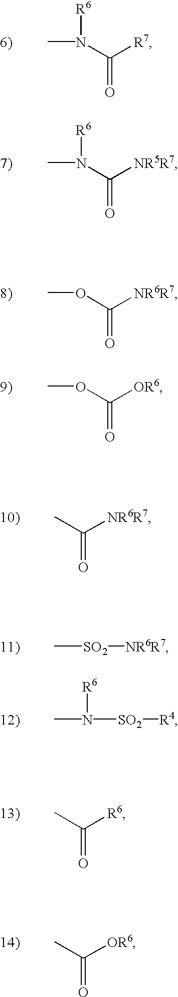

15) N₃, or

16) F; or

R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;

and R² and R³ are optionally attached to the same carbon atom;

R⁴ is selected from: C₁₋₄ alkyl, C₃₋₆ cycloalkyl, heterocycle, aryl, unsubstituted or substituted with:

a) C₁₋₄ alkoxy,

b) aryl or heterocycle,

c) halogen,

d) HO,

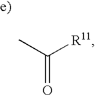

f) —SO₂R¹¹, or

g) N(R¹⁰)₂;

R⁵, R⁶ and R⁷ are independently selected from: H; C₁₋₄ alkyl, C₃₋₆ cycloalkyl, heterocycle, aryl, aroyl,-heteroaroyl, arylsulfonyl, heteroarylsulfonyl, unsubstituted or substituted with:

a) C₁₋₄ alkoxy,

b) aryl or heterocycle,

c) halogen,

d) HO,

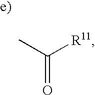

f) —SO₂R¹¹, or

g) N(R¹⁰)₂; or

R⁶ and R⁷ may be joined in a ring; and independently, R⁵ and R⁷ may be joined in a ring;

R⁸ is independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹⁰—, and

c) C₁-C₆ alkyl unsubstituted or substituted by unsubstituted or substituted aryl, unsubstituted or substituted heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹⁰OC(O)NH—;

R⁹ is selected from:

a) hydrogen,

b) C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹¹OC(O)NR¹—, and

c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹¹OC(O)NR¹⁰—;

R¹⁰ is independently selected from hydrogen, C₁-C₆ alkyl, benzyl, unsubstituted or substituted aryl and unsubstituted or substituted heterocycle;

R¹¹ is independently selected from C₁-C₆ alkyl unsubstituted or substituted aryl and unsubstituted or substituted heterocycle;

A¹ is selected from: a bond, —C(O)—, —C(O)NR¹⁰—, —NR¹⁰C(O)—, O, —N(R¹⁰)—, —S(O)₂N(R¹⁰)—, —N(R¹⁰)S(O)₂—, and S(O)ₘ;

A² is selected from: a bond, —C(O)—, —C(O)NR¹⁰—, —NR¹⁰C(O)—, O, —N(R¹⁰)—, —S(O)₂N(R¹⁰)—, —N(R¹⁰)S(O)₂—, S(O)ₘ and —C(R^{1d})₂—;

W is heteroaryl;

V is selected from:

a) heteroaryl, and

b) aryl;

X and Y are independently selected from: —C(O)—, —C(O)NR¹⁰—, —NR¹⁰C(O)—, —NR¹⁰C(O)—O—, —O—C(O)NR¹⁰—, —NR¹⁰C(O)NR¹⁰—, —C(O)NR¹⁰C(O)—, O, —N(R¹⁰)—, —S(O)₂N(R¹⁰)—, —N(R¹⁰)S(O)₂— and S(O)ₘ;

Z¹ is selected from: unsubstituted or substituted aryl and unsubstituted or substituted heteroaryl, wherein the substituted aryl or substituted heteroaryl is substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁴, or

g) —C(O)NR⁶R⁷,

2) aryl or heterocycle,

3) halogen,

4) OR⁶,

5) NR⁶R⁷,

6) CN,

7) NO₂,

8) CF₃,

9) —S(O)ₘR⁴,

10) —C(O)NR⁶R⁷, or

11) C₃-C₆ cycloalkyl;

Z² is selected from: a bond, unsubstituted or substituted aryl and unsubstituted or substituted heteroaryl, wherein the substituted aryl or substituted heteroaryl is substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁴, or

g) —C(O)NR⁶R⁷,

2) aryl or heterocycle,

3) halogen,

4) OR⁶,

5) NR⁶R⁷,

6) CN,

7) NO₂,

8) CF₃,

9) —S(O)ₘR⁴,

10) —C(O)NR⁶R⁷, or

11) C₃-C₆ cycloalkyl;

m is 0, 1 or 2;

n is 0, 1, 2, 3 or 4;

p is 0, 1, 2, 3 or 4;

q is 1 or 2;

r is 0 to 5;

s is independently 0, 1, 2 or 3;

t is 1, 2, 3 or 4; and

u is 4 or 5;

(f) a compound represented by formula (VI):

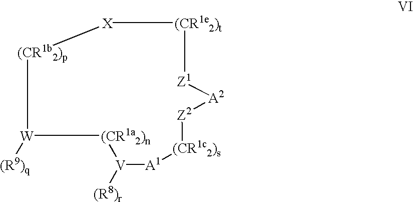

wherein:

R^{1a}, R^{1b}, R^{1c}, R^{1d} and R^{1e} are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, NO₂, (R¹⁰)₂N—C(NR¹⁰)—, R¹⁰C(O)—, R¹⁰OC(O)—, N₃, —N(R¹⁰)₂, or R¹¹OC(O)NR¹⁰—,

c) unsubstituted or substituted C₁-C₆ alkyl wherein the substitutent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂N—C(O)—, CN, (R¹⁰)₂N—C(NR¹⁰)—, R¹⁰C(O)—, R¹⁰OC(O)—, N₃, —N(R¹⁰)₂, and R¹¹OC(O)—NR¹⁰-;

R^{2a}, R^{2b}, R^{3a} and R^{3b} are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle,

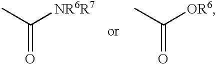

wherein the substituted group is substituted with one or more of:

1) aryl or heterocycle, unsubstituted or substituted with:

a) C₁₋₄ alkyl,

b) (CH₂)ₚOR⁶,

c) (CH₂)ₚNR⁶R⁷,

d) halogen,

e) CN,

2) C₃₋₆ cycloalkyl,

3) OR⁶,

4) SR⁴, S(O)R⁴, SO₂R⁴,

5) —NR⁶R⁷,

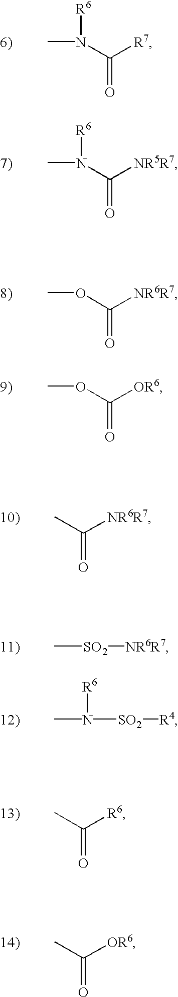

15) N₃, or

16) F; or

R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;

and R² and R³ are optionally attached to the same carbon atom;

R⁴ is selected from: C₁₋₄ alkyl, C₃₋₆ cycloalkyl, heterocycle, aryl, unsubstituted or substituted with:

a) C₁₋₄ alkoxy,

b) aryl or heterocycle,

c) halogen,

d) HO,

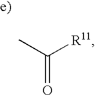

f) —SO₂R¹¹, or

g) N(R¹⁰)₂;

R⁵, R⁶ and R⁷ are independently selected from: H; C₁₋₄ alkyl, C₃₋₆ cycloalkyl, heterocycle, aryl, aroyl,-heteroaroyl, arylsulfonyl, heteroarylsulfonyl, unsubstituted or substituted with:

a) C₁₋₄ alkoxy,

b) aryl or heterocycle,

c) halogen,

d) HO,

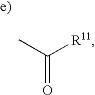

f) —SO₂R¹¹, or

g) N(R¹⁰)₂; or

R⁶ and R⁷ may be joined in a ring; and independently, R⁵ and R⁷ may be joined in a ring;

R⁸ is independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, perfluoroalkyl, F, Cl, Br, R¹⁰O—, R¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹⁰C(O)NR¹⁰—, and

c) C₁-C₆ alkyl unsubstituted or substituted by unsubstituted or substituted aryl, unsubstituted or substituted heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹⁰OC(O)NH—;

R⁹ is selected from:

a) hydrogen,

b) C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, R¹⁰OC(O)—, N₃, —N(R¹⁰)₂, or R¹⁰C(O)NR¹⁰—, and

c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, R¹⁰OC(O)—, N₃, —N(R¹⁰)₂, or R¹¹OC(O)NR¹⁰—;

R¹⁰ is independently selected from hydrogen, C₁-C₆ alkyl, benzyl, unsubstituted or substituted aryl and unsubstituted or substituted heterocycle;

R¹¹ is independently selected from C₁-C₆ alkyl unsubstituted or substituted aryl and unsubstituted or substituted heterocycle;

A¹ is selected from: a bond, —C(O)—, —C(O)NR¹⁰—, —NR¹⁰C(O)—, O, —N(R¹⁰)—, —S(O)₂N(R¹⁰)—, —N(R¹⁰)S(O)₂—, and S(O)ₘ;

A² is selected from: a bond, —C(O)—, —C(O)NR¹⁰—, —NR¹⁰C(O)—, O, —N(R¹⁰)—, —S(O)₂N(R¹⁰)—, —N(R¹⁰)S(O)₂—, S(O)ₘ and —C(R^{1d})₂—;

W is heteroaryl;

V is selected from:

a) heteroaryl, and

b) aryl;

X is selected from: —C(O)—, —C(O)NR¹⁰—, —NR¹⁰C(O)—, —NR¹⁰C(O)—O—, —O—C(O)NR¹⁰—, —NR¹⁰C(O)NR¹⁰—, —C(O)NR¹⁰C(O)—, O, —N(R¹⁰)—, —S(O)₂N(R¹⁰)—, —N(R¹⁰)S(O)₂— and S(O)ₘ;

Z¹ is selected from: unsubstituted or substituted aryl and unsubstituted or substituted heteroaryl, wherein the substituted aryl or substituted heteroaryl is substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁴, or

g) —C(O)NR⁶R⁷,

2) aryl or heterocycle,

3) halogen,

4) OR⁶,

5) NR⁶R⁷,

6) CN,

7) NO₂,

8) CF₃,

9) —S(O)ₘR⁴,

10) —C(O)NR⁶R⁷, or

11) C₃-C₆ cycloalkyl;

Z² is selected from: a bond, unsubstituted or substituted aryl and unsubstituted or substituted heteroaryl, wherein the substituted aryl or substituted heteroaryl is substituted with one or more of:

1) C₁₋₄ alkyl, unsubstituted or substituted with:

a) C₁₋₄ alkoxy,

b) NR⁶R⁷,

c) C₃₋₆ cycloalkyl,

d) aryl or heterocycle,

e) HO,

f) —S(O)ₘR⁴, or

g) —C(O)NR⁶R⁷,

2) aryl or heterocycle,

3) halogen,

4) OR⁶,

5) NR⁶R⁷,

6) CN,

7) NO₂,

8) CF₃,

9) —S(O)ₘR⁴,

10) —C(O)NR⁶R⁷, or

11) C₃-C₆ cycloalkyl;

m is 0, 1 or 2;

n is 0, 1, 2, 3 or 4;

p is 0, 1, 2, 3 or 4;

q is 1 or 2;

r is 0 to 5;

s is independently 0, 1, 2 or 3;

t is 1, 2, 3 or 4; and

u is 4 or 5;

(g) a compound represented by formula (VII):

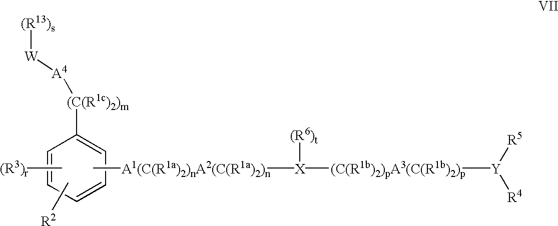

wherein:

R^{1a}, R^{1b} and R^{1c} are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, R⁸O—, R⁹S(O)_{q}—, CN, NO₂, R⁸C(O)—, R⁸OC(O)—, R⁸(C₁-C₆ alkyl)O—, N₃, N(R⁸)₂ or —OC(O)O-heteroaralkyl;

c) C₁-C₆ alkyl, unsubstituted or substituted by aryl, heterocyclic, C₃-C₁₀ cycloalkyl, C₂-C₆ alkenyl, C₂-C₆ alkynyl, R⁸O—, R⁹S(O)_{q}—, CN, R⁸C(O)—, R⁸OC(O)—, N₃, or R⁸C(O)O—;

R² is selected from:

a) hydrogen,

b) CN,

c) NO₂,

d) halogen,

e) aryl, unsubstituted or substituted,

f) heteroaryl, unsubstituted or substituted,

g) C₁-C₆ alkyl, unsubstituted or substituted,

h) N₃,

i) R⁹S(O)_{q},

j) R⁸HC═CH—,

k) R⁸C≡C—, and

l) OR⁸;

R³, R⁴ and R⁵ are independently selected from: H, CN, NO₂, halogen, unsubstituted or substituted C₁-C₆ alkyl, N₃, R⁹S(O)_{q}, HC≡C—, unsubstituted or substituted aryl, unsubstituted or substituted heterocycle, CF₃, CF₃O—, CF₃CH₂O—, C₃-C₁₀ cycloalkyl, OR⁸, N(R⁸)₂, —C(O)R⁸, —O(C₁-C₆ alkyl)OR⁸, —NHC(O)R⁸, aralkyl, heteroaralkyl, —(C₁-C₆ alkyl)OR⁸, —(C₁-C₆ alkyl)C(O)R⁸, —C═C—R⁸ CH═CH—R⁸ and

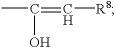

R⁶ is independently selected from:

a) hydrogen,

b) CN,

c) NO₂,

d) halogen,

e) aryl, unsubstituted or substituted,

f) heteroaryl, unsubstituted or substituted,

g) C₁-C₆ alkyl, unsubstituted or substituted,

h) R⁸O—,

i) N₃,

j) R⁹S(O)_{q}—,

k) —HC═CH₂,

l) —C≡CH,

m) CF₃,

n) R⁸O(C═O)—, and

o) R⁸(O═C)O—;

R⁸ is independently selected from hydrogen, unsubstituted or substituted C₁-C₆ alkyl, cycloalkyl, benzyl and unsubstituted or substituted aryl;

R⁹ is independently selected from H, unsubstituted or substituted C₁-C₆ alkyl, benzyl and unsubstituted or substituted aryl;

R¹³ is independently selected from H, unsubstituted or substituted C₁-C₆ alkyl, unsubstituted or substituted aryl, unsubstituted or substituted heterocycle, —(C₁-C₆ alkyl)OR⁸, —(C₁-C₆ alkyl)OC(O)(C₁-C₆ alkyl), —(C₁-C₆ alkyl)N(R⁸)₂, and —(C₁-C₆ alkyl)NHC(O)(C₁-C₆ alkyl)R⁸;

A¹, A² and A³ are independently selected from:

a) a bond,

b) —HC═CH—,

c) —C≡C—,

d) —O—,

e) —(C═O)—,

f) —O(C═O)—,

g) —(C═O)O—,

h) —NR⁸—,

i) —C(O)N(R⁸)—,

j) —N(R⁸)C(O)—,

k) —NHC(O)NH—,

l) —S(O)_{q}—,

m) —S(O)_{q}NH—, and

n) —NHS(O)_{q}—;

A⁴ is selected from a bond, C(O), C═CH₂, or spiro C₃-C₆ cycloalkyl;

W is selected from:

a) hydrogen,

b) heterocycle, and

c) aryl;

X is selected from:

a) aryl,

b) cycloalkyl,

c) heterocycle, and

d) a bond;

Y is selected from:

a) aryl, unsubstituted or substituted,

b) heterocycle, unsubstituted or substituted, and

c) cycloalkyl;

m is 0, 1, 2, 3 or 4;

n is 0, 1, 2, 3 or 4;

p is 0, 1, 2, 3 or 4;

q is 0, 1 or 2;

r is 0, 1, 2 or 3;

s is 0, 1, 2, 3 or 4; and;

t is 0, 1, 2 or 3;

provided that

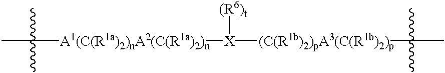

is not a bond;

(h) a compound represented by formula (VIII):

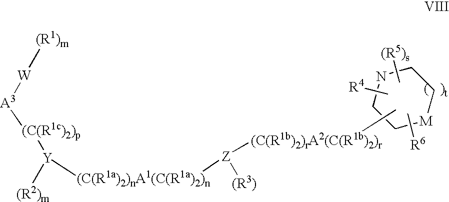

wherein:

R^{1a}, R^{1b} and R^{1c} are independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, R⁸O—, R⁹S(O)_{q}—, CN, NO₂, R⁸C(O)—, R⁸OC(O)—, N(R⁸)₂, (R⁸)₂NC(O)—, C(O)N(R⁸)₂—, or N₃;

c) C₁-C₆ alkyl, unsubstituted or substituted by unsubstituted or substituted aryl, unsubstituted or substituted heterocycle, C₃-C₁₀ cycloalkyl, C₂-C₆ alkenyl, C₂-C₆ alkynyl, R⁸O—, R⁹S(O)_{q}—, CN, R⁸C(O)—, R⁸OC(O)—, N(R⁸)₂, N₃, or R⁸C(O)O—;

R¹ is selected from:

a) H,

b) unsubstituted or substituted C₁-C₆ alkyl,

c) unsubstituted or substituted aryl,

d) unsubstituted or substituted heterocycle,

e) —(C₁-C₆ alkyl)N(R⁸)₂,

f) —R⁸C(O)R⁸,

g) —(C₁-C₆ alkyl)OR⁸,

h) —N(R⁸)₂,

i) —OR⁸,

j) —R⁸NHC(O)R⁸,

k) —R⁸C(O)N(R⁸)₂,

l) CF₃,

m) halo,

n) —C(O)OR⁸,

o) C₂-C₆ alkynyl,

p) C₂-C₆ alkenyl,

q) perfluoroalkyl,

r) N₃,

s) NO₂,

t) CN,

u) R⁹S(O)_{q}—;

R² is selected from:

a) hydrogen,

b) CN,

c) NO₂,

d) halogen,

e) aryl, unsubstituted or substituted,

f) heteroaryl, unsubstituted or substituted,

g) C₁-C₆ alkyl, unsubstituted or substituted,

h) OR⁸,

i) N₃,

j) R⁹S(O)_{q},

k) R⁸HC═CH—, and

l) R⁸C≡C—;

R³ is selected from:

a) H,

b) CN,

c) NO₂,

d) halogen,

e) C₁-C₆ alkyl, unsubstituted or substituted,

f) OR⁸,

g) aryl, unsubstituted or substituted,

h) heteroaryl, unsubstituted or substituted, and

i) CF₃;

R⁴ is selected from:

a) H,

b) ═O, or

c) ═S;

R⁵ is selected from:

a) H,

b) CN,

c) NO₂,

d) halogen,

e) C₁-C₆ alkyl, unsubstituted or substituted,

f) N₃,

g) R⁹S(O)_{q},

h) —HC═CH₂,

i) HC≡C—,

j) aryl, unsubstituted or substituted,

k) heterocycle, unsubstituted or substituted,

l) CF₃O—,

m) CF₃CH₂O—,

n) C₃-C₁₀ cycloalkyl,

o) CF₃,

p) —(C₁-C₆ alkyl)N(R⁸)₂,

q) —(C₁-C₆ alkyl)OR⁸,

r) OR⁸,

s) N(R⁸)₂,

t) —C(O)(C₁-C₆ alkyl), and

u) —(C₁-C₆ alkyl)C(O)R⁸;

R⁶ is selected from:

a) H,

b) C₁-C₆ alkyl, unsubstituted or substituted,

c) OR⁸, and

d) —C(O)(C₁-C₆ alkyl);

R⁸ is independently selected from hydrogen, unsubstituted or substituted C₁-C₆ alkyl, unsubstituted or substituted benzyl, unsubstituted or substituted heterocycle and unsubstituted or substituted aryl;

R⁹ is independently selected from unsubstituted or substituted C₁-C₆ alkyl, unsubstituted or substituted benzyl and unsubstituted or substituted aryl;

A¹ and A² are independently selected from:

a) a bond,

b) —HC═CH—,

c) —C≡C—,

d) O,

e) S(O)_{q},

f) OC(O),

g) C(O),

h) C(O)O, and

i) NR⁸;

A³ is selected from a bond, —C(═O),

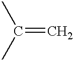

and C₃-C₆ cycloalkyl;

M is selected from CH₂, NH, O or S;

W is selected from:

a) hydrogen,

b) heterocycle, and

c) aryl;

Y is selected from:

a) aryl, and

b) heterocycle;

Z is selected from:

a) aryl,

b) heterocycle,

c) C₃-C₆ cycloalkyl, and

d) a bond;

m is 0, 1, 2, 3 or 4;

n is 0, 1, 2, 3 or 4;

p is 0, 1, 2, 3 or 4;

q is 0, 1 or 2;

r is 0, 1, 2, 3, or 4;

s is 0, 1, 2, 3 or 4;

t is 0, 1, 2 or 3;

(I) a compound represented by formula (VIIIA):

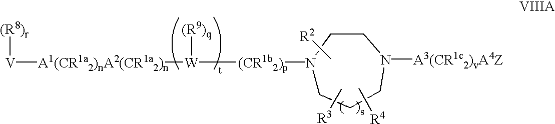

wherein:

R^{1a} and R^{1b} are independently selected from:

a) hydrogen,

b) aryl, heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, R¹⁰NC(O)—, (R¹⁰)₂NC(O)—, R¹⁰₂N—C(NR¹⁰)—, CN, NO₂, R¹⁰C(O)—, N₃, —N(R¹⁰)₂, or R¹¹OC(O)NR¹⁰—,

c) unsubstituted or substituted C₁-C₆ alkyl wherein the substituent on the substituted C₁-C₆ alkyl is selected from unsubstituted or substituted aryl, heterocyclic, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂, and R¹¹OC(O)—NR¹⁰—;

R^{1c} is independently selected from:

a) hydrogen,

b) unsubstituted or substituted aryl, unsubstituted or substituted heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, N₃, —N(R¹⁰)₂ or R¹¹OC(O)NR¹⁰—,

c) unsubstituted or substituted C₁-C₆ alkyl wherein the substituent on the substituted C₁-C₆ alkyl is selected from unsubstituted or substituted aryl, unsubstituted or substituted heterocycle, C₃-C₁₀ cycloalkyl, C₂-C₆ alkenyl, C₂-C₆ alkynyl, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂ and R¹¹OC(O)—NR¹⁰—;

or two R^{1c}s on the same carbon are combined with that carbon to form a C₄-C₆ cycloalkyl or C₆-C₁₀ multicyclic alkyl ring;

R² and R³ are independently selected from: H; unsubstituted or substituted C₁₋₈ alkyl, unsubstituted or substituted C₂₋₈ alkenyl, unsubstituted or substituted C₂₋₈ alkynyl, unsubstituted or substituted aryl, unsubstituted or substituted heterocycle,

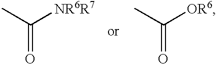

wherein the substituted group is substituted with one or more of:

1) aryl or heterocycle, unsubstituted or substituted with:

a) C₁₋₄ alkyl,

b) (CH₂)ₚOR⁶,

c) (CH₂)ₚNR⁶R⁷,

d) halogen,

e) CN,

f) aryl or heteroaryl,

g) perfluoro-C₁₋₄ alkyl,

h) SR^{6a}, S(O)R^{6a}, SO₂R^{6a},

2) C₃₋₆ cycloalkyl,

3) OR⁶,

4) SR^{6a}, S(O)R^{6a}, or SO₂R^{6a},

5) —N R⁶R⁷,

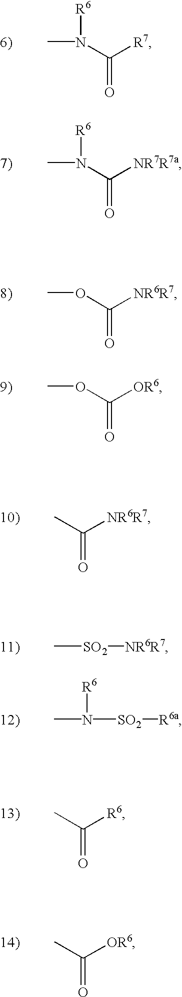

15) N₃,

16) F, or

17) perfluoro-C₁₋₄-alkyl; or

R² and R³ are attached to the same C atom and are combined to form —(CH₂)ᵤ— wherein one of the carbon atoms is optionally replaced by a moiety selected from: O, S(O)ₘ, —NC(O)—, and —N(COR¹⁰)—;

R⁴ is selected from H and CH₃;

and any two of R², R³ and R⁴ are optionally attached to the same carbon atom;

R⁶, R⁷ and R^{7a} are independently selected from: H; C₁₋₄ alkyl, C₃₋₆ cycloalkyl, heterocycle, aryl, aroyl, heteroaroyl, arylsulfonyl, heteroarylsulfonyl, unsubstituted or substituted with:

a) C₁₋₄ alkoxy,

b) aryl or heterocycle,

c) halogen,

d) HO,

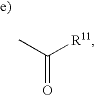

f) —SO₂R¹¹, or

g) N(R¹⁰)₂; or

R⁶ and R⁷ may be joined in a ring;

R⁷ and R^{7a} may be joined in a ring;

R^{6a} is selected from: C₁₋₄ alkyl, C₃₋₆ cycloalkyl, heterocycle, aryl, unsubstituted or substituted with:

a) C₁₋₄ alkoxy,

b) aryl or heterocycle,

c) halogen,

d) HO,

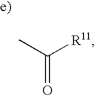

f) —SO₂R¹¹, or

g) N(R¹⁰)₂;

R⁸ is independently selected from:

a) hydrogen,

b) unsubstituted or substituted aryl, unsubstituted or substituted heterocycle, unsubstituted or substituted C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, N₃, —N(R¹⁰)₂, or R¹¹OC(O)NR¹⁰—, and

c) C₁-C₆ alkyl unsubstituted or substituted by aryl, cyanophenyl, heterocycle, C₃-C₁₀ cycloalkyl, C₂-C₆ alkenyl, C₂-C₆ alkynyl, perfluoroalkyl, F, Cl, Br, R¹⁰O—, R¹¹S(O)ₘ—, R¹⁰C(O)NH—, (R¹⁰)₂NC(O)—, R¹⁰₂N—C(NR¹⁰)—, CN, R¹⁰C(O)—, N₃, —N(R¹⁰)₂, or R¹⁰OC(O)NH—;

R⁹ is selected from:

a) hydrogen,

b) alkenyl, alkynyl, perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, NO₂, R¹⁰C(O)—, N₃, —N(R¹⁰)₂, or R¹¹OC(O)NR¹⁰—, and

c) C₁-C₆ alkyl unsubstituted or substituted by perfluoroalkyl, F, Cl, Br, R¹⁰O—, R¹¹S(O)ₘ—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂, or R¹¹OC(O)NR¹⁰—;

R¹⁰ is independently selected from hydrogen, C₁-C₆ alkyl, benzyl and aryl;

R¹¹ is independently selected from C₁-C₆ alkyl and aryl;

A¹ and A² are independently selected from: a bond, —CH═CH—, —C≡C—, —C(O)—, —C(O)NR¹⁰—, —NR¹⁰C(O)—, O, —N(R¹⁰)—, —S(O)₂N(R¹⁰)—, —N(R¹⁰)S(O)₂—, or S(O)ₘ;

A³ is selected from: —C(O)—, —C(O)NR¹⁰—, —C(O)O—, and S(O)ₘ;

A⁴ is selected from: a bond, O, and NR¹⁰;

V is selected from:

a) hydrogen,

b) heterocycle,

c) aryl,

d) C₁-C₂₀ alkyl wherein from 0 to 4 carbon atoms are replaced with a heteroatom selected from O, S, and N, and

e) C₂-C₂₀ alkenyl,

provided that V is not hydrogen if Al is S(O)ₘ and V is not hydrogen if A¹ is a bond, n is 0 and A² is S(O)ₘ;

W is a heterocycle;

Z is unsubstituted or substituted aryl or unsubstituted or substituted heteroaryl;

m is 0, 1 or 2;

n is 0, 1, 2, 3 or 4;

p is 0, 1, 2, 3 or 4;

q is 1 or 2;

r is 0 to 5, provided that r is 0 when V is hydrogen;

s is 0 or 1;

t is 0 or 1;

u is 4 or 5; and

v is 0, 1, 2 or 3; provided that v is not 0 if A³ is —C(O)— or S(O)ₘ;

or a pharmaceutically acceptable salt or optical isomer thereof.

Examples of compounds which inhibit prenyl protein transferase include the following:

2(S)-Butyl-1-(2,3-diaminoprop-1-yl)-1-(1-naphthoyl)piperazine;

1-(3-Amino-2-(2-naphthylmethylamino)prop-1-yl)-2(S)-butyl-4-(1-naphthoyl)piperazine;

2(S)-Butyl-1-{5-[1-(2-naphthylmethyl)]-4,5-dihydroimidazol}methyl-4-(1-naphthoyl)piperazine;

1-[5-(1-Benzylimidazol)methyl]-2(S)-butyl-4-(1-naphthoyl)piperazine;

1-{5-[1-(4-nitrobenzyl)]imidazolylmethyl}-2(S)-butyl-4-(1-naphthoyl)piperazine;

1-(3-Acetamidomethylthio-2(R)-aminoprop-1-yl)-2(S)-butyl-4-(1-naphthoyl)piperazine;

2(S)-Butyl-1-[2-(1-imidazolyl)ethyl]sulfonyl-4-(1-naphthoyl)piperazine;

2(R)-Butyl-1-imidazolyl-4-methyl-4-(1-naphthoyl)piperazine;

2(S)-Butyl-4-(1-naphthoyl)-1-(3-pyridylmethyl)piperazine;

1-2(S)-butyl-(2(R)-(4-nitrobenzyl)amino-3-hydroxypropyl)-4-(1-naphthoyl)piperazine;

1-(2(R)-Amino-3-hydroxyheptadecyl)-2(S)-butyl-4-(1-naphthoyl)piperazine;

2(S)-Benzyl-1-imidazolyl-4-methyl-4-(1-naphthoyl)piperazine;

1-(2(R)-Amino-3-(3-benzylthio)propyl)-2(S)-butyl-4-(1-naphthoyl)piperazine;

1-(2(R)-Amino-3-[3-(4-nitrobenzylthio)propyl])-2(S)-butyl-4-(1-naphthoyl)piperazine;

2(S)-Butyl-1-[(4-imidazolyl)ethyl]-4-(1-naphthoyl)piperazine;

2(S)-Butyl-1-[(4-imidazolyl)methyl]-4-(1-naphthoyl)piperazine;

2(S)-Butyl-1-[(1-naphth-2-ylmethyl)-1H-imidazol-5-yl)acetyl]-4-(1-naphthoyl)piperazine;

2(S)-Butyl-1-[(1-naphth-2-ylmethyl)-1H-imidazol-5-yl)ethyl]-4-(1-naphthoyl)piperazine;

1-(2(R)-Amino-3-hydroypropyl)-2(S)-butyl-4-(1-naphthoyl)piperazine;

1-(2(R)-Amino-4-hydroxybutyl)-2(S)-butyl-4-(1-naphthoyl)piperazine;

1-(2-Amino-3-(2-benzyloxyphenyl)propyl)-2(S)-butyl-4-(1-naphthoyl)piperazine;

1-(2-Amino-3-(2-hydroxyphenyl)propyl)-2(S)-butyl-4-(1-naphthoyl)piperazine;

1-[3-(4-imidazolyl)propyl]-2(S)-butyl-4-(1-naphthoyl)-piperazine;

2(S)-n-Butyl-4-(2,3-dimethylphenyl)-1-(4-imidazolylmethyl)-piperazin-5-one;

2(S)-n-Butyl-1-[1-(4-cyanobenzyl)imidazol-5-ylmethyl]-4-(2,3-dimethylphenyl)piperazin-5-one;

1-[1-(4-Cyanobenzyl)imidazol-5-ylmethyl]-4-(2,3-dimethylphenyl)-2(S)-(2-methoxyethyl)piperazin-5-one;

2(S)-n-Butyl-4-(1-naphthoyl)-1-[1-(1-naphthylmethyl)imidazol-5-ylmethyl]-piperazine;

2(S)-n-Butyl-4-(1-naphthoyl)-1-[1-(2-naphthylmethyl)imidazol-5-ylmethyl]-piperazine;

2(S)-n-Butyl-1-[1-(4-cyanobenzyl)imidazol-5-ylmethyl]-4-(1-naphthoyl)piperazine;

2(S)-n-Butyl-1-[1-(4-methoxybenzyl)imidazol-5-ylmethyl]-4-(1-naphthoyl)piperazine;

2(S)-n-Butyl-1-[1-(3-methyl-2-butenyl)imidazol-5-ylmethyl]-4-(1-naphthoyl)piperazine;

2(S)-n-Butyl-1-[1-(4-fluorobenzyl)imidazol-5-ylmethyl]-4-(1-naphthoyl)piperazine;

2(S)-n-Butyl-1-[1-(4-chlorobenzyl)imidazol-5-ylmethyl-4-(1-naphthoyl)piperazine;

1-[1-(4-Bromobenzyl)imidazol-5-ylmethyl]-2(S)-n-butyl-4-(1-naphthoyl)piperazine;

2(S)-n-Butyl-4-(1-naphthoyl)-1-[1-(4-trifluoromethylbenzyl)imidazol-5-ylmethyl]-piperazine;

2(S)-n-Butyl-1-[1-(4-methylbenzyl)imidazol-5-ylmethyl]-4-(1-naphthoyl)piperazine;

2(S)-n-Butyl-1-[1-(3-methylbenzyl)imidazol-5-ylmethyl]-4-(1-naphthoyl)piperazine;

1-[1-(4-Phenylbenzyl)imidazol-5-ylmethyl]-2(S)-n-butyl-4-(1-naphthoyl)piperazine;

2(S)-n-Butyl-4-(1-naphthoyl)-1-[1-(2-phenylethyl)imidazol-5-ylmethyl]-piperazine;

2(S)-n-Butyl-4-(1-naphthoyl)-1-[1-(4-trifluoromethoxy)imidazol-5-ylmethyl]piperazine;

1-{[1-(4-cyanobenzyl)-1H-imidazol-5-yl]acetyl}-2(S)-n-butyl-4-(1-naphthoyl)piperazine;

(S)-1-(3-Chlorophenyl)-4-[1-(4-cyanobenzyl)-5-imidazolylmethyl]-5-[2-(methanesulfonyl)ethyl]-2-piperazinone;

(S)-1-(3-Chlorophenyl)-4-[1-(4-cyanobenzyl)-5-imidazolylmethyl]-5-[2-(ethanesulfonyl)ethyl]-2-piperazinone;

(R)-1-(3-Chlorophenyl)-4-[1-(4-cyanobenzyl)-5-imidazolylmethyl]-5-[2-(ethanesulfonyl)methyl]-2-piperazinone;

(S)-1-(3-Chlorophenyl)-4-[1-(4-cyanobenzyl)-5-imidazolylmethyl]-5-[N-ethyl-2-acetamido]-2-piperazinone;

(±)-5-(2-Butynyl)-1-(3-chlorophenyl)-4-[1-(4-cyanobenzyl)-5-imidazolylmethyl]-2-piperazinone;

1-(3-Chlorophenyl)-4-[1-(4-cyanobenzyl)-5-imidazolylmethyl]-2-piperazinone;

5(S)-Butyl-4-[1-(4-cyanobenzyl-2-methyl)-5-imidazolylmethyl]-1-(2,3-dimethylphenyl)-piperazin-2-one;

4-[1-(2-(4-Cyanophenyl)-2-propyl)-5-imidazolylmethyl]-1-(3-chlorophenyl)-5(S)-(2-methylsulfonylethyl)piperazin-2-one;

5(S)-n-Butyl-4-[1-(4-cyanobenzyl)-5-imidazolylmethyl]-1-(2-methylphenyl)piperazin-2-one;

4-[1-(4-Cyanobenzyl)-5-imidazolylmethyl]-5(S)-(2-fluoroethyl)-1-(3-chlorophenyl)piperazin-2-one;

4-[3-(4-Cyanobenzyl)pyridin-4-yl]-1-(3-chlorophenyl)-5(S)-(2-methylsulfonylethyl)-piperazin-2-one;

4-[5-(4-Cyanobenzyl)-1-imidazolylethyl]-1-(3-chlorophenyl)piperazin-2-one;

4-{3-[4-(−2-Oxo-2-H-pyridin-1-yl)benzyl]-3-H-imidazol-4-ylmethyl}benzonitrile;

4-{3-[4-3-Methyl-2-oxo-2-H-pyridin-1-yl)benzyl]-3-H-imidazol-4-ylmethyl}benzonitrile;

4-{3-[4-(−2-Oxo-piperidin-1-yl)benzyl]-3-H-imidazol-4-ylmethyl]benzonitrile;

4-{3-[3-Methyl-4-(2-oxopiperidin-1-yl)-benzyl]-3-H-imidizol-4-ylmethyl}-benzonitrile;

(4-{3-[4-(2-Oxo-pyrrolidin-1-yl)-benzyl]-3H-imidizol-4-ylmethyl}-benzonitrile;

4-{3-[4-(3-Methyl-2-oxo-2-H-pyrazin-1-yl)-benzyl-3-H-imidizol-4-ylmethyl}-benzonitrile;

4-{(3-[2-Methoxy-4-(2-oxo-2-H-pyridin-1-yl)-benzyl]-3-H-imidizol-4-ylmethyl}-benzonitrile;

4-{1-[4-(5-Chloro-2-oxo-2H-pyridin-1-yl)-benzyl]-1H-pyrrol-2-ylmethyl}-benzonitrile;

4-[1-(2-Oxo-2H-[1,2′]bipyridinyl-5′-ylmethyl)-1H-pyrrol-2-ylmethyl]-benzonitrile;

4-[1-(5-Chloro-2-oxo-2H-[1,2′]bipyridinyl-5′-ylmethyl)-1H-pyrrol-2-ylmethyl]-benzonitrile;

4-[3-(2-Oxo-1-phenyl-1,2-dihydropyridin-4-ylmethyl)-3H-imidazol-4-ylmethyl]benzonitrile;

4-13-[1-(3-Chloro-phenyl)-2-oxo-1,2-dihydropyridin-4-ylmethyl]-3H-imidazol-4-ylmethyl}benzonitrile;

19,20-Dihydro-19-oxo-5H,17H-18,21-ethano-6,10:12,16-dimetheno-22H-imidazo[3,4-h][1,8,11,14]oxatriazacycloeicosine-9-carbonitrile;

19-Chloro-22,23-dihydro-22-oxo-5H-21,24-ethano-6,10-metheno-25H-dibenzo[b,e]imidazo[4,3-l][1,4,7,10,13]dioxatriazacyclononadecine-9-carbonitrile;

22,23-Dihydro-22-oxo-5H-21,24-ethano-6,10-metheno-25H-dibenzo[b,e]imidazo[4,3-l][1,4,7,10,13]dioxatriazacyclononadecine-9-carbonitrile;

20-Chloro-23,24-dihydro-23-oxo-5H-22,25-ethano-6,10:12,16-dimetheno-12H,26H-benzo[b]imidazo[4,3-i][1,17,4,7,10]dioxatriazacyclohemicosine-9-carbonitrile;

(S)-20-Chloro-23,24-dihydro-27-[2-(methylsulfonyl)ethyl]-23-oxo-5H-22,25-ethano-6,10:12,16-dimetheno-12H,26H-benzo[b]imidazo[4,3-i][1,17,4,7,10]dioxatriazacyclohemicosine-9-carbonitrile;

(±)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile;

(+)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile;

(−)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile;

5H, 17H,20H-18,21-Ethano-6,10:12,16-dimetheno-22H-imidazo[3,4-h][1,8,11,14]oxatriazacycloeicosin-20-one;

(±)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile;

(+) or (−)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile; (Enantiomer A)

(−) or (+)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile; (Enantiomer B)

(±)-19,20-Dihydro-19,22-dioxo-5H-18,21-ethano-12,14-etheno-6,10-metheno-22H-benzo[d]imidazo[4,3-k][1,6,9,12]oxatriazacyclooctadecine-9-carbonitrile;

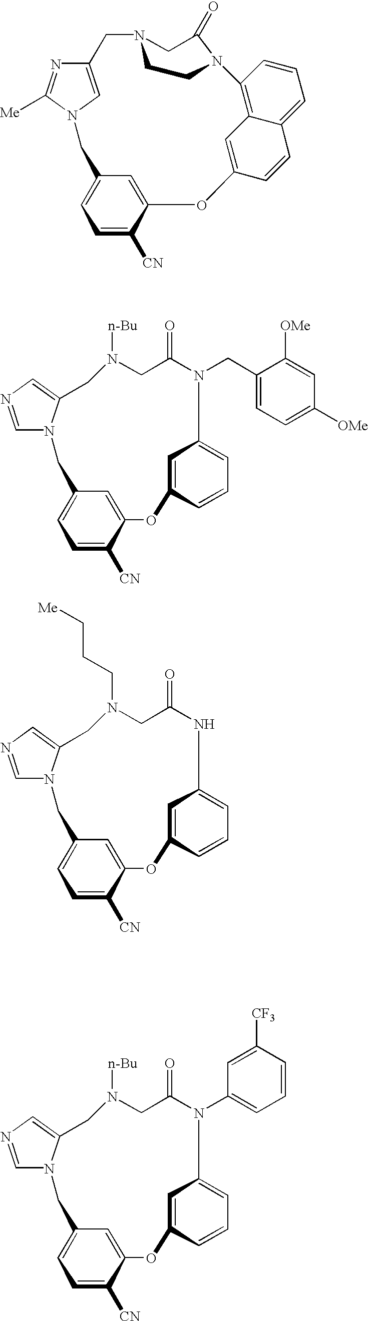

18,19-dihydro-19-oxo-5H,17H-6,10:12,16-dimetheno-1H-imidazo[4,3-c][1,11,4]dioxaazacyclononadecine-9-carbonitrile;

17,18-dihydro-18-oxo-5H-6,10:12,16-dimetheno-12H,20H-imidazo[4,3-c][1,11,4]dioxaazacyclooctadecine-9-carbonitrile;

(±)-17,18,19,20-tetrahydro-19-phenyl-5H-6,10:12,16-dimetheno-21H-imidazo[3,4-h][1,8,11]oxadiazacyclononadecine-9-carbonitrile;

21,22-dihydro-5H-6,10:12,16-dimetheno-23H-benzo[g]imidazo[4,3-l][1,8,11]oxadiazacyclononadecine-9-carbonitrile;

22,23-dihydro-23-oxo-5H,21H-6,10:12,16-dimetheno-24H-benzo[g]imidazo[4,3-m][1,8,12]oxadiazaeicosine-9-carbonitrile;

22,23-dihydro-5H,21H-6,10:12,16-dimetheno-24H-benzo[g]imidazo[4,3-m][1,8,11]oxadiazaeicosine-9-carbonitrile;

1-(3-trifluoromethoxyphenyl)-4-[1-(4-cyano-3-methoxybenzyl)-5-imidazolyl methyl]-2-piperazinone;

or a pharmaceutically acceptable salt, stereoisomer or optical isomer thereof.

Specific examples of a farnesyl-protein transferase inhibitor are 1-(3-Chlorophenyl)-4-[1-(4-cyanobenzyl)-5-imidazolylmethyl]-2-piperazinone;

(R)-1-(3-Chlorophenyl)-4-[1-(4-cyanobenzyl)-5-imidazolylmethyl]-5-[2-(ethanesulfonyl)methyl]-2-piperazinone;

4-[1-(5-Chloro-2-oxo-2H-[1,2′]bipyridinyl-5′-ylmethyl)-1H-pyrrol-2-ylmethyl]-benzonitrile; and

1-[N-(1-(4-cyanobenzyl)-5-imidazolylmethyl)-N-(4-cyanobenzyl)amino]-4-(phenoxy)benzene;

(±)-19,20-Dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile;

1-(3-trifluoromethoxyphenyl)-4-[1-(4-cyano-3-methoxybenzyl)-5-imidazolyl methyl]-2-piperazinone;

3-(biphenyl-4-ylmethoxy)-4-imidazol-1-ylmethyl-benzonitrile;

3-(biphenyl-4-yl-2-ethoxy)-4-imidazol-1-ylmethylbenzonitrile;

3-(biphenyl-3-ylmethoxy)-4-imidazol-1-ylmethyl-benzonitrile;

2-(biphenyl-4-ylmethoxy)-4-imidazol-1-ylmethyl-benzonitrile;

2-(biphenyl-4-yl-2-ethoxy)-4-imidazol-1-ylmethyl-benzonitrile;

1-tert-butoxycarbonyl-4-(3-chlorophenyl)-2(S)-[2-(2-cyano-5-imidazol-1-ylmethyl-phenoxy)ethyl]piperazine;

2-(3-chlorophenoxy)-4-imidazol-1-ylmethyl-benzonitrile;

2-(4-chlorophenyl-2-ethoxy)-4-imidazol-1-ylmethyl-benzonitrile;

2-(3-chlorophenyl-2-ethoxy)-4-imidazol-1-ylmethyl-benzonitrile;

2-(2-chlorophenyl-2-ethoxy)-4-imidazol-1-ylmethyl-benzonitrile;

2-(phenyl-2-ethoxy)-4-imidazol-1-ylmethyl-benzonitrile;

2-(3-chlorobenzyloxy)-4-imidazol-1-ylmethyl-benzonitrile;

2-(4-chlorobenzyloxy)-4-imidazol-1-ylmethyl-benzonitrile;

2-(2,4-dichlorobenzyloxy)-4-imidazol-1-ylmethyl-benzonitrile;

2-(benzyloxy)-4-imidazol-1-ylmethyl-benzonitrile;

2-(biphenyl-2-ylmethoxy)-4-imidazol-1-ylmethyl-benzonitrile;

2-(phenyl-4-butoxy)-4-imidazol-1-ylmethyl-benzonitrile;

2-(phenyl-3-propoxy)-4-imidazol-1-ylmethyl-benzonitrile;

2-(biphenyl-4-yl-2-ethoxy)-4-(1,2,4-triazol-1-yl)methyl-benzonitrile;

2-(biphenyl-4-yl-2-ethoxy)-4-(2-methyl-imidazol-1-yl)methyl-benzonitrile;

2-(biphenyl-4-yl-2-ethoxy)-4-benzimidazol-1-yl)methyl-benzonitrile;

4-imidazol-1-ylmethyl-2-(naphthalen-2-yloxy)-benzonitrile;

2-(3-cyanophenoxy)-4-imidazol-1-ylmethyl-benzonitrile;

2-(3-bromophenoxy)-4-imidazol-1-ylmethyl-benzonitrile;

2-(biphen-3-yloxy)-4-imidazol-1-ylmethyl-benzonitrile;

2-(biphen-4-yloxy)-4-imidazol-1-ylmethyl-benzonitrile;

2-(3-acetylphenoxy)-4-imidazol-1-ylmethyl-benzonitrile;

2-(2-acetylphenoxy)-4-imidazol-1-ylmethyl-benzonitrile;

2-(3-trifluoromethylphenoxy)-4-imidazol-1-ylmethyl-benzonitrile;

2-(3-methylphenoxy)-4-imidazol-1-ylmethyl-benzonitrile;

2-(2-methylphenoxy)-4-imidazol-1-ylmethyl-benzonitrile;

2-(4-methylphenoxy)-4-imidazol-1-ylmethyl-benzonitrile;

2-(3-methoxyphenoxy)-4-imidazol-1-ylmethyl-benzonitrile;

2-(2-methoxyphenoxy)-4-imidazol-1-ylmethyl-benzonitrile;

2-(4-methoxyphenoxy)-4-imidazol-1-ylmethyl-benzonitrile;

2-(3,5-dimethylphenoxy)-4-imidazol-1-ylmethyl-benzonitrile;

2-(3,4-dimethylphenoxy)-4-imidazol-1-ylmethyl-benzonitrile;

2-(3,5-dimethoxyphenoxy)-4-imidazol-1-ylmethyl-benzonitrile;

2-(1-naphthyloxy)-4-imidazol-1-ylmethyl-benzonitrile;

2-(2,4-dichlorophenoxy)-4-imidazol-1-ylmethyl-benzonitrile;

2-(3-fluorophenoxy)-4-imidazol-1-ylmethyl-benzonitrile;

2-(3-t-butylphenoxy)-4-imidazol-1-ylmethyl-benzonitrile;

2-[3-(N,N-diethylamino)phenoxy]-4-imidazol-1-ylmethyl-benzonitrile;

2-(3-n-propylphenoxy)-4-imidazol-1-ylmethyl-benzonitrile;

2-(2,3-dimethoxyphenoxy)-4-imidazol-1-ylmethyl-benzonitrile;

2-(2,3-dimethylphenoxy)-4-imidazol-1-ylmethyl-benzonitrile;

2-(3,4-dimethoxyphenoxy)-4-imidazol-1-ylmethyl-benzonitrile;

2-(2,5-dimethoxyphenoxy)-4-imidazol-1-ylmethyl-benzonitrile;

2-(3,4-dichlorophenoxy)-4-imidazol-1-ylmethyl-benzonitrile;

2-(2,4-dimethylphenoxy)-4-imidazol-1-ylmethyl-benzonitrile;

2-(4-chloro-2-methylphenoxy)-4-imidazol-1-ylmethyl-benzonitrile;

2-(5-chloro-2-methylphenoxy)-4-imidazol-1-ylmethyl-benzonitrile;

2-(2-chloro-4,5-dimethylphenoxy)-4-imidazol-1-ylmethyl-benzonitrile;

2-(5-hydroxymethyl-2-methoxyphenoxy)-4-imidazol-1-ylmethylbenzonitrile;

4-imidazol-1-ylmethyl-2-(3-phenylamino-phenoxy)-benzonitrile;

4-imidazol-1-ylmethyl-2-[3-(2-methylphenylamino)-phenoxy]-benzonitrile;

4-imidazol-1-ylmethyl-2-(3-phenoxy-phenoxy)-benzonitrile;

2-(2-benzoyl-phenoxy)-4-imidazol-1-ylmethyl-benzonitrile;

1-(5-chloro-2-methoxy-phenyl)-3-[3-(2-cyano-5-imidazol-1-ylmethyl-phenoxy)-phenyl]-urea;

1-(2,5-dimethoxy-phenyl)-3-[3-(2-cyano-5-imidazol-1-ylmethyl-phenoxy)-phenyl]-urea;

2-(3-benzyloxy-phenoxy)-4-imidazol-1-ylmethyl-benzonitrile;

2-(4-benzyloxy-phenoxy)-4-imidazol-1-ylmethyl-benzonitrile;

2-(2-benzyl-phenoxy)-4-imidazol-1-ylmethyl-benzonitrile;

2-(3-ethynyl-phenoxy)-4-imidazol-1-ylmethyl-benzonitrile;

2-(4-acetyl-3-methyl-phenoxy)-4-imidazol-1-ylmethyl-benzonitrile;

4-imidazol-1-ylmethyl-2-(1H-indazol-6-yloxy)-benzonitrile;

4-imidazol-1-ylmethyl-2-(5,6,7,8-tetrahydro-naphthalen-1-yloxy)-benzonitrile;

4-imidazol-1-ylmethyl-2-(8-oxo-5,6,7,8-tetrahydro-naphthalen-1-yloxy)-benzonitrile;

4-imidazol-1-ylmethyl-2-(1H-indol-7-yloxy)-benzonitrile;

4-imidazol-1-ylmethyl-2-(3-oxo-indan-4-yloxy)-benzonitrile;

4-imidazol-1-ylmethyl-2-(1H-indol-4-yloxy)-benzonitrile;

2-[3-(2-hydroxy-ethoxy)-phenoxy]-4-imidazol-1-ylmethyl-benzonitrile;

4-imidazol-1-ylmethyl-2-(4-imidazol-1-yl-phenoxy)-benzonitrile;

4′-(2-cyano-5-imidazol-1-ylmethyl-phenoxy)-biphenyl-4-carbonitrile;

N-[3-(2-cyano-5-imidazol-1-ylmethyl-phenoxy)-phenyl]-acetamide;

4-imidazol-1-ylmethyl-2-(9-oxo-9H-fluoren-4-yloxy)-benzonitrile;

3-(2-cyano-5-imidazol-1-ylmethyl-phenoxy)-Nphenyl-benzamide;

3-(2-cyano-5-imidazol-1-ylmethyl-phenoxy)-N-ethyl-N-phenyl-benzamide;

3-(2-cyano-5-imidazol-1-ylmethyl-phenoxy)-N-cyclopropylmethyl-N-phenyl-benzamide;

2-(5-chloro-pyridin-3-yloxy)-4-imidazol-1-ylmethyl-benzonitrile;

N-[3-(2-cyano-5-imidazol-1-ylmethyl-phenoxy)-phenyl]-benzenesulfonamide;

4-imidazol-1-ylmethyl-2-(indan-5-yloxy)-benzonitrile;

3-(9H-carbazol-2-yloxy)-4-imidazol-1-ylmethyl-benzonitrile;

4-imidazol-1-ylmethyl-2-(5,6,7,8-tetrahydro-naphthalen-2-yloxy)-benzonitrile;

4-imidazol-1-ylmethyl-2-(2-methoxy-4-propenyl-phenoxy)-benzonitrile;

4-imidazol-1-ylmethyl-2-[4-(3-oxo-butyl)-phenoxy]-benzonitrile;

2-(3-chlorophenoxy)-5-imidazol-1-ylmethyl-benzonitrile;

2-(4-chlorophenoxy)-4-imidazol-1-ylmethyl-benzonitrile;

2-(3,5-dichlorophenoxy)-4-imidazol-1-ylmethyl-benzonitrile;

2-(pyridin-3-yloxy)-4-imidazol-1-ylmethyl-benzonitrile;

2-(2-chlorophenoxy)-4-imidazol-1-ylmethyl-benzonitrile;

2-(3-chlorophenoxy)-5-(4-phenyl-imidazol-1-ylmethyl)-benzonitrile;

2-(biphen-2-yloxy)-4-imidazol-1-ylmethyl-benzonitrile;

2-(phenoxy)-4-imidazol-1-ylmethyl-benzonitrile;

2-(2-chloro-4-methoxyphenoxy)-4-imidazol-1-ylmethyl-benzonitrile;

2-(2-chlorophenylsulfanyl)-4-imidazol-1-ylmethyl-benzonitrile;

4-imidazol-1-ylmethyl-2-(naphthalen-2-ylsulfanyl)-benzonitrile;

2-(2,4-dichlorophenylsulfanyl)-4-imidazol-1-ylmethyl-benzonitrile;

2-(2,4-dichloro-benzenesulfinyl)-4-imidazol-1-ylmethyl-benzonitrile;

2-(2,4-dichloro-benzenesulfonyl)-4-imidazol-1-ylmethyl-benzonitrile;

2-(2-methyl-pyridin-3-yloxy)-4-imidazol-1-ylmethyl-benzonitrile;

2-(2,4-dimethyl-pyridin-3-yloxy)-4-imidazol-1-ylmethyl-benzonitrile;

2-(4-chloro-2-methoxyphenoxy)-4-imidazol-1-ylmethyl-benzonitrile;

2-(2-chlorophenoxy)-4-(5-methyl-imidazol-1-ylmethyl)-benzonitrile;

2-(2-chlorophenoxy)-4-(4-methyl-imidazol-1-ylmethyl)-benzonitrile;

2-(3-chloro-5-trifluoromethyl-pyridin-2-yloxy)-4-imidazol-1-ylmethyl-benzonitrile;

2-(2,4-dichlorophenoxy)-4-(2-methyl-imidazol-1-ylmethyl)-benzonitrile;

N-[3-(2-cyano-5-imidazol-1-ylmethyl-phenoxy)-phenyl]-benzamide;

2-[3-(2-cyano-5-imidazol-1-ylmethyl-phenoxy)-phenyl]-N-phenyl-acetamide;

4-imidazol-1-ylmethyl-2-(quinolin-6-yloxy)-benzonitrile;

4-imidazol-1-ylmethyl-2-(2-oxo-1,2-dihydro-quinolin-6-yloxy)-benzonitrile;

N-[3-(2-cyano-5-imidazol-1-ylmethyl-phenoxy)-phenyl]-2-phenyl-acetamide;

5-(2-cyano-5-imidazol-1-ylmethyl-phenoxy)-N-cyclohexyl-nicotinamide;

N-(3-chloro-phenyl)-5-(2-cyano-5-imidazol-1-ylmethyl-phenoxy)-nicotinamide;

2-(2,3-dimethoxyphenoxy)-4-(2,4-dimethyl-imidazol-1-ylmethyl)-benzonitrile;

4-(2-methyl-imidazol-1-ylmethyl)-2-(naphthalen-2-yloxy)-benzonitrile;

4-(1-imidazol-1-yl-1-methyl-ethyl)-2-(naphthalen-2-yloxy)-benzonitrile;

1-[4-iodo-3-(naphthalen-2-yloxy)-benzyl]-1H-imidazole; acetic acid 3-[3-(2-chloro-phenoxy)-4-cyano-benzyl]-3H-imidazol-4-ylmethyl ester;

2-(2-chloro-phenoxy)-4-(5-hydroxymethyl-imidazol-1-ylmethyl)-benzonitrile;

4-(5-aminomethyl-imidazol-1-ylmethyl)-2-(2-chloro-phenoxy)-benzonitrile;

N-{3-[4-cyano-3-(2,3-dimethoxy-phenoxy)-benzyl]-3H-imidazol-4-ylmethyl}-2-cyclohexyl-acetamide;

2-(3-chloro-phenoxy)-4-[(4-chloro-phenyl)-imidazol-1-yl-methyl]-benzonitrile;

2-(3-chloro-phenoxy)-4-[1-(4-chloro-phenyl)-2-hydroxy-1-imidazol-1-yl-ethyl]-benzonitrile;

2-(3-chloro-phenoxy)-4-[(4-chloro-phenyl)-hydroxy-(3H-imidazol-4-yl)-methyl]-benzonitrile;

2-(2,4-dichloro-phenylsulfanyl)-4-[5-(2-morpholin-4-yl-ethyl)-imidazol-1-ylmethyl]-benzonitrile;

2-(2,4-dichloro-phenoxy)-4-[5-(2-morpholin-4-yl-ethyl)-imidazol-1-ylmethyl]-benzonitrile;

4-[hydroxy-(3-methyl-3H-imidazol-4-yl)-methyl]-2-(naphthalen-2-yloxy)-benzonitrile;

4-[amino-(3-methyl-3H-imidazol-4-yl)-methyl]-2-(naphthalen-2-yloxy)-benzonitrile;

4-[1-hydroxy-1-(3-methyl-3H-imidazol-4-yl)-ethyl]-2-(naphthalen-2-yloxy)-benzonitrile;

4-[1-amino-1-(3-methyl-3H-imidazol-4-yl)-ethyl]-2-(naphthalen-2-yloxy)-benzonitrile hydrochloride;

3-{2-cyano-5-[1-amino-1-(3-methyl-3H-imidazol-4-yl)-ethyl]-phenoxy}-N-ethyl-N-phenyl-benzamide;

3-{2-cyano-5-[1-hydroxy-1-(3-methyl-3H-imidazol-4-yl)-ethyl]-phenoxy}-N-ethyl-N-phenyl-benzamide;

4-[1-hydroxy-1-(3-methyl-3H-imidazol-4-yl)-ethyl]-2-(3-phenylamino-phenoxy)-benzonitrile;

4-[1-hydroxy-1-(3-methyl-3H-imidazol-4-yl)-ethyl]-2-(3-phenoxy-phenoxy)-benzonitrile;

2-(3-benzoyl-phenoxy)-4-[1-hydroxy-1-(3-methyl-3H-imidazol-4-yl)-ethyl]-benzonitrile;

2-(3-tert-butyl-phenoxy)-4-[1-hydroxy-1-(3-methyl-3H-imidazol-4-yl)-ethyl]-benzonitrile;

2-(3-diethylamino-phenoxy)-4-[1-hydroxy-1-(3-methyl-3H-imidazol-4-yl)-ethyl]-benzonitrile;

2-(5-chloro-2-oxo-2H-[1,2′]bipyridinyl-5′-ylmethoxy)-4-imidazol-1-ylmethyl-benzonitrile;

4-Imidazol-1-ylmethyl-2-[2-(2-oxo-2H-pyridin-1-yl)-phenoxy]-benzonitrile;

4-Imidazol-1-ylmethyl-2-[3-(2-oxo-2H— pyridin-1-yl)-phenoxy]-benzonitrile;

4-Imidazol-1-ylmethyl-2-[4-(2-oxo-2H— pyridin-1-yl)-phenoxy]-benzonitrile;

4-imidazol-1-ylmethyl-2-[3-(2-oxo-piperidin-1-yl)-phenoxy]-benzonitrile;

4-imidazol-1-ylmethyl-2-[4-(2-oxo-piperidin-1-yl)-phenoxy]-benzonitrile;

4-imidazol-1-ylmethyl-2-[2-(3-methyl-2-oxo-piperidin-1-yl)-phenoxy]-benzonitrile;

4-imidazol-1-ylmethyl-2-(3-morpholin-4-yl-phenoxy)-benzonitrile;

4-imidazol-1-ylmethyl-2-(3-piperidin-1-ylmethyl-phenoxy)-benzonitrile;

2-[2-(3,3-dimethyl-2-oxo-piperidin-1-yl)-phenoxy]-4-imidazol-1-ylmethyl-benzonitrile;

2-[3-(3-ethyl-1-methyl-2-oxo-azepan-3-yl)-phenoxy]-4-imidazol-1-ylmethyl-benzonitrile;

2-[3-(3-ethyl-1-methyl-2-oxo-azepan-3-yl)-phenoxy]-4-(2-methyl-imidazol-1-yl)methyl-benzonitrile;

2-[3-(3-ethyl-1-methyl-2-oxo-azepan-3-yl)-phenoxy]-4-(5-methyl-imidazol-1-yl)methyl-benzonitrile;

2-[3-(3-ethyl-1-methyl-2-oxo-azepan-3-yl)-phenoxy]-4-(2,5-dimethyl-imidazol-1-yl)methyl-benzonitrile;

2-[3-(3-ethyl-1-methyl-2-oxo-azepan-3-yl)-phenoxy]-4-[1,2,4]triazol-4-ylmethyl-benzonitrile;

2-[3-(3-ethyl-1-methyl-2-oxo-azepan-3-yl)-phenoxy]-4-[1,2,4]triazol-1-ylmethyl-benzonitrile;

4-imidazol-1-ylmethyl-2-[3-(1-methyl-2-oxo-azepan-3-yl)-phenoxy]-benzonitrile;

4-imidazol-1-ylmethyl-2-[3-(1-methyl-2-oxo-azocan-3-yl)-phenoxy]-benzonitrile;

4-imidazol-1-ylmethyl-2-[3-(1-methyl-2-oxo-piperidin-3-yl)-phenoxy]-benzonitrile;

4-imidazol-1-ylmethyl-2-[3-(3-ethyl-1-methyl-2-oxo-piperidin-3-yl)-phenoxy]-benzonitrile;

4-imidazol-1-ylmethyl-2-[3-(2-oxo-azepan-3-yl)-phenoxy]-benzonitrile;

2-[3-(3-hydroxymethyl-1-methyl-2-oxo-azepan-3-yl)-phenoxy]-4-imidazol-1-ylmethyl-benzonitrile;

2-[3-(3-cyclopropylmethyl-1-methyl-2-oxo-azepan-3-yl)-phenoxy]-4-imidazol-1-ylmethyl-benzonitrile;

2-[4-bromo-3-(3-cyclopropylmethyl-1-methyl-2-oxo-azepan-3-yl)-phenoxy]-4-imidazol-1-ylmethyl-benzonitrile;

2-[3-(3-methoxymethyl-1-methyl-2-oxo-azepan-3-yl)-phenoxy]-4-imidazol-1-ylmethyl-benzonitrile;

2-[3-(3-ethyl-2-oxo-azepan-3-yl)-phenoxy]-4-imidazol-1-ylmethyl-benzonitrile;

2-[3-(3-ethyl-azepan-3-yl)-phenoxy]-4-imidazol-1-ylmethyl-benzonitrile;

2-[3-(1-acetyl-3-ethyl-azepan-3-yl)-phenoxy]-4-imidazol-1-ylmethyl-benzonitrile;

3-[3-(2-cyano-5-imidazol-1-ylmethyl-phenoxy)-phenyl]-3-ethyl-azepane-1-carboxylic acid-tert-butyl ester;

4-[5-(2-amino-ethyl)-2-methyl-imidazol-1-ylmethyl]-2-[3-(3-ethyl-1-methyl-2-oxo-azepan-3-yl)-phenoxy]-benzonitrile;

2-[3-(3-ethyl-1-methyl-2-oxo-azepan-3-yl)-phenoxy]-4-[2-methyl-5-(2-morpholin-4-yl-ethyl)-imidazol-1-ylmethyl]-benzonitrile;

N-[2-(3-{4-cyano-3-[3-(3-ethyl-1-methyl-2-oxo-azepan-3-yl)-phenoxy]-benzyl}-2-methyl-3H-imidazol-4-yl)-ethyl]-acetamide;

3-ethyl-3-[3-(3-imidazol-1-ylmethyl-phenoxy)-phenyl]-1-methyl-azepan-2-one;

2-[3-(3-ethyl-1-methyl-2-oxo-azepan-3-yl)-phenoxy]-4-(3-methyl-3-H-imidazol-4-ylmethyl)-benzonitrile;

2-[3-(3-ethyl-1-methyl-2-oxo-azepan-3-yl)-phenoxy]-4-(3H-imidazol-4-ylmethyl)-benzonitrile;

2-[3-(3-ethyl-1-methyl-2-oxo-azepan-3-yl)-phenoxy]-4-[hydroxy-(3-methyl-3-H-imidazol-4-yl)-methyl]-benzonitrile;

4-[amino-(3-methyl-3-H-imidazol-4-yl)-methyl]-2-[3-(3-ethyl-1-methyl-2-oxo-azepan-3-yl)-phenoxy]-benzonitrile;

2-[3-(3-ethyl-1-methyl-2-oxo-azepan-3-yl)-benzyl]-4-(3-methyl-3H-imidazole-4-carbonyl)-benzonitrile;

2-[3-(3-ethyl-1-methyl-2-oxo-azepan-3-yl)-phenoxy]-4-(hydroxy-pyridin-3-yl-methyl)-benzonitrile;

2-[3-(3-ethyl-1-methyl-2-oxo-azepan-3-yl)-phenoxy]-4-pyridin-3-ylmethyl-benzonitrile;

2-[3-(3-ethyl-1-methyl-2-oxo-azepan-3-yl)-phenoxy]-4-pyridin-2-ylmethyl-benzonitrile;

2-[3-(3-ethyl-1-methyl-2-oxo-azepan-3-yl)-phenoxy]-4-[1-hydroxy-1-(3-methyl-3H-imidazol-4-yl)-ethyl]-benzonitrile;

2-[3-(3-ethyl-1-methyl-2-oxo-azepan-3-yl)-phenoxy]-4-[1-amino-1-(3-methyl-3H-imidazol-4-yl)-ethyl]-benzonitrile;

1-[1-(4′-Cyanobenzyl) imidazol-5-ylmethyl]-4-[1-phenyl-1-cyclopentylcarbonyl]piperazine;

1-[1-(4′-Cyanobenzyl) imidazol-5-ylmethyl]-4-[Cyclohexylphenylacetyl]piperazine;

1-[1-(4′-Cyanobenzyl) imidazol-5-ylmethyl]-4-[1-(3-methoxyphenyl)-1-cyclopentylcarbonyl]piperazine;

1-[1-(4′-Cyanobenzyl) imidazol-5-ylmethyl]-4-[1-(3-phenoxyphenyl)-1-cyclopentylcarbonyl]piperazine;

1-[1-(4′-Cyano-3-fluorobenzyl) imidazol-5-ylmethyl]-4-[1-(3-hydroxyphenyl)-1-cyclohexylcarbonyl]piperazine;

1-[1-(4′-Cyanobenzyl) imidazol-5-ylmethyl]piperazine-4-carboxylic acid-(2,6-dimethoxy)benzyl ester;

1-[1-(4′-Cyanobenzyl) imidazol-5-ylmethyl]piperazine-4-(DL-2-hydroxy-2-(o-methoxyphenyl)) acetamide;

1-[1-(4′-Cyanobenzyl) imidazol-5-ylmethyl]-4-[1-(2,6-dimethylbenzyloxycarbonyl]piperazine;

1-[1-(4′-Cyanobenzyl) imidazol-5-ylmethyl]-4-[1-(2-methoxyphenyl)-1-cyclopentylcarbonyl]piperazine;

(+/−) 1-[1-(4′-Cyanobenzyl) imidazol-5-ylmethyl]-4-[1-(bicyclo[3.1.0]hex-3-yl)-1-(3-methoxyphenyl)-carbonyl]piperazine;

(R/S) 2 [4-((Phenyl)methyloxycarbonyl-1-piperazine)]-2-[1-(4′-cyanobenzyl)-2-methyl-5-imidazol]acetonitrile;

1-[1-(4′-methylbenzyl) imidazol-5-ylmethyl]-4-[1-(2,6-dimethylbenzyloxycarbonyl]piperazine;

1-[1-(4′-Cyanobenzyl) imidazol-5-ylmethyl]piperazine-4-carboxylic acid-(4-nitro)phenyl ester;

1-[1-(4-Cyanobenzyl) imidazol-5-ylmethyl]-4-[3-(4-fluorophenyl)-3-(tricyclo[3.3.1.1^{3 7}]dec-2-yl)-propionyl]piperazine;

2-(1-(4′-cyanobenzyl)imidazol-5-yl-2-[4-(phenylmethyloxy carbonyl)piperazin-1-yl]acetamide;

1-[1-(4′-cyanobenzyl) imidazol-5-ylmethyl]-4-[1-(2-methoxy-5-chlorobenzyloxycarbonyl]piperazine;

1-[1-(4′-cyanobenzyl) imidazol-5-ylmethyl]-4-[1-(pentafluororobenzyloxycarbonyl]piperazine;

1-[1-(4′-cyanobenzyl) imidazol-5-ylmethyl]-4-[1-(2-ethoxybenzyloxycarbonyl]piperazine;

1-[1-(4′-cyanobenzyl) imidazol-5-ylmethyl]-4-{1-[(2-methoxypyridin-3-yl)methyloxycarbonyl]}piperazine;

1-[1-(4′-cyanobenzyl) imidazol-5-ylmethyl]-4-[1-(2-trifluoromethoxybenzyloxycarbonyl]piperazine;

1-[1-(4′-cyanobenzyl) imidazol-5-ylmethyl]-4-[1-(2,3-methylenedioxybenzyloxycarbonyl]piperazine;

1-[1-(4′-Cyanobenzyl) imidazol-5-ylmethyl]piperazine-4-carboxylic acid benzyl ester;

1-[1-(4′-Cyanobenzyl) imidazol-5-ylmethyl]-piperazine-3-carboxylic acid-4-carboxylic acid benzyl ester;

1-[1-(4′-Cyanobenzyl) imidazol-5-ylmethyl]-3-methyl carboxy-piperazine-4-carboxylic acid benzyl ester;

1-[1-(4′-Cyanobenzyl) imidazol-5-ylmethyl]piperazine-4-(N-3-isopropenyl-1,1-dimethylbenzyl)carboxamide;

1-[(1-(4′-cyanobenzyl) imidazol-5-ylmethyl]-4-phenylmethanesulfonyl-(cis)-2,6-dimethylpiperazine;

2-((4′-cyanobenzyl)-5-imidazolyl))-2-[(4′-phenylmethyloxycarbonyl) piperazin-1′-yl]acetonitrile;

1-[1-(4′-Cyanobenzyl) imidazol-5-ylmethyl]-4-(2-tert-butyl-3-phenyl)propionyl piperazine;

1-[1-(4′-Cyanobenzyl) imidazol-5-ylmethyl]-4-[1-(4-methoxyphenyl)-1-cyclohexyl]carbonyl piperazine;

1-[1-1-(4′-Cyanobenzyl) imidazol-5-ylmethyl]-4-{1-[(2-ethoxypyridin-3-yl)methyloxycarbonyl]piperazine;

1-[1-(4′-Cyanobenzyl) imidazol-5-ylmethyl]-4-[1-(2-methanesulfonylbenzyloxycarbonyl) piperazine;

1-[1-(4′-Cyanobenzyl) imidazol-5-yl)-2-(ethoxybenzyl)]piperazine-4-carbamide;

[1-((1(4′-Cyanobenzyl)-2-methyl)imidazol-5-yl)-4-(benzyloxycarbonyl)]piperazine;

1-[1-(4′-Cyanobenzyl) imidazol-5-ylmethyl]piperazine-4-(N-3-methylbenzyl)carboxamide;

1-[1-(4′-Cyanobenzyl) imidazol-5-ylmethyl]piperazine-4-(N-2-chlorobenzyl)carboxamide;

1-[1-(4′-Cyanobenzyl)imidazol-5-yl)-(2-methoxybenzyl)]piperazine-4-carboxamide;

1-[1-(4-Cyanobenzyl)imidazol-5-yl)-(3-methoxy-6-chlorobenzyl)]piperazine-4-carboxamide;

1-[1-(4′-Cyanobenzyl)imidazol-5-yl)-(2-methyl-5-chlorobenzyl)]piperazine-4-carboxamide;

1-[1-(4′-Cyanobenzyl)imidazol-5-yl)-(3-phenylpropyl)]piperazine-4-carboxamide;

1-[1-(4′-Cyanobenzyl)imidazol-5-yl)-(2,5-dimethylbenzyl)]piperazine-4-carbamide;

1-[1-(4′-Cyanobenzyl)imidazole-5-ylmethyl]-4-benzyloxycarbonyl)-(trans)-2,5-dimethylpiperazine;

1-[(4-cyanophenyl)methylimidazol-5-ylmethyl]piperazine-4-2,4-dimethylbenzyloxycarbonyl;

1-[(4-cyanophenyl)methylimidazol-5-ylmethyl]piperazine-4-(2-methylbenzyloxycarbonyl);

1-[(4-cyanophenyl)methylimidazol-5-ylmethyl]piperazine-4-(4′-acetamidobenzyloxycarbonyl);

1-[(4-cyanophenyl)methylimidazol-5-ylmethyl]piperazine-4-[(3′-methylbenzyloxycarbonyl);

1-[(4-cyanophenyl)methylimidazol-5-ylmethyl]piperazine-4-(2′-methoxybenzyloxycarbonyl);

1-[(4-cyanophenyl)methylimidazol-5-ylmethyl]piperazine-4-(3′-methoxybenzyloxycarbonyl);

1-[(4-cyanophenyl)methylimidazol-5-ylmethyl]piperazine-4-(1-oxypyridine-3-methyloxycarbonyl);

1-[(4-cyanophenyl)methylimidazol-5-ylmethyl]piperazine-4-(3-pyridinemethyloxycarbonyl);

1-[(4-cyanophenyl)methylimidazol-5-ylmethyl]piperazine-4-(4′-pyridinemethyloxycarbonyl);

1-[(4-cyanophenyl)methylimidazol-5-ylmethyl]piperazine-4-(2′,5′-dimethylbenzyloxycarbonyl);

1-[(4-cyanophenyl)methylimidazol-5-ylmethyl]piperazine-4-[(1,3-benzodioxolan-5-methyl)oxycarbonyl];

or a pharmaceutically acceptable salt or optical isomer thereof.

Compounds which are described as inhibitors of farnesyl-protein transferase and may therefore useful in the present invention, and methods of synthesis thereof, can be found in the following patents, pending applications and publications, which are herein incorporated by reference:

WO 95/32987 published on Dec. 7, 1995;

U.S. Pat. No. 5,420,245;

U.S. Pat. No. 5,523,430;

U.S. Pat. No. 5,532,359;

U.S. Pat. No. 5,510,510;

U.S. Pat. No. 5,589,485

U.S. Pat. No. 5,602,098;

European Pat. Publ. 0 618 221;

European Pat. Publ. 0 675 112;

European Pat. Publ. 0 604 181;

European Pat. Publ. 0 696 593;

WO 94/19357;

WO 95/08542;

WO 95/11917;

WO 95/12612;

WO 95/12572;

WO 95/10514 and U.S. Pat. No. 5,661,152;

WO 95/10515;

WO 95/10516;

WO 95/24612;

WO 95/34535;

WO 95/25086;

WO 96/05529;

WO 96/06138;

WO 96/06193;

WO 96/16443;

WO 96/21701;

WO 96/21456;

WO 96/22278;

WO 96/24611;

WO 96/24612;

WO 96/05168;

WO 96/05169;

WO 96/00736 and U.S. Pat. No. 5,571,792 granted on Nov. 5, 1996;

WO 96/17861;

WO 96/33159;

WO 96/34850;

WO 96/34851;

WO 96/30017;

WO 96/30018;

WO 96/30362;

WO 96/30363;

WO 96/31111;

WO 96/31477;

WO 96/31478;

WO 96/31501;

WO 97/00252;

WO 97/03047;

WO 97/03050;

WO 97/04785;

WO 97/02920;

WO 97/17070;

WO 97/23478;

WO 97/26246;

WO 97/30053;

WO 97/44350;

WO 97/43437;

WO 97/49700;

WO 98/00409;

WO 98/00411;

WO 98/02436;

WO 98/04545;

WO 98/09641;

WO 98/07692;

WO 98/11091;

WO 98/11092;

WO 98/11093;

WO 98/11096;

WO 98/11097;

WO 98/11098;

WO 98/11099;

WO 98/11100;

WO 98/11106;

WO 98/15556;

WO 98/17629;

WO 98/20001;

WO 98/27109;

WO 98/29390;

WO 98/30558;

WO 98/32741;

WO 98/34921;

WO 98/38162;

GB 2323841;

GB 2323842;

GB 2323783;

WO 98/40383;

WO 98/42676;

WO 98/46625;

WO 98/49157;

WO 98/50029;

WO 98/50030;

WO 98/50031;

EP 810223;

KR 97/006208; and

U.S. Pat. No. 5,532,359 granted on Jul. 2, 1996.

The following compounds which are inhibitors of farnesyl-protein transferase are particularly useful in the methods of treatment described herein:

(+)-6-[amino(4-chlorophenyl)(1-methyl-1H-imidazol-5-yl)methyl]-4-(3-chlorophenyl)-1-methyl-2(1H)-quinolinone (Compound J)

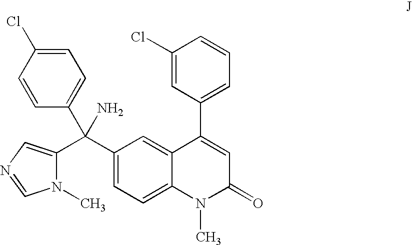

(−)-6-[amino(4-chlorophenyl)(1-methyl-1H-imidazol-5-yl)methyl]-4-(3-chlorophenyl)-1-methyl-2(1H)-quinolinone (Compound J-A; designated “comp. 74” in WO 97/21701)

(+)-6-[amino(4-chlorophenyl)(1-methyl-1H-imidazol-5-yl)methyl]-4-(3-chlorophenyl)-1-methyl-2(1H)-quinolinone (Compound J-B; designated “comp. 75” in WO 97/21701)

or a pharmaceutically acceptable salt thereof. The syntheses of these compounds are specifically described in PCT Publication WO 97/21701, in particular on pages 19-28. The preferred compound among these compounds to use in combination with a PSA conjugate is Compound J-B.

The following compound which is an inhibitor of farnesyl-protein transferase is particularly useful in the methods of treatment described herein:

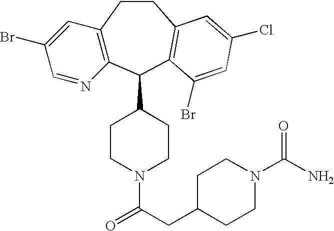

or a pharmaceutically acceptable salt thereof. The synthesis of this compound is specifically described in PCT Publication WO 97/23478, in particular on pages 18-56. In WO 97/23478, the above compound is designated compound “39.0” and is specifically described in Example 10.

Compounds which are inhibitors of farnesyl-protein transferase and are therefore useful in the present invention, and methods of synthesis thereof, can be found in the following patents, pending applications and publications, which are herein incorporated by reference:

U.S. Pat. No. 5,238,922 granted on Aug. 24, 1993;

U.S. Pat. No. 5,340,828 granted on Aug. 23, 1994;

U.S. Pat. No. 5,480,893 granted on Jan. 2, 1996;

U.S. Pat. No. 5,352,705 granted on Oct. 4, 1994;

U.S. Pat. No. 5,504,115 granted on Apr. 2, 1996;

U.S. Pat. No. 5,536,750 granted on Jul. 16, 1996;

U.S. Pat. No. 5,504,212 granted on Apr. 2, 1996;

U.S. Pat. No. 5,439,918 granted on Aug. 8, 1995;

U.S. Pat. No. 5,686,472 granted on Nov. 11, 1997;

U.S. Pat. No. 5,736,539 granted on Apr. 4, 1998;

U.S. Pat. No. 5,576,293 granted on Nov. 19, 1996;

U.S. Pat. No. 5,468,733 granted on Nov. 21, 1995;

WO 96/06609 (Mar. 3, 1996) and U.S. Ser. No. 08/298,478 filed on Aug. 24, 1994;

U.S. Pat. No. 5,585,359 granted on Dec. 17, 1996;

U.S. Pat. No. 5,523,456 granted on Jun. 4, 1996;

U.S. Pat. No. 5,661,161 granted on Aug. 26, 1997;

U.S. Pat. No. 5,571,835 granted on Nov. 5, 1996;

U.S. Pat. No. 5,491,164 granted on Feb. 13, 1996;

U.S. Pat. No. 5,652,257 granted on Jul. 29, 1997;

U.S. Pat. No. 5,631,280 granted on May 20, 1997;

U.S. Pat. No. 5,578,629 granted on Nov. 26, 1996;

U.S. Pat. No. 5,627,202 granted on May 6, 1997;

U.S. Pat. No. 5,856,326 granted on Jan. 5, 1999; WO 96/30343 (Oct. 3, 1996);

U.S. Pat. No. 5,624,936 granted on Apr. 29, 1997; U.S. Pat. No. 5,534,537 granted on Jul. 9, 1996;

U.S. Pat. No. 5,710,171 granted on Apr. 29, 1997;

WO 96/39137 (Dec. 12, 1996); U.S. Ser. No. 08/468,160 filed on Jun. 6, 1995; U.S. Ser. No. 08/652,055 filed on May 23, 1996; U.S. Ser. No. 08/960,248 filed Oct. 29, 1997;

U.S. Pat. No. 5,703,241 granted on Dec. 30, 1997;

WO 97/18813; U.S. Ser. No. 08/749,254 filed on Nov. 15, 1996;

WO 97/27854 (Aug. 7, 1997); U.S. S No. 60/010,799 filed on Jan. 30, 1996; U.S. Ser. No. 08/786,520 filed on Jan. 21, 1997; U.S. Ser. No. 09/015,823 filed on Jan. 29, 1998;

WO 97/27752 (Aug. 7, 1997); U.S. S No. 60/010,860 filed on Jan. 30, 1996; U.S. Ser. No. 08/784,556 filed on Jan. 21, 1997; U.S. Ser. No. 09/030,223 filed on Feb. 25, 1998;

WO 97/27853 (Aug. 7, 1997); U.S. S No. 60/011,081 filed on Jan. 30, 1996; U.S. Ser. No. 08/786,519 filed on Jan. 21, 1997;

WO 97/27852 (Aug. 7, 1997); U.S. S No. 60/010,798 filed on Jan. 30, 1996; U.S. Ser. No. 08/786,516 filed on Jan. 21, 1997;

WO 97/36888 (Oct. 9, 1997); U.S. S No. 60/014,587 filed on Apr. 3, 1996; U.S. Ser. No. 08/823,919 filed on Mar. 25, 1997;

WO 97/36889 (Oct. 9, 1997); U.S. S No. 60/014,589 filed on Apr. 3, 1996; U.S. Ser. No. 08/823,923 filed on Mar. 25, 1997;

WO 97/36876 (Oct. 9, 1997); U.S. S No. 60/014,592 filed on Apr. 3, 1996; U.S. Ser. No. 08/834,671 filed on Apr. 1, 1997;

WO 97/36593 (Oct. 9, 1997); U.S. S No. 60/014,593 filed on Apr. 3, 1996; U.S. Ser. No. 08/827,485, filed on Mar. 27, 1997;

WO 97/36879 (Oct. 9, 1997); U.S. S No. 60/014,594 filed on Apr. 3, 1996; U.S. Ser. No. 08/823,920 filed on Mar. 25, 1997;

WO 97/36583 (Oct. 9, 1997); U.S. S No. 60/014,668 filed on Apr. 3, 1996; U.S. Ser. No. 08/824,588 filed on Mar. 26, 1997;

WO 97/36592 (Oct. 9, 1997); U.S. S No. 60/014,775 filed on Apr. 3, 1996; U.S. Ser. No. 08/826,292 filed on Mar. 27, 1997;

WO 97/36584 (Oct. 9, 1997); U.S. S No. 60/014,776 filed on Apr. 3, 1996; U.S. Ser. No. 08/824,427 filed on Mar. 26, 1997; U.S. S No. 60/014,777 filed on Apr. 3, 1996; U.S. Ser. No. 08/826,317 filed on Mar. 27, 1997;

WO 97/38665 (Oct. 23, 1997); U.S. S No. 60/014,791 filed on Apr. 3, 1996; U.S. Ser. No. 08/831,308 filed on Apr. 1, 1997;

WO 97/36591 (Oct. 9, 1997); U.S. S No. 60/014,792 filed on Apr. 3, 1996; U.S. Ser. No. 08/827,482, filed on Mar. 27, 1997;

WO 97/36605 (Oct. 9, 1997); U.S. S No. 60/014,793 filed on Apr. 3, 1996; U.S. Ser. No. 08/823,934 filed on Mar. 25, 1997;

WO 97/37877 (Oct. 9, 1997); U.S. S No. 60/014,794 filed on Apr. 3, 1996; U.S. Ser. No. 08/834,675 filed on Apr. 1, 1997;

WO 97/37900 (Oct. 9, 1997); U.S. S No. 60/014,798 filed on Apr. 3, 1996; U.S. Ser. No. 08/823,929 filed on Mar. 25, 1997;

WO 97/36891 (Oct. 9, 1997); U.S. S No. 60/014,774 filed on Apr. 3, 1996; U.S. Ser. No. 08/826,291 filed on Mar. 27, 1997;

WO 97/36886 (Oct. 9, 1997); U.S. S No. 60/022,332 filed on Jul. 24, 1996; U.S. Ser. No. 08/823,919, filed on Mar. 27, 1997;

WO 97/36881 (Oct. 9, 1997); U.S. S No. 60/022,340 filed on Jul. 24, 1996; U.S. Ser. No. 08/827,486, filed on Mar. 27, 1997;

WO 97/36585 (Oct. 9, 1997); U.S. S No. 60/022,341 filed on Jul. 24, 1996; U.S. Ser. No. 08/826,251 filed on Mar. 27, 1997;

WO 97/36898 (Oct. 9, 1997); U.S. S No. 60/022,342 filed on Jul. 24, 1996; U.S. Ser. No. 08/825,293 filed on Mar. 27, 1997;

WO 97/36897 (Oct. 9, 1997); U.S. S No. 60/022,558 filed on Jul. 24, 1996; U.S. Ser. No. 08/827,476, filed on Mar. 27, 1997;

WO 97/36874 (Oct. 9, 1997);

WO 97/36585 (Oct. 9, 1997); U.S. S No. 60/022,586 filed on Jul. 24, 1996, U.S. Ser. No. 08/827,484, filed on Mar. 27, 1997;

WO 97/36890 (Oct. 9, 1997); U.S. S No. 60/022,587 filed on Jul. 24, 1996; U.S. Ser. No. 08/831,105 filed on Apr. 1, 1997;

WO 97/36901 (Oct. 9, 1997); U.S. S No. 60/022,647 filed on Jul. 24, 1996; U.S. Ser. No. 08/827,483, filed on Mar. 27, 1997;

U.S. S No. 60/032,126 filed on Dec. 5, 1996; U.S. Ser. No. 08/985,732, filed on Dec. 4, 1997;

U.S. S No. 60/032,428 filed on Dec. 5, 1996; U.S. Ser. No. 08/985,124, filed on Dec. 4, 1997;

U.S. S No. 60/032,578 filed on Dec. 5, 1996; U.S. Ser. No. 08/985,337, filed on Dec. 4, 1997;

U.S. S No. 60/032,579 filed on Dec. 5, 1996; U.S. Ser. No. 08/985,320, filed on Dec. 5, 1997;

U.S. S No. 60/033,990, filed on Dec. 30, 1996; U.S. Ser. No. 08/995,744, filed on Dec. 22, 1997;

U.S. S No. 60/033,991, filed on Dec. 30, 1996; U.S. Ser. No. 08/985,124, filed on Dec. 5, 1997;

U.S. S No. 60/057,097, filed on Aug. 27, 1997; U.S. Ser. No. 09/140,919, filed on Aug. 26, 1998;

U.S. S No. 60/057,080, filed on Aug. 27, 1997; U.S. Ser. No. 09/140,584, filed on Aug. 26, 1998;

U.S. S No. 60/062,660, filed on Oct. 8, 1997; U.S. Ser. No. 09/167,180, filed on Oct. 6, 1998;

U.S. S No. 60/064,342, filed on Oct. 17, 1997; U.S. Ser. No. 08/, filed on Oct. 13, 1998;

U.S. S No. 60/091,629, filed on Jul. 2, 1998;

U.S. S No. 60/091,596, filed on Jul. 2, 1998;

U.S. S No. 60/091,513, filed on Jul. 2, 1998;

U.S. S No. 60/122,968, (Case 20288PV) filed on Mar. 3, 1999;

U.S. S No. 60/122,970, filed on Mar. 3, 1999;

U.S. S No. 60/122,768, filed on Mar. 3, 1999;

U.S. S No. 60/122,771, filed on Mar. 3, 1999; and

U.S. S No. 60/123,620, filed on Mar. 3, 1999.

PSA conjugates that are useful in the methods of the instant invention and are identified by the properties described hereinabove include:

a) a compound represented by the formula IX:

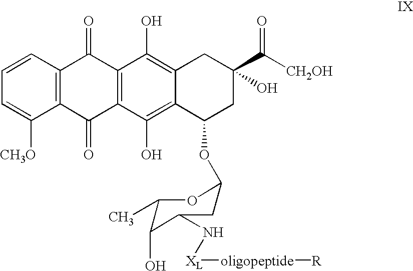

wherein:

oligopeptide is an oligopeptide which is specifically recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine;

R is hydrogen or —(C═O)R¹; and

R¹ is C₁-C₆-alkyl or aryl,

or the pharmaceutically acceptable salt thereof;

b) a compound represented by the formula X:

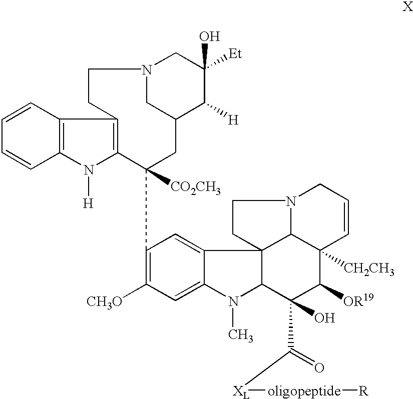

wherein:

oligopeptide is an oligopeptide which is specifically recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine; or

X_{L} is —NH—(CH₂)ₙ—NH—

R is hydrogen or —(C═O)R¹;

R¹ is C₁-C₆-alkyl or aryl;

R¹⁹ is hydrogen or acetyl; and

n is 1, 2, 3, 4 or 5,

or the pharmaceutically acceptable salt thereof;

c) a compound represented by the formula XI:

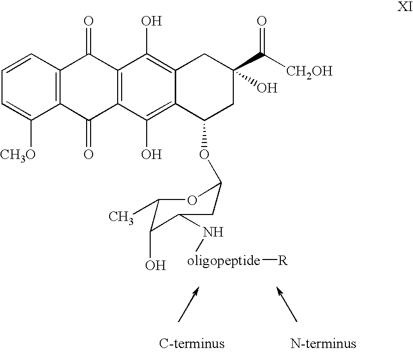

wherein:

oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula:

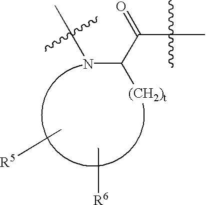

and wherein

the C-terminus carbonyl is covalently bound to the amine of doxorubicin;

R is selected from

a) hydrogen,

b) —(C═O)R^{1a},

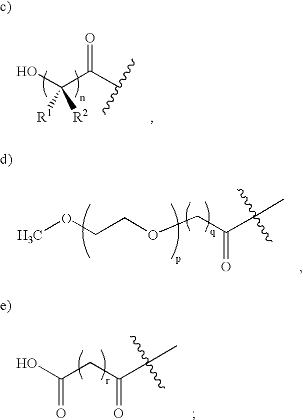

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl;

R⁵ is selected from HO— and C₁-C₆ alkoxy;

R⁶ is selected from hydrogen, halogen, C₁-C₆ alkyl, HO— and C₁-C₆ alkoxy; and

n is 1, 2, 3 or 4;

p is zero or an integer between 1 and 100;

q is 0 or 1, provided that if p is zero, q is 1;

r is an integer between 1 and 10; and

t is 3 or 4;

or a pharmaceutically acceptable salt thereof;

d) a compound represented by the formula X:

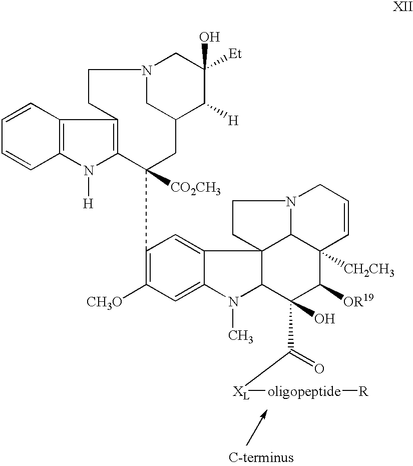

wherein:

oligopeptide is an oligopeptide which is specifically recognized by the free prostate specific antigen (PSA) and is capable of being proteolytically cleaved by the enzymatic activity of the free prostate specific antigen, and the oligopeptide comprises a cyclic amino acid of the formula:

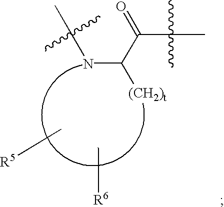

XL is —NH—(CH₂)ᵤ—NH—

R is selected from

a) hydrogen,

b) —(C═O)R^{1a},

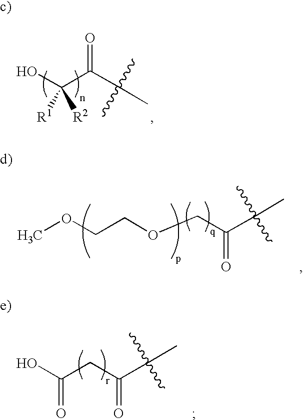

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl,

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is 0 or 1, provided that if p is zero, q is 1;

r is 1, 2 or 3;

t is 3 or 4;

u is 1, 2, 3, 4 or 5,

or the pharmaceutically acceptable salt thereof;

e) a compound represented by the formula XI:

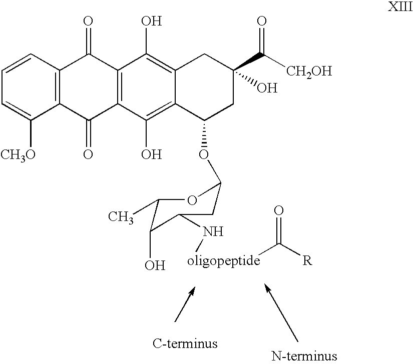

wherein:

oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;

R is selected from

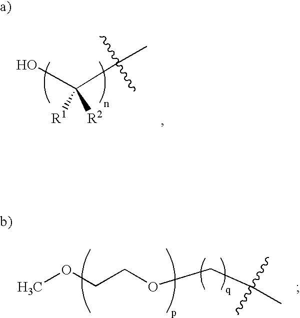

R¹ and R² are independently selected from: hydrogen, OH, C₁-C₆ alkyl, C₁-C₆ alkoxy, C₁-C₆ aralkyl and aryl;

n is 1, 2, 3 or 4;

p is zero or an integer between 1 and 100;

q is 0 or 1, provided that if p is zero, q is 1;

or the pharmaceutically acceptable salt thereof;

f) a compound represented by the formula XIV:

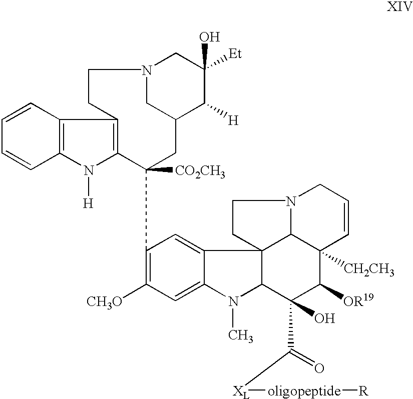

wherein:

oligopeptide is an oligopeptide which is specifically recognized by the free prostate specific antigen (PSA) and is capable of being proteolytically cleaved by the enzymatic activity of the free prostate specific antigen;

X_{L} is —NH—(CH₂)ᵣ—NH—

R is selected from

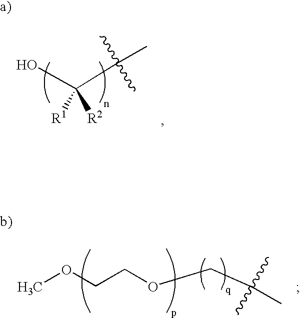

R¹ and R² are independently selected from: hydrogen, OH, C₁-C₆ alkyl, C₁-C₆ alkoxy, C₁-C₆ aralkyl and aryl;

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is 0 or 1, provided that if p is zero, q is 1;

r is 1, 2, 3, 4 or 5,

or the pharmaceutically acceptable salt thereof;

g) a compound represented by the formula XV:

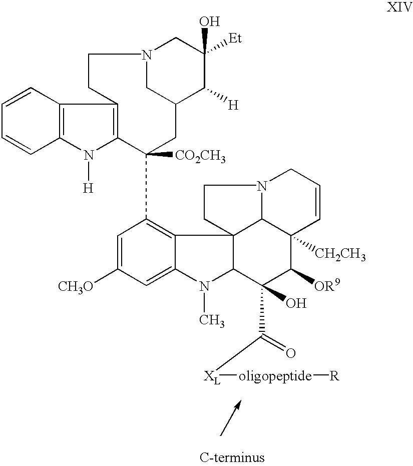

wherein:

oligopeptide is an oligopeptide which is specifically recognized by the free prostate specific antigen (PSA) and is capable of being proteolytically cleaved by the enzymatic activity of the free prostate specific antigen,

X_{L} is —NH—(CH₂)ᵤ—W—(CH₂)ᵤ—NH—

R is selected from

a) hydrogen,

b) —(C═O)R^{1a},

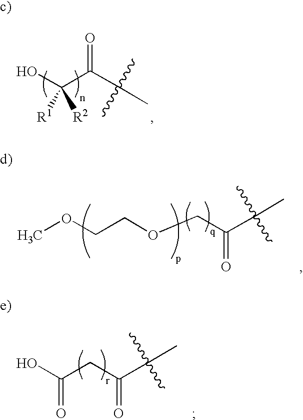

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

R⁹ is hydrogen, (C₁-C₃ alkyl)-CO, or chlorosubstituted (C₁-C₃ alkyl)-CO;

W is selected from cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3,

or the pharmaceutically acceptable salt thereof; and

h) a compound represented by the formula XVI:

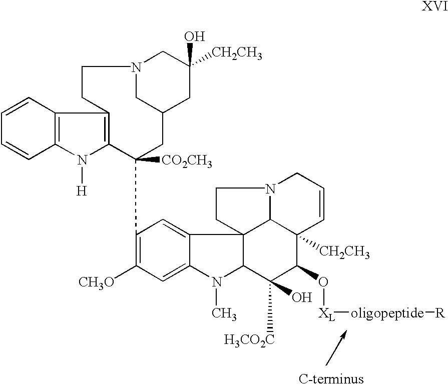

wherein:

oligopeptide is an oligopeptide which is specifically recognized by the free prostate specific antigen (PSA) and is capable of being proteolytically cleaved by the enzymatic activity of the free prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R is selected from

a) hydrogen,

b) —(C═O)R¹ a

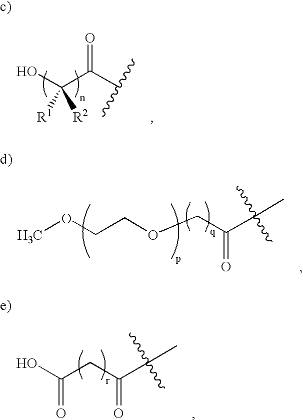

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

R⁹ is hydrogen, (C₁-C₃ alkyl)-CO, or chlorosubstituted (C₁-C₃ alkyl)-CO;

W is selected from a branched or straight chain C₁-C₆-alkyl, cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3;

or the pharmaceutically acceptable salt or optical isomer thereof.

Examples of compounds which are PSA conjugates include the following:

i)

i)

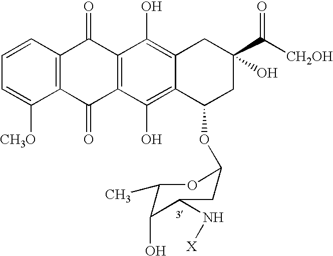

| | |
|---|---|
| wherein X is: | |
| AsnLysIleSerTyrGlnSer- | (SEQ.ID.NO.: 14), |
| AsnLysIleSerTyrGlnSerSer- | (SEQ.ID.NO.: 15), |
| AsnLysIleSerTyrGlnSerSerSer- | (SEQ.ID.NO.: 16), |
| AsnLysIleSerTyrGlnSerSerSerThr- | (SEQ.ID.NO.: 17), |
| AsnLysIleSerTyrGlnSerSerSerThrGlu- | (SEQ.ID.NO.: 18), |
| AlaAsnLysIleSerTyrGlnSerSerSerThrGlu- | (SEQ.ID.NO.: 19), |
| Ac-AlaAsnLysIleSerTyrGlnSerSerSerThr- | (SEQ.ID.NO.: 20), |
| Ac-AlaAsnLysIleSerTyrGlnSerSerSerThrLeu- | (SEQ.ID.NO.: 21), |
| Ac-AlaAsnLysAlaSerTyrGlnSerAlaSerThrLeu- | (SEQ.ID.NO.: 22), |
| Ac-AlaAsnLysAlaSerlyrGlnSerAlaSerLeu- | (SEQ.ID.NO.: 23), |
| Ac-AlaAsnLysAlaSerTyrGlnSerSerSerLeu- | (SEQ.ID.NO.: 24), |
| Ac-AlaAsnLysAlaSerTyrGlnSerSerLeu- | (SEQ.ID.NO.: 25), |
| Ac-SerTyrGlnSerSerSerLeu- | (SEQ.ID.NO.: 26), |
| Ac-hArgTyrGlnSerSerSerLeu- | (SEQ.ID.NO.: 27). |
| Ac-LysTyrGlnSerSerSerLeu- | (SEQ.ID.NO.: 28), |
| Ac-LysTyrGlnSerSerNle- | (SEQ.ID.NO.: 29), |
| ii) | |
| 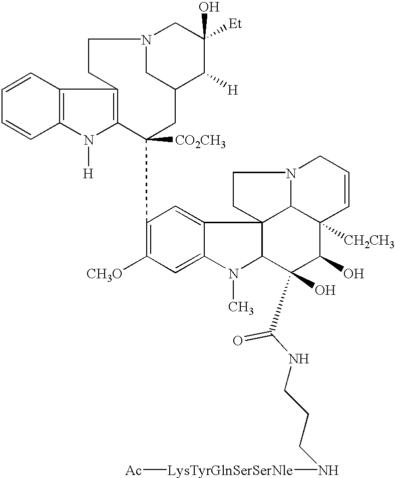 | (SEQ.ID.NO.: 30), |
| 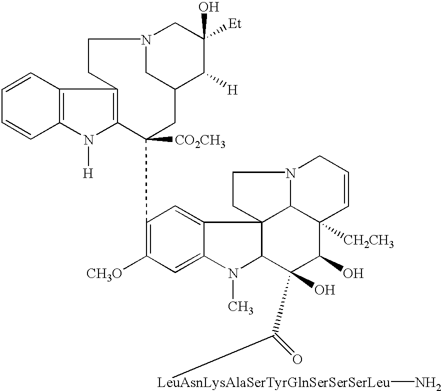 | (SEQ.ID.NO.: 31), |
| iii) | |
| 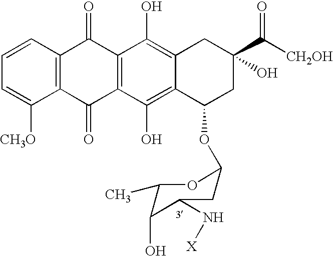 | |
| wherein X is: | |
| 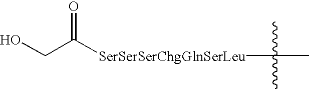 | (SEQ.ID.NO.: 32), |
| 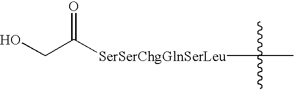 | (SEQ.ID.NO.: 33), |
| 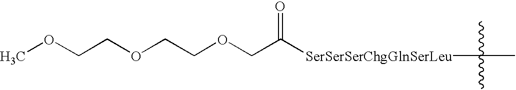 | (SEQ.ID.NO.: 34), |
| iv) | |
| 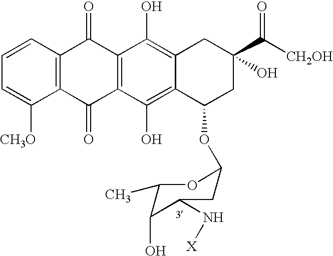 | |
| wherein X is: | |
| 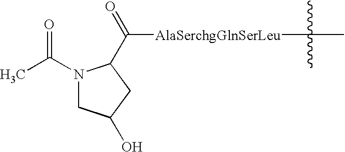 | (SEQ.ID.NO. 35,) |
| 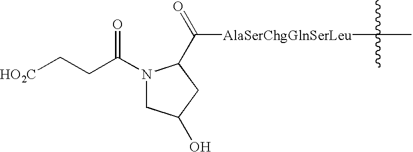 | (SEQ.ID.NO. 36,) |
| 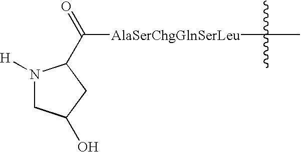 | (SEQ.ID.NO.: 37), |
| 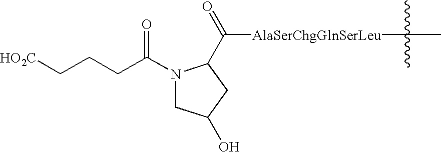 | (SEQ.ID.NO.: 38), |
| 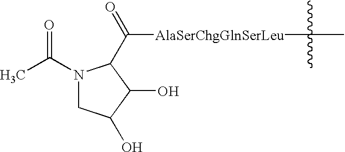 | (SEQ.ID.NO.: 39), |
| 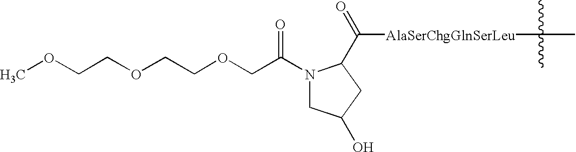 | (SEQ.ID.NO.: 40), |
| 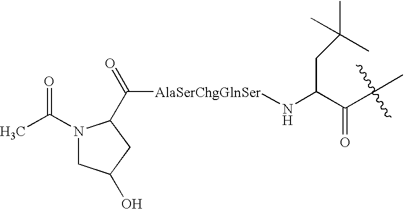 | (SEQ.ID.NO.: 41), |
| v) | |
| 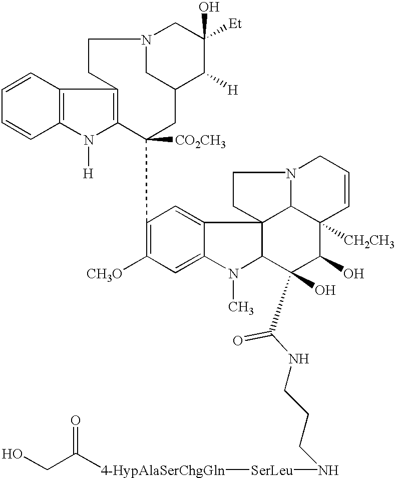 | (SEQ.ID.NO.: 42), |
| 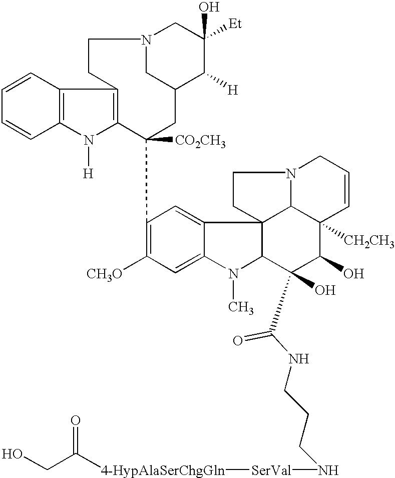 | (SEQ.ID.NO.: 43), |
| 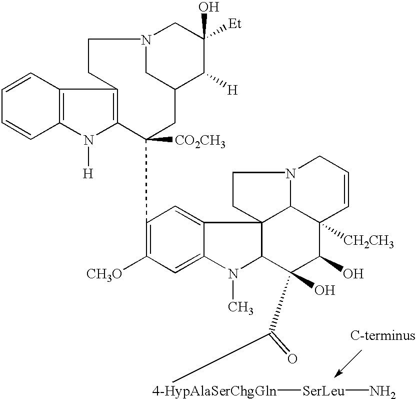 | (SEQ.ID.NO.: 44), |
| vi) | |
| 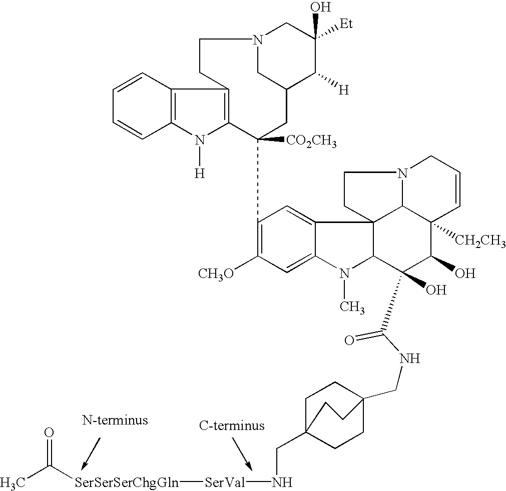 | (SEQ.ID.NO.: 45), |
| 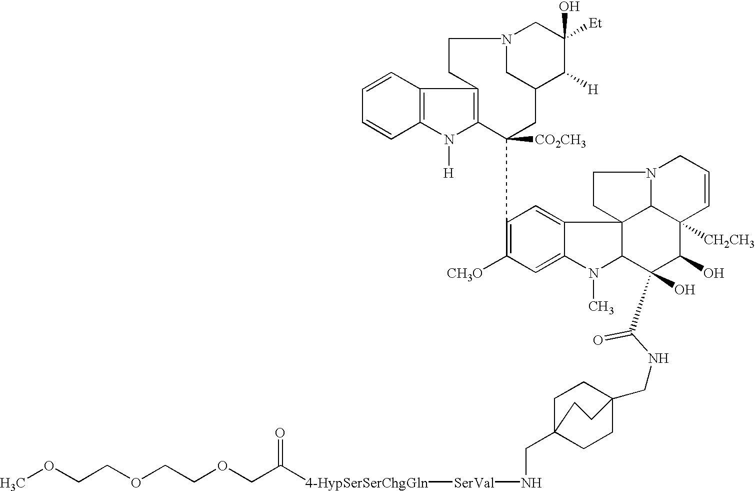 | (SEQ.ID.NO.: 46), |
| 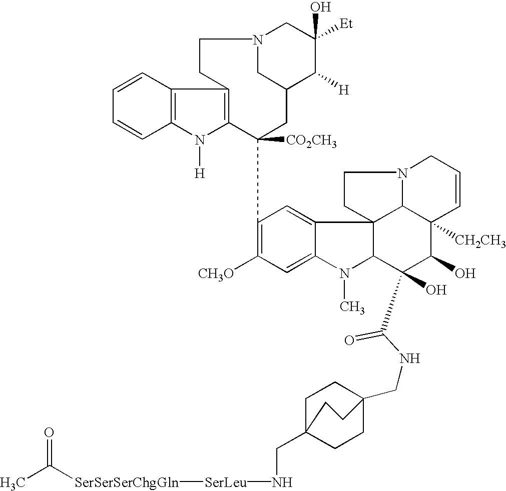 | (SEQ.ID.NO.: 47), |
| 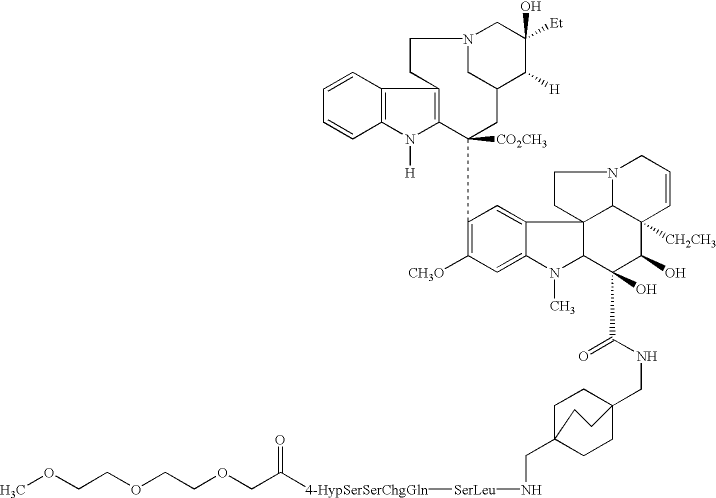 | (SEQ.ID.NO.: 48), |
| vii) | |
| 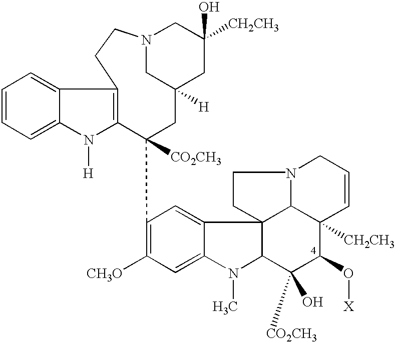 | |
| wherein X is | |
| 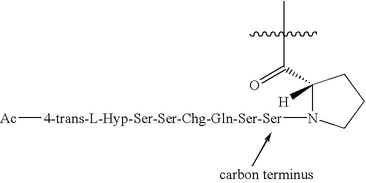 | (SEQ.ID.NO.: 49) |
| 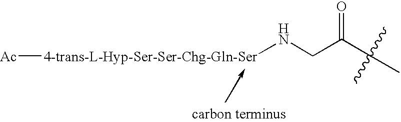 | (SEQ.ID.NO.: 50) |
| 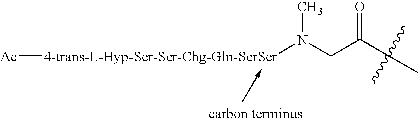 | (SEQ.ID.NO.: 51) |
| 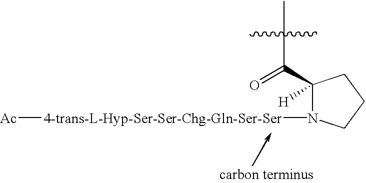 | (SEQ.ID.NO.: 52) |
| 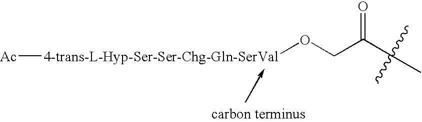 | (SEQ.ID.NO.: 53) |
| 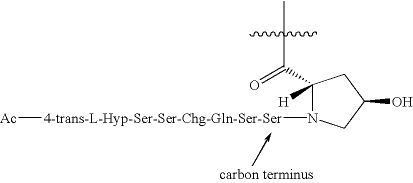 | (SEQ.ID.NO.: 54) |
| 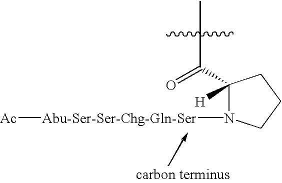 | (SEQ.ID.NO.: 55) |
| 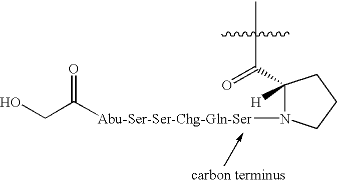 | (SEQ.ID.NO.: 56) |
| 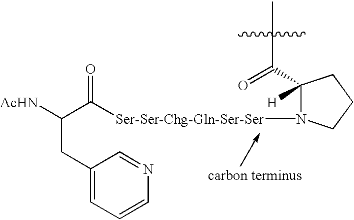 | (SEQ.ID.NO.: 57) |
| 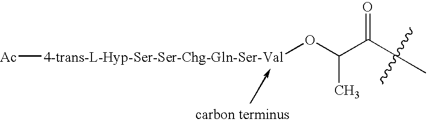 | (SEQ.ID.NO.: 58) |
| 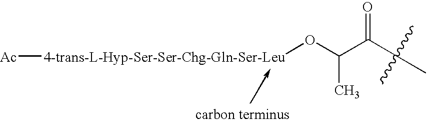 | (SEQ.ID.NO.: 59) |

or the pharmaceutically acceptable salt or optical isomer thereof.

Compounds which are PSA conjugates and are therefore useful in the present invention, and methods of synthesis thereof, can be found in the following patents, pending applications and publications, which are herein incorporated by reference:

U.S. Pat. No. 5,599,686 granted on Feb. 4, 1997;

WO 96/00503 (Jan. 11, 1996); U.S. Ser. No. 08/404,833 filed on Mar. 15, 1995; U.S. Ser. No. 08/468,161 filed on Jun. 6, 1995;

U.S. Pat. No. 5,866,679 granted on Feb. 2, 1999;

WO 98/10651 (Mar. 19, 1998); U.S. Ser. No. 08/926,412 filed on Sep. 9, 1997;

WO 98/18493 (May 7, 1998); U.S. Pat. No. 5,948,750 granted on Sep. 7, 1999;

U.S. Ser. No. 09/112,656 filed on Jul. 9, 1998; U.S. S No. 60/052,195 filed on Jul. 10, 1997; and

U.S. Ser. No. 09/193,365 filed on Nov. 17, 1998; U.S. S No. 60/067,110 filed on Dec. 2, 1997.

Compounds which are described as prodrugs wherein the active therapeutic agent is release by the action of enzymatically active PSA and therefore may be useful in the present invention, and methods of synthesis thereof, can be found in the following patents, pending applications and publications, which are herein incorporated by reference:

WO 98/52966 (Nov. 26, 1998).

All patents, publications and pending patent applications identified are hereby incorporated by reference.

With respect to the compounds of formulas I-a through VI and VIIIA the following definitions apply:

The term “alkyl” refers to a monovalent alkane (hydrocarbon) derived radical containing from 1 to 15 carbon atoms unless otherwise defined. It may be straight, branched or cyclic. Preferred straight or branched alkyl groups include methyl, ethyl, propyl, isopropyl, butyl and t-butyl. Preferred cycloalkyl groups include cyclopentyl and cyclohexyl.

When substituted alkyl is present, this refers to a straight, branched or cyclic alkyl group as defined above, substituted with 1-3 groups as defined with respect to each variable.

Heteroalkyl refers to an alkyl group having from 2-15 carbon atoms, and interrupted by from 1-4 heteroatoms selected from O, S and N.

The term “alkenyl” refers to a hydrocarbon radical straight, branched or cyclic containing from 2 to 15 carbon atoms and at least one carbon to carbon double bond. Preferably one carbon to carbon double bond is present, and up to four non-aromatic (non-resonating) carbon-carbon double bonds may be present. Examples of alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, isoprenyl, farnesyl, geranyl, geranylgeranyl and the like. Preferred alkenyl groups include ethenyl, propenyl, butenyl and cyclohexenyl. As described above with respect to alkyl, the straight, branched or cyclic portion of the alkenyl group may contain double bonds and may be substituted when a substituted alkenyl group is provided.

The term “alkynyl” refers to a hydrocarbon radical straight, branched or cyclic, containing from 2 to 15 carbon atoms and at least one carbon to carbon triple bond. Up to three carbon-carbon triple bonds may be present. Preferred alkynyl groups include ethynyl, propynyl and butynyl. As described above with respect to alkyl, the straight, branched or cyclic portion of the alkynyl group may contain triple bonds and may be substituted when a substituted alkynyl group is provided.

Aryl refers to aromatic rings e.g., phenyl, substituted phenyl and like groups as well as rings which are fused, e.g., naphthyl and the like. Aryl thus contains at least one ring having at least 6 atoms, with up to two such rings being present, containing up to 10 atoms therein, with alternating (resonating) double bonds between adjacent carbon atoms. The preferred aryl groups are phenyl and naphthyl. Aryl groups may likewise be substituted as defined below. Preferred substituted aryls include phenyl and naphthyl substituted with one or two groups. With regard to the farnesyl transferase inhibitors, “aryl” is intended to include any stable monocyclic, bicyclic or tricyclic carbon ring(s) of up to 7 members in each ring, wherein at least one ring is aromatic. Examples of aryl groups include phenyl, naphthyl, anthracenyl, biphenyl, tetrahydronaphthyl, indanyl, phenanthrenyl and the like.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one additional carbon atom is optionally replaced by a heteroatom selected from O or S, and in which from 1 to 3 additional carbon atoms are optionally replaced by nitrogen heteroatoms. The heteroaryl group is optionally substituted with up to three groups.

Heteroaryl thus includes aromatic and partially aromatic groups which contain one or more heteroatoms. Examples of this type are thiophene, purine, imidazopyridine, pyridine, oxazole, thiazole, oxazine, pyrazole, tetrazole, imidazole, pyridine, pyrimidine, pyrazine and triazine. Examples of partially aromatic groups are tetrahydro-imidazo[4,5-c]pyridine, phthalidyl and saccharinyl, as defined below.

With regard to the farnesyl transferase inhibitors, the term heterocycle or heterocyclic, as used herein, represents a stable 5- to 7-membered monocyclic or stable 8- to 11-membered bicyclic or stable 11-15 membered tricyclic heterocycle ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydro-benzothienyl, dihydrobenzothiopyranyl, dihydrobenzothio-pyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, 2-oxopiperazinyl, 2-oxopiperidinyl, 2-oxopyrrolidinyl, piperidyl, piperazinyl, pyridyl, pyridyl N-oxide, pyridonyl, pyrazinyl, pyrazolidinyl, pyrazolyl, pyrimidinyl, pyrrolidinyl, pyrrolyl, quinazolinyl, quinolinyl, quinolinyl N-oxide, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. Preferably, heterocycle is selected from imidazolyl, 2-oxopyrrolidinyl, piperidyl, pyridyl and pyrrolidinyl.

With regard to the farnesyl transferase inhibitors, the terms “substituted aryl”, “substituted heterocycle” and “substituted cycloalkyl” are intended to include the cyclic group which is substituted with 1 or 2 substitutents selected from the group which includes but is not limited to F, Cl, Br, CF₃, NH₂, N(C₁-C₆ alkyl)₂, NO₂, CN, (C₁-C₆ alkyl)O—, —OH, (C₁-C₆ alkyl)S(O)M-, (C₁-C₆ alkyl)C(O)NH—, H₂N—C(NH)—, (C₁-C₆ alkyl)C(O)—, (C₁-C₆ alkyl)OC(O)—, N₃,(C₁-C₆ alkyl)OC(O)NH— and C₁-C₂₀ alkyl.

When R² and R³ are combined to form —(CH₂)ᵤ—, cyclic moieties are formed. Examples of such cyclic moieties include, but are not limited to:

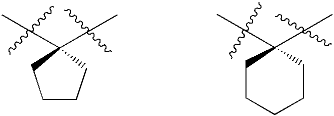

In addition, such cyclic moieties may optionally include a heteroatom(s). Examples of such heteroatom-containing cyclic moieties include, but are not limited to:

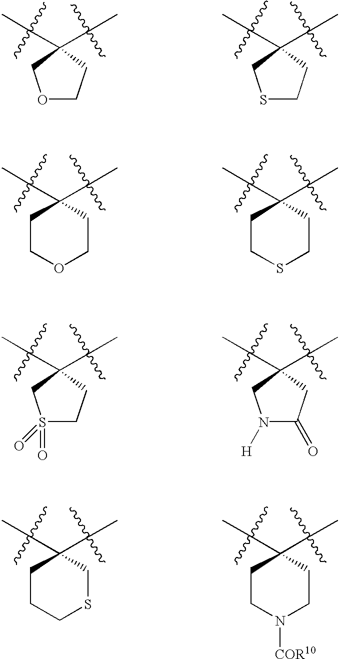

The term C₄-C₆ cycloalkyl in the definition of R^{1c} wherein two R^{1c}s are combined is illustrated by the following:

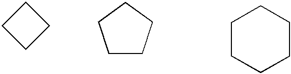

The term C₆-C₁₀ “multicyclic alkyl ring” in the definition of R^{1c} wherein two R^{1c}s are combined is intended to include polycyclic saturated and unsaturated aliphatic hydrocarbon groups having the specified number of carbon atoms. Examples of such cycloalkyl groups includes, but are not limited to:

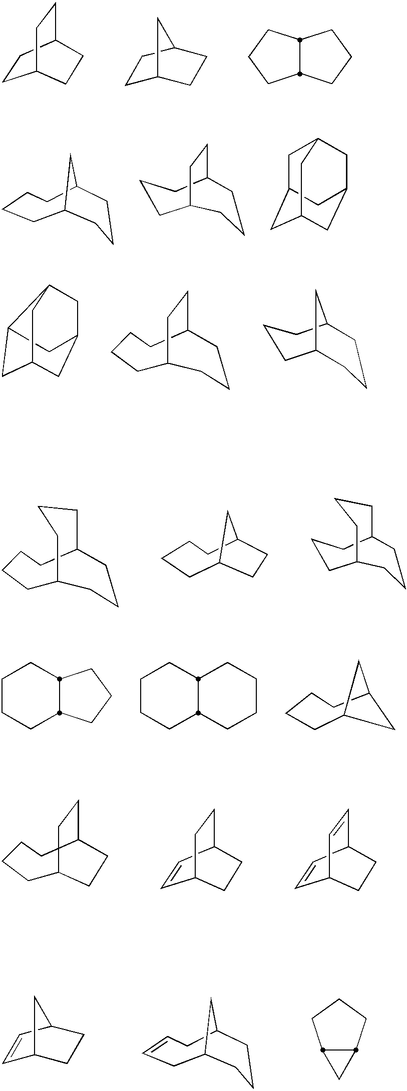

The compounds used in the present method may have asymmetric centers and occur as racemates, racemic mixtures, and as individual diastereomers, with all possible isomers, including optical isomers, being included in the present invention. Unless otherwise specified, named amino acids are understood to have the natural “L” stereoconfiguration.

With respect to the farnesyl-protein transferase inhibitors of the formula II, the substituent illustrated by the structure:

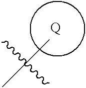

represents a 4, 5, 6 or 7 membered heterocyclic ring which comprises a nitrogen atom through which Q is attached to Y and 0-2 additional heteroatoms selected from N, S and O, and which also comprises a carbonyl, thiocarbonyl, —C(═NR¹³)— or sulfonyl moiety adjacent to the nitrogen atom attached to Y and includes the following ring systems:

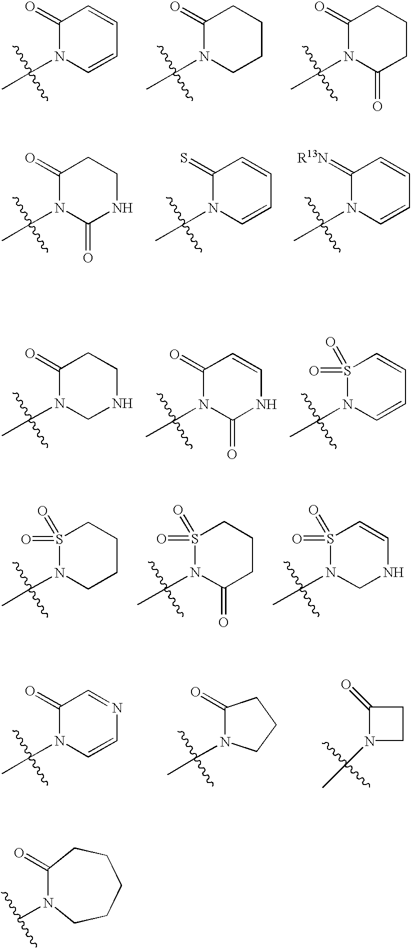

It is understood that such rings may be substituted by R^{6a}, R^{6b}, R^{6c}, R^{6d} and/or R^{6e} as defined hereinabove.

With respect to the farnesyl-protein transferase inhibitors of the formula II, the moiety described as

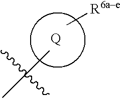

where any two of R^{6a}, R^{6b}, R^{6c}, R^{6d} and R^{6e} on adjacent carbon atoms are combined to form a diradical selected from —CH═CH—CH═CH, —CH═CH—CH—, —(CH₂)₄— and —(CH₂)₄— includes, but is not limited to, the following structures:

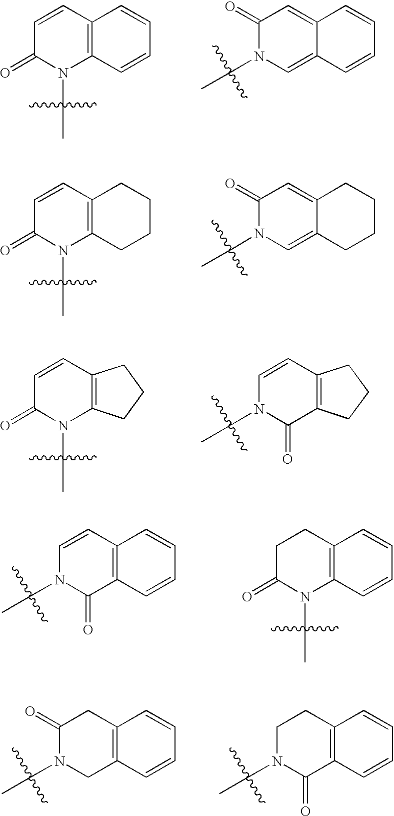

It is understood that such fused ring moieties may be further substituted by the remaining R^{6a}, R^{6b}, R^{6c}, R^{6d} and/or R^{6e} as defined hereinabove.

With respect to the farnesyl-protein transferase inhibitors of the formula II, the substituent illustrated by the structure:

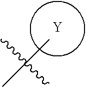

represents a 5, 6 or 7 membered carbocyclic ring wherein from 0 to 3 carbon atoms are replaced by a heteroatom selected from N, S and O, and wherein Y is attached to Q through a carbon atom and includes the following ring systems:

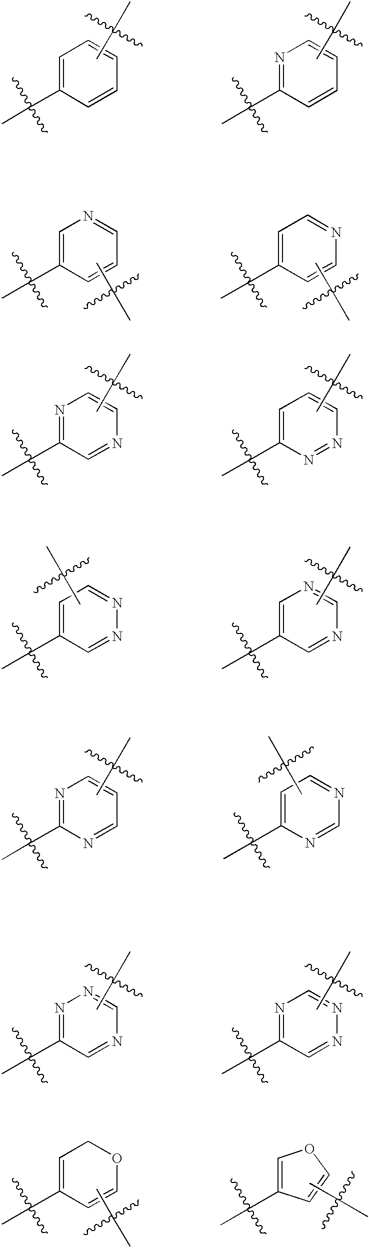

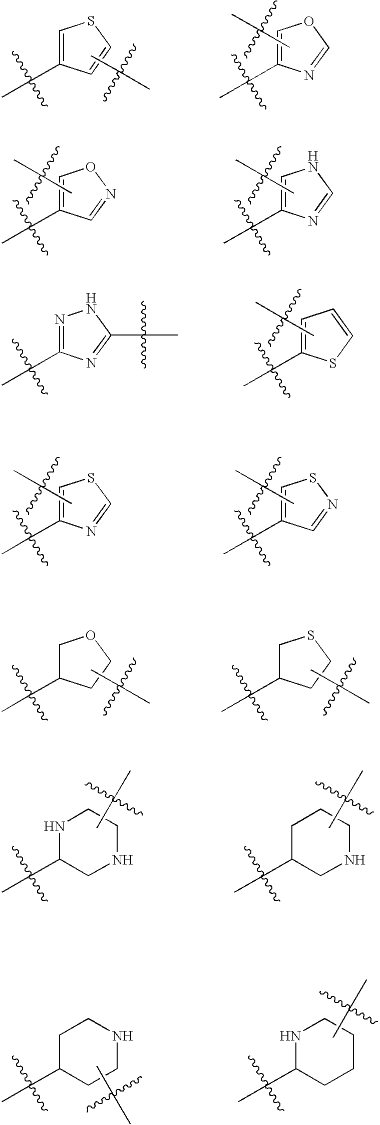

With respect to the farnesyl-protein transferase inhibitors of the formula III, the substituent illustrated by the structure:

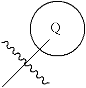

represents a 4, 5, 6 or 7 membered heterocyclic ring which comprises a nitrogen atom through which Q is attached to Y and 0-2 additional heteroatoms selected from N, S and O, and which also comprises a carbonyl, thiocarbonyl, —C(═NR¹³)— or sulfonyl moiety adjacent to the nitrogen atom attached to Y and includes the following ring systems:

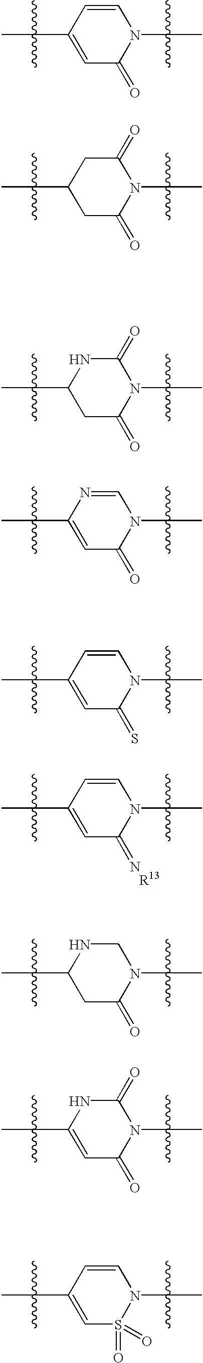

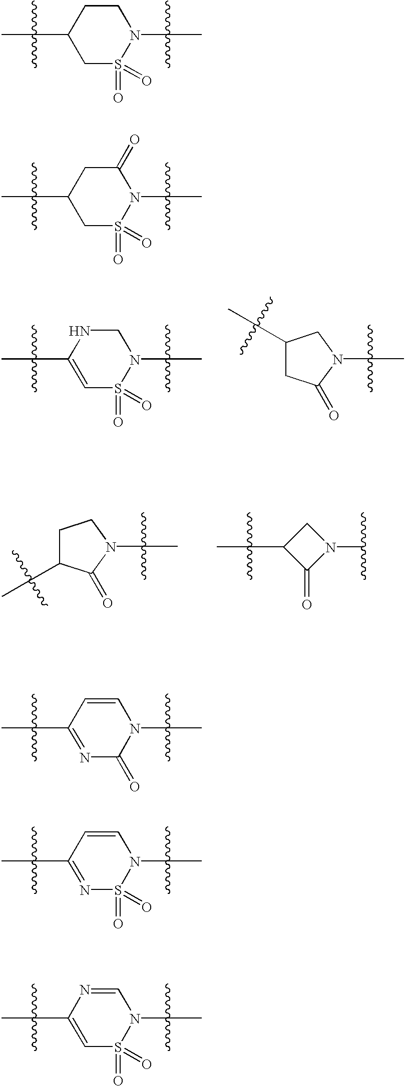

With respect to the farnesyl-protein transferase inhibitors of the formula III, the substituent illustrated by the structure:

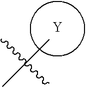

represents a 5-, 6- or 7-membered carbocyclic ring wherein from 0 to 3 carbon atoms are replaced by a heteroatom selected from N, S and O, and wherein Y is attached to Q through a carbon atom and includes the following ring systems:

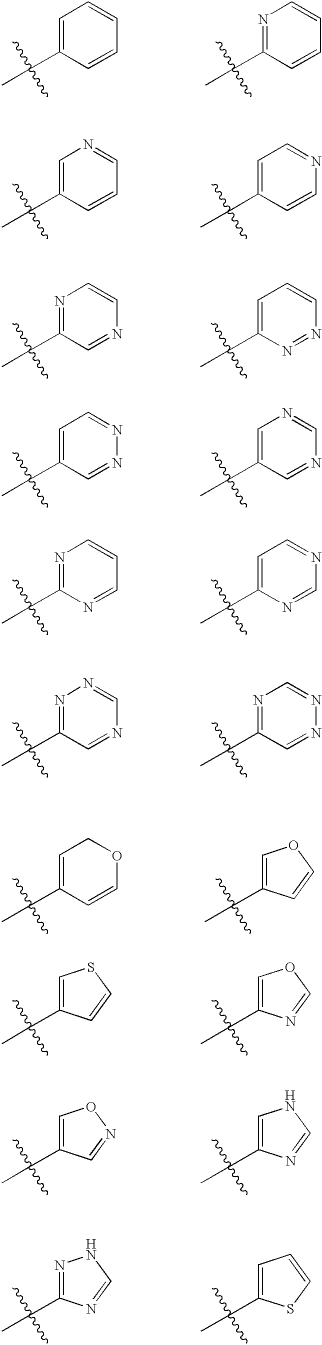

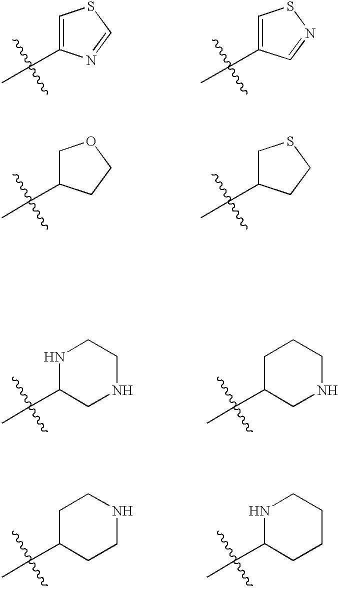

With respect to the farnesyl-protein transferase inhibitors of the formula III, the moiety described as

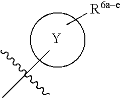

where any two of R^{6a}, R^{6b}, R^{6c}, R^{6d} and R^{6e} on adjacent carbon atoms are combined to form a diradical selected from —CH═CH—CH═CH, —CH═CH—CH—, —(CH₂)₄— and —(CH₂)₄— includes, but is not limited to, the following structures:

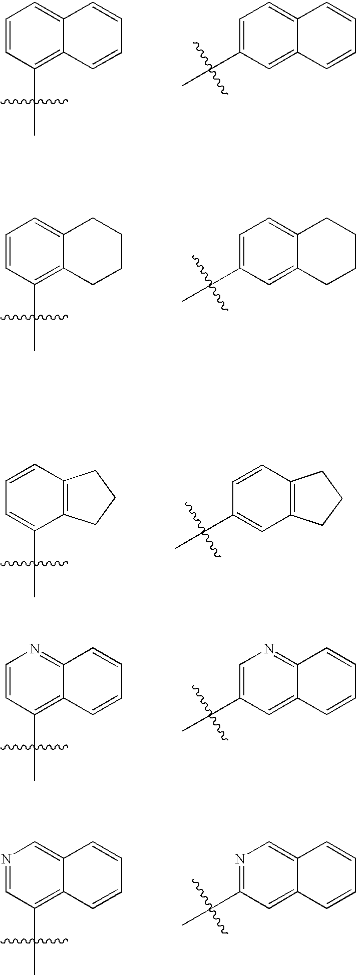

It is understood that such fused ring moieties may be further substituted by the remaining R^{6a}, R^{6b}, R^{6c}, R^{6d} and/or R^{6e} as defined hereinabove.

When R² and R³ are combined to form —(CH₂)ᵤ—, cyclic moieties are formed. Examples of such cyclic moieties include, but are not limited to:

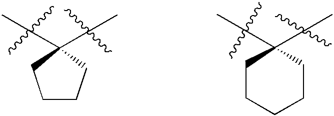

In addition, such cyclic moieties may optionally include a heteroatom(s). Examples of such heteroatom-containing cyclic moieties include, but are not limited to:

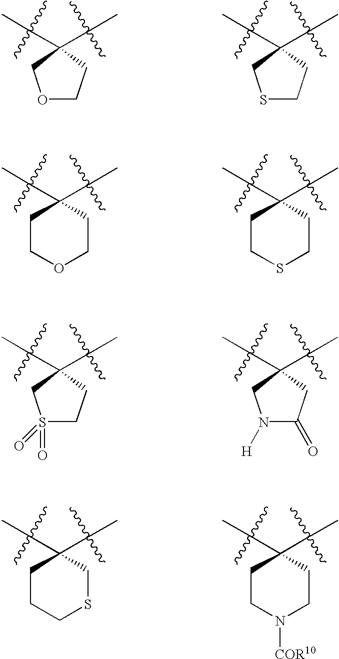

When R⁶ and R⁷, R⁷ and R^{7a}, or are combined to form —(CH₂)ᵤ—, cyclic moieties are formed. Examples of such cyclic moieties include, but are not limited to:

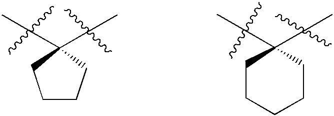

With respect to the compounds of formulas VII through XIV the following definitions apply:

As used herein, “alkyl” and the alkyl portion of aralkyl and similar terms, is intended to include both branched and straight-chain saturated aliphatic hydrocarbon groups having the specified number of carbon atoms; “alkoxy” represents an alkyl group of indicated number of carbon atoms attached through an oxygen bridge.

As used herein, “cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like.

“Halogen” or “halo” as used herein means fluoro, chloro, bromo and iodo.

As used herein, “aryl,” and the aryl portion of aralkyl and aroyl, is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

As used herein, the term “hydroxylated” represents substitution on a substitutable carbon of the ring system being so described by a hydroxyl moiety. As used herein, the term “polyhydroxylated” represents substitution on two or more substitutable carbon of the ring system being so described by 2, 3 or 4 hydroxyl moieties.

As used herein, the term “chlorosubstituted C₁-C₃-alkyl-CO—” represents a acyl moiety having the designated number of carbon atoms attached to a carbonyl moiety wherein one of the carbon atoms is substituted with a chlorine. Example of such chlorosubstituted elements include but are not limited to chloroacetyl, 2-chloropropionyl, 3-chloropropionyl and 2-chlorobutyroyl.

As used herein, the term “PEG” represents certain polyethylene glycol containing substituents having the designated number of ethyleneoxy subunits. Thus the term PEG(2) represents

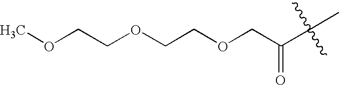

and the term PEG(6) represents

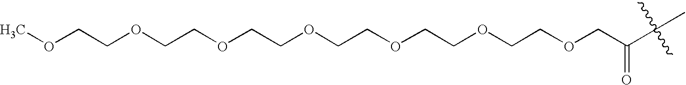

As used herein, the term “(d)(2,3-dihydroxypropionyl)” represents the following structure:

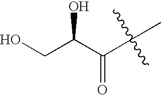

As used herein, the term “(2R,3S) 2,3,4-trihydroxybutanoyl” represents the following structure:

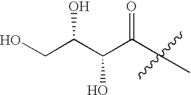

As used herein, the term “quinyl” represents the following structure:

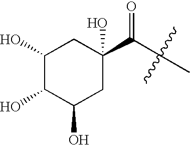

or the diastereomer thereof.

As used herein, the term “cotininyl” represents the following structure:

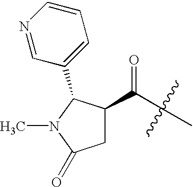

or the diastereomer thereof.

As used herein, the term “allyl” represents the following structure:

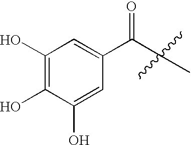

As used herein, the term μ4-ethoxysquarate” represents the following structure:

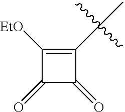

The structure

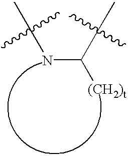

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

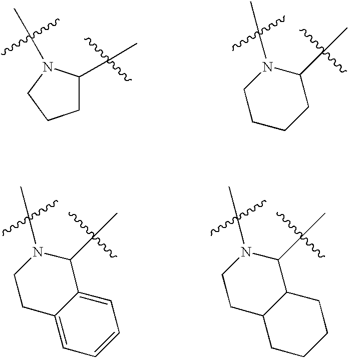

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

It is intended that the definition of any substituent or variable (e.g., R¹⁰, Z, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art as well as those methods set forth below.

The pharmaceutically acceptable salts of the compounds of this invention can be synthesized from the compounds of this invention which contain a basic moiety by conventional chemical methods. Generally, the salts are prepared by reacting the free base with stoichiometric amounts or with an excess of the desired salt-forming inorganic or organic acid in a suitable solvent or various combinations of solvents.available Na-Z-L-2,3-diaminopropionic acid (Fluka) as a starting material is preferred.

Abbreviations used in the description of the chemistry and in the Examples that follow are:

| | |
|---|---|
| Ac₂O | Acetic anhydride; |
| Boc | t-Butoxycarbonyl; |
| DBU | 1,8-diazabicyclo[5.4.0]undec-7-ene; |
| DMAP | 4-Dimethylaminopyridine; |
| DME | 1,2-Dimethoxyethane; |
| DMF | Dimethylformamide; |
| EDC | 1-(3-dimethylaminopropyl)-3-ethyl-carbodiimide- |
| | hydrochloride; |
| HOBT | 1-Hydroxybenzotriazole hydrate; |
| Et₃N | Triethylamine; |
| EtOAc | Ethyl acetate; |
| FAB | Fast atom bombardment; |
| HOOBT | 3-Hydroxy-1,2,2-benzotriazin-4(3H)-one; |
| HPLC | High-performance liquid chromatography; |
| MCPBA | m-Chloroperoxybenzoic acid; |
| MsCl | Methanesulfonyl chloride; |
| NaHMDS | Sodium bis(trimethylsilyl)amide; |
| Py | Pyridine; |
| TFA | Trifluoroacetic acid; |
| THF | Tetrahydrofuran. |

The compounds are useful in various pharmaceutically acceptable salt forms. The term “pharmaceutically acceptable salt” refers to those salt forms which would be apparent to the pharmaceutical chemist. i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients in combination with pharmaceutically acceptable carriers.

Pharmaceutically acceptable salts include conventional non-toxic salts or quarternary ammonium salts formed, e.g., from non-toxic inorganic or organic acids. Non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, sulfanilic, 2-acetoxybenzoic, fumaric, toluenesulfonic, methanesulfonic, ethane disulfonic, oxalic, isethionic, trifluoroacetic and the like.

The pharmaceutically acceptable salts of the present invention can be synthesized by conventional chemical methods. Generally, the salts are prepared by reacting the free base or acid with stoichiometric amounts or with an excess of the desired salt-forming inorganic or organic acid or base, in a suitable solvent or solvent combination.

The farnesyl transferase inhibitors of formula (I-a) through (I-c) can be synthesized in accordance with Schemes 1-16, in addition to other standard manipulations such as ester hydrolysis, cleavage of protecting groups, etc., as may be known in the literature or exemplified in the experimental procedures. These Schemes and other Schemes that illustrate reactions that may be useful in the preparation of inhibitors of formulae (I-a) through (I-c) are disclosed in U.S. Pat. No. 5,856,326, which is hereby incorporated by reference.

Substituents R, R^{a} and R^{b}, as shown in the Schemes, represent the substituents R², R³, R⁴, and R⁵; however their point of attachment to the ring is illustrative only and is not meant to be limiting. The compounds referred to in the Synopsis of Schemes 1-16 by Roman numerals are numbered starting sequentially with I and ending with 45.

These reactions may be employed in a linear sequence to provide the compounds of the invention or they may be used to synthesize fragments which are subsequently joined by the alkylation reactions described in the Schemes.

Synopsis of Schemes 1-16:

The requisite intermediates are in some cases commercially available, or can be prepared according to literature procedures, for the most part. In Scheme 1, for example, the synthesis of 2-alkyl sub-stituted piperazines is outlined, and is essentially that described by J. S. Kiely and S. R. Priebe in Organic Preparations and Proceedings Int., 1990, 22, 761-768. Boc-protected amino acids I, available commercially or by procedures known to those skilled in the art, can be coupled to N-benzyl amino acid esters using a variety of dehydrating agents such as DCC (dicyclohexycarbodiimide) or EDC.HCl (1-ethyl-3-(3-dimethylaminopropyl)carbodiimide hydrochloride) in a solvent such as methylene chloride, chloroform, dichloroethane, or in dimethylformamide. The product II is then deprotected with acid, for example hydrogen chloride in chloroform or ethyl acetate, or trifluoroacetic acid in methylene chloride, and cyclized under weakly basic conditions to give the diketopiperazine III. Reduction of III with lithium aluminum hydride in refluxing ether gives the piperazine IV, which is protected as the Boc derivative V. The N-benzyl group can be cleaved under standard conditions of hydrogenation, e.g., 10% palladium on carbon at 60 psi hydrogen on a Parr apparatus for 24-48 h. The product VI can be treated with an acid chloride, or a carboxylic acid under standard dehydrating conditions to furnish the carboxamides VII; a final acid deprotection as previously described gives the intermediate VIII (Scheme 2).

The protected piperazine intermediate VII can be reductively alkylated with other aldehydes such as 1-trityl-4-imidazolyl-carboxaldehyde or 1-trityl-4-imidazolylacetaldehyde, to give products such as IX (Scheme 3). The trityl protecting group can be removed from IX to give X, or alternatively, IX can first be treated with an alkyl halide then subsequently deprotected to give the alkylated imidazole XI. Alternatively, the intermediate VIII can be acylated or sulfonylated by standard techniques. As shown in Scheme 4, the imidazole acetic acid XII can be converted to the acetate XIV by standard procedures, and XIV can be first reacted with an alkyl halide, then treated with refluxing methanol to provide the regiospecifically alkylated imidazole acetic acid ester XV. Hydrolysis and reaction with piperazine VIII in the presence of condensing reagents such as 1-(3-dimethylaminopropyl)-3-ethylcarbodiimide (EDC) leads to acylated products such as XVII.

Depending on the identity of the amino acid I, various side chains can be incorporated into the piperazine. For example when I is the Boc-protected n-benzyl ester of aspartic acid, the intermediate diketopiperazine XVIII where n=1 and R=benzyl is obtained, as shown in Scheme 5. Subsequent lithium aluminum hydride reduction reduces the ester to the alcohol XIX, which can then be reacted with a variety of alkylating agents such as an alkyl iodide, under basic conditions, for example, sodium hydride in dimethylformamide or tetrahydrofuran. The resulting ether XX can then be carried on to final products as described in Schemes 3-4.

N-Aryl piperazines can be prepared as described in Scheme 6. An aryl amine XXI is reacted with bis-chloroethyl amine hydrochloride (XXII) in refluxing n-butanol to furnish compounds XXIII. The resulting piperazines XXIII can then be carried on to final products as described in Schemes 3-4.

Piperazin-5-ones can be prepared as shown in Scheme 7. Reductive amination of Boc-protected amino aldehyde XXIV (prepared from 1 as described previously) gives rise to compound XXV. This is then reacted with bromoacetyl bromide under Schotten-Baumann conditions; ring closure is effected with a base such as sodium hydride in a polar aprotic solvent such as dimethylformamide to give XXVI. The carbamate protecting group is removed under acidic conditions such as trifluoroacetic acid in methylene chloride, or hydrogen chloride gas in methanol or ethyl acetate, and the resulting piperazine can then be carried on to final products as described in Schemes 3-4.

The isomeric piperazin-3-ones can be prepared as described in Scheme 8. The imine formed from arylcarboxamides XXVII and 2-aminoglycinal diethyl acetal (XXVIII) can be reduced under a variety of conditions, including sodium triacetoxyborohydride in dichloroethane, to give the amine XXIX. Amino acids I can be coupled to amines XXIX under standard conditions, and the resulting amide XXX when treated with aqueous acid in tetrahydrofuran can cyclize to the unsaturated XXXI. Catalytic hydrogenation under standard conditions gives the requisite intermediate XXXII, which is elaborated to final products as described in Schemes 3-4.

Access to alternatively substituted piperazines is described in Scheme 9. Following deprotection with trifluoroacetic acid, the N-benzyl piperazine V can be acylated with an aryl carboxylic acid. The resulting N-benzyl aryl carboxamide XXXIII can be hydrogenated in the presence of a catalyst to give the piperazine carboxamide XXXIV which can then be carried on to final products as described in Schemes 3-4.

The aldehyde XXIV from Scheme 7 can also be reductively alkylated with an aniline as shown in Scheme 10. The product XXXV can be converted to a piperazinone by acylation with chloroacetyl chloride to give XXXVI, followed by base-induced cyclization to XXXVII. Deprotection, followed by reductive alkylation with a protected imidazole carboxaldehyde leads to XXXVIII, which can be alkylation with an arylmethylhalide to give the imidazolium salt IXL. Final removal of protecting groups by either solvolysis with a lower alkyl alcohol, such as methanol, or treatment with triethylsilane in methylene chloride in the presence of trifluoroacetic acid gives the final product XL.

Scheme 11 illustrates the use of an optionally substituted homoserine lactone XLI to prepare a Boc-protected piperazinone XLII. Intermediate XLII may be deprotected and reductively alkylated or acylated as illustrated in the previous Schemes. Alternatively, the hydroxyl moiety of intermediate XLII may be mesylated and displaced by a suitable nucleophile, such as the sodium salt of ethane thiol, to provide an intermediate XLIII. Intermediate XLII may also be oxidized to provide the carboxylic acid on intermediate XLIV, which can be utilized form an ester or amide moiety.

Amino acids of the general formula XLVI which have a sidechain not found in natural amino acids may be prepared by the reactions illustrated in Scheme 12 starting with the readily prepared imine XLV.

Schemes 13-16 illustrate syntheses of suitably substituted aldehydes useful in the syntheses of the instant compounds wherein the variable W is present as a pyridyl moiety. Similar synthetic strategies for preparing alkanols that incorporate other heterocyclic moieties for variable W are also well known in the art.

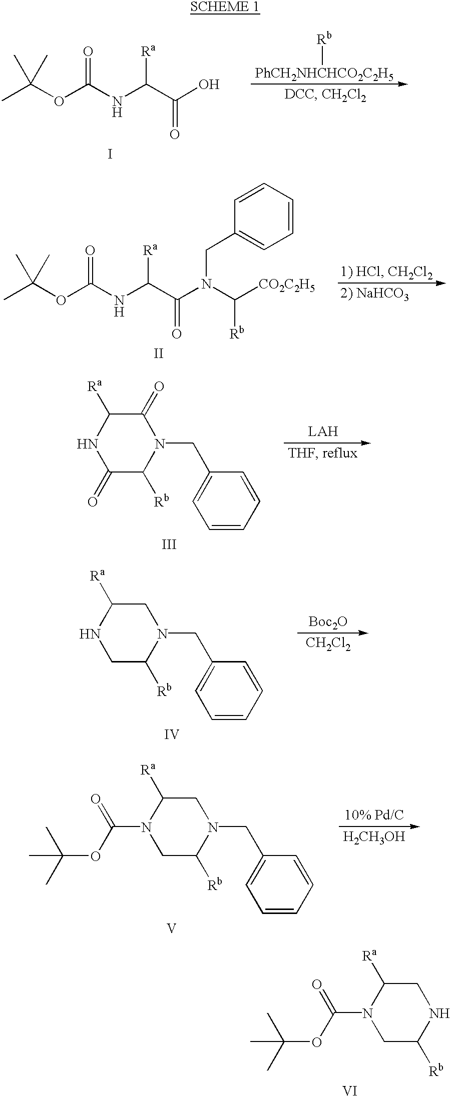

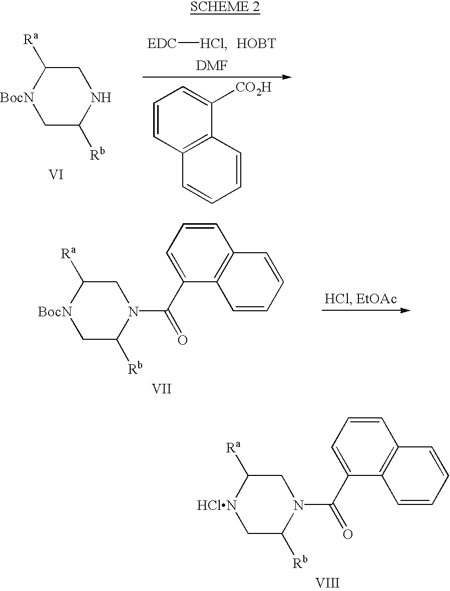

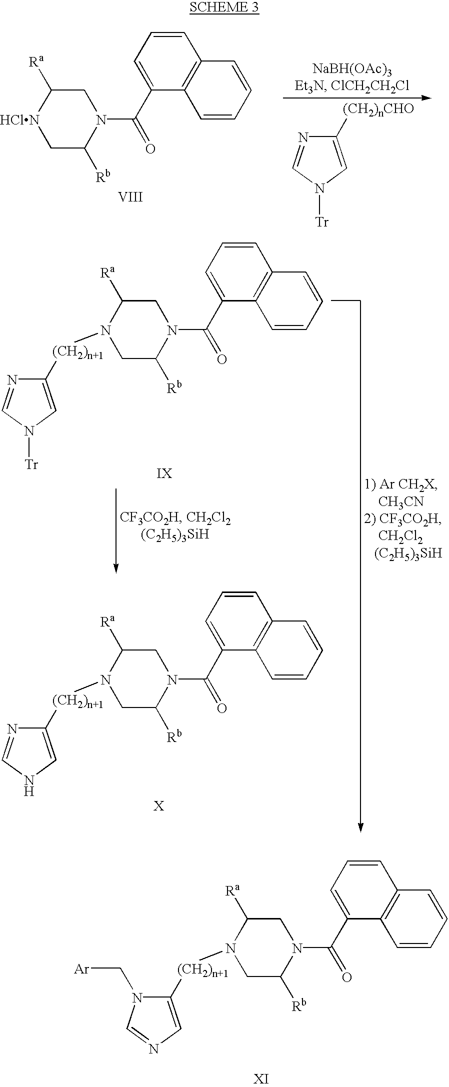

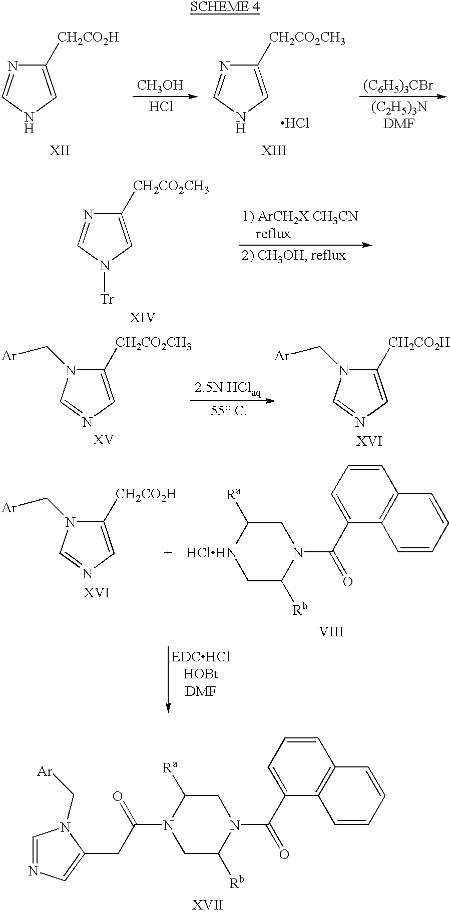

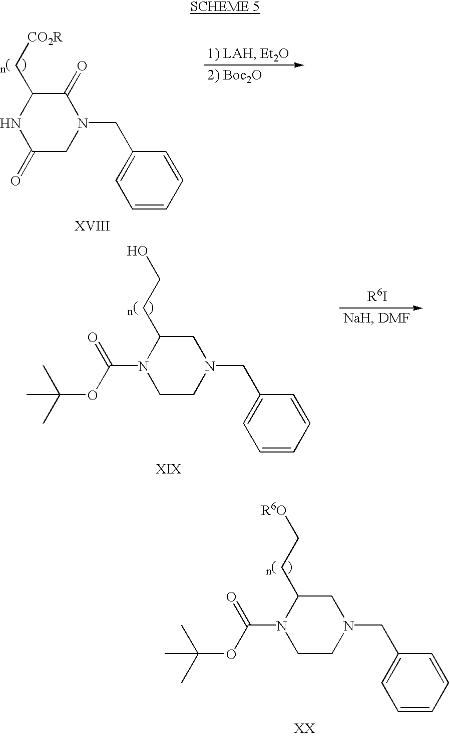

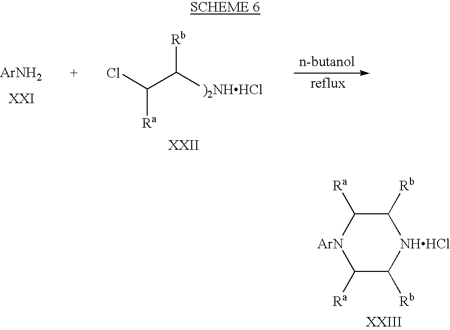

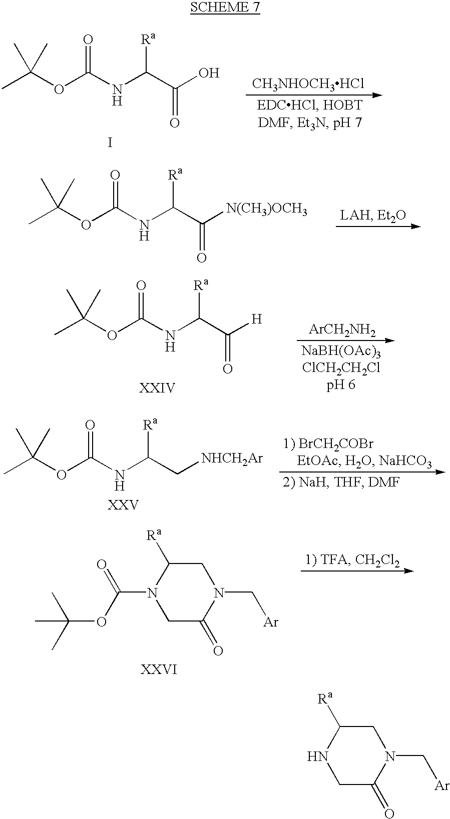

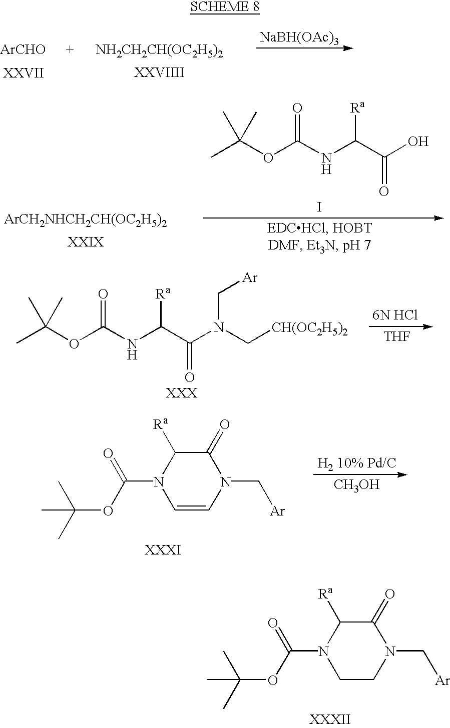

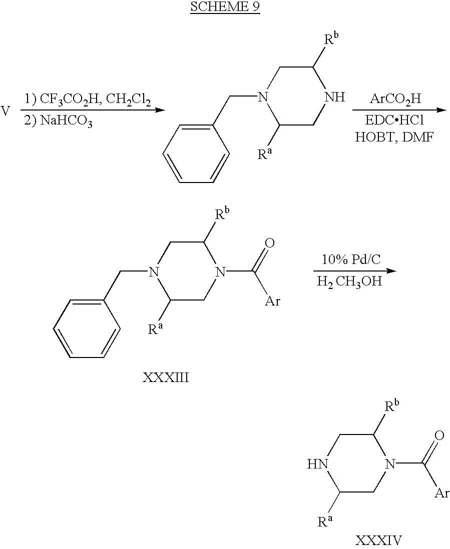

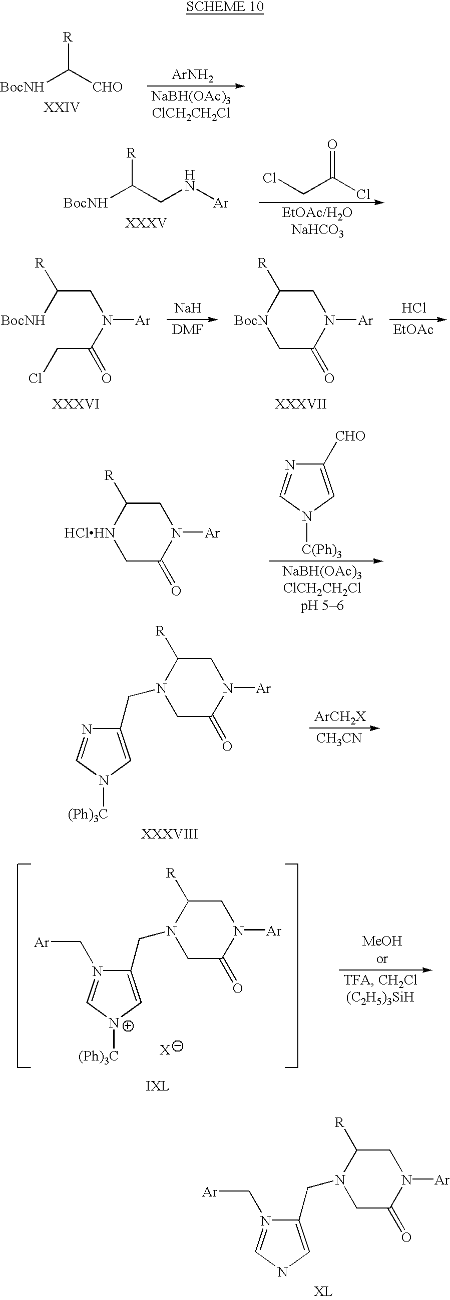

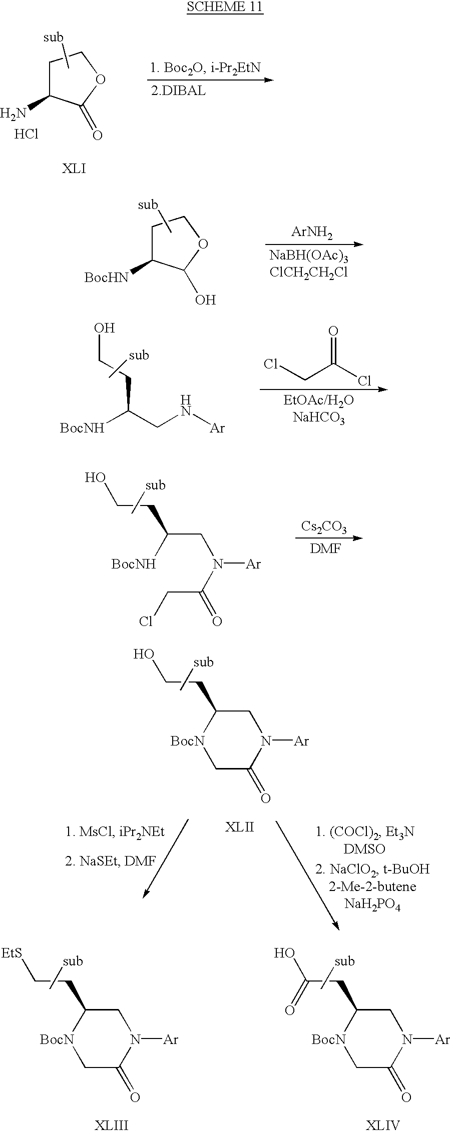

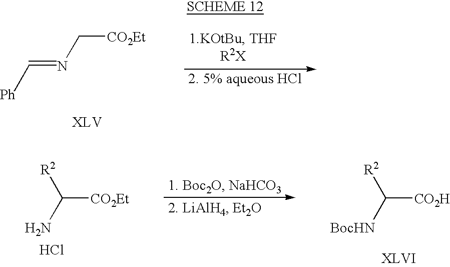

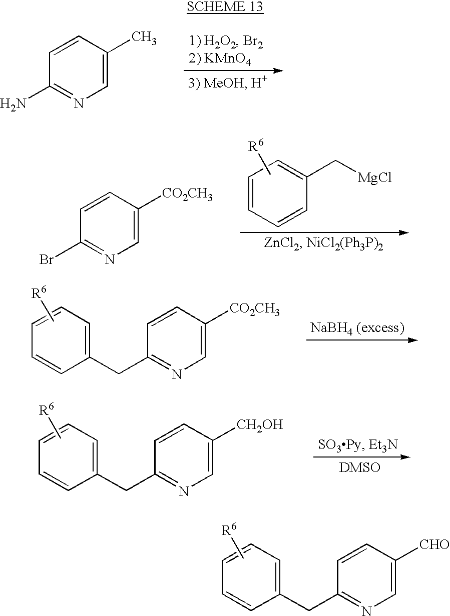

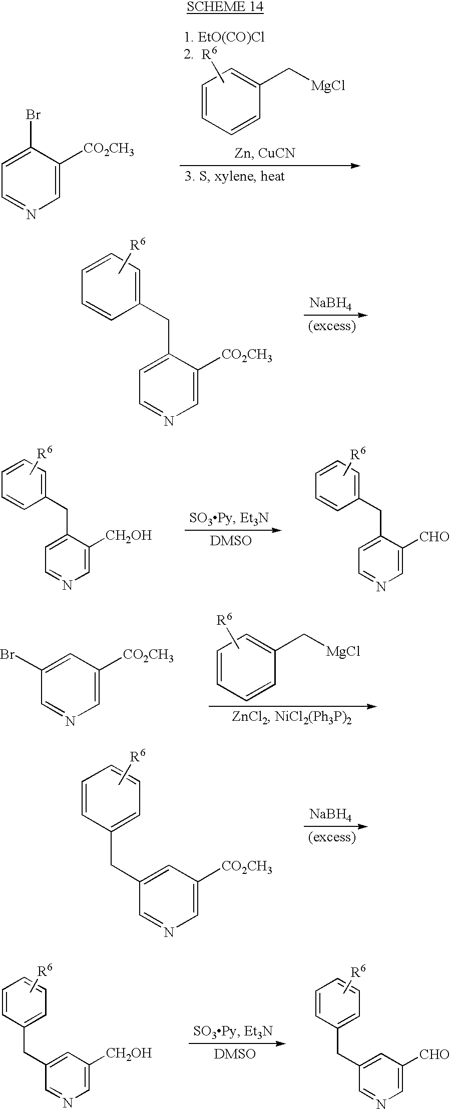

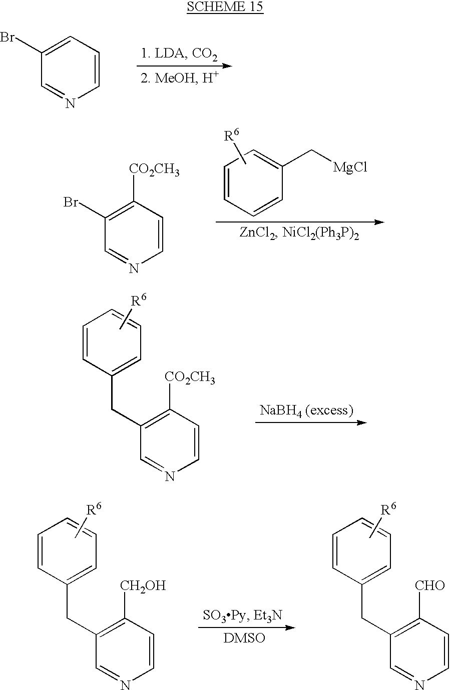

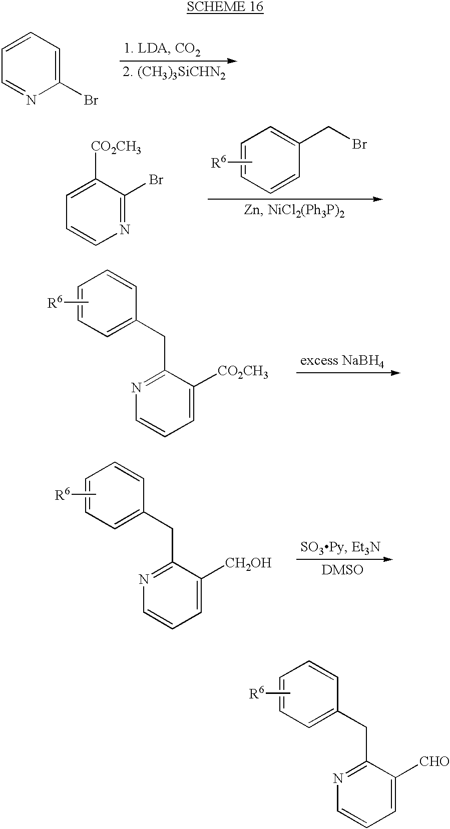

The farnesyl transferase inhibitors of formula (II) can be synthesized in accordance with Schemes 17-22, in addition to other standard manipulations such as ester hydrolysis, cleavage of protecting groups, etc., as may be known in the literature or exemplified in the experimental procedures. These Schemes and other Schemes that illustrate reactions that may be useful in the preparation of inhibitors of formula II are disclosed in PCT Publication No. WO 98/29119 (Jul. 9, 1998), which is hereby incorporated by reference.

Substituents R³, R⁶ and R⁸, as shown in the Schemes, represent the substituents R³, R⁴, R⁵, R^{6a}, R^{6b}, R^{6c}, R^{6d}, R^{6e} and R⁸ as described for formula II; although only one such R³, R⁶ or R⁸ is present in the intermediates and products of the schemes, it is understood that the reactions shown are also applicable when such aryl or heterocyclic moieties contain multiple substituents. The compounds referred to in the Synopsis of Schemes 17-22 by numerals are numbered starting sequentially with 1 and ending with 16.

These reactions may be employed in a linear sequence to provide the compounds of the invention or they may be used to synthesize fragments which are subsequently joined by the alkylation reactions described in the Schemes. Aryl-aryl coupling is generally described in “Comprehensive Organic Functional Group Transformations,” Katritsky et al. eds., pp 472-473, Pergamon Press (1995).

Synopsis of Schemes 17-22:

The requisite intermediates are in some cases commercially available, or can be prepared according to literature procedures. Schemes 17-22 illustrate synthesis of the instant bicyclic compounds which incorporate a preferred benzylimidazolyl side chain. Thus, in Scheme 17, for example, a bicyclic intermediate that is not commercially available may be synthesized by methods known in the art. Thus, a suitably substituted pyridinone 1 may be reacted under coupling conditions with a suitably substituted iodobenzyl alcohol to provide the intermediate alcohol 2. The intermediate alcohol 2 may converted to the corresponding bromide 3. The bromide 3 may be coupled to a suitably substituted benzylimidazolyl 4 to provide, after deprotection, the instant compound 5.

Schemes 18-20 illustrate methods of synthesizing related or analogous key alcohol intermediates, which can then be processed as described in Scheme 17. Thus, Scheme 18 illustrates pyridinonylpyridyl alcohol forming reactions starting with the suitably substituted iodonicotinate 6.

Scheme 19 illustrates preparation of the intermediate alcohol 9 wherein the terminal lactam ring is saturated. Acylation of a suitably substituted 4-aminobenzyl alcohol 7 with a suitably substituted brominated acyl chloride provides the bisacylated intermediate 8. Closure of the lactam ring followed by saponifiaction of the remaining acyl group provides the intermediate alcohol. Preparation of the homologous saturated lactam 10-is illustrated in Scheme 20.

Scheme 21 illustrates synthesis of an instant compound wherein a non-hydrogen R^{9b} is incorporated in the instant compound. Thus, a readily available 4-substituted imidazole 11 may be selectively iodinated to provide the 5-iodoimidazole 12. That imidazole may then be protected and coupled to a suitably substituted benzyl moiety to provide intermediate 13. Intermediate 13 can then undergo the alkylation reactions that were described hereinabove.

Compounds of the instant invention wherein the A¹(CR¹₂)ₙA²(CR¹₂)ₙ linker is a substituted methylene may be synthesized by the methods shown in Scheme 22. Thus, the N-protected imidazolyl iodide 14 is reacted, under Grignard conditions with a suitably protected benzaldehyde to provide the alcohol 15. Acylation, followed by the alkylation procedure illustrated in the Schemes above (in particular, Scheme 17) provides the instant compound 16. If other R¹ substituents are desired, the acetyl moiety can be manipulated as illustrated in the Scheme.

Other suitably substituted aldehydes such as those described in Schemes 13-16 hereinabove may be utilized in the syntheses of the instant compounds of the formula II. Similar synthetic strategies for preparing alkanols that incorporate other heterocyclic moieties for variable W are also well known in the art.

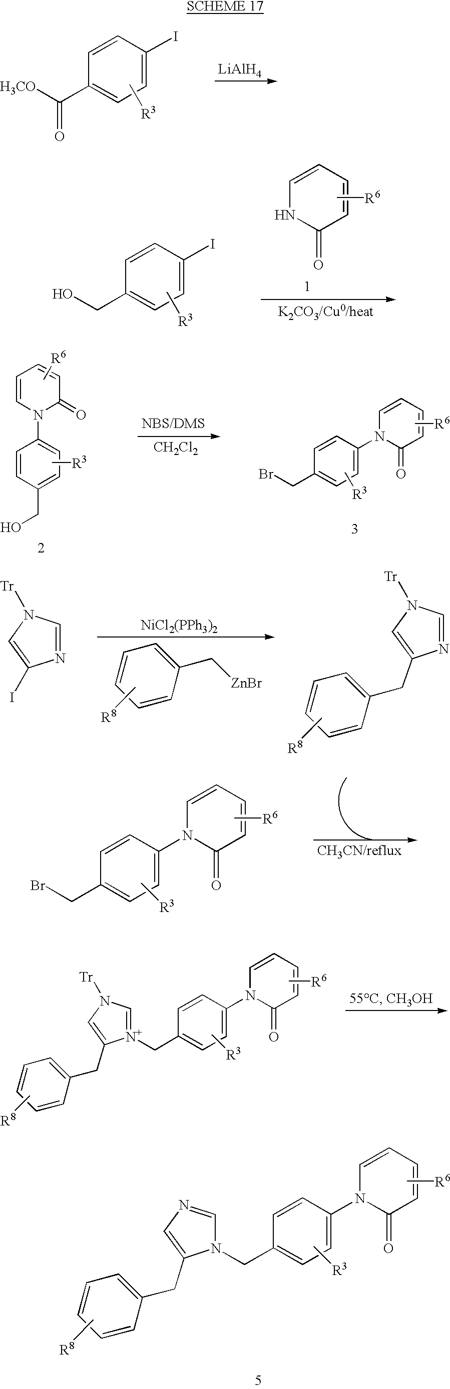

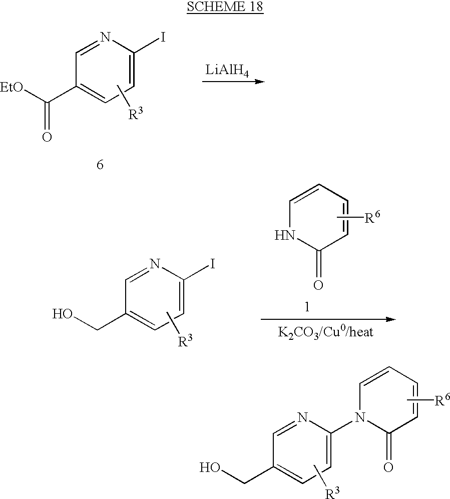

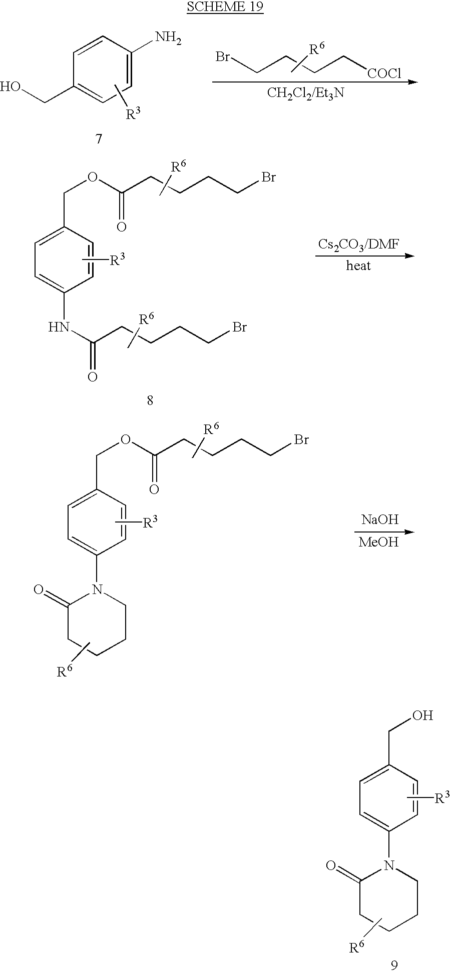

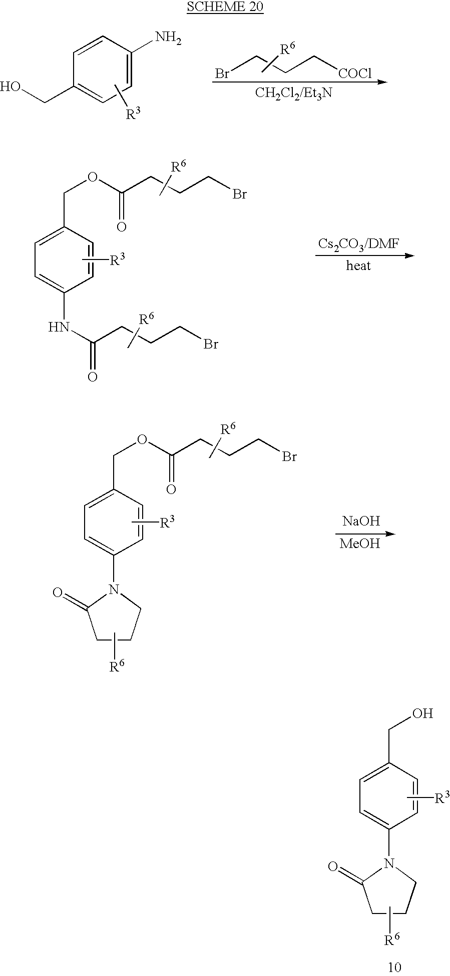

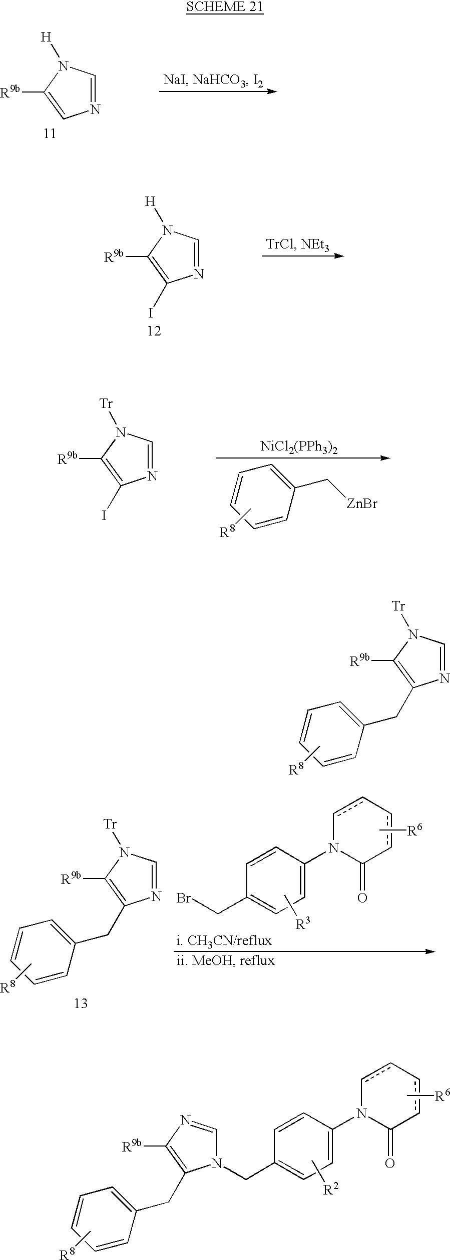

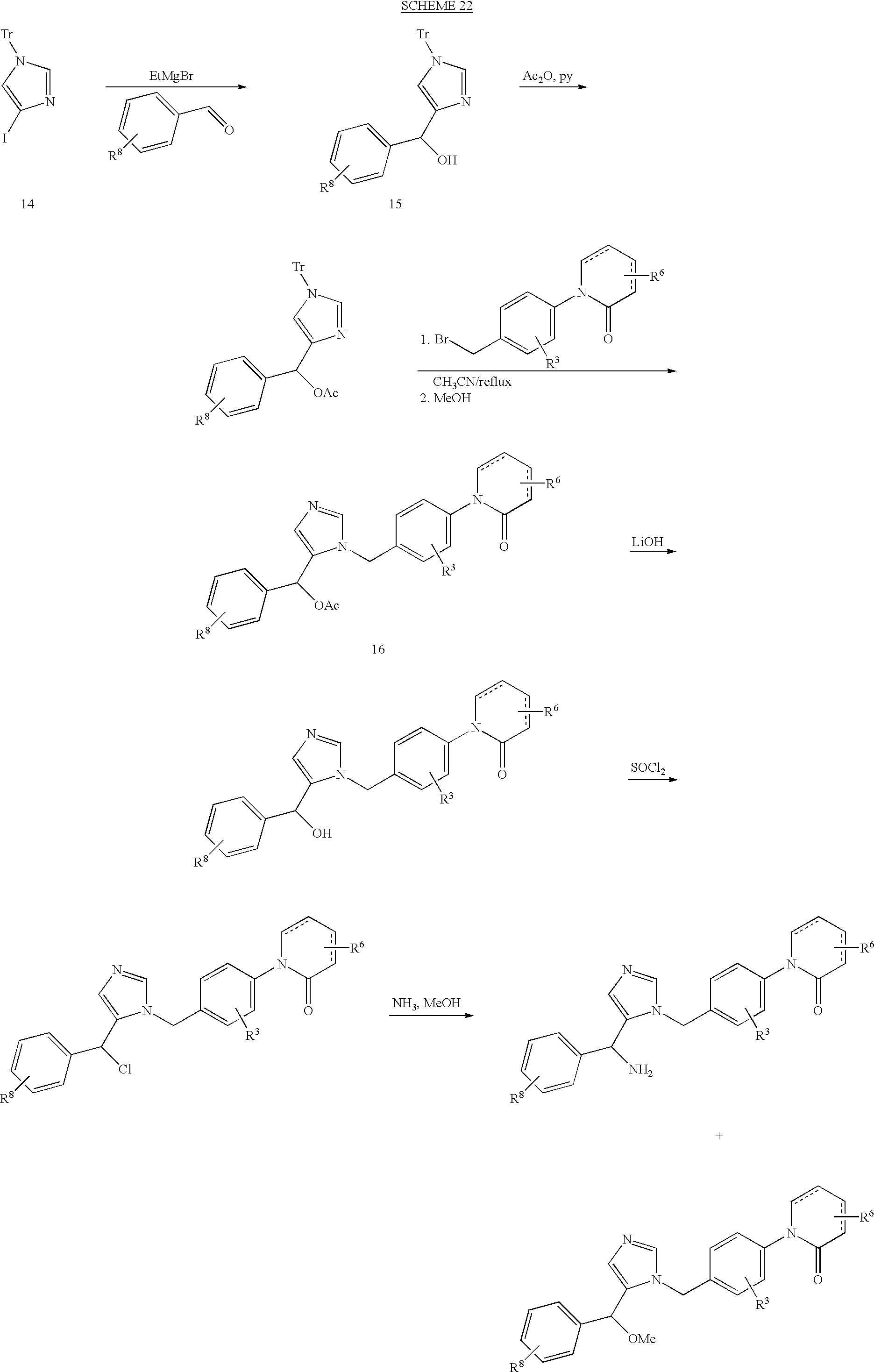

The farnesyl transferase inhibitors of formula (III) can be synthesized in accordance with Schemes 23-24, in addition to other standard manipulations such as ester hydrolysis, cleavage of protecting groups, etc., as may be known in the literature or exemplified in the experimental procedures. These Schemes and other Schemes that illustrate reactions that may be useful in the preparation of inhibitors of formula III are disclosed in PCT Publication No. WO 98/28980 (Jul. 9, 1998), which is hereby incorporated by reference.

Substituents R³, R⁶ and R⁸, as shown in the Schemes, represent the substituents R³, R⁴, R⁵, R^{6a}, R^{6b}, R^{6c}, R^{6d}, R^{6e} and R⁸ as described for formula III; although only one such R³, R⁶ or R⁸ is present in the intermediates and products of the schemes, it is understood that the reactions shown are also applicable when such aryl or heterocyclic moieties contain multiple substituents. The compounds referred to in the Synopsis of Schemes 23-24 by numerals are numbered starting sequentially with 17 and ending with 22.

These reactions may be employed in a linear sequence to provide the compounds of the invention or they may be used to synthesize fragments which are subsequently joined by the alkylation reactions described in the Schemes. The reactions described in the Schemes are illustrative only and are not meant to be limiting. Other reactions useful in the preparation of heteroaryl moieties are described in “Comprehensive Organic Chemistry, Volume 4: Heterocyclic Compounds” ed. P. G. Sammes, Oxford (1979) and references therein.

Synopsis of Schemes 23-24:

The requisite intermediates are in some cases commercially available, or can be prepared according to literature procedures. Schemes 23-24 illustrate synthesis of the instant bicyclic compounds which incorporate a preferred benzylimidazolyl side-chain. Thus, in Scheme 23, for example, a bicyclic intermediate that is not commercially available may be synthesized by methods known in the art. Thus, a suitably substituted pyridinonyl alcohol 18 may be synthesized starting from the corresponding isonicotinate 17 according to procedures described by Boekelhiede and Lehn (J. Org. Chem., 26:428-430 (1961)). The alcohol is then protected and reacted under Ullmann coupling conditions with a suitably substituted phenyl iodide, to provide the intermediate bicyclic alcohol 19. The intermediate alcohol 19 may converted to the corresponding bromide 20. The bromide 20 may be coupled to a suitably substituted benzylimidazolyl 21 to provide, after deprotection, the instant compound 22.

Scheme 24 illustrates methods of synthesizing related halide intermediates, which can then be processed as described in Scheme 23. Thus, Scheme 24 illustrates preparation of a pyridylpyridinonyl halide and thienylpyridinonyl halide starting with the suitably substituted halogenated heterocycles.

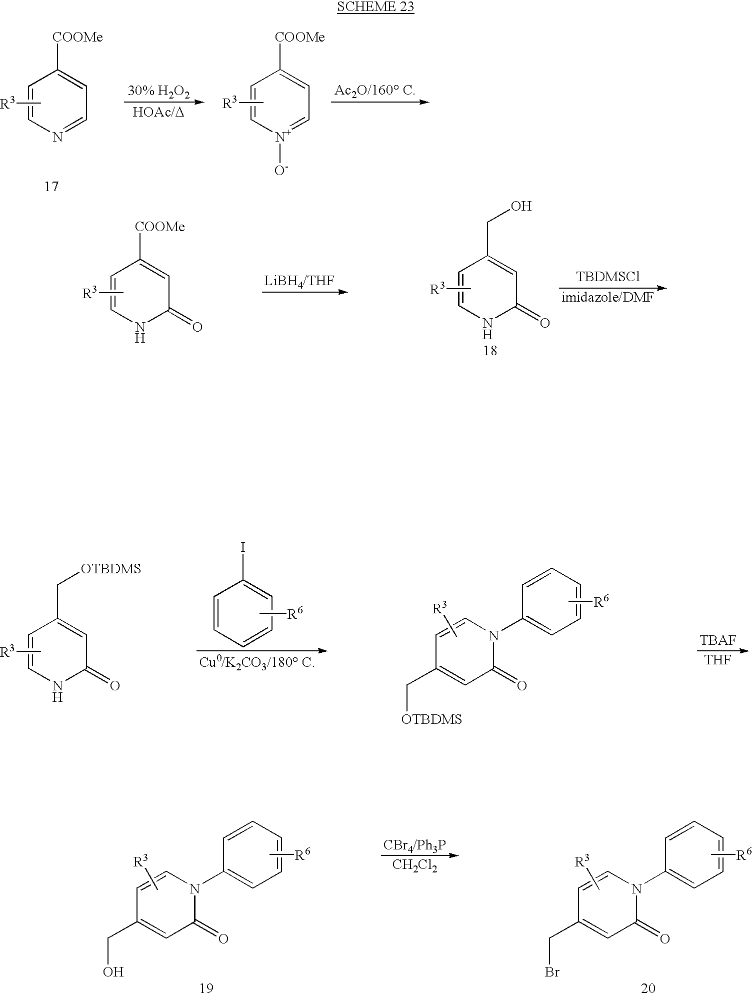

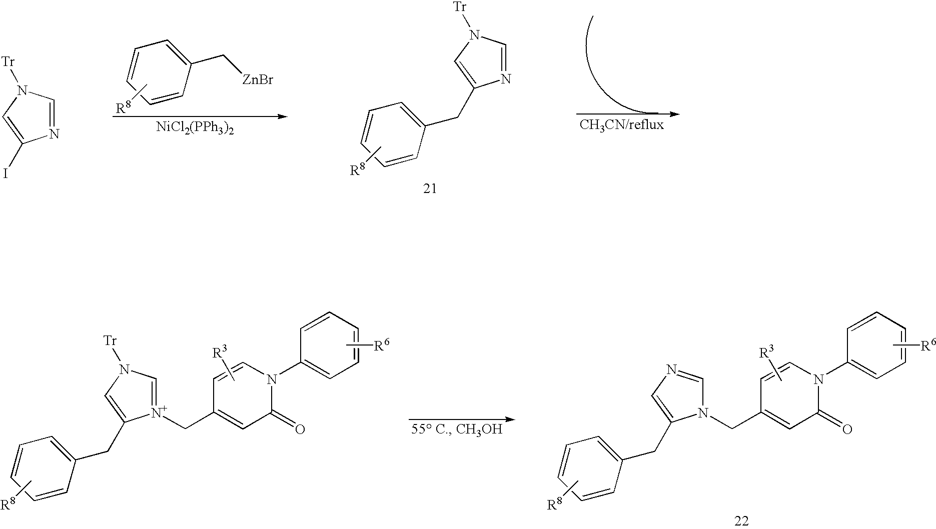

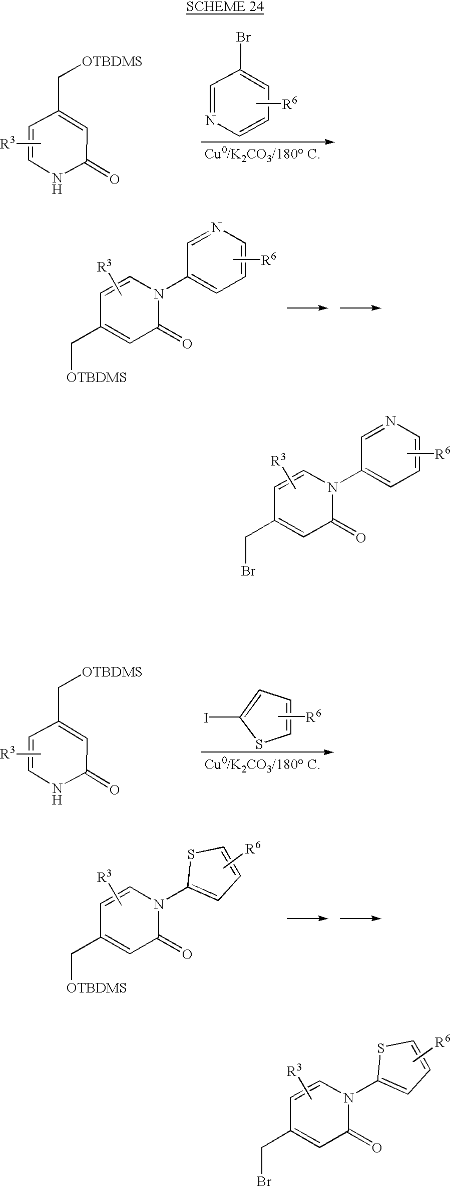

The farnesyl transferase inhibitors of formula (IV) can be synthesized in accordance with Schemes 25-46, in addition to other standard manipulations such as ester hydrolysis, cleavage of protecting groups, etc., as may be known in the literature or exemplified in the experimental procedures. Substituents R, R^{a}, R^{b} and R^{sub}, as shown in the Schemes, represent the substituents R², R³, R⁴, and R⁵, and substituents on Z¹ and Z²; however their point of attachment to the ring is illustrative only and is not meant to be limiting. The compounds referred to in the Synopsis of Schemes 25-46 by Roman numerals are numbered starting sequentially with I and ending with XLVI.

These reactions may be employed in a linear sequence to provide the compounds of the invention or they may be used to synthesize fragments which are subsequently joined by the alkylation reactions described in the Schemes.

Synopsis of Schemes 25-46:

The requisite intermediates are in some cases commercially available, or can be prepared according to literature procedures. In Scheme 25, for example, the synthesis of macrocyclic compounds of the instant invention containing suitably substituted piperazines and the preferred benzylimidazolyl moiety is outlined. Preparation of the substituted piperazine intermediate is essentially that described by J. S. Kiely and S. R. Priebe in Organic Preparations and Proceedings Int., 1990, 22, 761-768. Boc-protected amino acids I, available commercially or by procedures known to those skilled in the art, can be coupled to N-benzyl amino acid esters using a variety of dehydrating agents such as DCC (dicyclohexycarbodiimide) or EDC.HCl (1-ethyl-3-(3-dimethylaminopropyl)carbodiimide hydrochloride) in a solvent such as methylene chloride, chloroform, dichloroethane, or in dimethylformamide. The product II is then deprotected with acid, for example hydrogen chloride in chloroform or ethyl acetate, or trifluoroacetic acid in methylene chloride, and cyclized under weakly basic conditions to give the diketopiperazine III. Reduction of III with lithium aluminum hydride in refluxing ether gives the piperazine IV, which may then be deprotected by catalytic reduction to provide intermediate V. Intermediate V may then be coupled to intermediate VII, prepared from 4-imidazolylacetic acid VI in several step as illustrated. Once the amide bond is formed to yield the intermediate VIII, cesium carbonate nucleophilic aromatic substitution reaction conditions result in an intramolecular cyclization to yield compound IX of the instant invention. This cyclization reaction depends on the presence of an electronic withdrawing moiety (such as nitro, cyano, and the like) either ortho or para to the fluorine atom.

Scheme 26 illustrates the synthesis of instant compounds wherein an amido bond is formed between the piperazine nitrogen and the linker to the Y group. Thus, the protected piperazine X is coupled to a naphthoic acid having a suitably positioned benzyloxy moiety. Consecutive removal of the Boc and benzyl protecting groups provided intermediate XI, which may be coupled to a suitably substituted 1-benzylimidazole aldehyde XII to give intermediate XIII. Intramolecular cyclization takes place as previously described using the cesium carbonate conditions to provide instant compound XIV.

Scheme 27 illustrates the preparation of instant compounds which incorporate a piperazinone moiety in the macrocyclic ring. Thus the suitably substituted benzyloxybenzyl mesylate XV is reacted with a 4-protected 2-piperazinone XVI to provide the 1-benzyl-2-piperazinone intermediate XVII. Intermediate XVII is doubly deprotected in the presence of Boc anhydride to provide the N-Boc protected piperazinone, which is deprotected to give intermediate XVIII. Reductive N-alkylation of intermediate XVIII with a suitably substituted 1-benzylimidazole aldehyde XII provides intermediate XIX, which can undergo intramolecular cyclization under the cesium carbonate conditions to give compound XX of the instant invention.

Synthesis of compounds of the invention characterized by direct attachment of an aryl moiety to the piperazinone moiety and incorporation of a third aromatic carbocyclic moiety into the macrocycle is illustrated in Scheme 28. A benzyloxyphenoxyanaline XIII, prepared in three steps from a suitably substituted 2-benzyloxyphenol XXI and a suitably substituted nitrochlorobenzene XXII, is reacted with chloroacetyl chloride to provide intermediate XXIV. Intermediate XXIV is reacted with a suitably substituted ethanolamine and the resulting amido alcohol cyclized to form the piperazinone moiety of intermediate XXV. Intermediate XXV is reductively alkylated as described in Schemes 26 and 27 to provide intermediate XXVI. Deprotection, followed by intramolecular cyclization provides compound XXVII of the instant invention.

Scheme 29 illustrates expansion of the macrocyclic ring to a “18-membered” system by utilizing a suitably substituted 3-benzyloxyphenol XXVIII in the place of the 2-benzyloxyphenol XXI. Scheme 29 also illustrates the use of a reduced amino acid (such as methioninol) to provide substitution specifically at the 5-position of the piperazinone moiety.

Scheme 30 illustrates that the synthetic strategy of building the piperazinone onto a alcoholic aromatic amine can also be utilized to prepare compounds of the instant invention wherein a naphthyl group forms part of the macrocyclic backbone.

Scheme 31 illustrates the synthetic strategy that is employed when the R⁸ substitutent is not an electronic withdrawing moiety either ortho or para to the fluorine atom. In the absence of the electronic withdrawing moiety, the intramolecular cyclization can be accomplished via an Ullmann reaction. Thus, the imidazolylmethyl-acetate XXXII is treated with a suitably substituted halobenzylbromide to provide the 1-benzylimidazolyl intermediate XXXIII. The acetate functionality of intermediate XXXIII was converted to an aldehyde which was then reductively coupled to intermediate XVIII, prepared as illustrated in Scheme 27. Coupling under standard Ullmann conditions provided compound XXXIV of the instant invention.

Illustrative examples of the preparation of compounds of the instant invention that incorporate a 2,5-diketopiperazine moiety and a 2,3-diketopiperazine moiety are shown in Schemes 32-33 and Schemes 34-35 respectively.

Scheme 36 illustrates the manipulation of a functional group on a side chain of an intermediate 2,5-diketopiperazine. The side chain of intermediate IIIa, obtained as illustrated in Scheme 29 from protected aspartic acid, may be comprehensively reduced and reprotected to afford intermediate XXXV, which can deprotected or can be alkylated first followed by deprotection to provide intermediate IVa having an ether sidechain. The intermediate IVa can be incorporated into the reaction sequence illustrated in Scheme 25.

Scheme 37 illustrates direct preparation of a symmetrically substituted piperazine intermediate from a suitably substituted analine (such as intermediate XXIII from Scheme 28) and a suitably substituted bis-(chloroethyl)amine XXXVII. The intermediate XXXVIII can be utilized in the reaction sequence illustrated in Scheme 25 to produce compound IXL of the instant invention Preparation of a substituted piperazinone intermediate XVIIIa starting from a readily available N-protected amino acid XL is illustrated in Scheme 38.

Scheme 39 illustrates preparation of an intermediate piperazinone compound XLI having a substituent at the 3-position that is derived from the starting protected amino acid XL.

Incorporation of a spirocyclic moiety (for example, when R² and R³ are combined to form a ring) is illustrated in Scheme 40.

Scheme 41 illustrates the use of an optionally substituted homoserine lactone XLII to prepare a Boc-protected piperazinone XLIII. Intermediate XLIII may be deprotected and reductively alkylated or acylated as illustrated in the previous Schemes. Alternatively, the hydroxyl moiety of intermediate XLIII may be mesylated and displaced by a suitable nucleophile, such as the sodium salt of ethane thiol, to provide an intermediate XLIV. Intermediate XLIII may also be oxidized to provide the carboxylic acid on intermediate XLV, which can be utilized form an ester or amide moiety.

Amino acids of the general formula XL which have a sidechain not found in natural amino acids may be prepared by the reactions illustrated in Scheme 42 starting with the readily prepared imine XLVI.

Other suitably substituted aldehydes such as those described in Schemes 43-46 hereinabove may be utilized in the syntheses of the instant compounds of the formula IV wherein the moiety “W” is a pyridyl. Similar synthetic strategies for preparing alkanols that incorporate other heterocyclic moieties for variable W are also well known in the art.

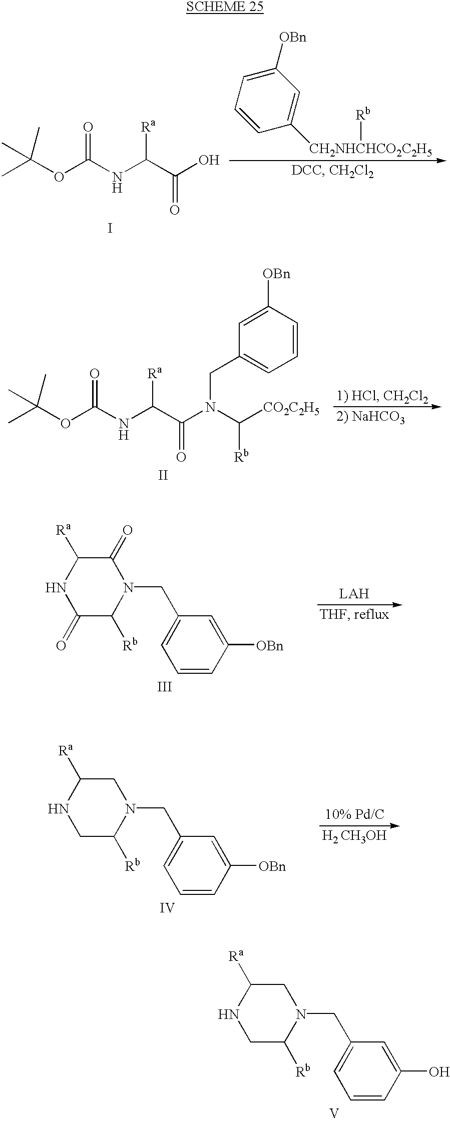

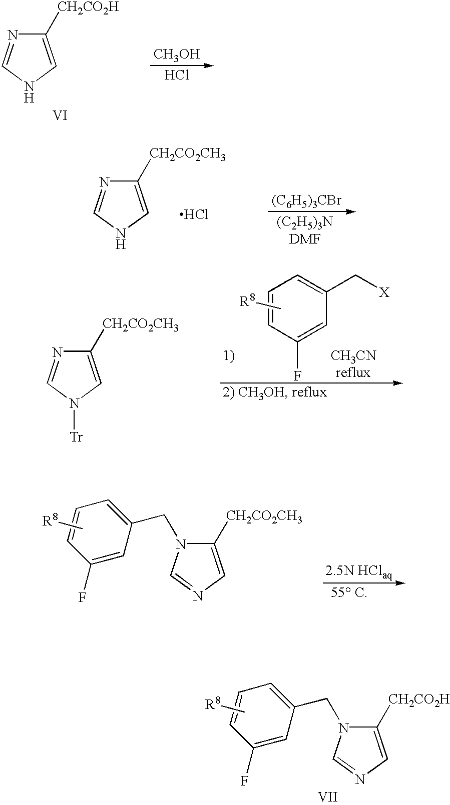

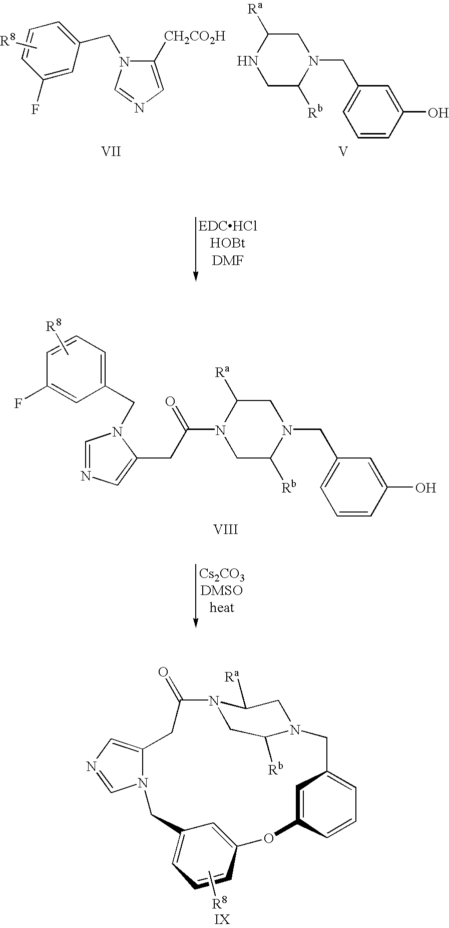

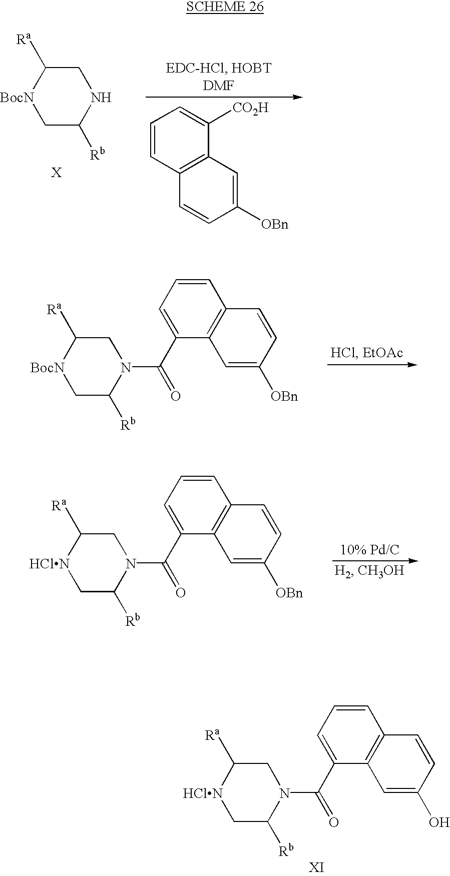

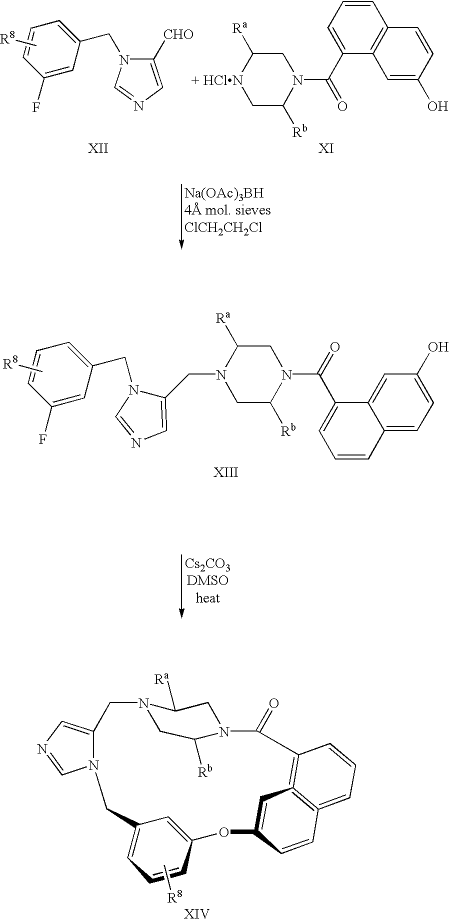

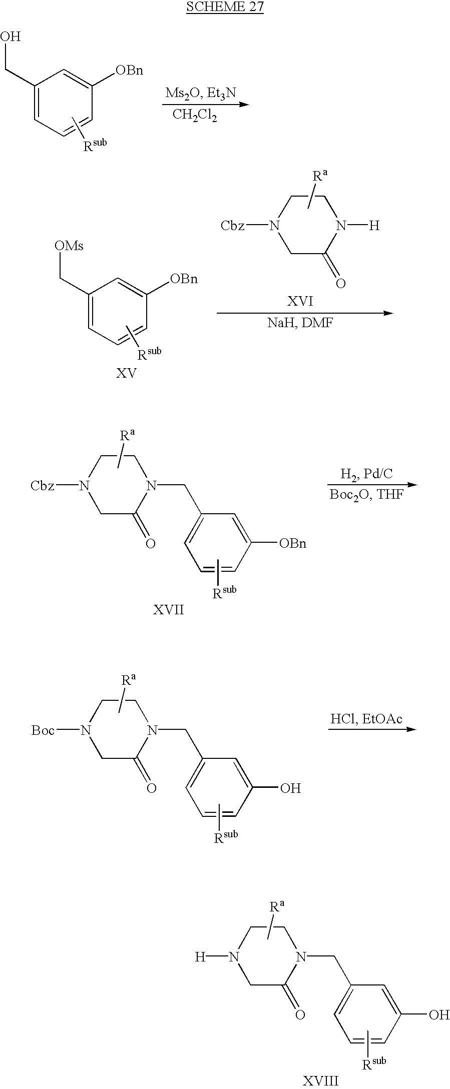

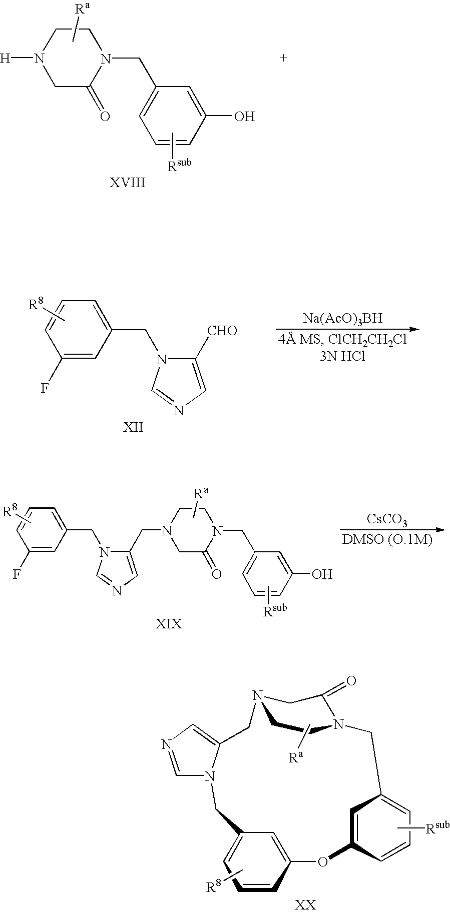

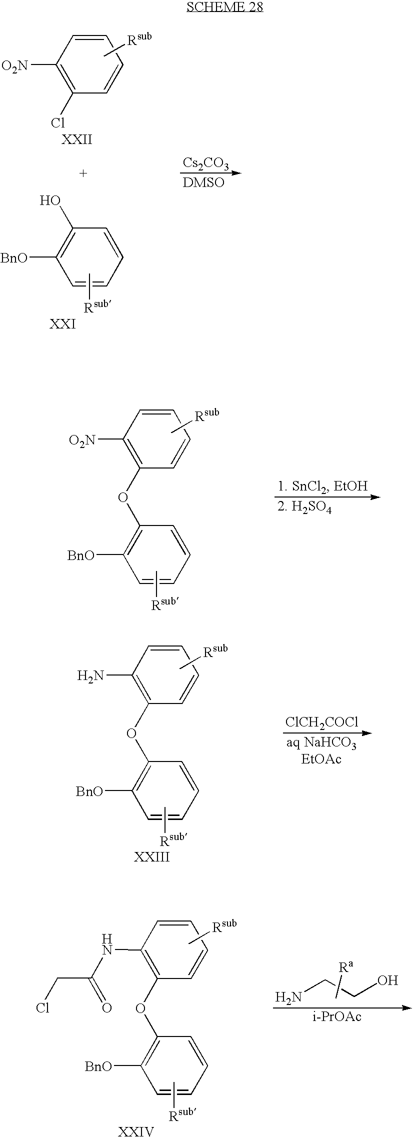

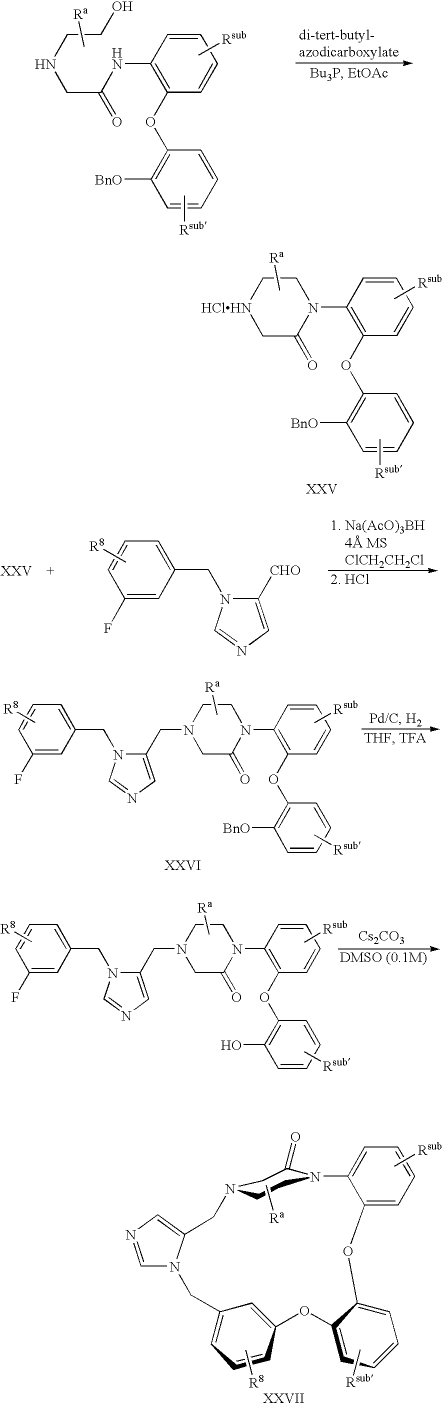

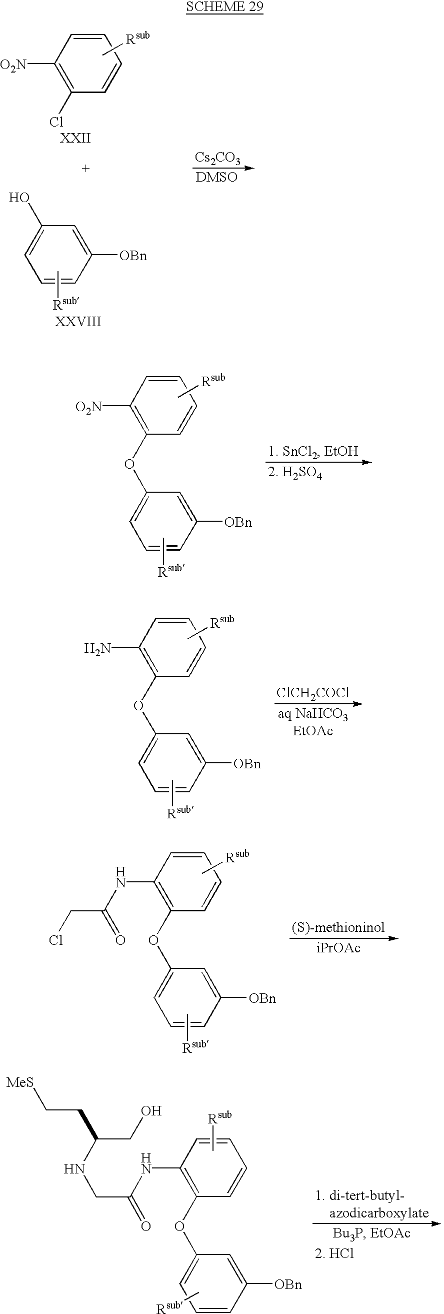

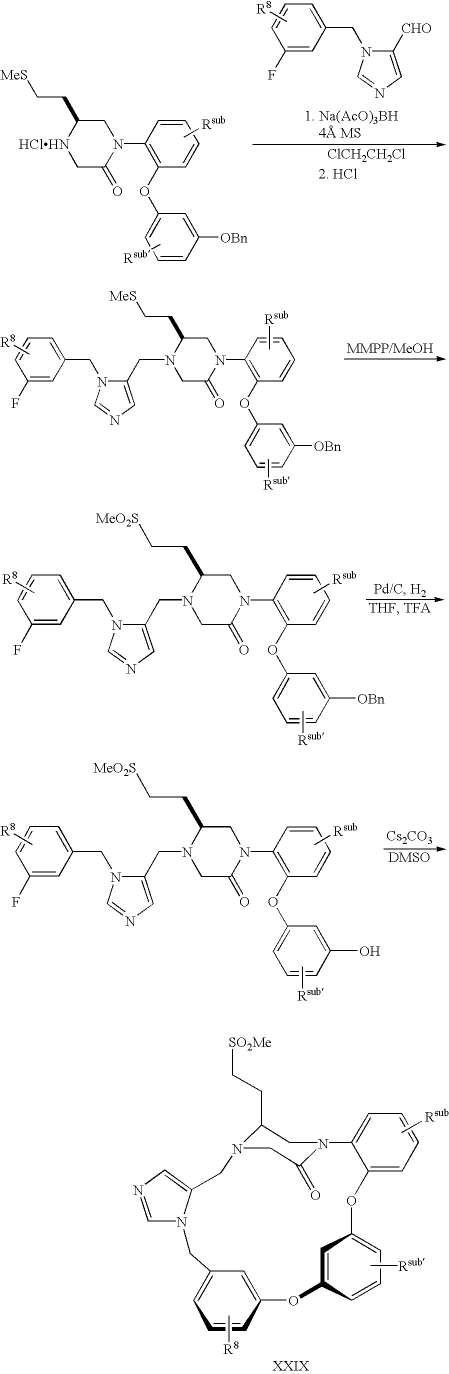

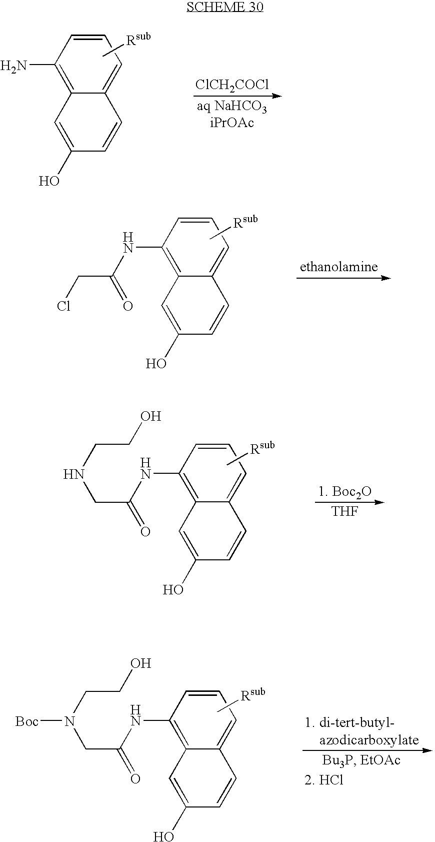

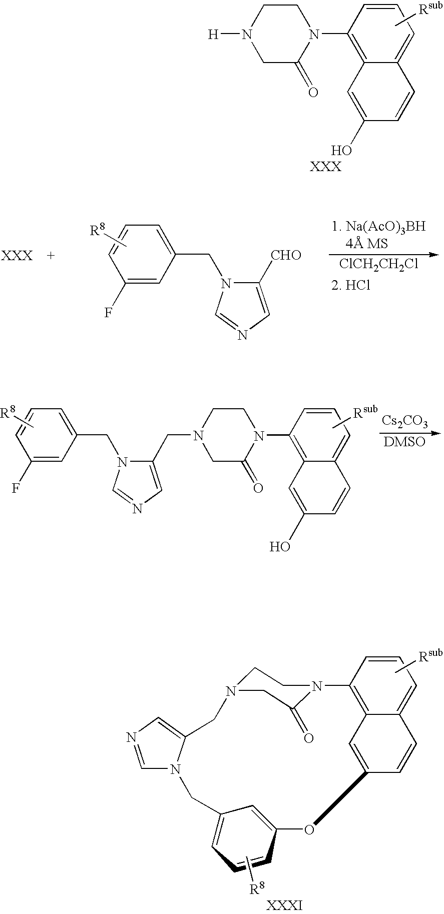

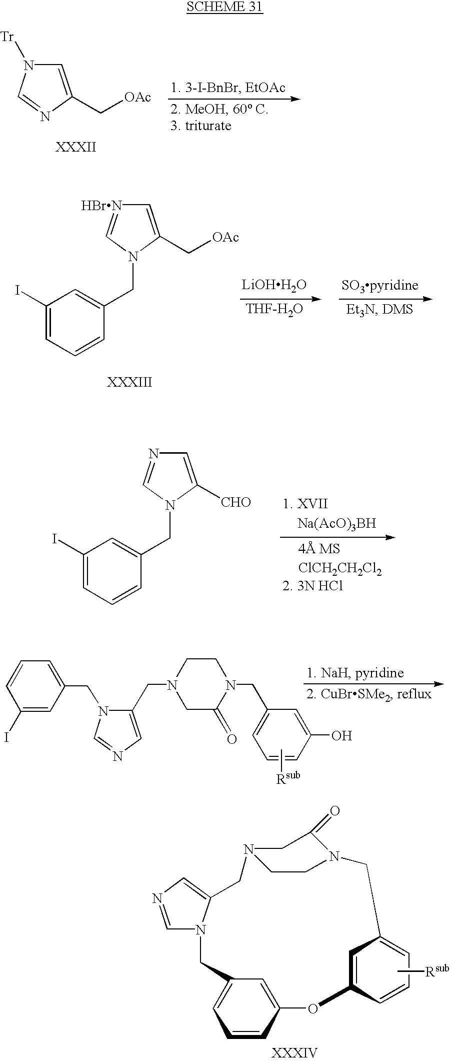

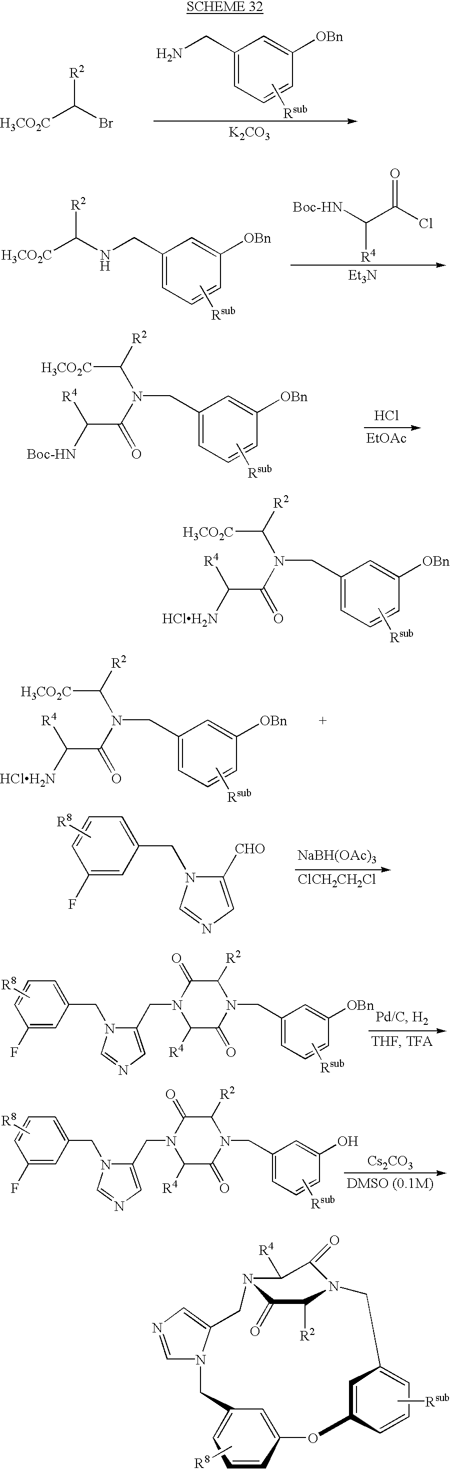

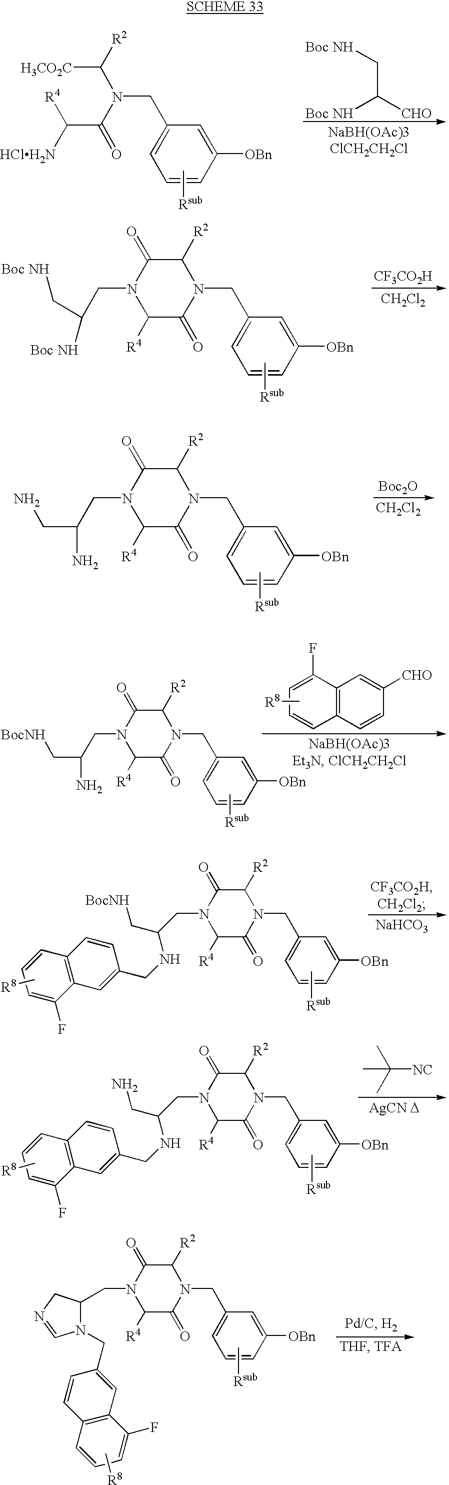

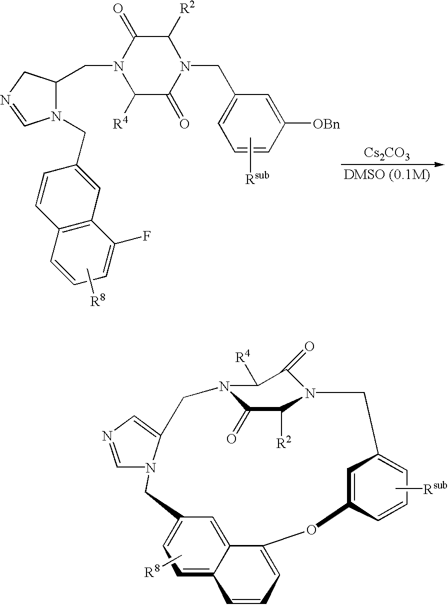

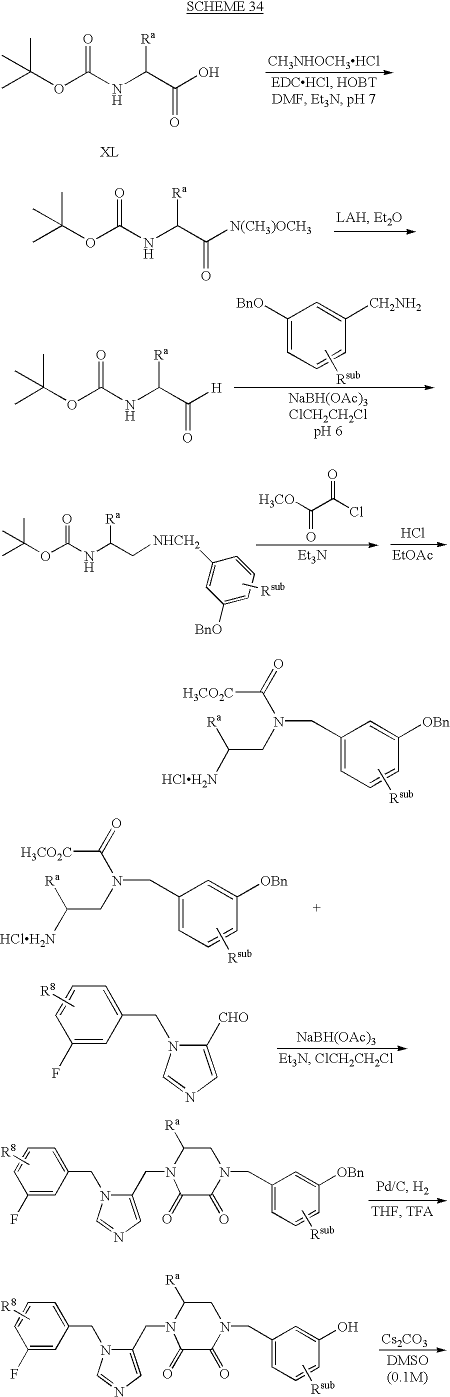

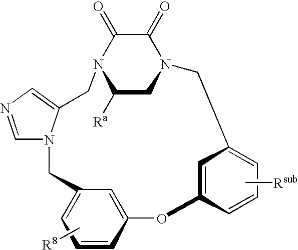

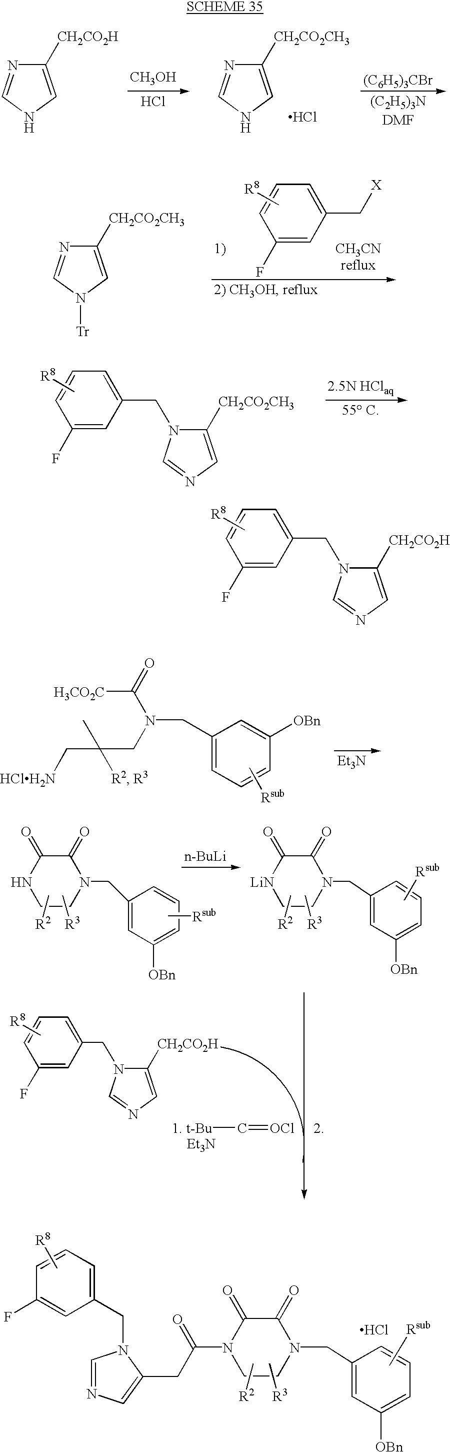

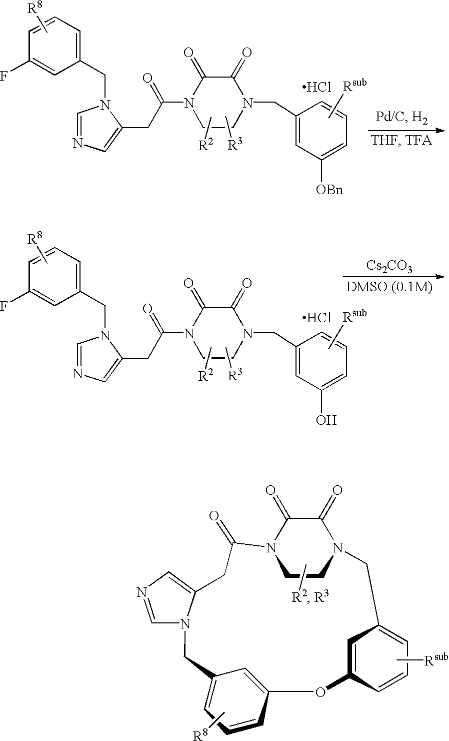

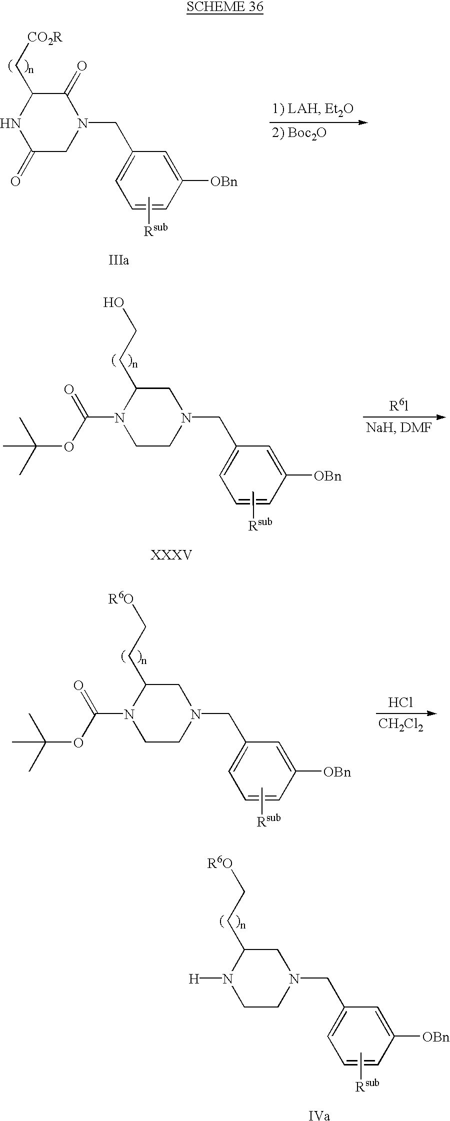

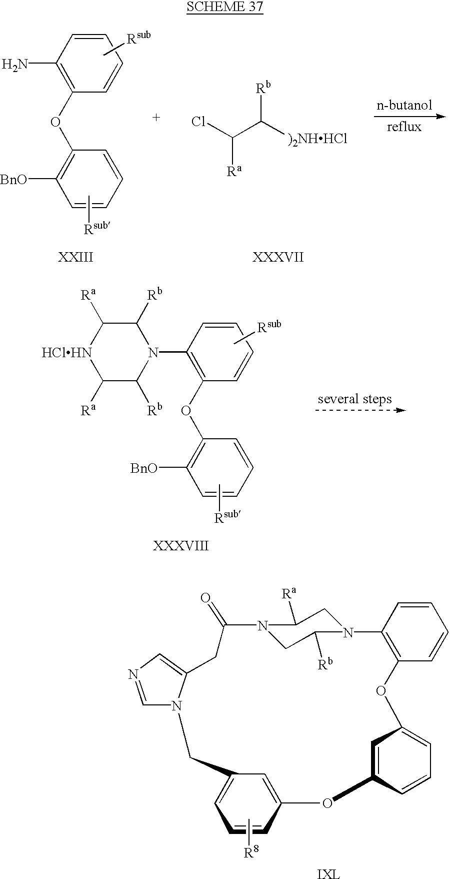

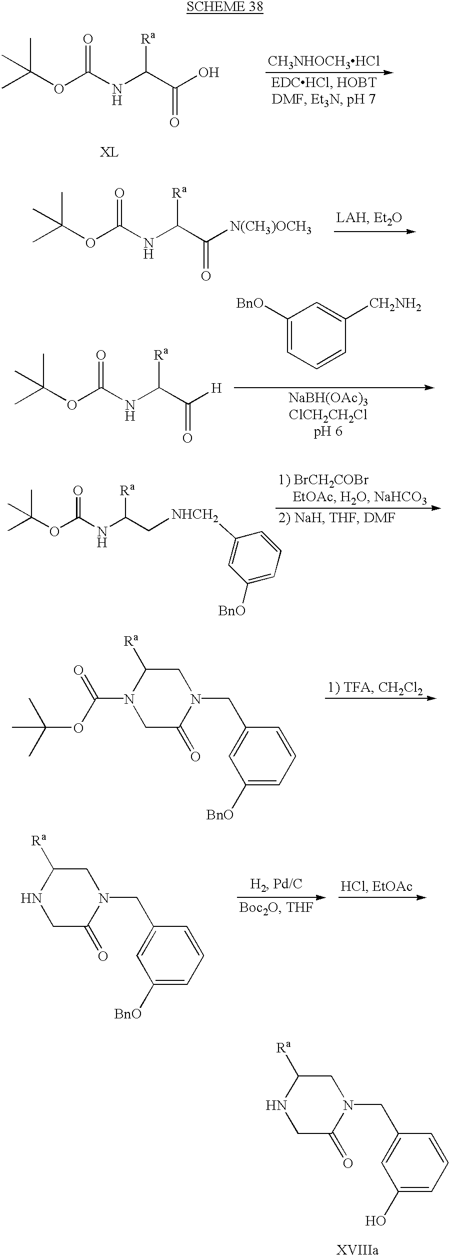

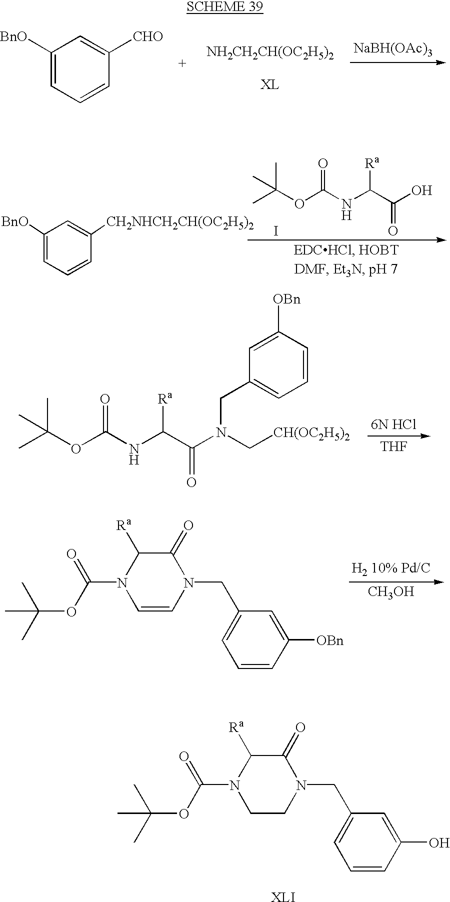

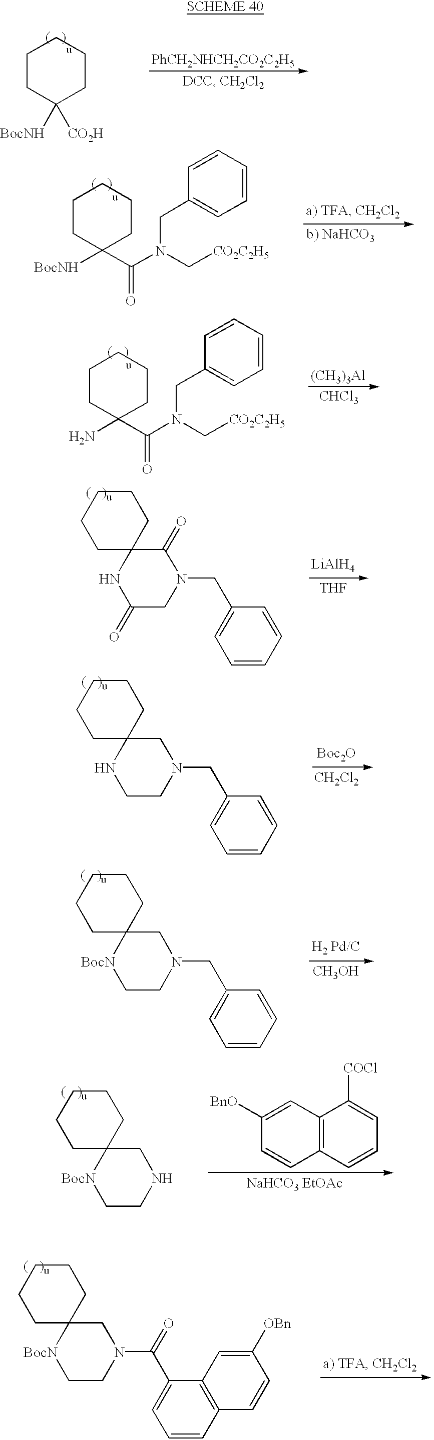

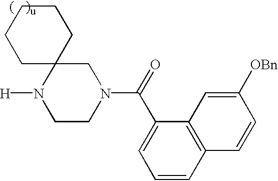

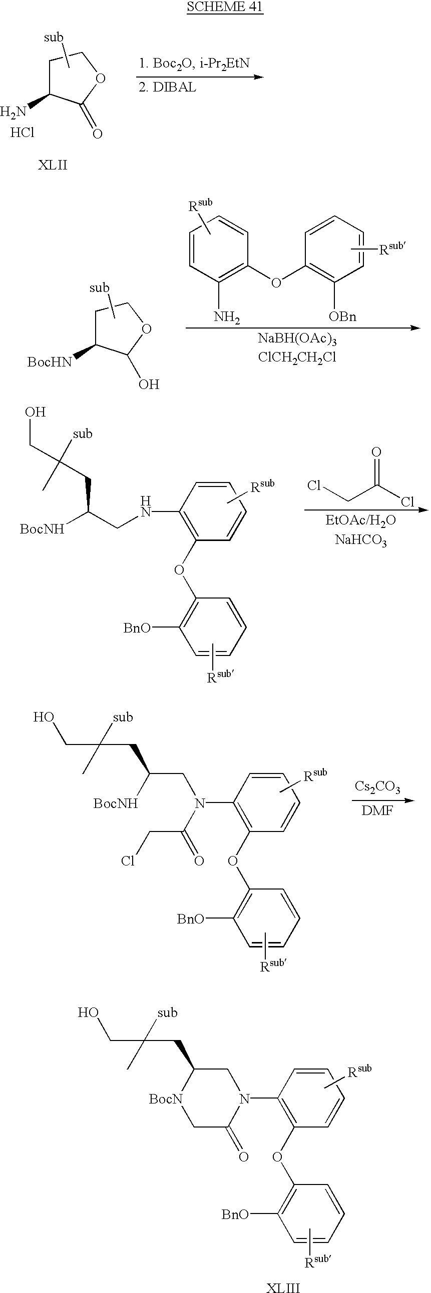

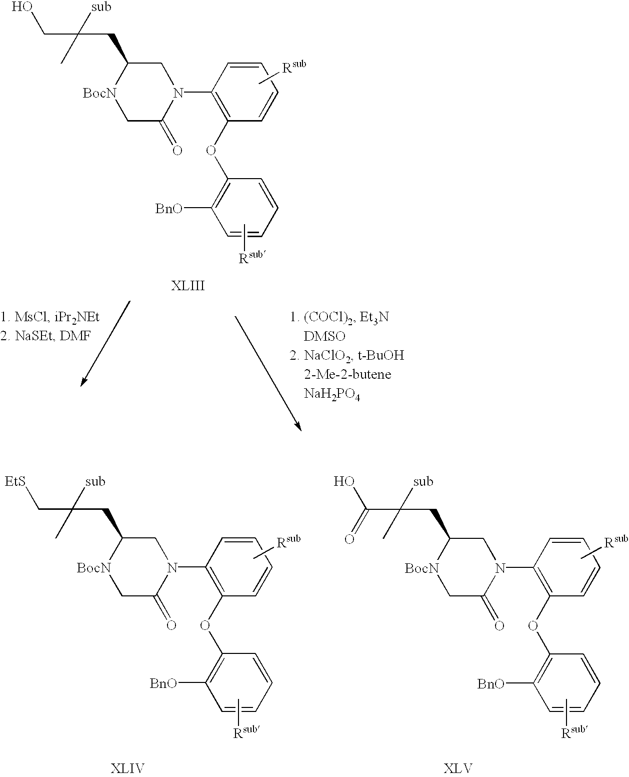

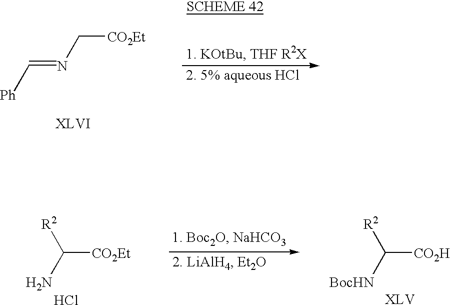

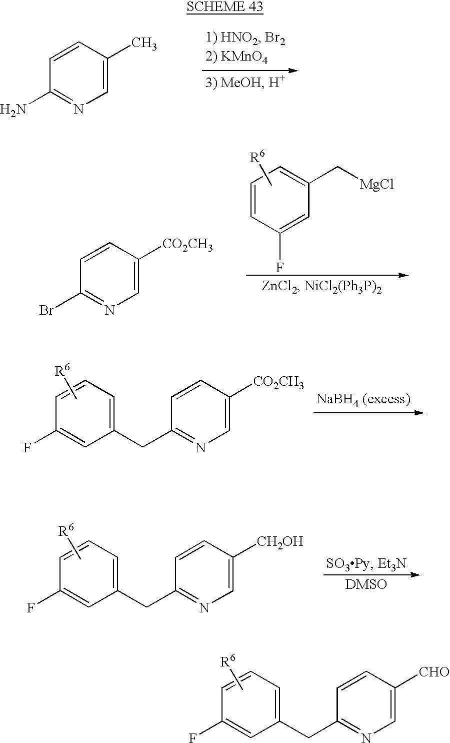

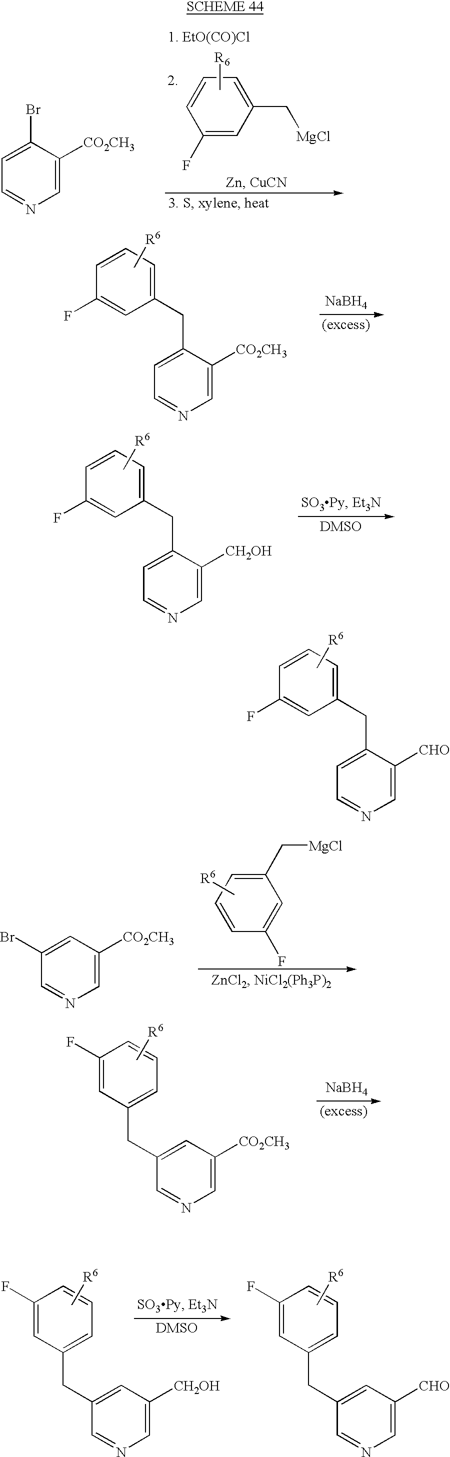

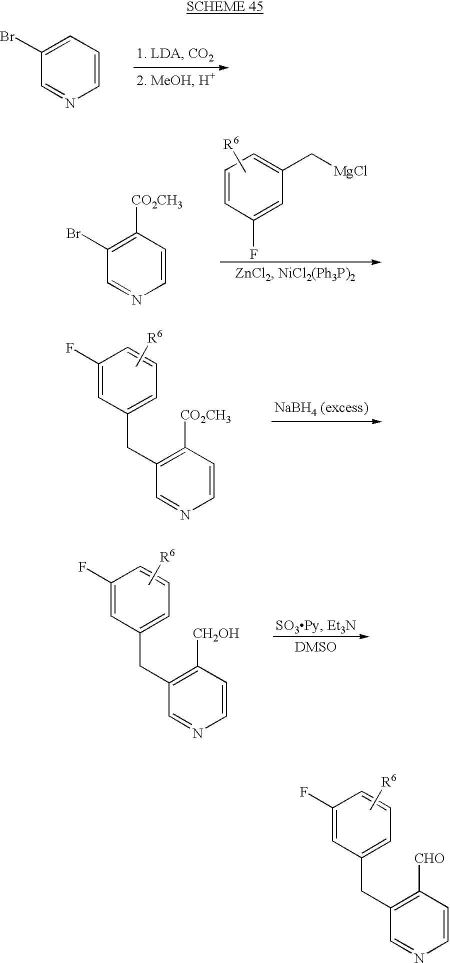

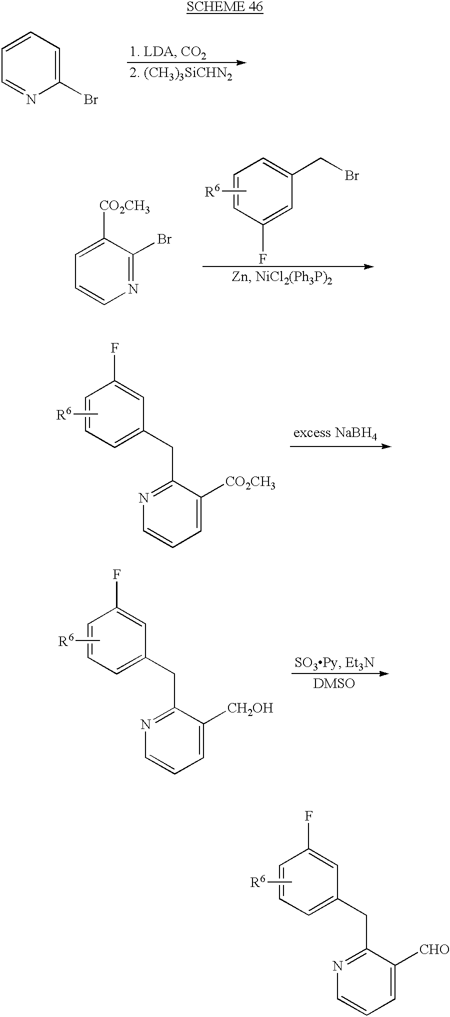

The farnesyl transferase inhibitors of formula (V) can be synthesized in accordance with Schemes 47-51, in addition to other standard manipulations such as ester hydrolysis, cleavage of protecting groups, etc., as may be known in the literature or exemplified in the experimental procedures. Substituents R, R^{a}, R^{b} and R^{sub}, as shown in the Schemes, represent the substituents R², R³, R⁴, and R⁵, and substituents on Z¹ and Z²; however their point of attachment to the ring is illustrative only and is not meant to be limiting. The compounds referred to in the Synopsis of Schemes 47-51 by Roman numerals are numbered starting sequentially with I and ending with XX.

These reactions may be employed in a linear sequence to provide the compounds of the invention or they may be used to synthesize fragments which are subsequently joined by the alkylation reactions described in the Schemes.

Synopsis of Schemes 47-51:

The requisite intermediates are in some cases commercially available, or can be prepared according to literature procedures. In Scheme 47, for example, the synthesis of a key intermediate in the preparation of macrocyclic compounds of the instant invention containing the preferred benzylimidazolyl moiety is outlined. A suitably substituted fluorotoluene I is brominated and reacted with an imidazolylmethyl acetate to form the intermediate II. Reduction, followed by oxidation provided the aldehyde III which is then reductively alkylated with a suitably substituted amine to provide the intermediate IV.

Scheme 48 illustrates the synthesis of a compound of the instant invention which utilizes intermediate IV. Thus, a suitably substituted hydroxyanaline V is N-protected, for example with by reductive alkylation with 2,4-dimethoxybenzaldehyde, and the resulting secondary amine is reacted with a suitably substituted chloroacetyl chloride to provide intermediate VI. Intermediate VI is then reacted with the imidazolylmethylamine IV to provide the protected amide VII. Intermediate VII may then undergo a cesium carbonate nucleophillic aromatic substitution reaction resulting in an intramolecular cyclization to yield compound VIII of the instant invention. This cyclization reaction depends on the presence of an electronic withdrawing moiety (such as nitro, cyano, and the like) either ortho or para to the fluorine atom. Compound VIII may be N-deprotected to provide instant compound IX, which may itself be further elaborated, for example by boronic acid coupling to give compound X of the instant invention.

Syntheses of compounds of the instant invention wherein the linker “X” is an ether linkage are illustrated in Scheme 49. Thus, the protected amide VI is reacted with a suitably substituted sodium benzylimidazolyl methoxide to provide intermediate XI, intramolecular cyclization as previously described, followed by deprotection provides the instant compound XII, which can be further elaborated as shown.

Scheme 50 illustrates syntheses of instant compounds wherein the linker “X” is an amido linkage. Thus, the primary amine XIII, homologous to intermediate IV is reacted with a suitably substituted bromoacetyl bromide, followed by a reaction with a nucleophile, such as a suitably substituted O-protected hydroxythiophenol. The resulting intermediate XIV is deprotected and intramolecular cyclization as previously described provides compound XV of the instant invention. The sulfur moiety in compound XV also may be oxidized to provide instant compound XVI.

Scheme 51 illustrates the synthetic strategy that is employed when the R⁸ substitutent is not an electronic withdrawing moiety either ortho or para to the fluorine atom. In the absence of the electronic withdrawing moiety, the intramolecular cyclization can be accomplished via an Ullmann reaction. Thus, the previously described aldehyde III can be converted to the homologous amine XVII. Amine XVII is then reacted with the previously described chloroacetamide VI to provide intermediate XVIII. Intramolecular cyclization may then be affected under Ullmann reaction to provide intermediate XIX, which may be deprotected and reduced to provide the diamino macrocycle of the instant invention XX.

Schemes 43-46 hereinabove illustrate syntheses of suitably substituted aldehydes useful in the syntheses of the instant compounds wherein the variable W is present as a pyridyl moiety. Similar synthetic strategies for preparing alkanols that incorporate other heterocyclic moieties for variable W are also well known in the art.

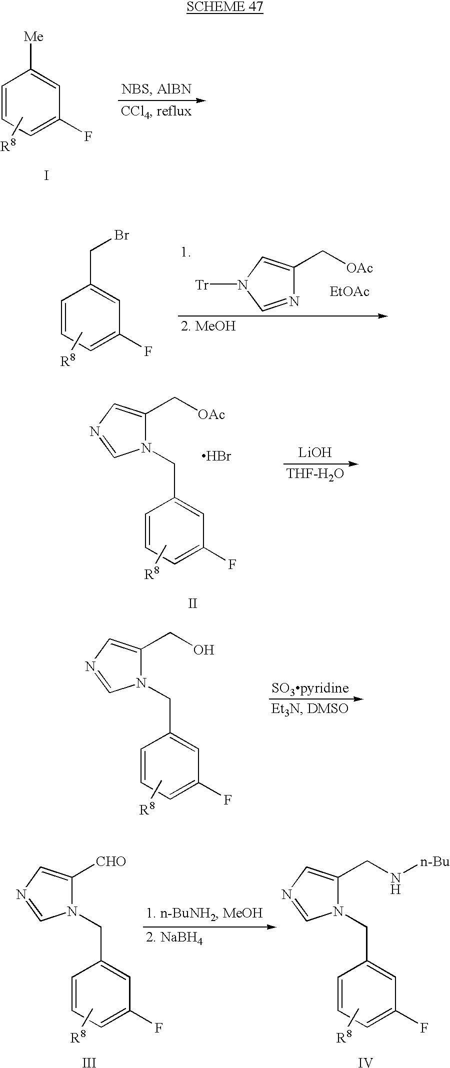

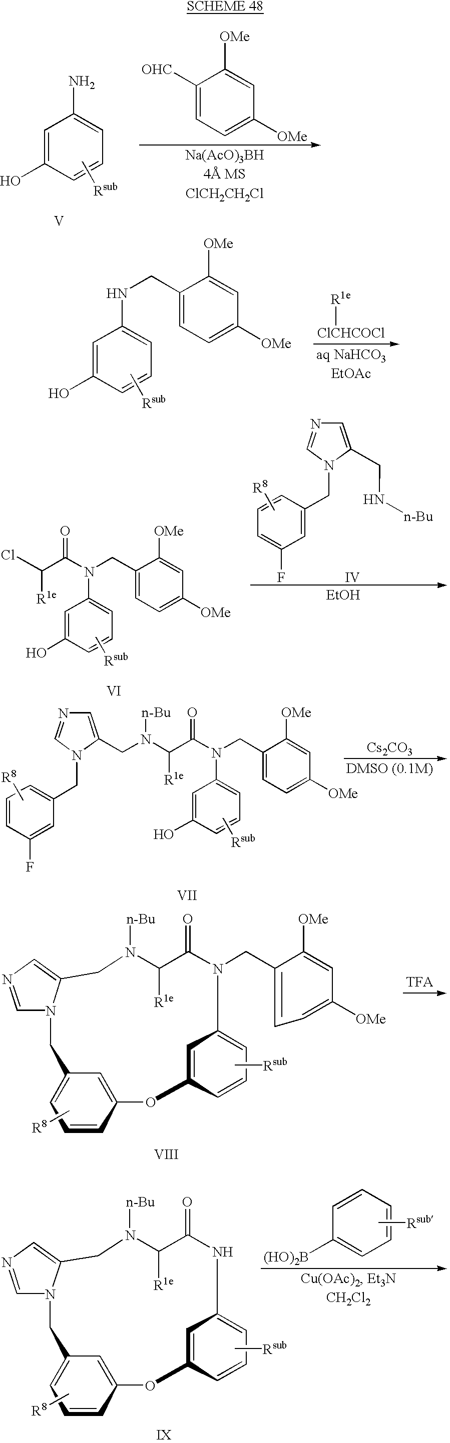

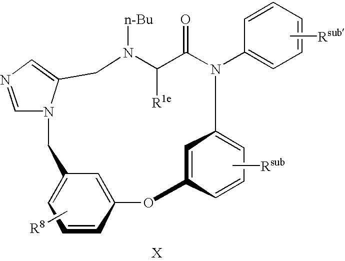

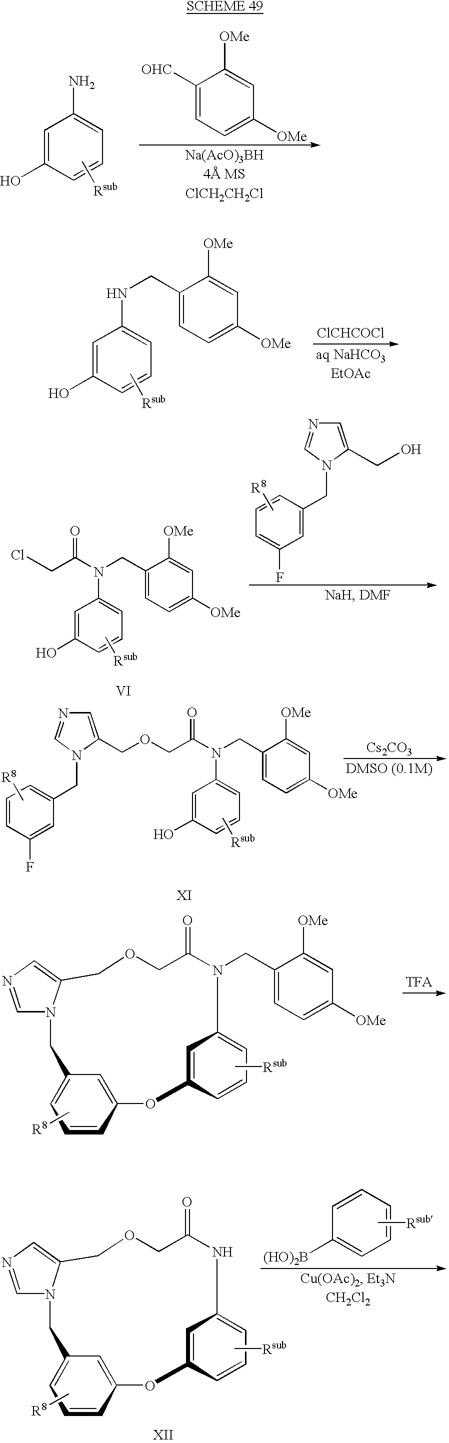

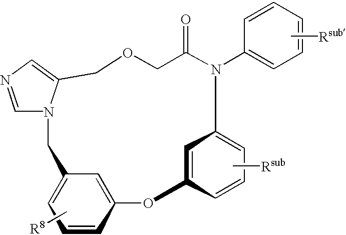

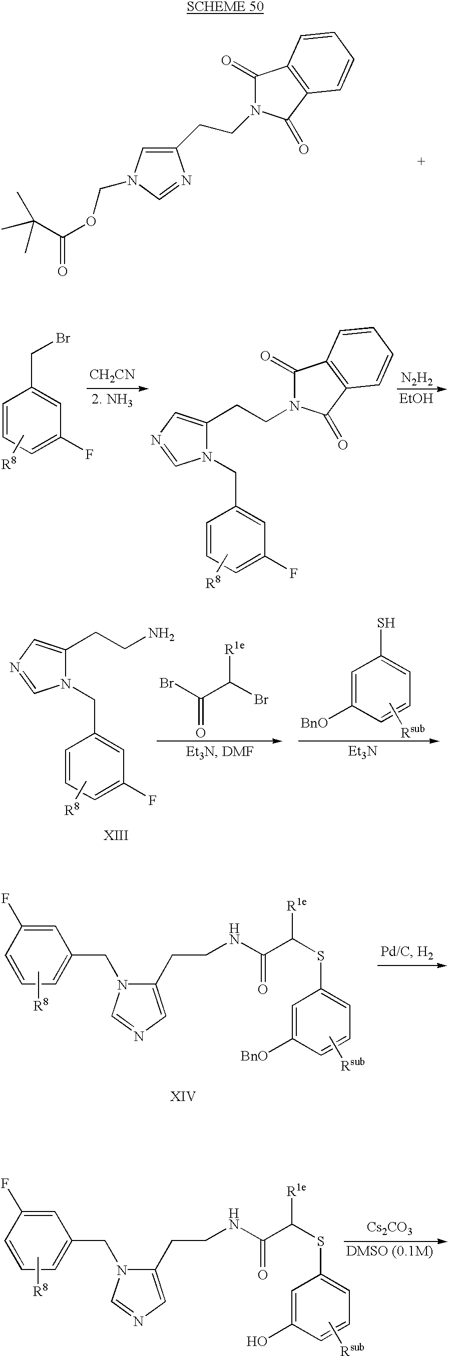

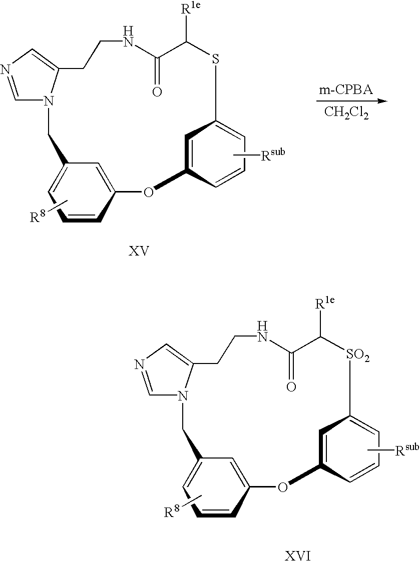

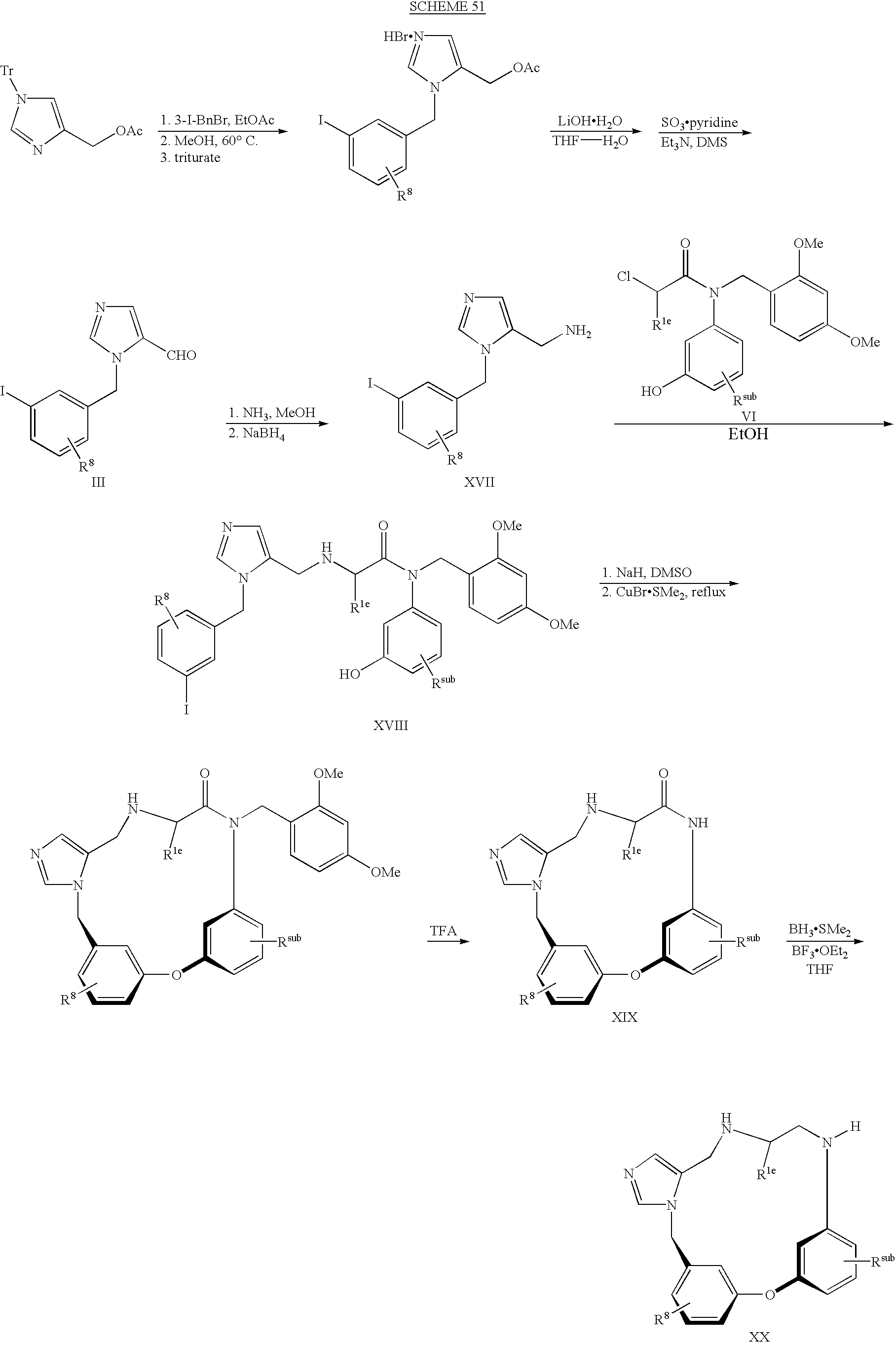

The farnesyl transferase inhibitors of formula (VI) can be synthesized in accordance with Schemes 52-57, in addition to other standard manipulations such as ester hydrolysis, cleavage of protecting groups, etc., as may be known in the literature or exemplified in the experimental procedures. Substituents R, R^{a}, R^{b} and R^{sub}, as shown in the Schemes, represent the substituents R², R³, R⁴, and R⁵, and substituents on Z¹ and Z²; however their point of attachment to the ring is illustrative only and is not meant to be limiting. The compounds referred to in the Synopsis of Schemes 52-57 by Roman numerals are numbered starting sequentially with I and ending with XVIII.

These reactions may be employed in a linear sequence to provide the compounds of the invention or they may be used to synthesize fragments which are subsequently joined by the alkylation reactions described in the Schemes.

Synopsis of Schemes 52-57:

The requisite intermediates are in some cases commercially available, or can be prepared according to literature procedures. For example, syntheses of instant compounds wherein the linker “X” is an sulfonamido linkage is illustrated in Scheme 52. Thus, a suitably substituted benzylimidazolyl containing amine I is prepared as illustrated. A suitably substituted benzyl alcohol II is converted to the corresponding benzylsulfinylchloride III. Reaction of intermediate III with the primary amine I provides the sulfinamido intermediate IV. That intermediate can be oxidized to the sulfonamide, the alcohol moiety can then be deprotected and previously described intramolecular cyclization provides compound V of the instant invention.

Instant compounds wherein the variable “V” is other than a phenyl moiety can be prepared as illustrated in Scheme 53. Thus, a suitably substituted fluoronaphthylmethyl bromide VII may be reacted with an imidazolyl alkylacetate to provide intermediate VIII. The alcohol moiety of intermediate VIII can be deprotected and then reacted with a suitably substituted phenyl isocyanate to provide the carbamate IX, which may then be optionally N-alkylated, followed by deprotection and intramolecular cyclization to provide compound XI of the instant invention.

Synthesis of compounds of the instant invention wherein variables “Z¹” and “Z²” are both phenyl moieties and the linker “X” is a amido moiety is illustrated in Scheme 54. Scheme 55 illustrates preparation of the corresponding instant compound wherein linker “X” is a urea moiety by reacting the isocyanate derived from intermediate I and the phenoxyanaline XIII described in Scheme 54. Synthesis of compounds of the instant invention wherein variable “Z¹” is a naphthyl moiety and the linker “X” is a amido moiety is illustrated in Scheme 56.

Scheme 57 illustrates the synthetic strategy that is employed when the R⁸ substitutent is not an electronic withdrawing moiety either ortho or para to the fluorine atom. In the absence of the electronic withdrawing moiety, the intramolecular cyclization can be accomplished via an Ullmann reaction. Thus, the aldehyde XIV can be converted to the homologous amine XV. Amine XV is then reacted with the previously described benzyloxybenzoic acid XVI to provide intermediate XVII. Intramolecular cyclization may then be affected under Ullmann reaction conditions to provide the amido macrocycle of the instant invention XVIII.

Other suitably substituted aldehydes such as those described in Schemes 43-46 hereinabove may be utilized in the syntheses of the instant compounds of the formula VI wherein the moiety “W” is pyridyl. Similar synthetic strategies for preparing alkanols that incorporate other heterocyclic moieties for variable W are also well known in the art.

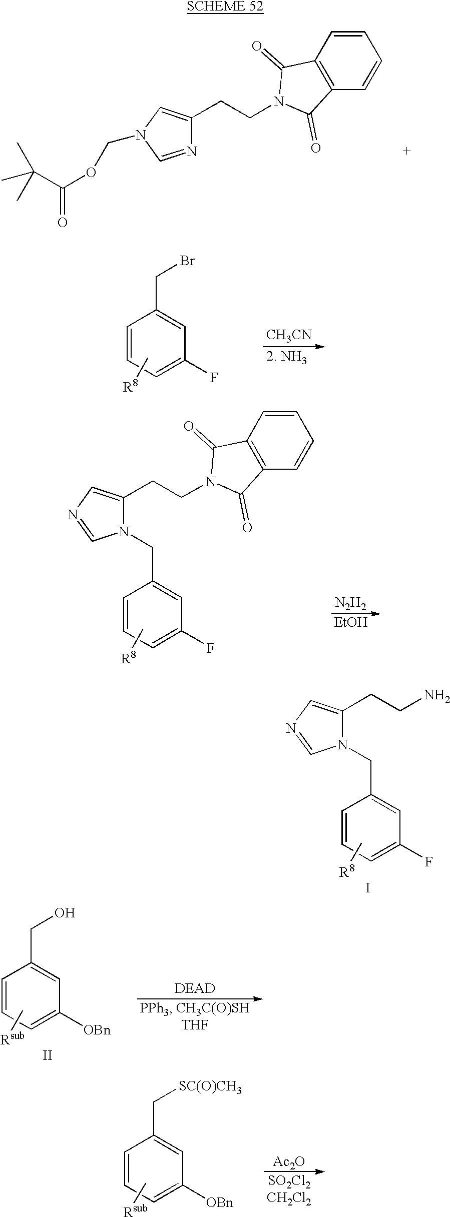

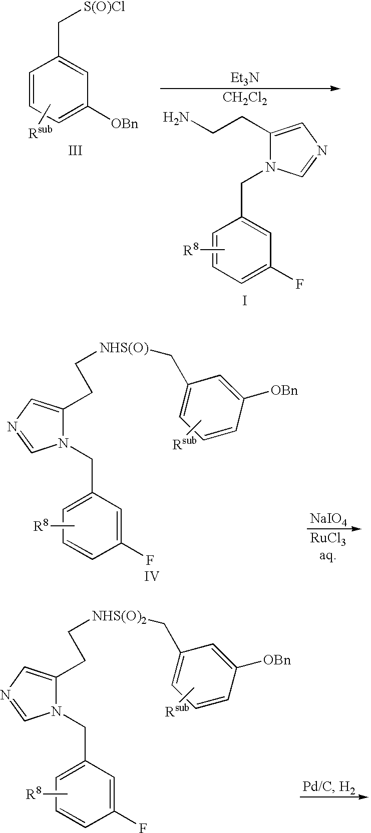

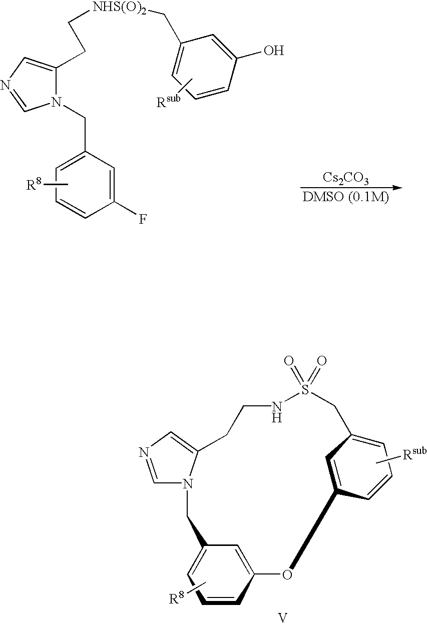

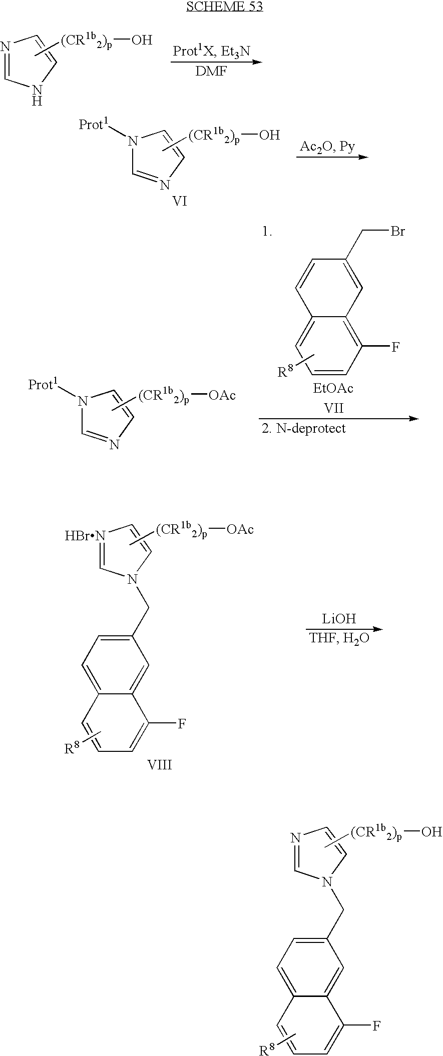

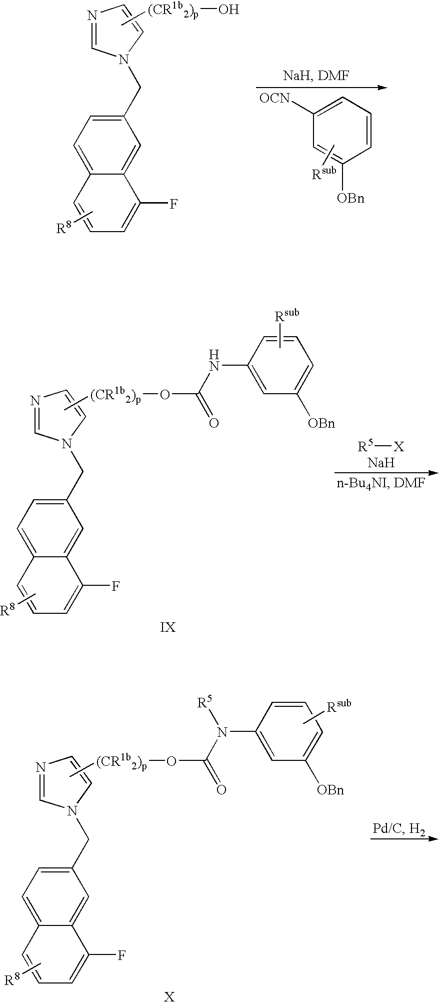

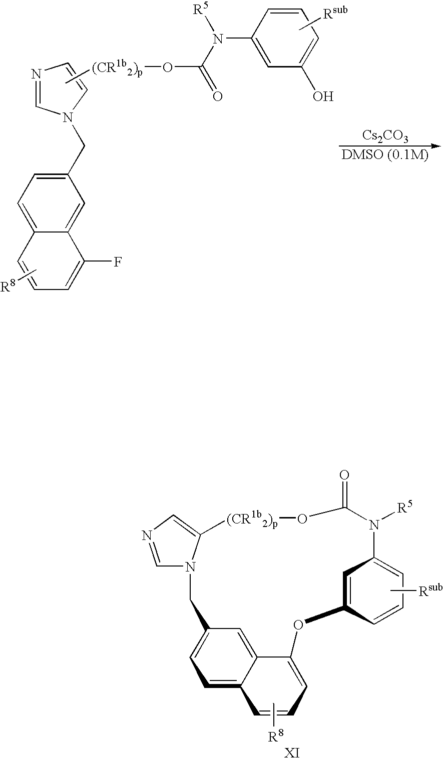

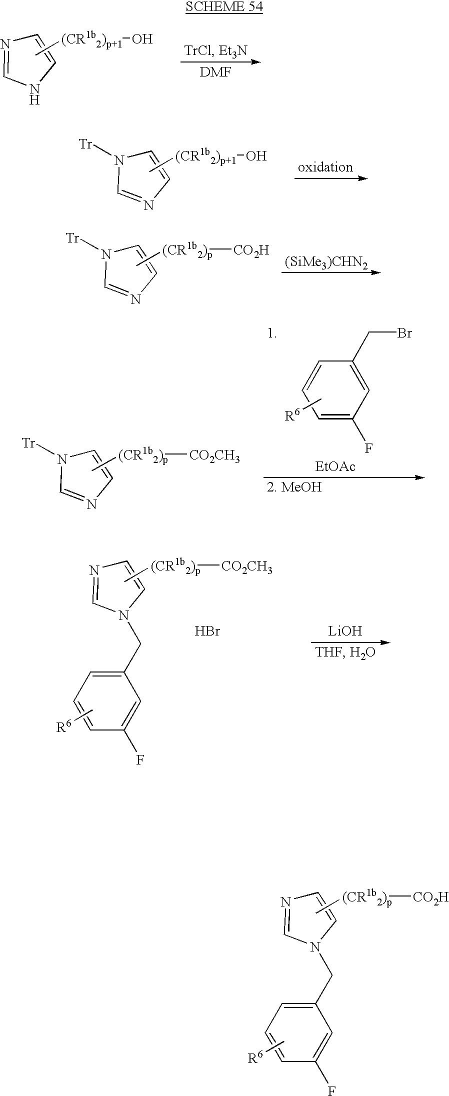

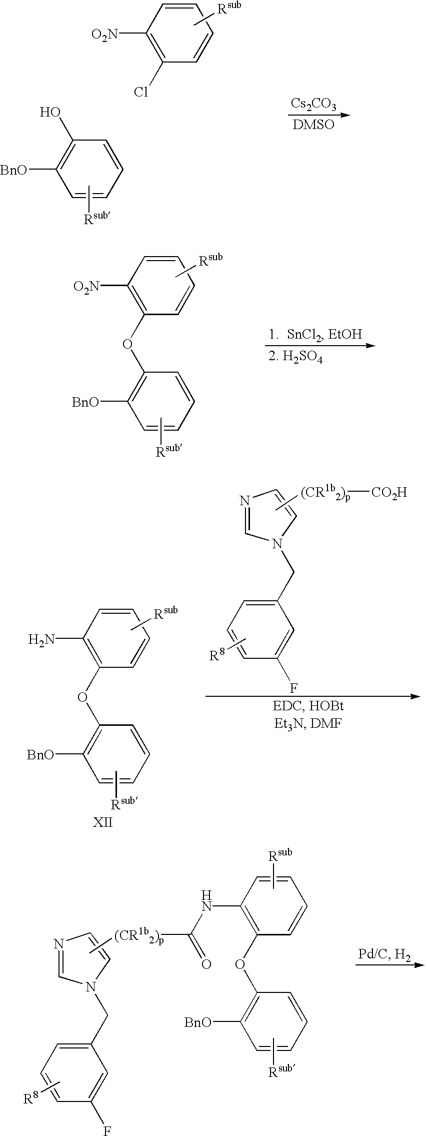

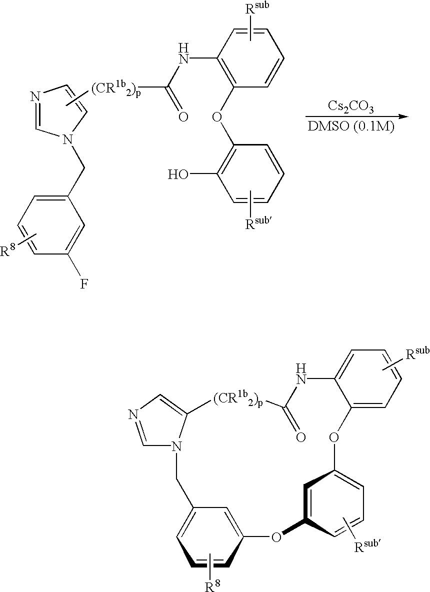

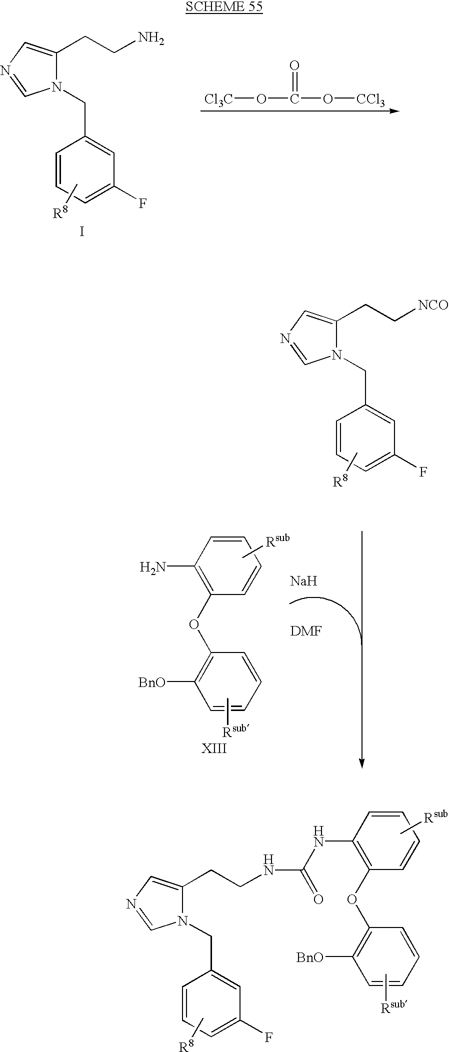

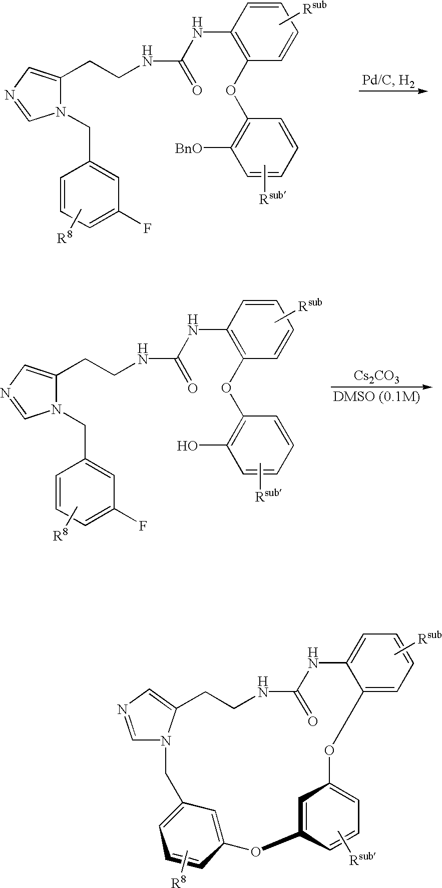

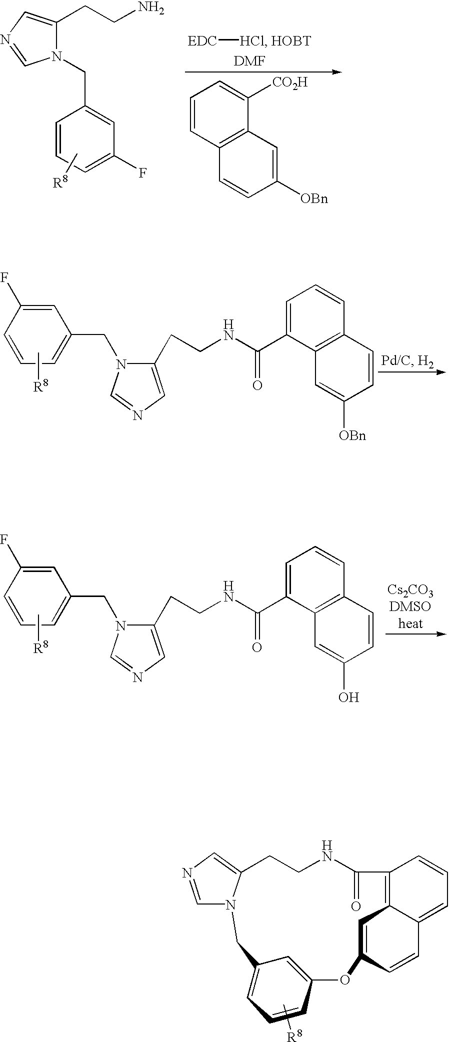

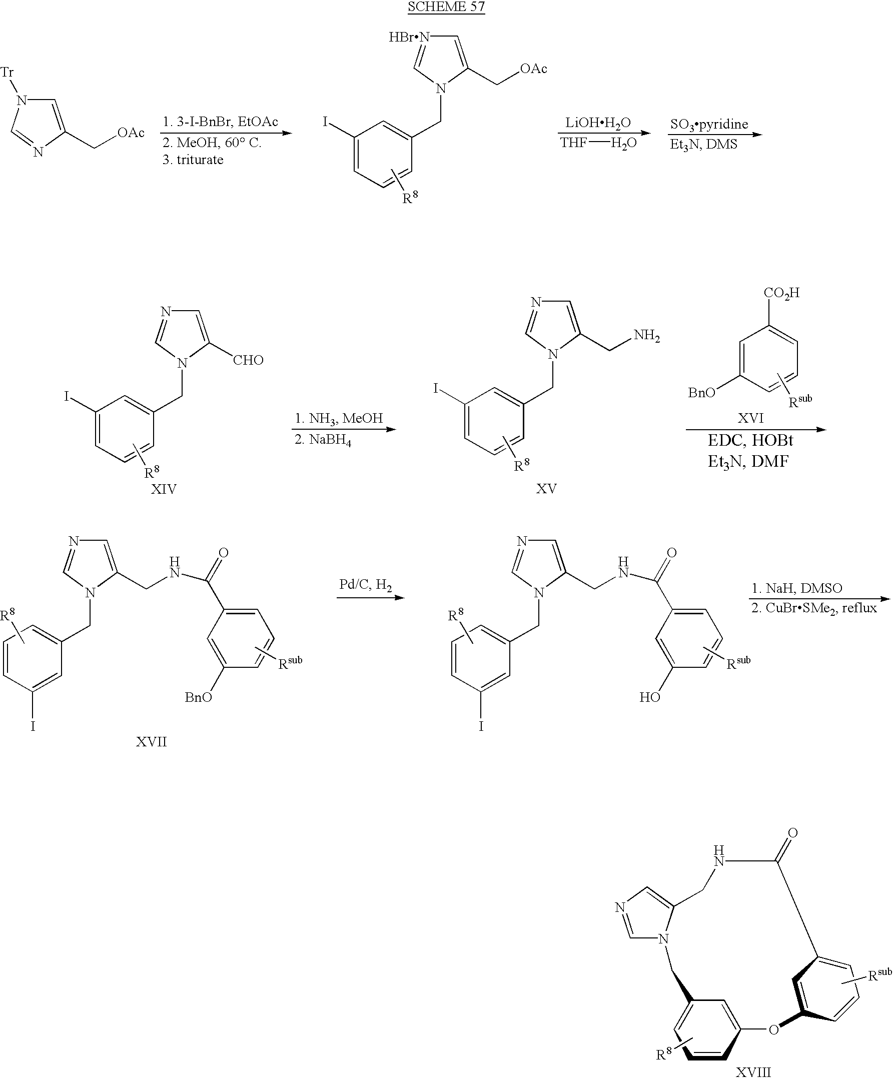

The prenyl transferase inhibitors of formula (VII) can be synthesized in accordance with Schemes 58-66, in addition to other standard manipulations such as ester hydrolysis, cleavage of protecting groups, etc., as may be known in the literature or exemplified in the experimental procedures. The compounds referred to in the Synopsis of Schemes 58-66 by Roman numerals are numbered starting sequentially with II and ending with XXXX.

These reactions may be employed in a linear sequence to provide the compounds of the invention or they may be used to synthesize fragments which are subsequently joined by the alkylation reactions described in the Schemes.

Synopsis of Schemes 58-66

Schemes 58-66 describe the synthesis of compounds of formulae VII. The starting materials can be obtained from commercial sources or they can be obtained using standard transformations (e.g. esterification of the hydroxy acid) from commercially available materials.

In Scheme 58, amino-hydroxybenzoates of type II can be converted to the corresponding iodide III by treatment with acidic aqueous NaNO₂ followed by the addition of KI. The phenol may then be alkylated by treatment with a base such as NaH or Cs₂CO₃ in an organic solvent (for example DMF) followed by the addition of an electrophile to yield IV. Reduction of the ester of IV using, for example, LiBH₄ in THF then yields the alcohol V which can in turn be treated with Zn(CN)₂ in DMF and a palladium catalyst to give VI. The alcohol of VI can be converted into a leaving group of VII in a number of ways. One such procedure involves reaction of the alcohol with a sulfonyl chloride in the presence of an organic base (e.g. triethylamine) in an organic solvent such as dichloromethane. A second method requires the reaction of the alcohol with CBr₄ and a phosphine such as triphenyl phosphine in an organic solvent such as dichloromethane. A third method involves reaction of the alcohol with N-bromosuccinimide and dimethyl sulfide in dichloromethane. The reaction of VII with imidazole in a polar solvent such as DMF then affords compounds of formula IA. In addition, VII upon reaction with 4-iodo-1-tritylimidazole in THF with 1,2-dibromoethane, Zn and NiCl₂(PPh₃)₂ and subsequent methanolysis may yield compounds of formula IB.

Scheme 59 shows an alternative route for the conversion of III into VI employing chemical transformations described above.

In Scheme 60, the phenol X can be converted to the corresponding triflate XI using trifluoromethane sulfonic anhydride in an organic solvent such as dichloromethane with an organic base such as triethylamine. The triflate may then be converted to the nitrile XII, the ester reduced to XIII and the alcohol transformed to a leaving group as shown in XIV using previously described reactions. Treatment of XIV as above would then produce compounds of formula IC or ID.

An alternative route for the synthesis of compounds of formula IA and IB is given in Scheme 61. Methyl-hydroxybenzoic acids of structure XV may be bis alkylated by treatment with a base such as NaH or Cs₂CO₃ in an organic solvent (for example DMF) followed by the addition of an electrophile to yield XVI saponification using aqueous hydroxide then affords the acid XVII. Acid XVII is then converted to the primary amide XIX via the acid chloride XVIII (prepared with thionyl chloride in a solvent such as toluene then a reaction with ammonia in, for example, chloroform). Treatment of XIX with thionyl chloride in DMF results in the nitrile XX which can be brominated at the benzylic position using, for example, N-bromosuccinimide and benzoyl peroxide in carbon tetrachloride. Transformations, as before, then yield IA or IB.

An alternative route for the synthesis of compounds of formula IA and IB is shown in Scheme 62 which incorporates the reaction steps described above but alters the order of these transformations to give XXIV which is converted to IA by treatment with a halide or mesylate.

Two routes to compounds containing a diaryl ether linkage as illustrated in XXVII are described in Scheme 85. The bromo fluoride XXV is transformed to the fluoro nitrile with zinc cyanide, then converted to ID in a series of transformations described in previous schemes.

Scheme 64 illustrates yet another route for the synthesis of compounds of formula IA.

Schemes 65 and 66 describe routes for the preparation of compounds XXXIX and XXXX which contain a heteroatom at the benzylic position between W and the phenyl ring of IA.

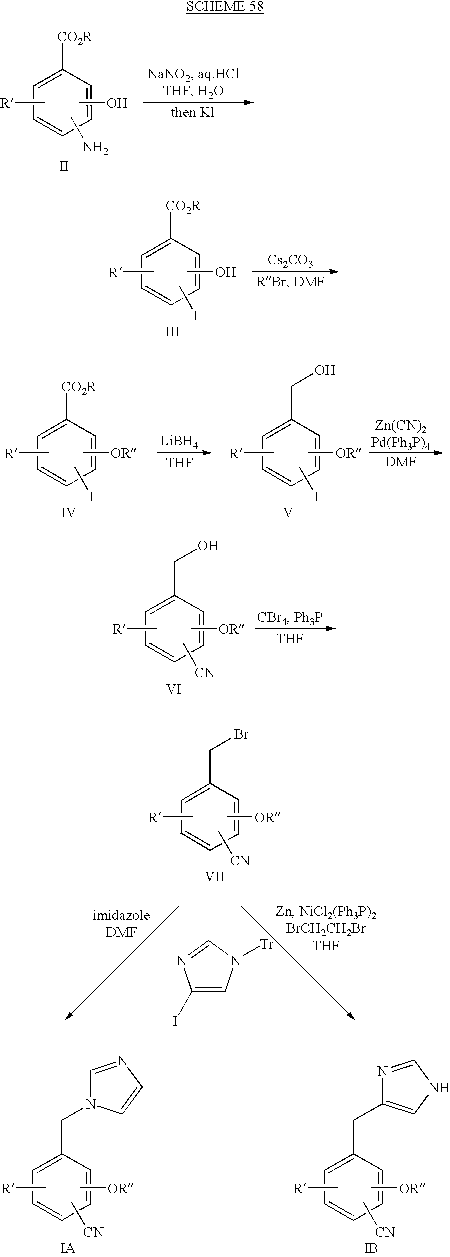

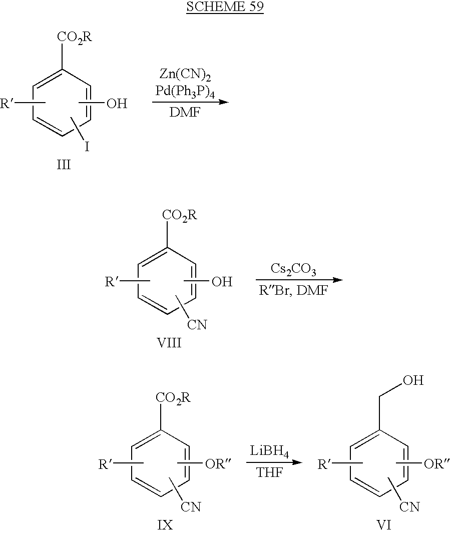

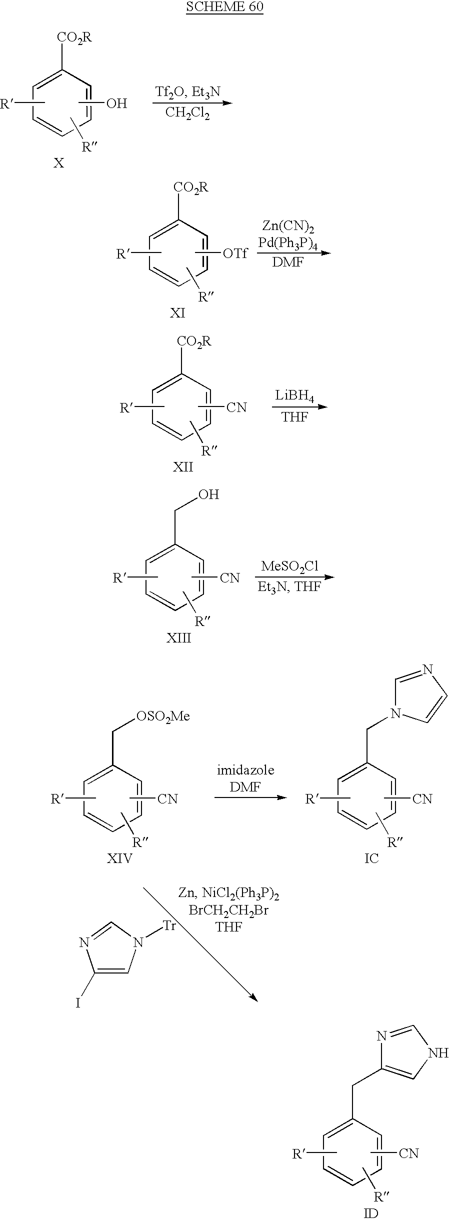

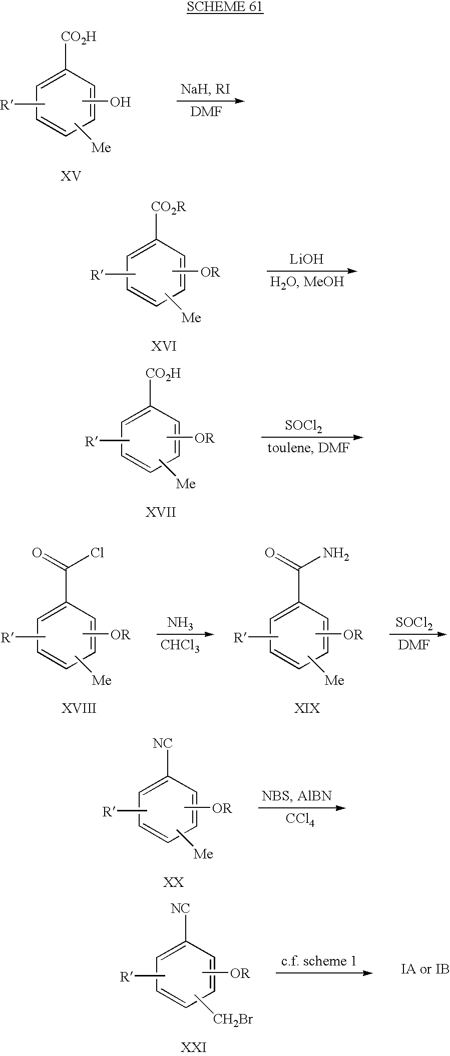

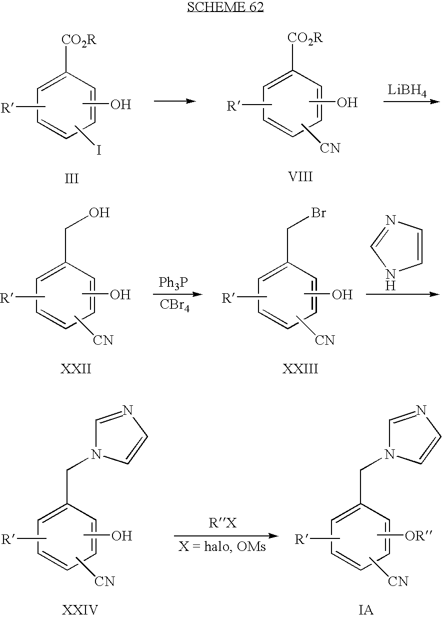

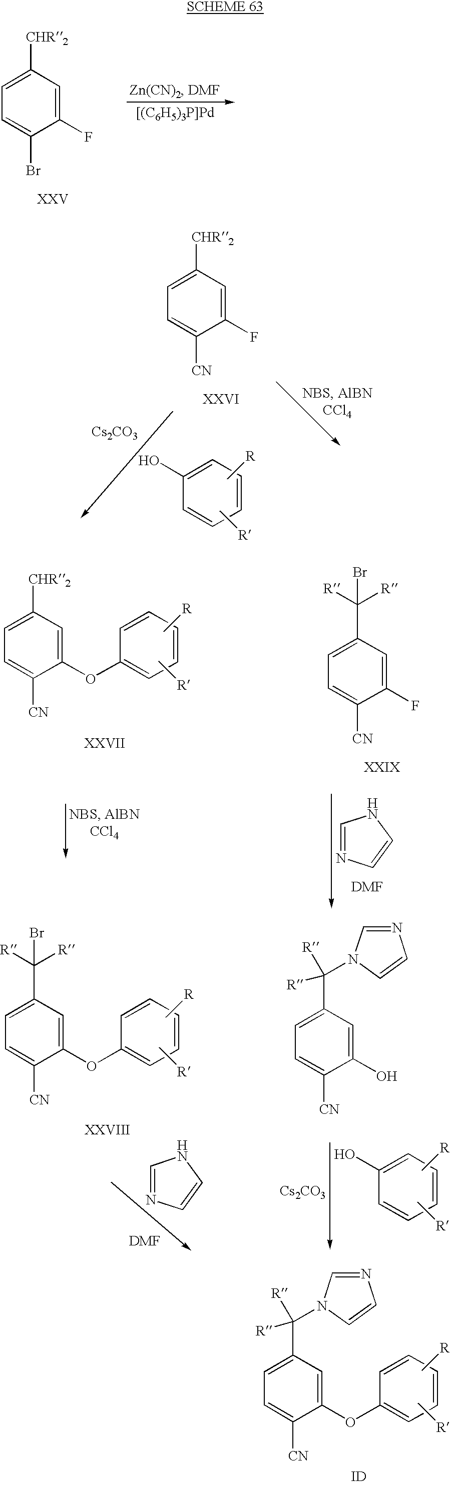

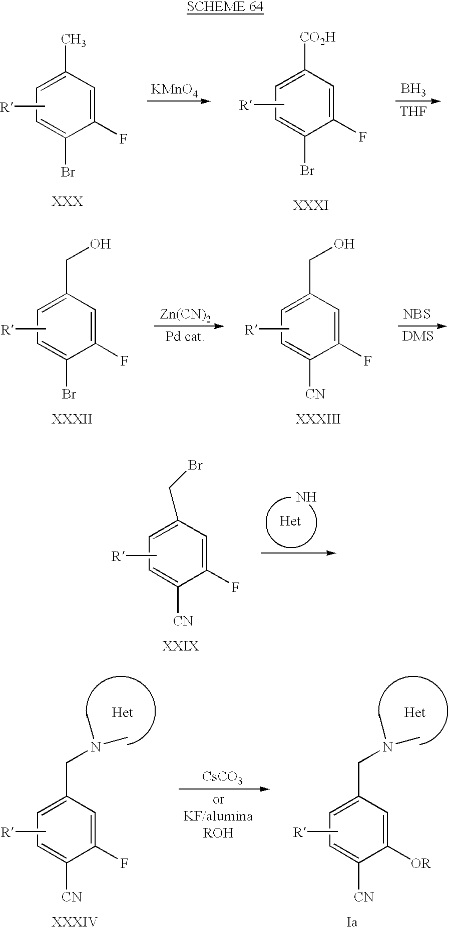

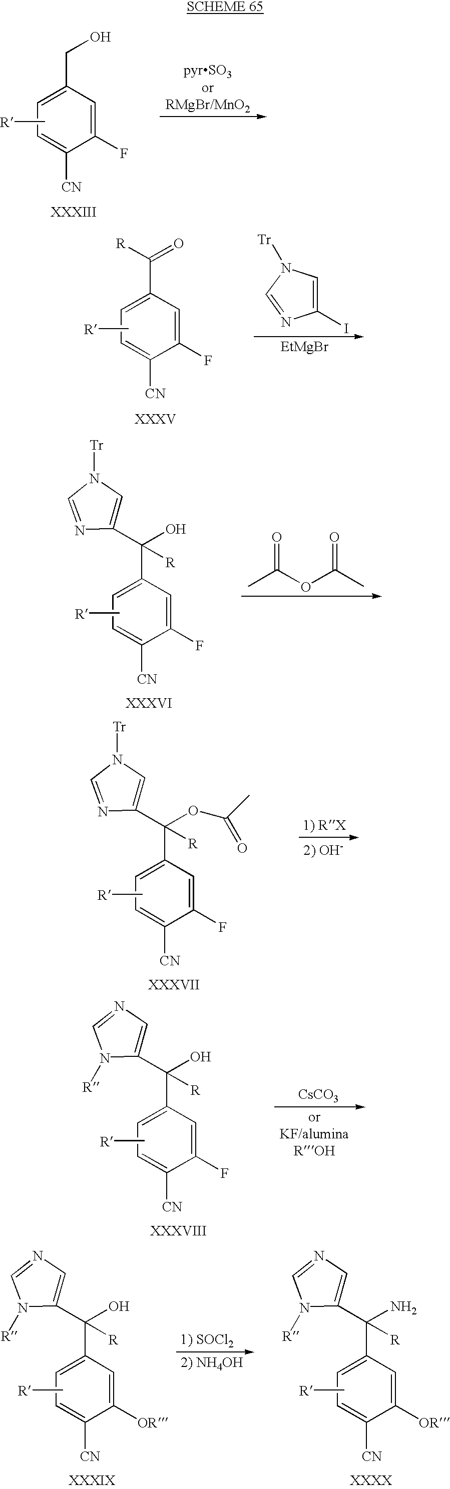

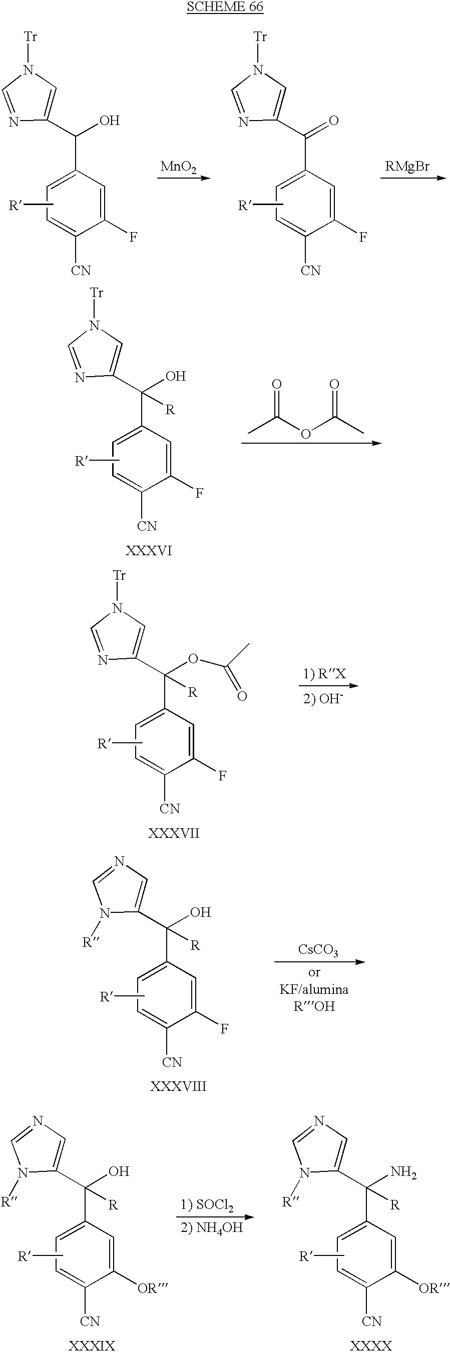

In Schemes 58-66, it is understood that:

R independently represents R^{1c} or its protected precursors thereof;

R′ independently represents R³ or its protected precursors thereof;

R″ independently represents R¹³ or its protected precursors thereof;

R′″ independently represents the following moiety:

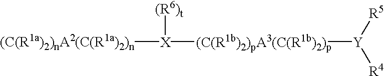

The farnesyl transferase inhibitors of formula (VIII) can be synthesized in accordance with Schemes 67-73, in addition to other standard manipulations such as ester hydrolysis, cleavage of protecting groups, etc., as may be known in the literature or exemplified in the experimental procedures. The compounds referred to in the Synopsis of Schemes 67-73 by Roman numerals are numbered starting sequentially with II and ending with XXX.

These reactions may be employed in a linear sequence to provide the compounds of the invention or they may be used to synthesize fragments which are subsequently joined by the alkylation reactions described in the Schemes.

Synopsis of Schemes 67-73

Schemes 67-73 describe the synthesis of compounds of formula VIII. The starting materials can be obtained from commercial sources or they can be obtained using standard transformations (e.g. esterification of the hydroxy acid) from commercially available materials.

In Scheme 67, amino-hydroxybenzoates of type I can be converted to the corresponding iodide II by treatment with acidic aqueous NaNO₂ followed by the addition of KI. The phenol may then be alkylated by treatment with a base such as NaH or Cs₂CO₃ in an organic solvent (for example DMF) followed by the addition of an electrophile to yield III. Reduction of the ester of III using, for example, LiBH₄ in THF then yields the alcohol IV which can in turn be treated with Zn(CN)₂ in DMF and a palladium catalyst to give V. The alcohol of V can be converted into a leaving group of VI in a number of ways. One such procedure involves reaction of the alcohol with a sulfonyl chloride in the presence of an organic base (e.g. triethylamine) in an organic solvent such as dichloromethane. A second method requires the reaction of the alcohol with CBr₄ and a phosphine such as triphenyl phosphine in an organic solvent such as dichloromethane. A third method involves reaction of the alcohol with N-bromosuccinimide and dimethyl sulfide in dichloromethane. The reaction of VI with imidazole in a polar solvent such as DMF then affords compounds of formula VII. In addition, VI upon reaction with 4-iodo-1-tritylimidazole in THF with 1,2-dibromoethane, Zn and NiCl₂(PPh₃)₂ gives a compound of formula VIII. Subsequent methanolysis of VIII may yield compounds of formula IX. Treating a compound of formula VIII with a suitably substituted alkyl or aralkyl halide or mesylate, followed by methanolysis yields a compound of formula X.

An alternative route for the synthesis of compounds of formula VII is shown in Scheme 68. Iodo-hydroxybenzoic acids of structure II may be converted to the corresponding cyano XI. The ester may be reduced by treating XI with LiBH₄ (and THF) to produce the alcohol XII. The alcohol can be converted into a leaving group by reacting the alcohol with CBr₄ and a phosphine, such as triphenyl phosphine (in an organic solvent such as dichloromethane). The reaction of XIII with imidazole affords XIV, which can be converted to VII by treatment with a suitably substituted alkyl or aralkyl halide or mesylate.

Scheme 69 illustrates one route for the synthesis of compounds of formula XXI. The bromotoluene XV can be treated with KMnO₄ to yield the bromo-fluorobenzoic acid XVI. The acid can be reduced with treatment of XVI with BH₃ in THF to give the bromobenzene XVII. The bromobenzene XVII can be converted to the corresponding cyanobenzene XVIII by treating XVII with Zn(CN)₂ and a palladium catalyst. The alcohol of intermediate XVIII can be converted into a leaving group by reacting the alcohol with NBS and DMS to produce XIX. Treatment of XIX with a nitrogen-containing heterocycle yields XX. Compounds of formula XXI can be obtained by treating XX with CsCO₃ or KF/Alumina and a phenol.

Schemes 70 and 71 describe routes for the preparation of compounds XXVI and XXVII wherein R^{c} is a non-hydrogen substituent.

Schemes 72 and 73 illustrate the synthesis of compounds of formula XXIX and XXX, using techniques previously described above.

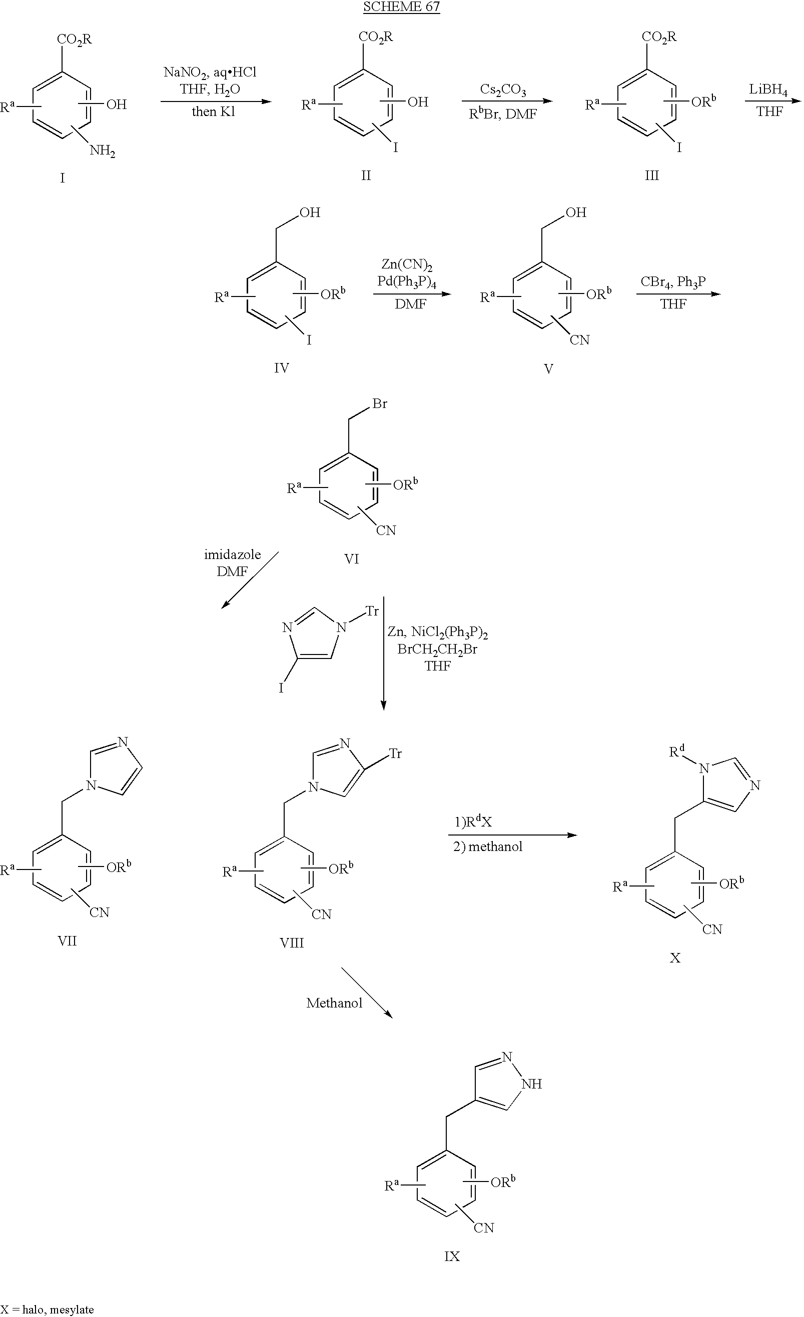

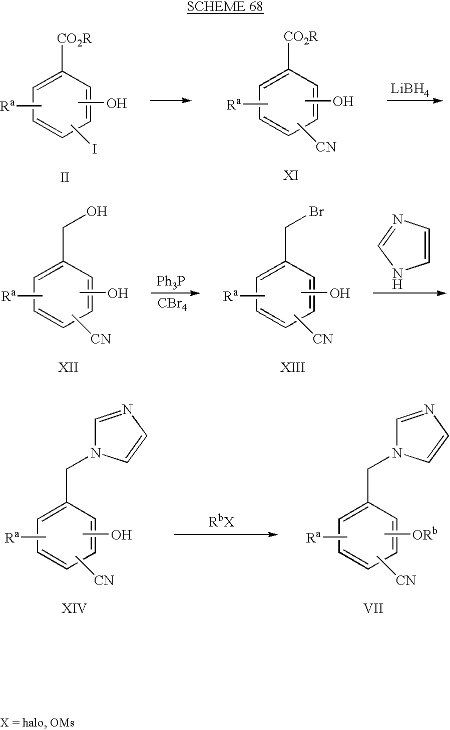

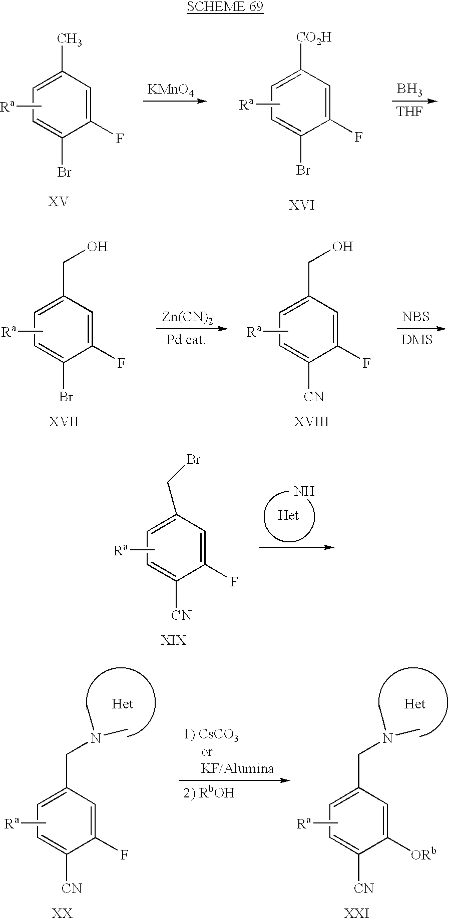

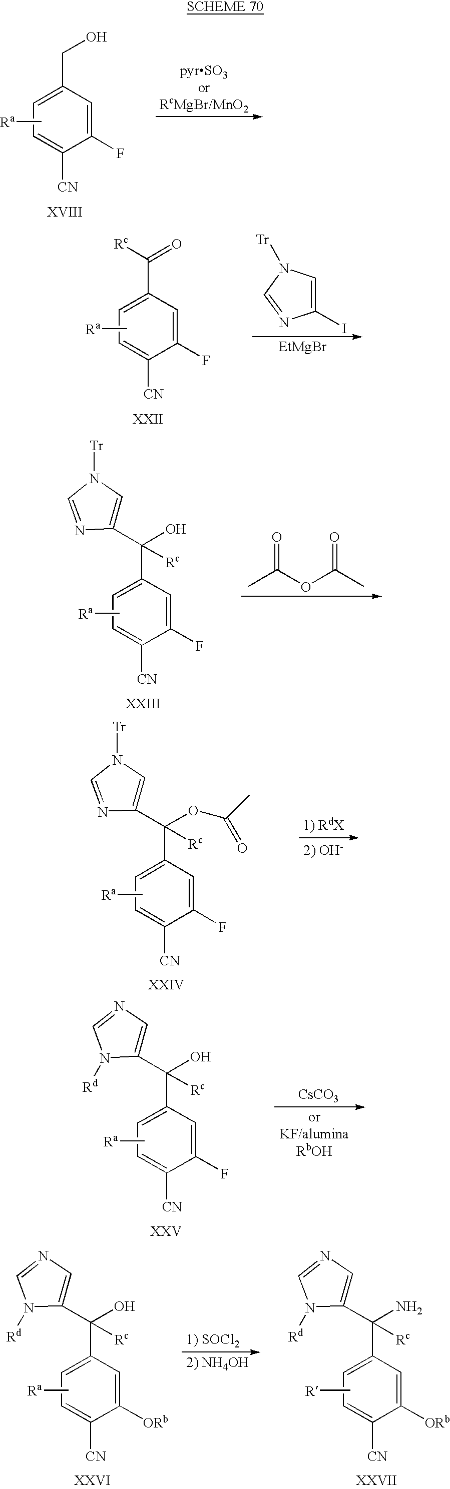

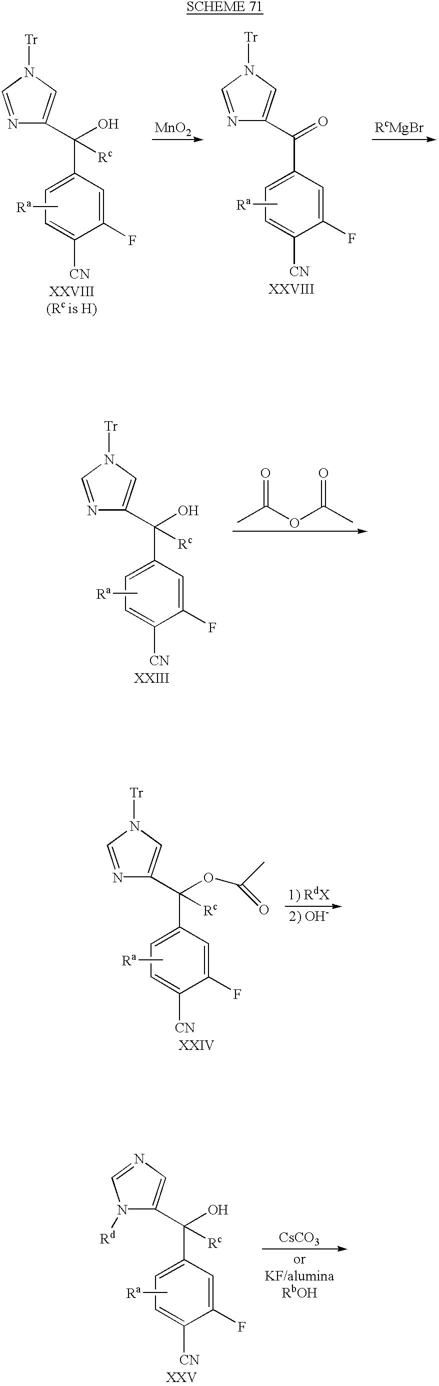

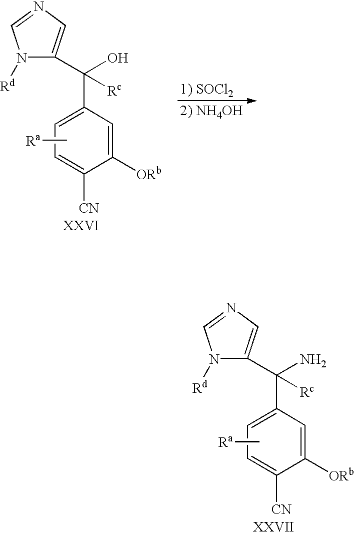

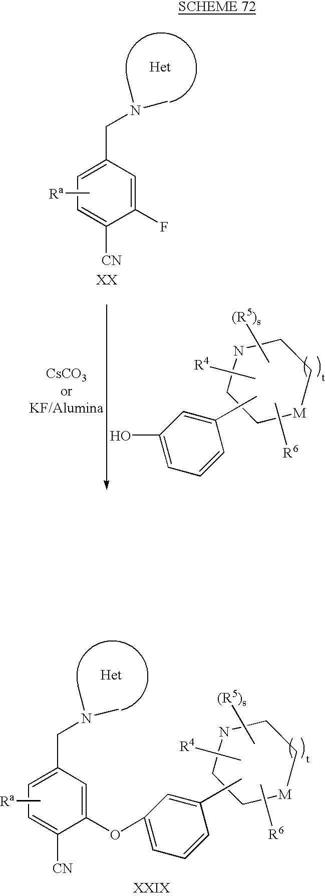

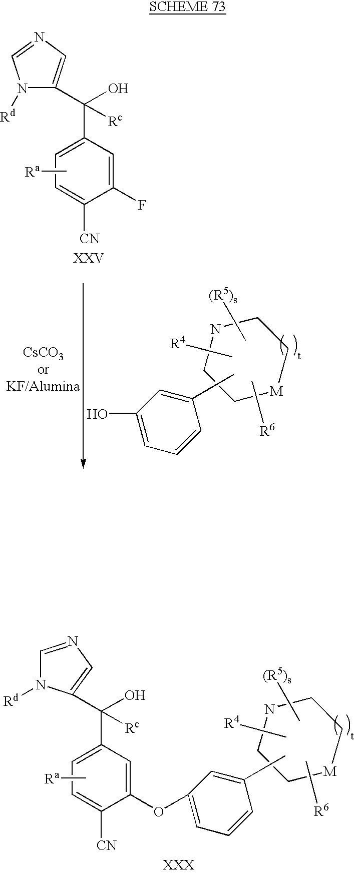

In Schemes 67-73, it is understood that:

R independently represents an alkyl or an aryl;

R^{a} independently represents R² or protected precursors thereof;

R^{b} independently represents the following moiety:

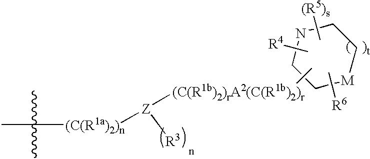

R^{c} independently represents R^{1c} or protected precursors thereof; and

R^{d} independently represents R¹ or protected precursors thereof.

The prenyl transferase inhibitors of formula (VII) can be synthesized in accordance with Schemes 74-90, in addition to other standard manipulations such as ester hydrolysis, cleavage of protecting groups, etc., as may be known in the literature or exemplified in the experimental procedures. The compounds referred to in the Synopsis of Schemes 74-90 by Roman numerals are numbered starting sequentially with II and ending with LII.

Reactions used to generate the compounds of this invention are prepared by employing reactions as shown in the Schemes 74-90, in addition to other standard manipulations such as ester hydrolysis, cleavage of protecting groups, etc., as may be known in the literature or exemplified in the experimental procedures. Substituents R, R^{a} and R^{b}, as shown in the Schemes, represent the substituents R², R³ and R⁴; however their point of attachment to the ring is illustrative only and is not meant to be limiting. Substituent Z′, as shown in the Schemes, represents the substituent Z as defined hereinabove or a protected precursor thereof.

These reactions may be employed in a linear sequence to provide the compounds of the invention or they may be used to synthesize fragments which are subsequently joined by the alkylation reactions described in the Schemes.

Synopsis of Schemes 74-90:

The requisite intermediates are in some cases commercially available, or can be prepared according to literature procedures, for the most part. In Scheme 74, for example, the synthesis of N-protected substituted piperazines is outlined. Boc-protected amino acids I, available commercially or by procedures known to those skilled in the art, can be coupled to N-benzyl amino acid esters using a variety of dehydrating agents such as DCC (dicyclohexycarbodiimide) or EDC.HCl (1-ethyl-3-(3-dimethylaminopropyl)carbodiimide hydrochloride) in a solvent such as methylene chloride, chloroform, dichloroethane, or dimethylformamide. The product II is then deprotected with acid, for example hydrogen chloride in chloroform or ethyl acetate, or trifluoroacetic acid in methylene chloride, and cyclized under weakly basic conditions to give the diketopiperazine III. Reduction of III with lithium aluminum hydride in a refluxing ether gives the piperazine IV, which is protected as the Boc derivative V. The N-benzyl group can be cleaved under standard conditions of hydrogenation, e.g., 10% palladium on carbon at 60 psi hydrogen on a Parr apparatus for 24-48 h. The product VI can be reacted with a suitably substituted carboxylic acid to provide the piperazine VII (Scheme 75); a final acid deprotection as previously described gives the intermediate VIII (Scheme 75). The intermediate VIII can itself be reductively alkylated with a variety of aldehydes, such as IX. The aldehydes can be prepared by standard procedures, such as that described by O. P. Goel, U. Krolls, M. Stier and S. Kesten in Organic Syntheses, 1988, 67, 69-75, from the appropriate amino acid (Scheme 76). The reductive alkylation can be accomplished at pH 5-7 with a variety of reducing agents, such as sodium triacetoxyborohydride or sodium cyanoborohydride in a solvent such as dichloroethane, methanol or dimethylformamide. The product X can be deprotected to give the final compounds XI with trifluoroacetic acid in methylene chloride. The final product XI is isolated in the salt form, for example, as a trifluoroacetate, hydrochloride or acetate salt, among others. The product diamine XI can further be selectively protected to obtain XII, which can subsequently be reductively alkylated with a second aldehyde to obtain XIII. Removal of the protecting group, and conversion to cyclized products such as the dihydroimidazole XV can be accomplished by literature procedures.

As shown in Scheme 77, the piperazine intermediate VIII can be reductively alkylated with other aldehydes such as 1-trityl-4-imidazolyl-carboxaldehyde or 1-trityl-4-imidazolylacetaldehyde, to give products such as XVI. The trityl protecting group can be removed from XVI to give XVII, or alternatively, XVI can first be treated with an alkyl halide then subsequently deprotected to give the alkylated imidazole XVIII. Alternatively, the intermediate VIII can be acylated or sulfonylated by standard techniques.

Incorporation of a hydroxyl moiety on the sidechain carbon alpha to the amide carbonyl of compounds of the formula XVIII can be accomplished as illustrated in Scheme 78. A suitably substituted primary alcohol XIX undergoes a one carbon homologation, via a Swern oxidation, nitrile addition and hydrolysis, to provide the substituted hydroxyacetic acid XX. The trifluoromethyl ketal is formed and reacted with the previously described protected piperazine VI to provide, following deprotection, the intermediate XXI. Intermediate XXI can undergo a variety of reactions at its unsubstituted nitrogen. For example, treatment of XXI with a suitably substituted imidazolylmethyl halide to provide the instant compound XXII.

Scheme 79 illustrates incorporation of an arylalkoxycarbonyl or heteroarylalkoxycarbonyl moiety onto the piperazine nitrogen. Thus a suitably substituted alcohol XXIII is reacted with nitrophenylchloroformate to provide the intermediate XXIV, which is reacted with a suitably substituted piperazine to provide the instant compound XXV. An analogous reaction sequence alternatively provides the corresponding aminocarbonyl substitution on the piperazine nitrogen, as shown in Scheme 80.

Scheme 81 illustrates the preparation of compounds analogous to compound XXXI wherein the alcohol utilized in the first step is a suitably substituted phenol. The scheme also illustrates the incorporation of an indole moiety for the substituent W in place of the preferred benzylimidazolyl moiety.

Scheme 82 illustrates synthesis of an instant compound wherein a non-hydrogen R^{9b} is incorporated in the instant compound. Thus, a readily available 4-substituted imidazole XXVI may be selectively iodinated to provide the 5-iodoimidazole XXVII. That imidazole may then be protected and coupled to a suitably substituted benzyl moiety to provide intermediate XXVIII. Attachment of the imidazolyl nitrogen via an ethyl linker to the piperazine nitrogen of intermediate XXI, described above, provides the instant compound XXIX.

Compounds of the instant invention wherein the A¹(CR^{1a}₂)ₙA²(CR^{1a}₂)ₙ linker is oxygen may be synthesized by methods known in the art, for example as shown in Scheme 83. The suitably substituted phenol XXX may be reacted with methyl N-(cyano)methanimidate to provide the 4-phenoxyimidazole XXXI. After selective protection of one of the imidazolyl nitrogens, the intermediate XXXII can undergo alkylation reactions as described for the benzylimidazoles in Scheme 81.

If the piperazine VIII is reductively alkylated with an aldehyde which also has a protected hydroxyl group, such as XXXIII in Scheme 84, the protecting groups can be subsequently removed to unmask the hydroxyl group. The Boc protected amino alcohol XXXIV can then be utilized to synthesize 2-aziridinylmethylpiperazines such as XXXV.

Reaction Scheme 85 provides an illustrative example of the synthesis of compounds of the instant invention wherein the substituents R² and R³ are combined to form —(CH₂)ᵤ—. For example, 1-aminocyclohexane-1-carboxylic acid XLV can be converted to the spiropiperazine XLVI essentially according to the procedures outlined in Schemes 74 and 75. The piperazine intermediate XLVI can be deprotected as before, and carried on to final products as described in Schemes 76-82. It is understood that reagents utilized to provide the imidazolylalkyl substituent may be readily replaced by other reagents well known in the art and readily available to provide other N-substituents on the piperazine.

Amino acids of the general formula LI which have a sidechain not found in natural amino acids may be prepared by the reactions illustrated in Scheme 86 starting with the readily prepared imine LII.

Other suitably substituted aldehydes such as those described in Schemes 13-16 hereinabove may be utilized in the syntheses of the instant compounds of the formula VIIIA. Similar synthetic strategies for preparing alkanols that incorporate other heterocyclic moieties for variable W are also well known in the art. For example, Scheme 87 illustrates the preparation of the corresponding quinoline aldehyde.

Scheme 88 depicts a general method for synthesizing a key intermediate useful in the preparation of preferred embodiments of the instant invention wherein V is phenyl and W is imidazole. A piperazine moiety can be readily added to this benzyl-imidazole intermediate as set forth in Scheme 89.

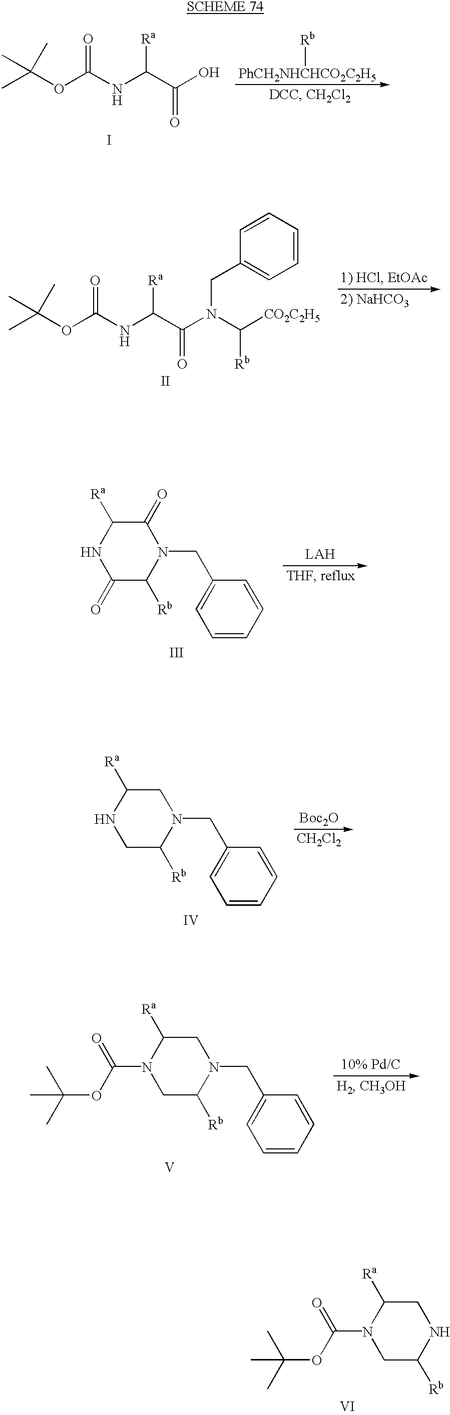

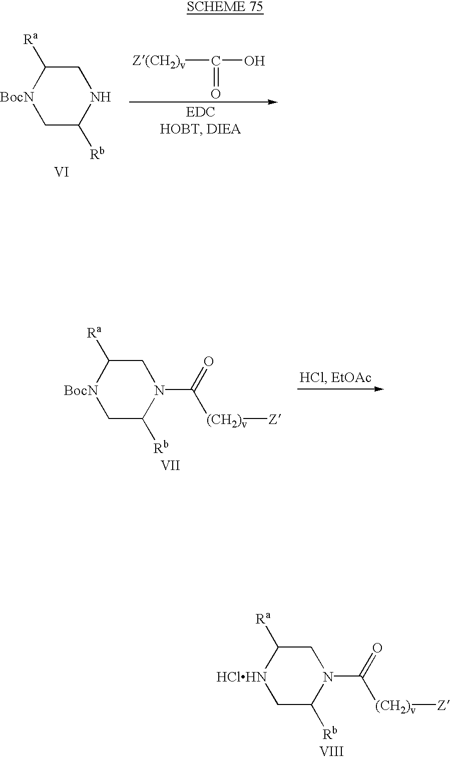

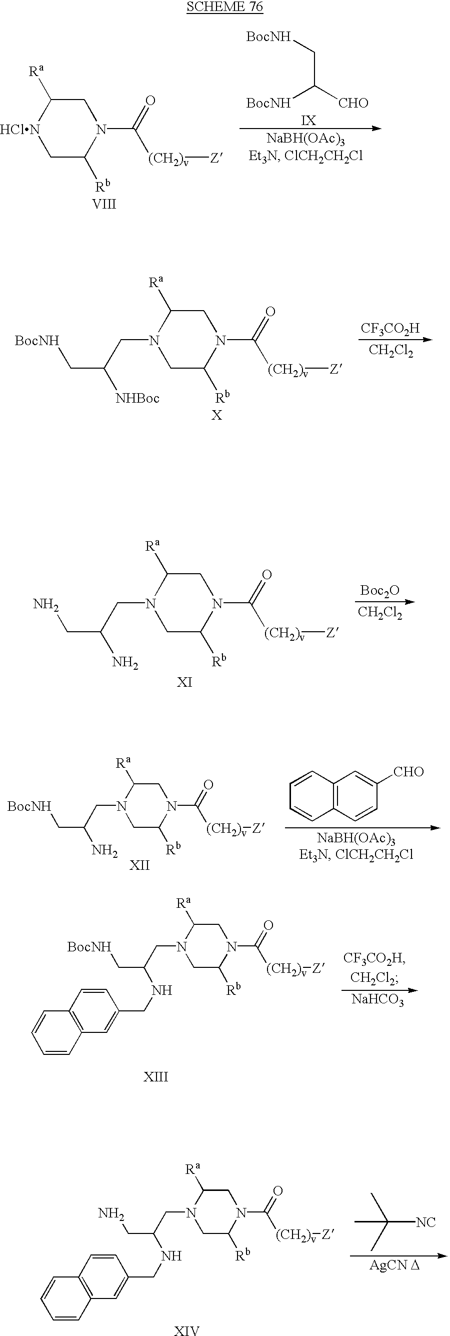

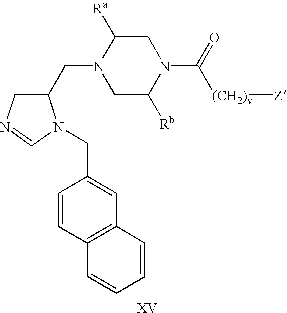

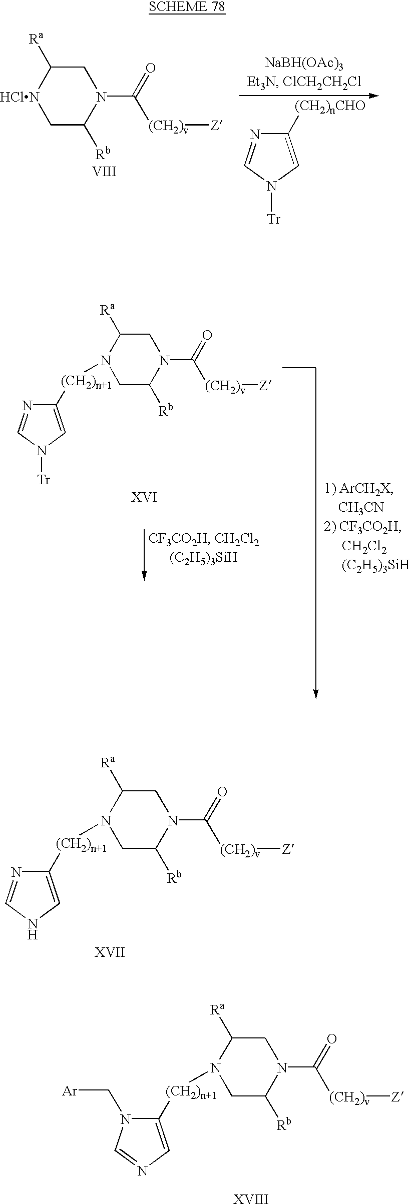

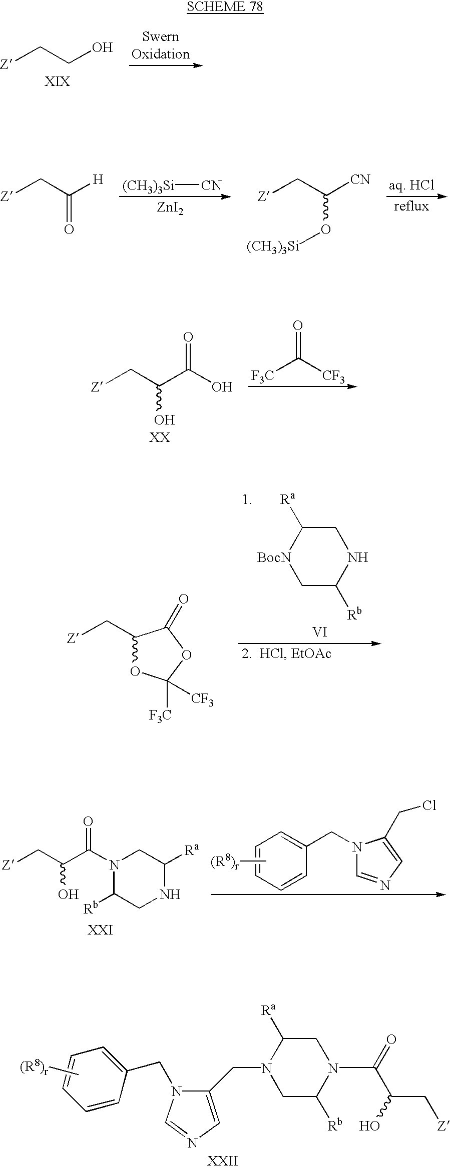

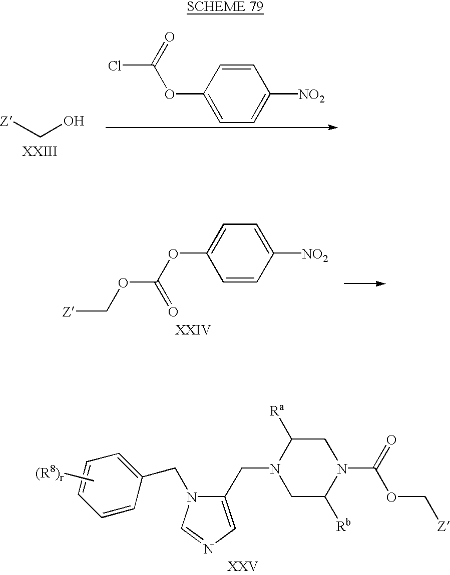

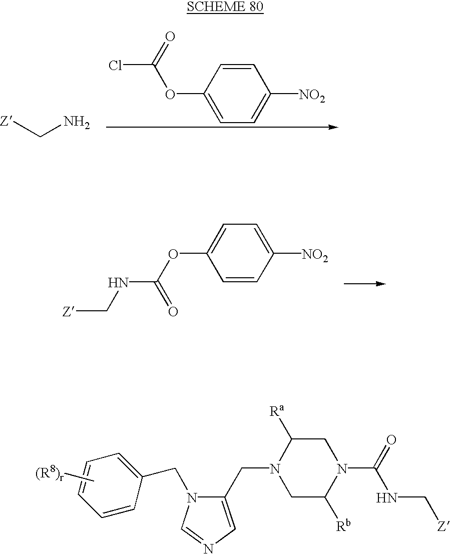

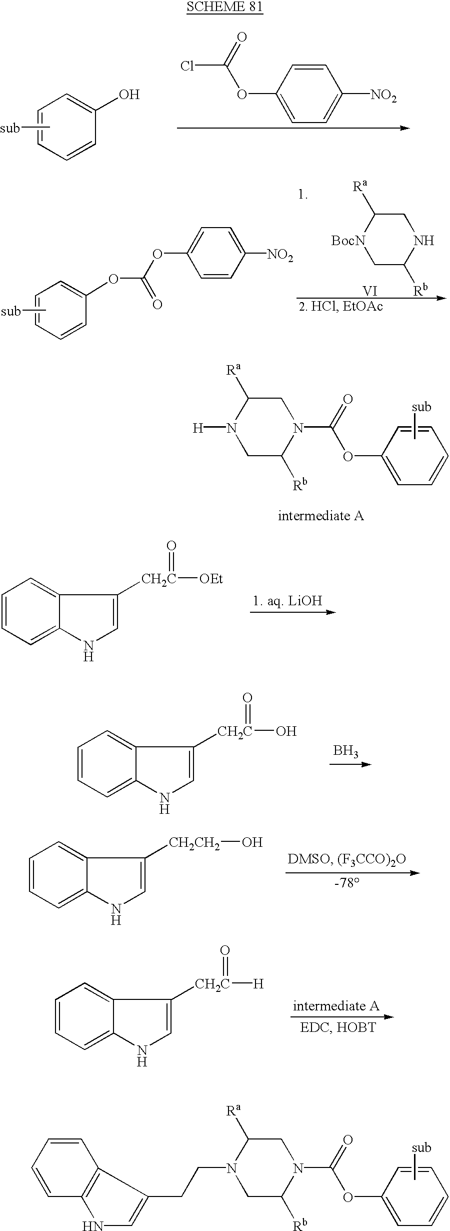

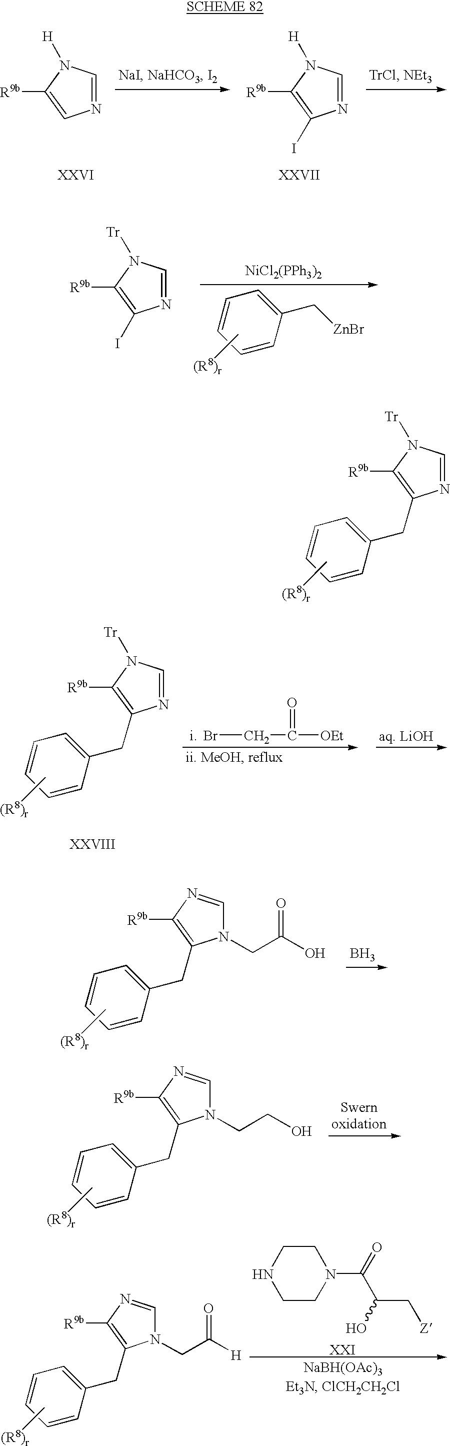

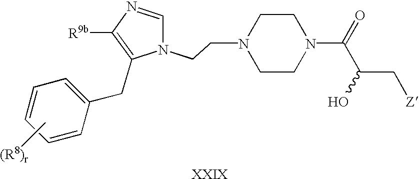

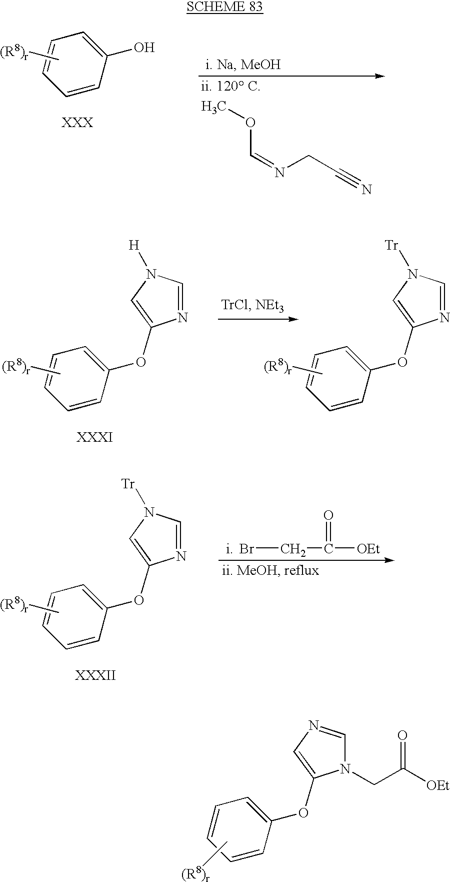

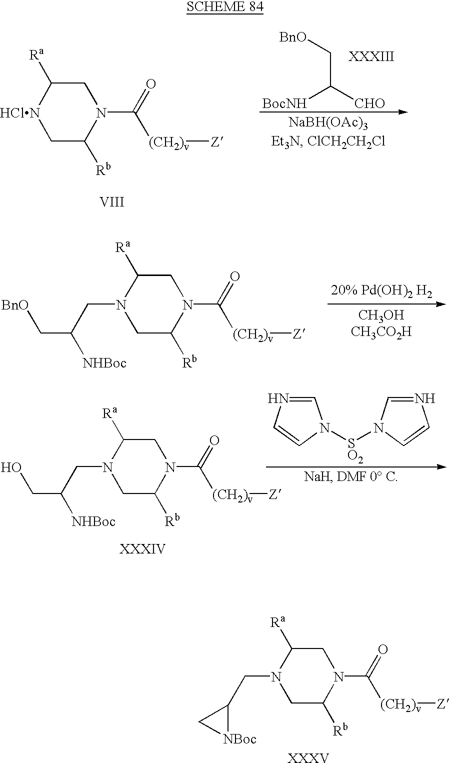

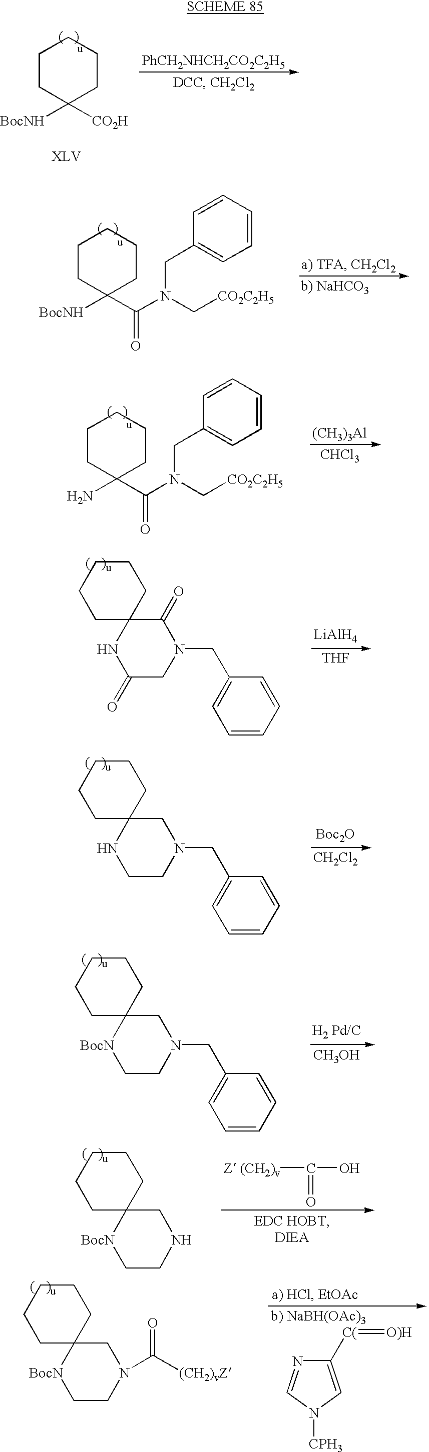

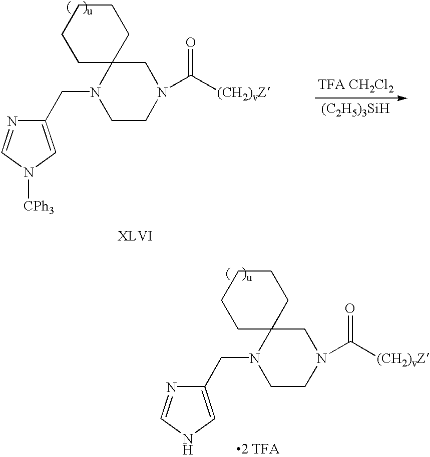

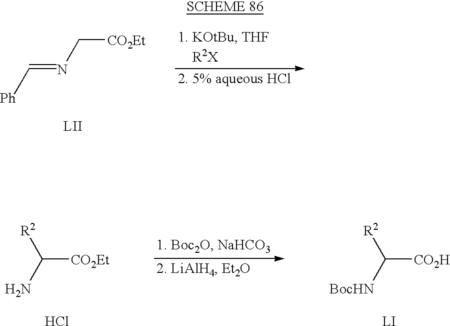

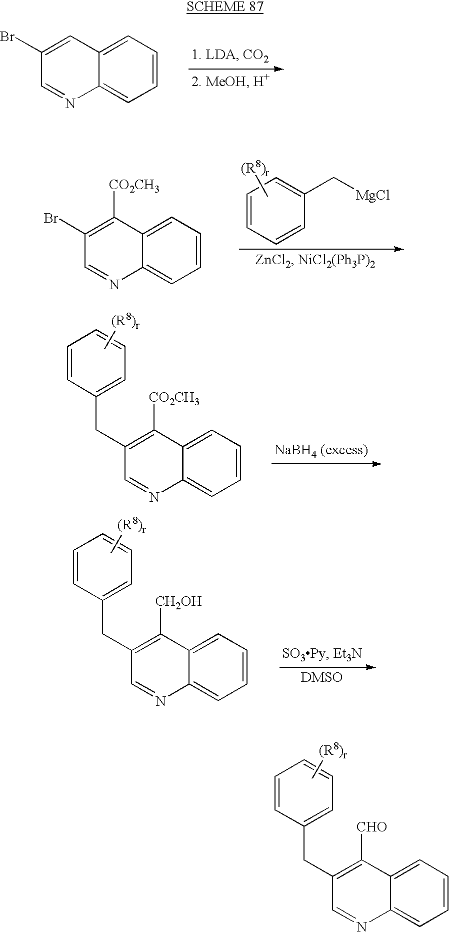

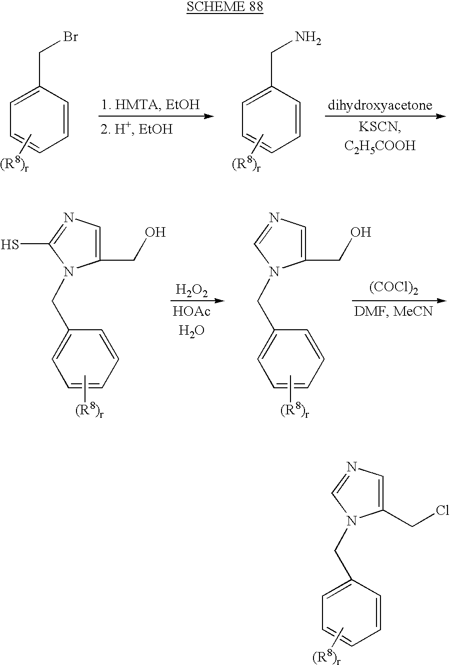

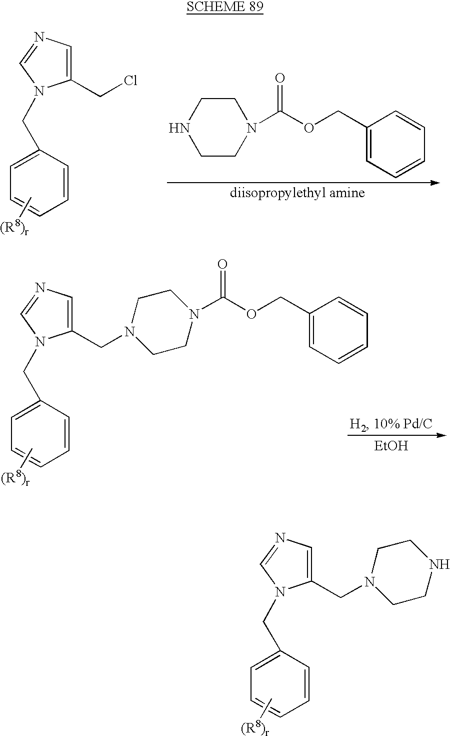

The PSA conjugates of formulae IX, X and XIII can be synthesized in accordance with Schemes 90-102, in addition to other standard manipulations such as ester hydrolysis, cleavage of protecting groups, etc., as may be known in the literature or exemplified in the experimental procedures.

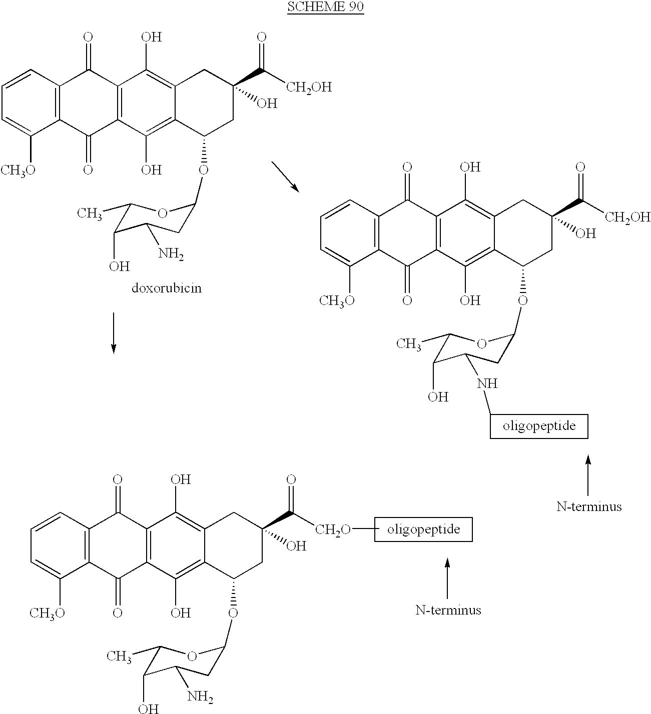

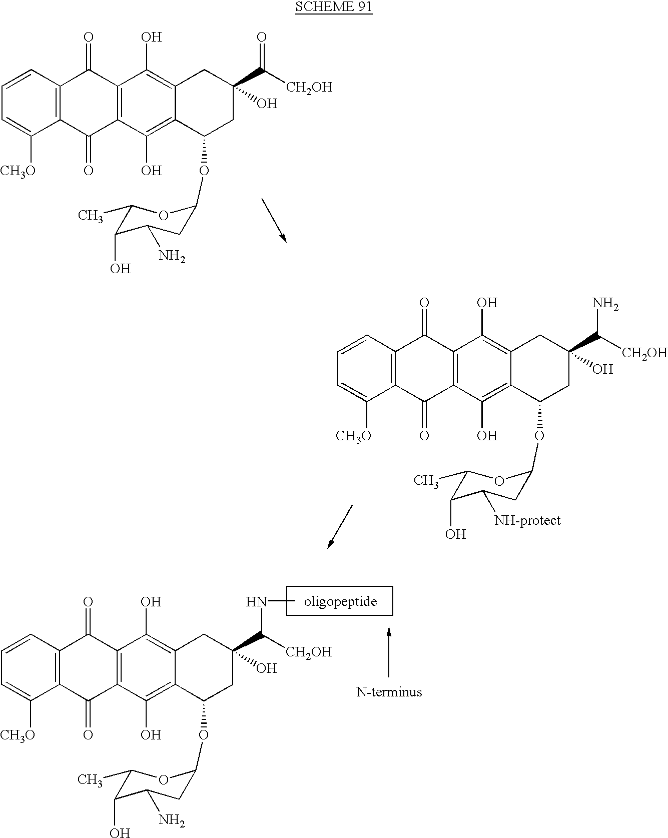

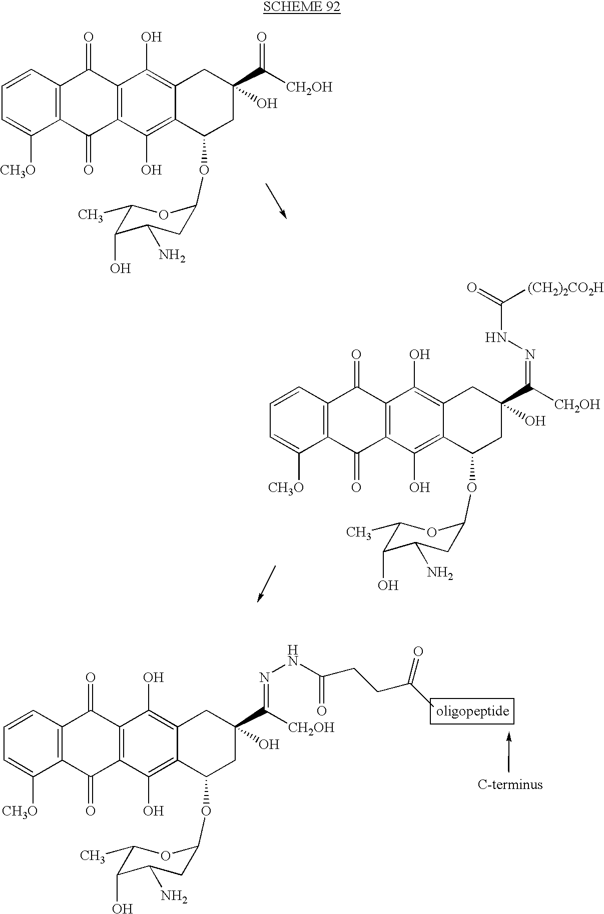

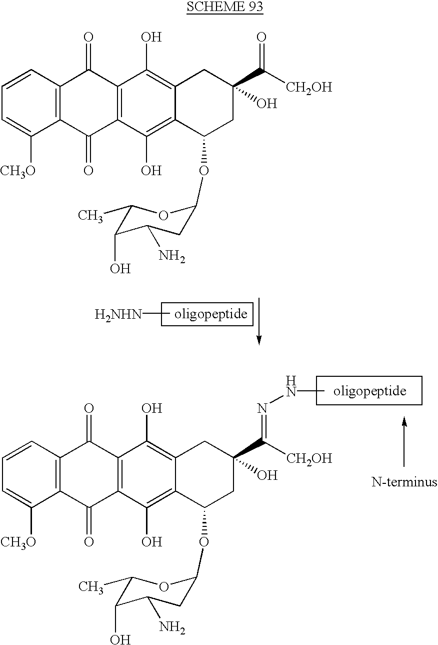

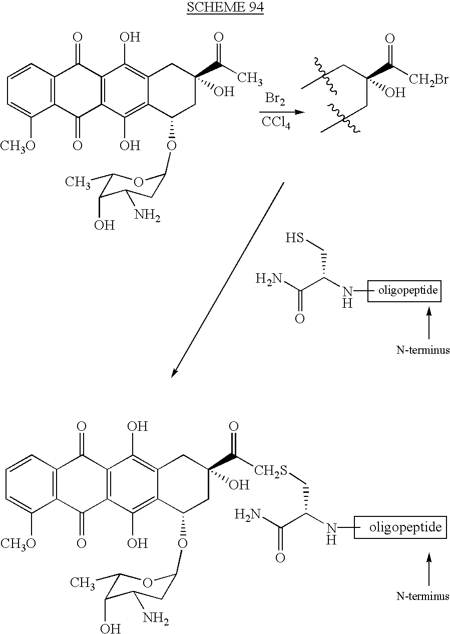

Scheme 95 illustrates preparation of conjugates utilized in the instant method of treatment wherein the oligopeptides are combined with the vinca alkaloid cytotoxic agent vinblastine. Attachment of the N-terminus of the oligopeptide to vinblastine is illustrated (S. P. Kandukuri et al. J. Med. Chem. 28:1079-1088 (1985)).

Scheme 96 illustrates preparation of conjugates of the oligopeptides of the instant invention and the vinca alkaloid cytotoxic agent vinblastine wherein the attachment of vinblastine is at the C-terminus of the oligopeptide. The use of the 1,3-diaminopropane linker is illustrative only; other spacer units between the carbonyl of vinblastine and the C-terminus of the oligopeptide are also envisioned. Furthermore, Scheme 96 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 80 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the treatment of prostate cancer.

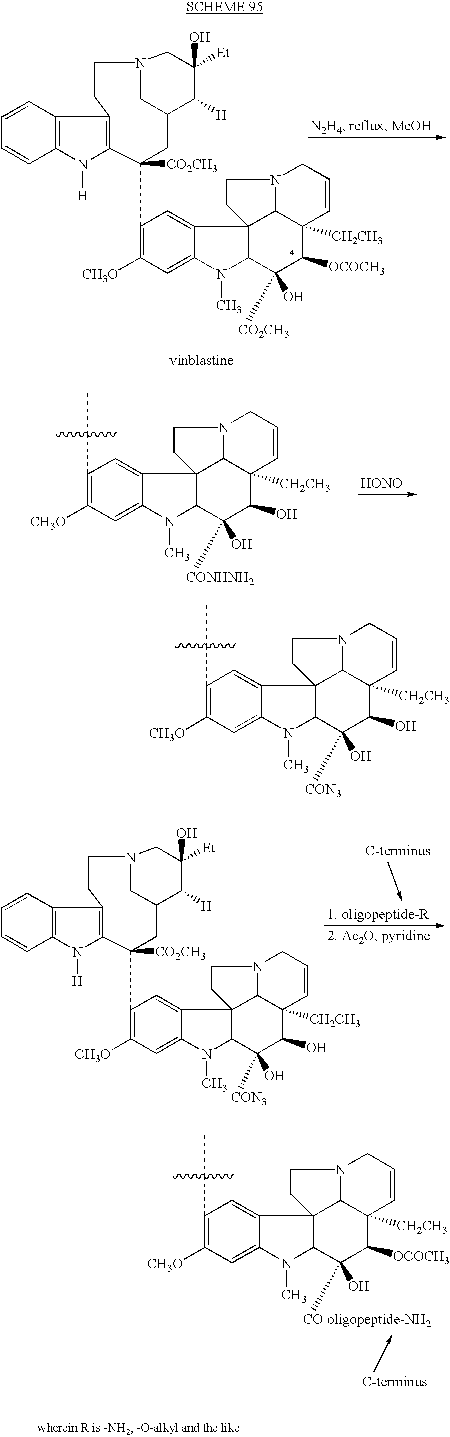

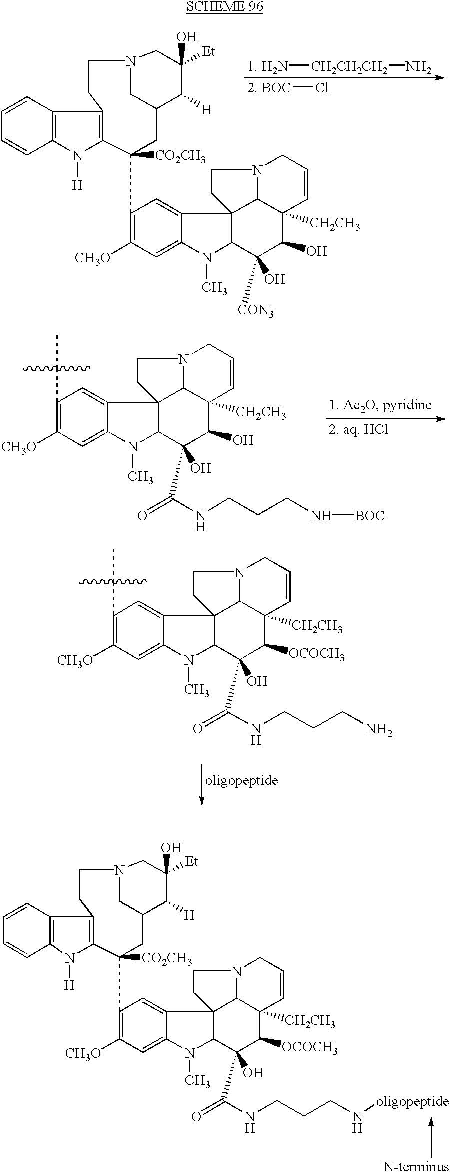

The PSA conjugates of formula XI and XII can be synthesized in accordance with Schemes 97-101, in addition to other standard manipulations such as ester hydrolysis, cleavage of protecting groups, etc., as may be known in the literature or exemplified in the experimental procedures.

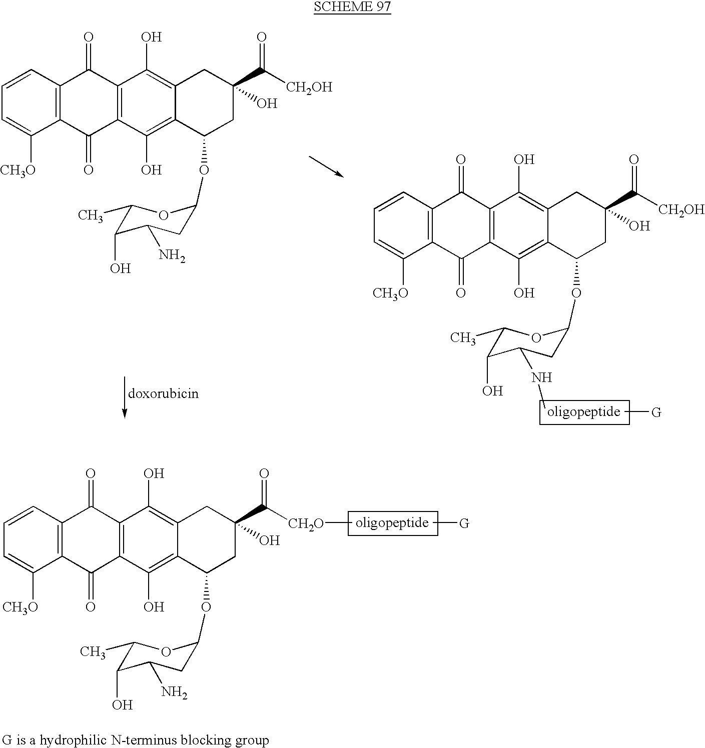

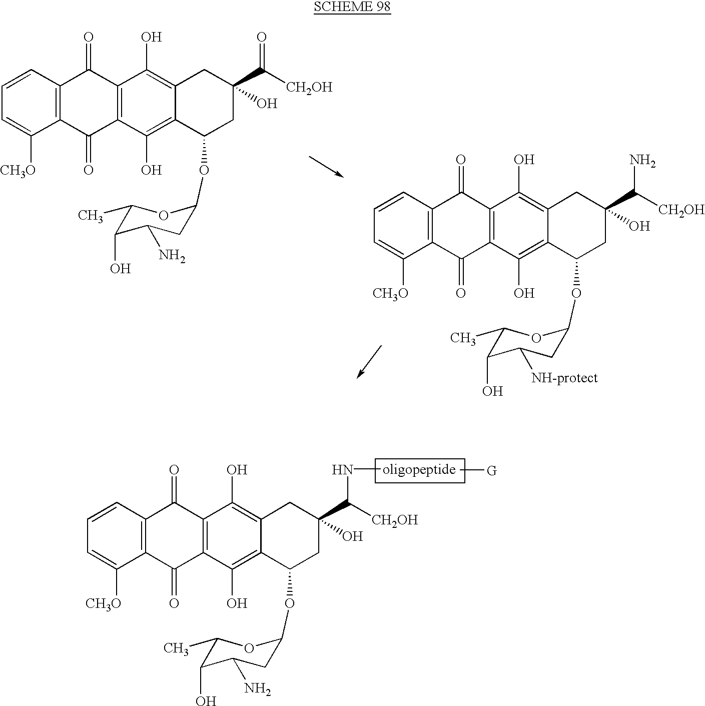

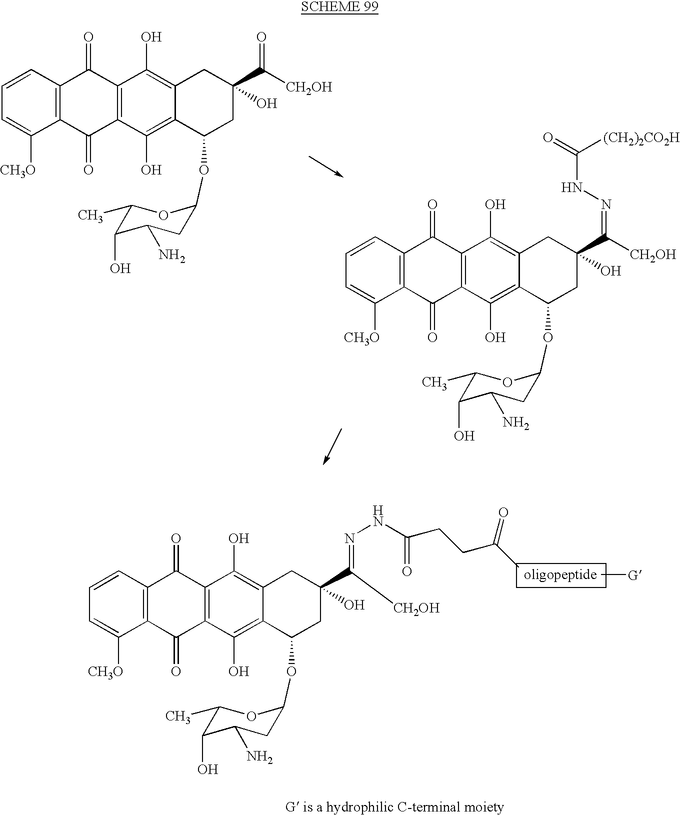

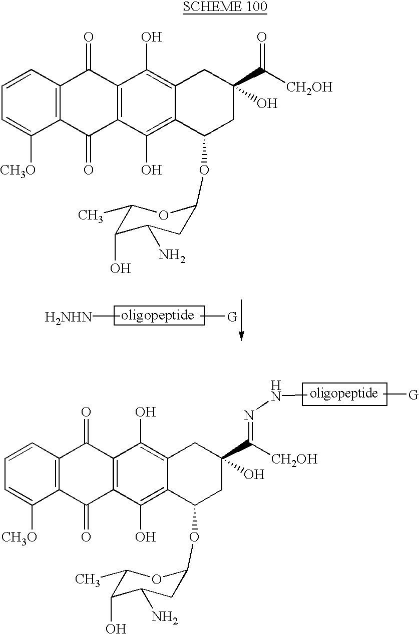

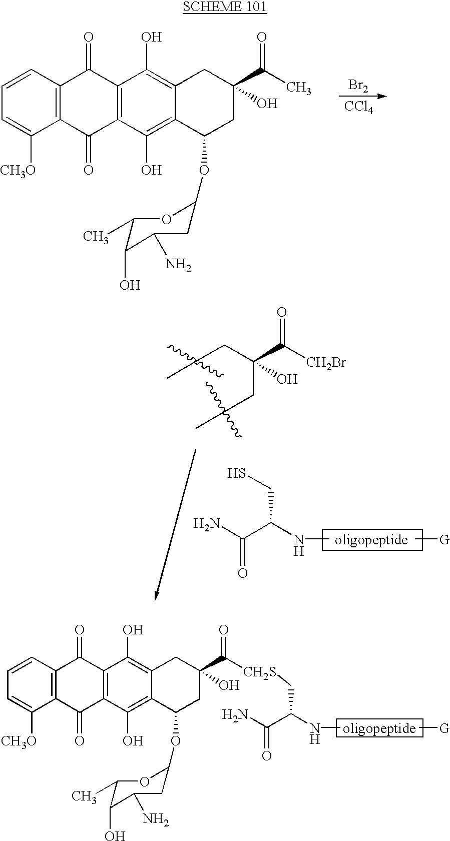

Scheme 102 illustrates preparation of PSA conjugates of the formula XIV wherein the attachment of vinblastine is at the C-terminus of the oligopeptide. Furthermore, Scheme 102 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 102 of protecting the primary amine of the linker and reacting the intermediate with acetic anhydride; followed by deprotection of the amine. Conjugation of the oligopeptide at other positions and functional groups of vinblastine may be readily accomplished by one of ordinary skill in the art and is also expected to provide compounds useful in the treatment of prostate cancer.

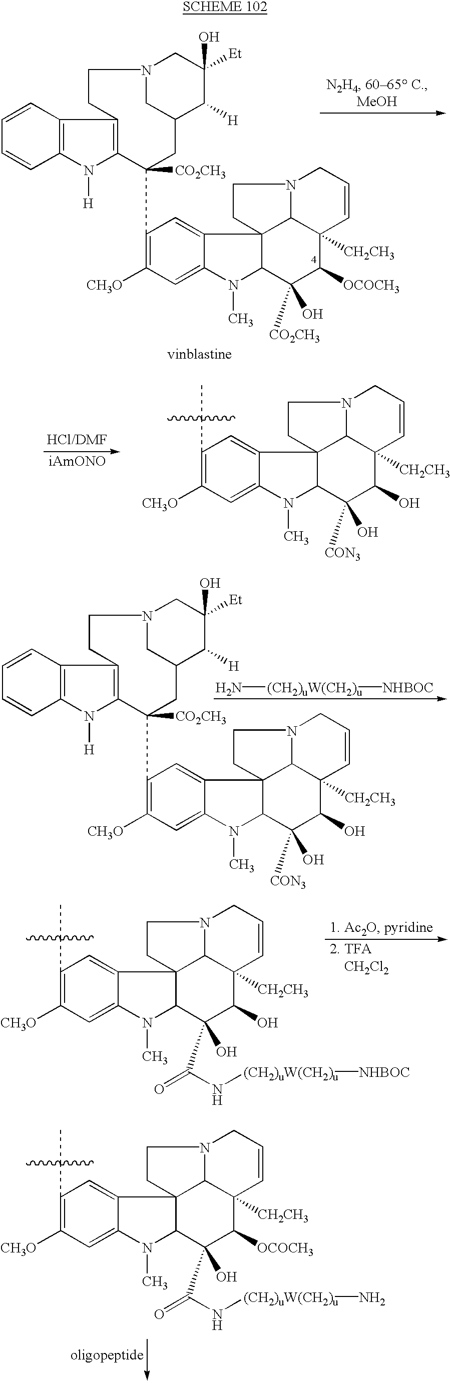

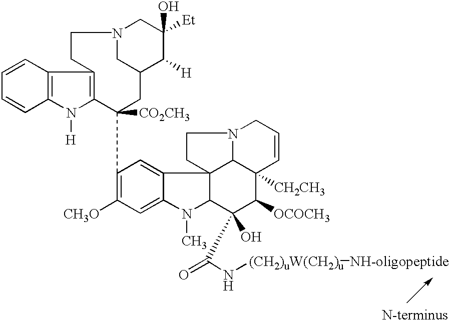

The PSA conjugates of formula XV can be synthesized in accordance with Schemes 103-104, in addition to other standard manipulations such as ester hydrolysis, cleavage of protecting groups, etc., as may be known in the literature or exemplified in the experimental procedures.

Reaction Scheme 103 illustrates preparation of conjugates of the oligopeptides of the instant invention and the vinca alkaloid cytotoxic agent vinblastine wherein the attachment of the oxygen of the 4-desacetylvinblastine is at the C-terminus of the oligopeptide. While other sequences of reactions may be useful in forming such conjugates, it has been found that initial attachment of a single amino acid to the 4-oxygen and subsequent attachment of the remaining oligopeptide sequence to that amino acid is a preferred method. It has also been found that 3,4-dihydro-3-hydroxy-4-oxo-1,2,3-benzotriazine (ODHBT) may be utilized in place of HOAt in the final coupling step.

Reaction Scheme 104 illustrates preparation of conjugates of the oligopeptides of the instant invention wherein a hydroxy alkanolyl acid is used as a linker between the vinca drug and the oligopeptide.

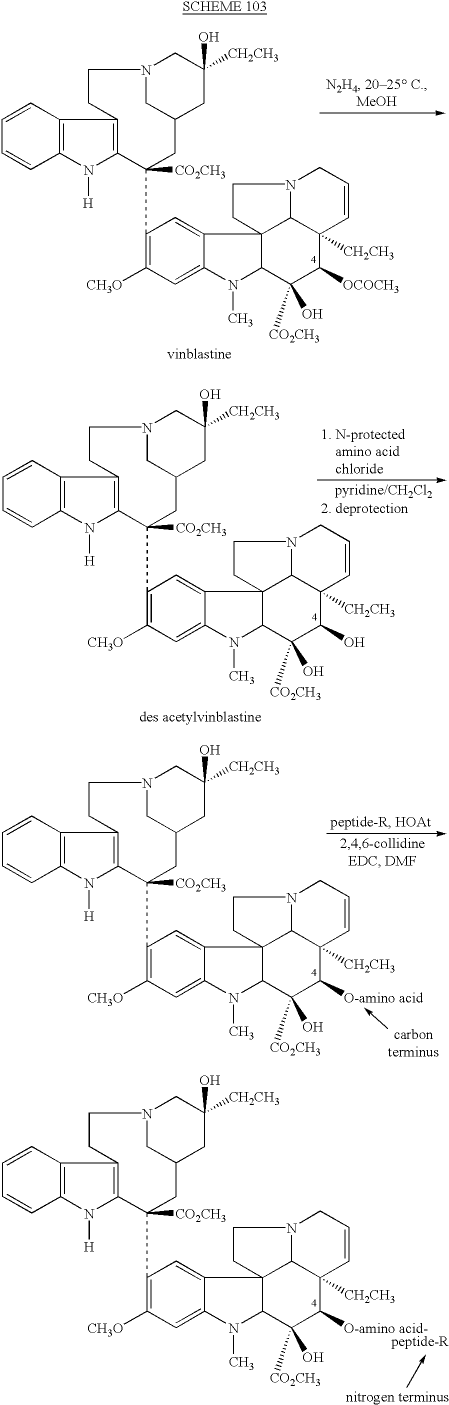

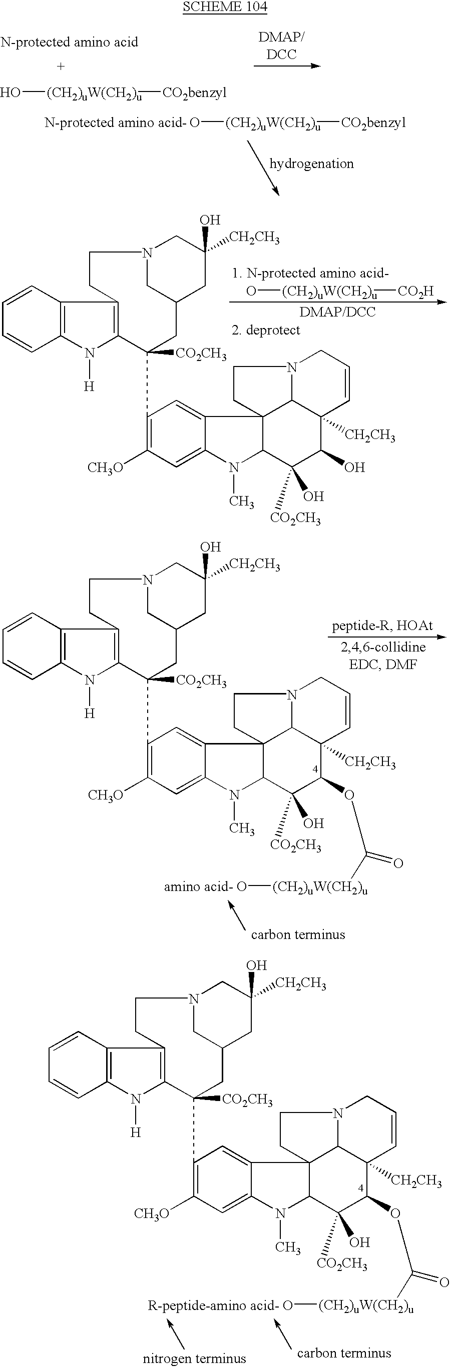

### EXAMPLES

Examples provided are intended to assist in a further understanding of the invention. Particular materials employed, species and conditions are intended to be further illustrative of the invention and not limitative of the reasonable scope thereof.

The standard workup referred to in the examples refers to solvent extraction and washing the organic solution with 10% citric acid, 10% sodium bicarbonate and brine as appropriate. Solutions were dried over sodium sulfate and evaporated in vacuo on a rotary evaporator.

### Example 1

(S)-1-(3-chlorophenyl)-4-[1-(4-cyanobenzyl)-imidazolylmethyl]-5-[2-(methanesulfonyl)ethyl]-2-piperazinone Dihydrochloride

Step A: 1-triphenylmethyl-4-(hydroxymethyl)-imidazole

To a solution of 4-(hydroxymethyl)imidazole hydrochloride (35.0 g, 260 mmol) in 250 mL of dry DMF at room temperature was added triethylamine (90.6 mL, 650 mmol). A white solid precipitated from the solution. Chlorotriphenylmethane (76.1 g, 273 mmol) in 500 mL of DMF was added dropwise. The reaction mixture was stirred for 20 hours, poured over ice, filtered, and washed with ice water. The resulting product was slurried with cold dioxane, filtered, and dried in vacuo to provide the titled product as a white solid which was sufficiently pure for use in the next step.

Step B: 1-triphenylmethyl-4-(acetoxymethyl)-imidazole

Alcohol from Step A (260 mmol, prepared above) was suspended in 500 mL of pyridine. Acetic anhydride (74 mL, 780 mmol) was added dropwise, and the reaction was stirred for 48 hours during which it became homogeneous. The solution was poured into 2 L of EtOAc, washed with water (3×1 L), 5% aq. HCl soln. (2×1 L), sat. aq. NaHCO₃, and brine, then dried (Na₂SO₄), filtered, and concentrated in vacuo to provide the crude product. The acetate was isolated as a white powder which was sufficiently pure for use in the next reaction.

Step C: 1-(4-cyanobenzyl)-5-(acetoxymethyl)-imidazole Hydrobromide

A solution of the product from Step B (85.8 g, 225 mmol) and a-bromo-p-tolunitrile (50.1 g, 232 mmol) in 500 mL of EtOAc was stirred at 60° C. for 20 hours, during which a pale yellow precipitate formed. The reaction was cooled to room temperature and filtered to provide the solid imidazolium bromide salt. The filtrate was concentrated in vacuo to a volume 200 mL, reheated at 60° C. for two hours, cooled to room temperature, and filtered again. The filtrate was concentrated in vacuo to a volume 100 mL, reheated at 60° C. for another two hours, cooled to room temperature, and concentrated in vacuo to provide a pale yellow solid. All of the solid material was combined, dissolved in 500 mL of methanol, and warmed to 60° C. After two hours, the solution was reconcentrated in vacuo to provide a white solid which was triturated with hexane to remove soluble materials. Removal of residual solvents in vacuo provided the titled product hydrobromide as a white solid which was used in the next step without further purification.

Step D: 1-(4-cyanobenzyl)-5-(hydroxymethyl)-imidazole

To a solution of the acetate from Step C (50.4 g, 150 mmol) in 1.5 L of 3:1 THF/water at 0° C. was added lithium hydroxide monohydrate (18.9 g, 450 mmol). After one hour, the reaction was concentrated in vacuo, diluted with EtOAc (3 L), and washed with water, sat. aq. NaHCO₃ and brine. The solution was then dried (Na₂SO₄), filtered, and concentrated in vacuo to provide the crude product as a pale yellow fluffy solid which was sufficiently pure for use in the next step without further purification.

Step E: 1-(4-cyanobenzyl)-5-imidazolecarboxaldehyde

To a solution of the alcohol from Step D (21.5 g, 101 mmol) in 500 mL of DMSO at room temperature was added triethylamine (56 mL, 402 mmol), then SO₃-pyridine complex (40.5 g, 254 mmol). After 45 minutes, the reaction was poured into 2.5 L of EtOAc, washed with water (4×1 L) and brine, dried (Na₂SO₄), filtered, and concentrated in vacuo to provide the aldehyde as a white powder which was sufficiently pure for use in the next step without further purification.

Step F: (S)-2-(tert-butoxycarbonylamino)-N-methoxy-N-methyl-4-(methylthio)butanamide

L-N-Boc-methionine (30.0 g, 0.120 mol), N,O-dimethyl-hydroxylamine hydrochloride (14.1 g, 0.144 mol), EDC hydrochloride (27.7 g, 0.144 mol) and HOBT (19.5 g, 0.144 mol) were stirred in dry DMF (300 mL) at 20° C. under nitrogen. More N,O-dimethylhydroxylamine hydrochloride (2.3 g, 23 mmol) was added to obtain pH 7-8. The reaction was stirred overnight, the DMF distilled to half the original volume under high vacuum, and the residue partitioned between ethyl acetate and sat. NaHCO₃ soln. The organic phase was washed with saturated sodium bicarbonate, water, 10% citric acid, and brine, and dried with sodium sulfate. The solvent was removed in vacuo to give the title compound.

Step G: (S)-2-(tert-butoxycarbonylamino)-4-(methylthio)butanal

A suspension of lithium aluminum hydride (5.02 g, 0.132 mol) in ether (500 mL) was stirred at room temperature for one hour. The solution was cooled to −50° C. under nitrogen, and a solution of the product from Step F (39.8 g, ca. 0.120 mol) in ether (200 mL) was added over 30 min, maintaining the temperature below −40° C. When the addition was complete, the reaction was warmed to 5° C., then recooled to −45° C. Analysis by tlc revealed incomplete reaction. The solution was rewarmed to 5° C., stirred for 30 minutes, then cooled to −50° C. A solution of potassium hydrogen sulfate (72 g, 0.529 mol) in 200 mL water was slowly added, maintaining the temperature below −20° C. The mixture was wasmed to 5° C., filtered through Celite, and concentrated in vacuo to provide the title aldehyde.

Step H: (S)-2-(tert-butoxycarbonylamino)-N-(3-chlorophenyl)-4-(methylthio)butanamine

To a solution of 3-chloroaniline (10.3 mL, 97.4 mmol), the product from Step G (23.9 g, 97.4 mmol), and acetic acid (27.8 mL, 487 mmol) in dichloroethane (250 mL) under nitrogen was added sodium triacetoxyborohydride (41.3 g, 195 mmol). The reaction was stirred overnight, then quenched with saturated sodium bicarbonate solution. The solution was diluted with CHCl₃, and the organic phase was washed with water, 10% citric acid and brine. The solution was dried over sodium sulfate and concentrated in vacuo to provide the crude product (34.8 g) which was chromatographed on silica gel with 20% ethyl acetate in hexane to obtain the title compound.

Step I: (S)-4-(tert-butoxycarbonyl)-1-(3-chlorophenyl)-5-[2-(methylthio)ethyl]piperazin-2-one

A solution of the product from Step H (22.0 g, 63.8 mmol) in ethyl acetate (150 mL) was vigorously stirred at 0° C. with saturated sodium bicarbonate (150 mL). Chloroacetyl chloride (5.6 mL, 70.2 mmol) was added dropwise, and the reaction stirred at 0° C. for 2 h. The layers were separated, and the ethyl acetate phase was washed with 10% citric acid and saturated brine, and dried over sodium sulfate. After concentration in vacuo, the resulting crude product (27.6 g) was dissolved in DMF (300 mL) and cooled to 0° C. under argon. Cesium carbonate (63.9 g, 196 mmol) was added, and the reaction was stirred for two days, allowing it to warm to room temperature. Another portion of cesium carbonate (10 g, 30 mmol) was added, and the reaction was stirred for 16 hours. The DMF was distilled in vacuo, and the residue partitioned between ethyl acetate and water. The organic phase was washed with saturated brine, and dried over sodium sulfate. The crude product was chromatographed on silica gel with 20-25% ethyl acetate in hexane to obtain the title compound.

Step J: (S)-4-(tert-butoxycarbonyl)-1-(3-chlorophenyl)-5-[2-(methanesulfonyl)ethyl]piperazin-2-one

A solution of the product from Step I (14.2 g, 37 mmol) in methanol (300 mL) was cooled to 0° C., and a solution of magnesium monoperoxyphthalate (54.9 g, 111 mmol) in 210 mL MeOH was added over 20 minutes. The ice bath was removed, and the solution was allowed to warm to room temperature. After 45 minutes, the reaction was concentrated in vacuo to half the original volume, then quenched by the addition of 2N Na₂S203 soln. The solution was poured into EtOAc and sat NaHCO₃ solution, and the organic layer was washed with brine, dried (Na₂SO₄), filtered, and concentrated in vacuo to provide the crude sulfone. This material was chromatographed on silica gel with 60-100% ethyl acetate in hexane to obtain the titled compound.

Step K: (S)-1-(3-chlorophenyl)-5-[2-(methanesulfonyl)ethyl]piperazin-2-one

Through a solution of Boc-protected piperazinone from Step J (1.39 g, 3.33 mmol) in 30 mL of EtOAc at 0° C. was bubbled anhydrous HCl gas. The saturated solution was stirred for 35 minutes, then concentrated in vacuo to provide the hydrochloride salt as a white powder. This material was suspended in EtOAc and treated with dilute aqueous NaHCO₃ solution. The aqueous phase was extracted with EtOAc, and the combined organic mixture was washed with brine, dried (Na₂SO₄), filtered, and concentrated in vacuo. The resulting amine was reconcentrated from toluene to provide the titled material suitable for use in the next step.

Step L: (S)-1-(3-chlorophenyl)-4-[1-(4-cyanobenzyl)imidazolylmethyl]-5-[2-(methanesulfonyl)-ethyl]-2-piperazinone Dihydrochloride

To a solution of the amine from Step K (898 mg, 2.83 mmol) and imidazole carboxaldehyde from Step E (897 mg, 4.25 mmol) in 15 mL of 1,2-dichloroethane was added sodium triacetoxyborohydride (1.21 g, 5.7 mmol). The reaction was stirred for 23 hours, then quenched at 0° C. with sat. NaHCO₃ solution. The solution was poured into CHCl₃, and the aqueous layer was back-extracted with CHCl₃. The combined organics were washed with brine, dried (Na₂SO₄), filtered, and concentrated in vacuo. The resulting product was purified by silica gel chromatography (95:5:0.5-90:10:0 EtOAc:MeOH:NH₄Cl), and the resultant product was taken up in EtOAc/methanol and treated with 2.1 equivalents of 1 M HCl/ether solution. After concentrated in vacuo, the product dihydrochloride was isolated as a white powder.

### Example 2

1-(3-chlorophenyl)-4-[1-(4-cyanobenzyl)imidazolyl-methyl]-2-piperazinone dihydrochloride

Step A: N-(3-chlorophenyl)ethylenediamine Hydrochloride

To a solution of 3-chloroaniline (30.0 mL, 284 mmol) in 500 mL of dichloromethane at 0° C. was added dropwise a solution of 4 N HCl in 1,4-dioxane (80 mL, 320 mmol HCl). The solution was warmed to room temperature, then concentrated to dryness in vacuo to provide a white powder. A mixture of this powder with 2-oxazolidinone (24.6 g, 282 mmol) was heated under nitrogen atmosphere at 160° C. for 10 hours, during which the solids melted, and gas evolution was observed. The reaction was allowed to cool, forming the crude diamine hydrochloride salt as a pale brown solid.

Step B: N-(tert-butoxycarbonyl)-N′-(3-chlorophenyl) Ethylenediamine

The amine hydrochloride from Step A (ca. 282 mmol, crude material prepared above) was taken up in 500 mL of THF and 500 mL of sat. aq. NaHCO₃ soln., cooled to 0° C., and di-tert-butylpyrocarbonate (61.6 g, 282 mmol) was added. After 30 h, the reaction was poured into EtOAc, washed with water and brine, dried (Na₂SO₄), filtered, and concentrated in vacuo to provide the titled carbamate as a brown oil which was used in the next step without further purification.

Step C: N-[2-(tert-butoxycarbamoyl)ethyl]—N-(3-chlorophenyl)-2-chloroacetamide

A solution of the product from Step B (77 g, ca. 282 mmol) and triethylamine (67 mL, 480 mmol) in 500 mL of CH₂Cl₂ was cooled to 0° C. Chloroacetyl chloride (25.5 mL, 320 mmol) was added dropwise, and the reaction was maintained at 0° C. with stirring. After 3 h, another portion of chloroacetyl chloride (3.0 mL) was added dropwise. After 30 min, the reaction was poured into EtOAc (2 L) and washed with water, sat. aq. NH₄Cl soln, sat. aq. NaHCO₃ soln., and brine. The solution was dried (Na₂SO₄), filtered, and concentrated in vacuo to provide the chloroacetamide as a brown oil which was used in the next step without further purification.

Step D: 4-(tert-butoxycarbonyl)-1-(3-chlorophenyl)-2-piperazinone

To a solution of the chloroacetamide from Step C (ca. 282 mmol) in 700 mL of dry DMF was added K2CO₃ (88 g, 0.64 mol). The solution was heated in an oil bath at 70-75° C. for 20 hours, cooled to room temperature, and concentrated in vacuo to remove ca. 500 mL of DMF. The remaining material was poured into 33% EtOAc/hexane, washed with water and brine, dried (Na₂SO₄), filtered, and concentrated in vacuo to provide the product as a brown oil. This material was purified by silica gel chromatography (25-50% EtOAc/hexane) to yield pure product, along with a sample of product (ca. 65% pure by HPLC) containing a less polar impurity.

Step E: 1-(3-chlorophenyl)-2-piperazinone

Through a solution of Boc-protected piperazinone from Step D (17.19 g, 55.4 mmol) in 500 mL of EtOAc at −78° C. was bubbled anhydrous HCl gas. The saturated solution was warmed to 0° C., and stirred for 12 hours. Nitrogen gas was bubbled through the reaction to remove excess HCl, and the mixture was warmed to room temperature. The solution was concentrated in vacuo to provide the hydrochloride as a white powder. This material was taken up in 300 mL of CH₂Cl₂ and treated with dilute aqueous NaHCO₃ solution. The aqueous phase was extracted with CH₂Cl₂ (8×300 mL) until tlc analysis indicated complete extraction. The combined organic mixture was dried (Na₂SO₄), filtered, and concentrated in vacuo to provide the titled free amine as a pale brown oil.

Step F: 1-(3-chlorophenyl)-4-[1-(4-cyanobenzyl)imidazolylmethyl]-2-piperazinone Dihydrochloride

To a solution of the amine from Step E (55.4 mmol, prepared above) in 200 mL of 1,2-dichloroethane at 0° C. was added 4 Å powdered molecular sieves (10 g), followed by sodium triacetoxyborohydride (17.7 g, 83.3 mmol). The imidazole carboxaldehyde from Step E of Example 4 (11.9 g, 56.4 mmol) was added, and the reaction was stirred at 0° C. After 26 hours, the reaction was poured into EtOAc, washed with dilute aq. NaHCO₃, and the aqueous layer was back-extracted with EtOAc. The combined organics were washed with brine, dried (Na₂SO₄), filtered, and concentrated in vacuo. The resulting product was taken up in 500 mL of 5:1 benzene:CH₂Cl₂, and propyl-amine (20 mL) was added. The mixture was stirred for 12 hours, then concentrated in vacuo to afford a pale yellow foam. This material was purified by silica gel chromatography (2-7% MeOH/CH₂Cl₂), and the resultant white foam was taken up in CH₂Cl₂ and treated with 2.1 equivalents of 1 M HCl/ether solution. After concentrated in vacuo, the product dihydrochloride was isolated as a white powder.

### Example 2A

1-(3-chlorophenyl)-4-[1-(4-cyanobenzyl)imidazolyl-methyl]-2-piperazinone Hydrochloride (Compound A)

Step 1: Preparation of p-Cyanobenzylamine.H₃P₄ Salt

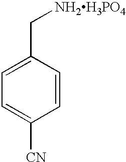

A slurry of HMTA in 2.5 L EtOH was added gradually over about 30 min to about 60 min to a stirred slurry of cyanobenzyl-bromide in 3.5 L EtOH and maintained at about 48-53° C. with heating & cooling in a 22L neck flask (small exotherm). Then the transfer of HMTA to the reaction mixture was completed with the use of 1.0 L EtOH. The reaction mixture was heated to about 68-73° C. and aged at about 68-73° C. for about 90 min. The reaction mixture was a slurry containing a granular precipitate which quickly settled when stirring stopped.

The mixture was cooled to a temperature of about 50° C. to about 55° C. Propionic acid was added to the mixture and the mixture was heated and maintained at a temperature of about 50° C. to about 55° C. Phosphoric acid was gradually added over about 5 min to about 10 min, maintaining the reaction mixture below about 65° C. to form a precipitate-containing mixture. Then the mixture was gradually warmed to about 65° C. to about 70° C. over about 30 min and aged at about 65° C. to about 70° C. for about 30 min. The mixture was then gradually cooled to about 20-25° C. over about 1 hour and aged at about 20-25° C. for about 1 hour.

The reaction slurry was then filtered. The filter cake was washed four times with EtOH, using the following sequence, 2.5 L each time. The filter cake was then washed with water five times, using 300 mL each time. Finally, the filter cake was washed twice with MeCN (1.0 L each time) and the above titled compound was obtained.

Step 2: Preparation of 1-(4-Cyanobenzyl)-2-Mercapto-5-Hydroxymethylimidazole

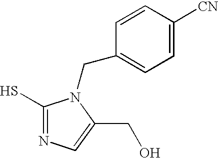

7% water in acetonitrile (50 mL) was added to a 250 mL roundbottom flask. Next, an amine phosphate salt (12.49 g), prepared as described in Step 1, was added to the flask. Next potassium thiocyanate (6.04 g) and dihydroxyacetone (5.61 g) was added. Lastly, propionic acid (10.0 mL) was added. Acetonitrile/water 93:7 (25 mL) was used to rinse down the sides of the flask. This mixture was then heated to 60° C., aged for about 30 minutes and seeded with 1% thioimidazole. The mixture was then aged for about 1.5 to about 2 hours at 60° C. Next, the mixture was heated to 70° C., and aged for 2 hours. The temperature of the mixture was then cooled to room temperature and was aged overnight. The thioimidazole product was obtained by vacuum filtration. The filter cake was washed four times acetonitrile (25 mL each time) until the filtrates became nearly colorless: Then the filter cake was washed three times with water (approximately 25-50 mL each time) and dried in vacuo to obtain the above-identified compound.

Step 3: Preparation of 1-(4-Cyanobenzyl)-5-Hydroxymethylimidazole

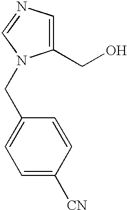

A 1L flask with cooling/heating jacket and glass stirrer (Lab-Max) was charged with water (200 mL) at 25° C. The thioimidazole (90.27 g), prepared as described in Step 2, was added, followed by acetic acid (120 mL) and water (50 mL) to form a pale pink slurry. The reaction was warmed to 40° C. over 10 minutes. Hydrogen peroxide (90.0 g) was added slowly over 2 hours by automatic pump maintaining a temperature of 35-45° C. The temperature was lowered to 25° C. and the solution aged for 1 hour.

The solution was cooled to 20° C. and quenched by slowly adding 20% aqueous Na₂SO₃ (25 mL) maintaining the temperature at less than 25° C. The solution was filtered through a bed of DARCO G-60 (9.0 g) over a bed of SolkaFlok (1.9 g) in a sintered glass funnel. The bed was washed with 25 mL of 10% acetic acid in water.

The combined filtrates were cooled to 15° C. and a 25% aqueous ammonia was added over a 30 minute period, maintaining the temperature below 25° C., to a pH of 9.3. The yellowish slurry was aged overnight at 23° C. (room temperature). The solids were isolated via vacuum filtration. The cake (100 mL wet volume) was washed with 2×250 mL 5% ammonia (25%) in water, followed by 100 mL of ethyl acetate. The wet cake was dried with vacuum/N₂ flow and the above-titled compound was obtained.

¹H NMR (250 MHz, CDCl₃): δ 7.84-7.72 (d, 2H), 7.31-7.28 (d, 2H), 6.85 (s, 1H), 5.34 (s, 2H), 5.14-5.11 (t, 1H), 4.30-4.28 (d, 2H), 3.35 (s, 1H).

Step 4: Preparation of 1-(4-cyanobenzyl)-5-chloromethylimidazole HCl Salt

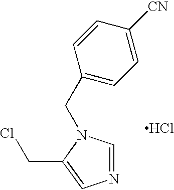

1-(4-Cyanobenzyl)-5-hydroxymethylimidazole (1.0 kg), prepared as described in above in Step 3, was slurried with DMF (4.8 L) at 22° C. and then cooled to −5° C. Thionyl chloride (390 mL) was added dropwise over 60 min during which time the reaction temperature rose to a maximum of 9° C. The solution became nearly homogeneous before the product began to precipitate from solution. The slurry was warmed to 26° C. and aged for 1 h.

The slurry was then cooled to 5° C. and 2-propanol (120 mL) was added dropwise, followed by the addition of ethyl acetate (4.8 L). The slurry was aged at 5° C. for 1 h before the solids were isolated and washed with chilled ethyl acetate (3×1 L). The product was dried in vacuo at 40° C. overnight to provide the above-titled compound.

¹H NMR (250 MHz DMSO-d₆): δ 9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d₆): δ_{c} 139.7, 137.7, 132.7, 130.1, 128.8, 120.7, 118.4, 111.2, 48.9, 33.1.

Step 5: Preparation of 1-(4-Cyanobenzyl)-5-Chloromethyl Imidazole HCl Salt via Addition of Hydroxymethylimidazole to Vilsmeier Reagent

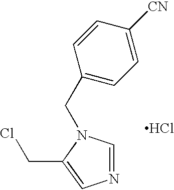

To an ice cold solution of dry acetonitrile (3.2 L, 15 L/Kg hydroxymethylimidazole) was added 99% oxalyl chloride (101 mL, 1.15 mol, 1.15 equiv.), followed by dry DMF (178 mL, 2.30 mol, 2.30 equiv.), at which time vigorous evolution of gas was observed. After stirring for about 5 to 10 min following the addition of DMF, solid hydroxymethylimidazole (213 g, 1.00 mol), as described above in Example 7, was added gradually. After the addition, the internal temperature was allowed to warm to a temperature of about 23° C. to about 25° C. and stirred for about 1 to 3 hours. The mixture was filtered, then washed with dry acetonitrile (400 mL displacement wash, 550 mL slurry wash, and a 400 mL displacement wash). The solid was maintained under a N₂ atmosphere during the filtration and washing to prevent hydrolysis of the chloride by adventitious H₂O. This yielded approximately 93 to about 96% crystalline form of the chloromethylimidazole hydrochloride.

¹H NMR (250 MHz DMSO-d₆): δ 9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d₆): δ_{c} 139.7, 137.7, 132.7, 130.1, 128.8, 120.7, 118.4, 111.2, 48.9, 33.1.

Step 6: Synthesis of the Amide Alcohol (1)

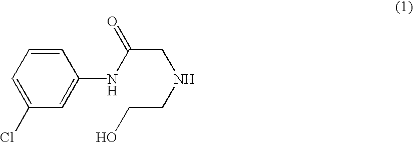

At 22° C., 3-chloroaniline (50.0 g) was combined with 460 ml isopropyl acetate and 20% aqueous potassium bicarbonate (72.5 g dissolved in 290 ml water). The biphasic mixture was cooled to 5° C. and chloroacetyl chloride (42 ml) was added dropwise over 30 minutes, keeping the internal temperature below 10° C. The reaction mixture was warmed to 22° C. over 30 min. The aqueous layer was removed at 22° C. and ethanolamine (92 ml) was added rapidly. The reaction mixture was warmed to 55° C. over 30 minutes and aged for 1 hour. At 55° C., 140 ml water was added with 30 ml isopropyl acetate to the reaction mixture. The biphasic reaction mixture was agitated for 15 minutes at 55° C. The layers were allowed to settle and the aqueous layer was removed. The organic layer was cooled to 45° C. and seed was added. The mixture was cooled to 0° C. over 1 hour and aged for 1 hour. The solids were filtered and washed with chilled isopropyl acetate (2×75 ml). The solids were dried in vacuo at 40° C. for 18 hours to provide about an 83.5% yield of the amide alcohol (1).

¹H NMR (300 MHz; DMSO-d₆) δ 7.85 (t, 1H 2.0 Hz), 7.52 (m, 1H), 7.32 (t, 1H, 8.0 Hz), 4.5-4.8 (br s, 1H), 3.47 (t, 1H, 5.5 Hz), 3.30 (s, 1H), 2.60 (t, 1H 5.0 Hz).

¹³C NMR (75.4 MHz; DMSO-d₆) δ_{c} 170.9, 140.1, 133.0, 130.3, 122.8 118.5, 117.5, 60.3, 52.7, 51.5.

Step 7: Synthesis of 1-(3-Chlorophenyl)-2-Piperazinone Hydrochloride with DIAD

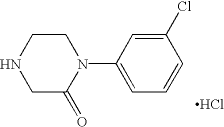

58 mL of EtOAc was charged to an N₂-purged flask. Tributylphosphine (28.3 mL, 113.8 mmol) was added, via syringe, and the solution was cooled to about −10° C. DIAD (22.4 mL, 113.8 mmol) was added dropwise over 30 minutes, maintaining the temperature at <0° C. The above mixture was cannulated into a slurry of an amide alcohol (20.0 g, 87.5 mmol), prepared as described above in Step 6, in 117 mL EtOAc over 20 minutes, maintaining the temperature at <0° C. The reaction was warmed to room temperature over 25 minutes. 99% conversion was observed by LC assay. Water (0.55 mL) was then added, and the reaction was warmed to 40° C. The solution was seeded with 200 mg of authentic material, and 1.0 eq. HCl (4.0 N in abs. EtOH) was added dropwise over 2 hours. The slurry was cooled to 0° C. over 2 hours and aged at 0° C. for 1 hour. The mixture was filtered, and the cake was washed with chilled EtOAc (3×16 mL). The cake was dried in vacuo overnight at 40° C. to afford about a 77% yield of the above-titled compound.

Step 8: Preparation of 1-(3-chlorophenyl)-4-[1-(4-cyanobenzyl)-5-imidazolylmethyl]-2-piperazinone.H₂O (Crystal Form I)

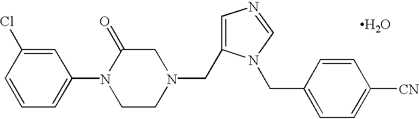

A 50 L four-neck flask, equipped with a mechanical stirrer, cooling bath, teflon-coated thermocouple, and nitrogen inlet was charged with 4.0 L of acetonitrile. Then 4-cyanobenzyl-chloromethylimidazole hydrochloride (958 g, 3.36 mol), prepared as described in Step 4, piperazinone hydrochloride (883 g, 3.54 mol), prepared as described in Step 7, and the remaining 1.25 L of acetonitrile were added to the flask at room temperature. Disopropylethylamine (1.99 L, 11.4 mol) was added to the mixture. The bulk of the solid dissolved immediately upon addition of diisopropylethylamine, leaving a slightly turbid solution.

After stirring 30 min, the solution was cooled to 0° C. over 60 min. The solution was stirred 26 h at 0° C., then warmed to 20° C. over 20 min. Water (2 L) was added to the slightly turbid solution over 20 min. Authentic seed was added to 8 L of water, which was subsequently added to the solution over 70 min. Additional water (17 L) was added over 90 min, and the mixture was aged 60 min thereafter. The temperature throughout the addition and aging was from about 20° C. to about 22° C. The mixture was filtered through a polypropylene filter pot. The crystals were washed with 1:5 acetonitrile/water. The crystalline solid was dried by passage of nitrogen through the filter cake (36 h) to provide the above-titled compound.

¹³C NMR (62.9 MHz, CDCl₃): δ 165.2, 142.7, 142.1, 139.4, 134.8, 133.0, 131.0, 130.2, 127.3, 127.1, 126.3, 126.0, 123.9, 118.1, 112.0, 57.7, 50.6, 49.9, 148.8, 148.3.

Step 9: Preparation of 1-(3-chlorophenyl)-4-[1-(4-cyanobenzyl)-5-imidazolylmethyl]-2-piperazinone.HCl

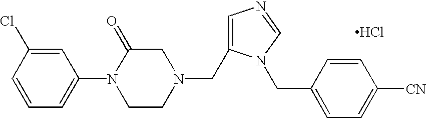

An IPA/toluene mixture (7 L) is made up as a 69:31 wt % ratio by mixing IPA (3.90 Kg, 4.97 L) and toluene (1.76 Kg, 2.03 L).

A pre-weighed 1 L graduated cylinder was charged with IPA (500 mL, 392 g). The cylinder was cooled to 0° C. Gaseous HCl was bubbled into the IPA until a volume change of roughly +80 mL was observed. The new weight of the cylinder and its contents indicated that 140 g HCl (3.84 moles) had been charged, making up a 6.62 M solution (or 7.22 molal solution). An aliquot (500 mL, 458 g) was transferred to a 5 L flask. To this solution was added toluene (192 mL, 166 g) and the 69:31 IPA/toluene mixture (2.07 Liters, 1.7 Kg).

A 22 L flask was charged with the free base form of 1-(3-chlorophenyl)-4-[1-(4-cyanobenzyl)-5-imidazolylmethyl]-2-piperazinone, prepared as described above in Step 8. The 69:31 IPA/toluene mixture (11.0 L) was added to this flask, which resulted in dissolution of the solid. The solution was heated to 40° C. The hot solution was filtered through an in-line filter into a pre-heated (40° C.) 22 L flask. The dissolution flask was further washed with the 69:31 IPA/toluene solution (0.5 L), which was transferred to the crystallization flask through the in-line filter. The in-line filter was replaced with a 4 L addition funnel.

The 1.21 M HCl solution (1.93 L, 1.63 Kg, 2.34 moles, 0.99 equiv.) was charged to the addition funnel. A fraction of the HCl solution (0.19 Liters, 0.23 moles, 0.10 equiv.) was added to the solution of free base over 10 min, whereupon the solution was seeded. After aging the thin slurry for 10-15 min, the remaining HCl solution was added over 2 h. The thick mixture was cooled to −10° C. over 2 h, aged for 30 min, then filtered. The crystals were washed with ice-cold 69:31 IPA/toluene and was then washed three times with ice-cold IPA. The crystals were dried under vacuum with a nitrogen stream and the above-titled compound was obtained.

### Example 3

4-[1-(5-Chloro-2-oxo-2H-[1,2′]bipyridinyl-5′-ylmethyl)-1H-pyrrol-2-ylmethyl]-benzonitrile

Step 1: 5-Chloro-5′-methyl-[1,2′]bipyridinyl-2-one

5-Chloro-2-pyridinol (2.26 g, 17.4 mmol), 2-bromo-5-methylpyridine (3.00 g, 17.4 mmol), copper (0.022 g, 0.35 mmol) and K2CO₃ (2.66 g, 19.2 mmol) were heated at 180° C. for 16 hrs. The brown reaction mixture was cooled, diluted with EtOAc and washed with saturated NaHCO₃. The aqueous layer was extracted with EtOAc (2×) and the combined organic extracts were washed with brine, dried (Na₂SO₄) and evaporated in vacuo. The residue was chromatographed (silica gel, EtOAc: CH₂Cl₂ 20:80 to 50:50 gradient elution) to afford the title compound as a white solid.

¹H NMR (400 MHz, CDCl₃) δ 8.37 (s, 1H), 7.96(d, J=3.0 Hz, 1H), 7.83 (d, J=8.4 Hz, 1H), 7.65(dd, J=2.4 and 8.2 Hz, 1H), 7.32(dd, J=2.9 and 9.7 Hz, 1H), 6.61(d, J=9.7 Hz, 1H) and 2.39(s,3H)ppm.

Step 2: 5′-Bromomethyl-5-chloro-[1,2′]bipyridinyl-2-one

A solution of the pyridine from Step 1(1.00 g, 4.53 mmol), N-bromosuccinimide (0.81 g, 4.53 mmol) and AIBN (0.030 g, 0.18 mmol) in CCl₄ (40 mL) was heated at reflux for 2 hrs. The solids were filtered and the filtrate collected. The solvent was evaporated in vacuo and the residue chromatographed (silica gel, EtOAc: CH₂Cl₂ 25:75 to 50:50 gradient elution) to afford the title bromide.

¹H NMR (400 MHz, CDCl₃) δ 8.55 (s, 1H), 8.04(d, J=2.9 Hz, 1H), 8.01 (d, J=8.4 Hz, 1H), 7.88 (dd, J=2.4 and 8.6 Hz, 1H), 7.34(dd, J=2.9 and 9.8 Hz, 1H), 6.61(d, J=9.9 Hz, 1H) and 4.51 (s,2H) ppm.

Step 3: 4-[1-(5-Chloro-2-oxo-2H-[1,2′]bipyridinyl-5′-ylmethyl)-1H-pyrrol-2-ylmethyl]-benzonitrile Hydrochloride

The bromide from Step 2 (0.750 g, 2.50 mmol) and the 4-(1-trityl-1H-imidazol-4-ylmethyl)-benzonitrile (1.06 g, 2.50 mmol) in CH₃CN (10 mL) were heated at 60° C. The reaction was cooled to room temperature and the solids collected by filtration and washed with EtOAc (10 mL). The solid was suspended in methanol (50 mL) and heated at reflux for 1 hr, cooled and the solvent evaporated in vacuo. The sticky residue was stirred in EtOAc (40 mL) for 4 hrs and the resulting solid hydrobromide salt collected by filtration and washed with EtOAc (40 mL) and dried in vacuo. The hydrobromide salt was partitioned between sat. NaHCO₃ and CH₂Cl₂ and extracted with CH₂Cl₂. The organic extracts were dried (Na₂SO₄) and evaporated in vacuo. The residue was chromatographed (silica gel, MeOH: CH₂Cl₂ 4:96 to 5:95 gradient elution) to afford the free base which was converted to the hydrochloride salt to afford the title compound as a white solid.

¹H NMR (400 MHz, CD₃OD) δ 9.11 (s, 1H), 8.35 (s, 1H), 8.03(d, J=2.9 Hz, 1H), 7.83 (d, J=8.4 Hz, 1H), 7.76 (dd, J=2.4 and 9.6 Hz, 1H), 7.68-7.58 (m, 3H), 7.48 (s, 1H), 7.31(d, J=8.6 Hz, 2H), 6.68 (d, J=9.3 Hz, 1H), 5.53 (s, 2H) and 4.24 (s, 2H) ppm.

Analysis: Calc for C₂₂H₁₆N₅OCl: 1.75HCl, 0.15EtOAc C, 56.69; H, 3.99; N, 14.62 Found: C, 56.72; H, 4.05; N, 14.54.

### Example 4

Preparation of (R)-1-(3-chlorophenyl)-4-[1-(4-cyanobenzyl)-5-imidazolylmethyl]-5-[2-(ethanesulfonyl)methyl]-2-piperazinone dihydrochloride

Step A: Preparation of (R)-2-(tert-butoxycarbonylamino)-N-(3-chlorophenyl)-3-[(triphenylmethyl)thio]-1-propanamine

To a solution of 3-chloroaniline (0.709 mL. 6.70 mmol) in 30 mL of dichloromethane at room temperature was added 1.2 g of crushed 4 Å molecular sieves. Sodium triacetoxyborohydride (3.55 g, 16.7 mmol) was added, followed by dropwise addition of N-methylmorpholine to achieve a pH of 6.5. L-S-Trityl-N-Boc-cysteinal (3.15 g, 7.04 mmol) (prepared according to S. L. Graham et al. J. Med. Chem., (1994) Vol. 37, 725-732) was added, and the solution was stirred for 48 hours. The reaction was quenched with sat. aq. NaHCO₃, diluted with EtOAc, and the layers were separated. The organic material was washed with brine, dried (Na₂SO₄), filtered, and concentrated in vacuo to provide an oil which was purified by silica gel chromatography (15% EtOAc/hexane) to give the title amine.

Step B: Preparation of (R)-N-[2-(tert-butoxycarbonylamino)-3-((triphenylmethyl)thio)propyl-2-chloro-N-(3-chlorophenyl)acetamide

The aniline derivative from Step A (2.77 g, 4.95 mmol) was dissolved in 73 mL of EtOAc and 73 mL of sat. NaHCO₃ soln., then cooled to 0° C. With vigorous stirring, chloroacetyl chloride (0.533 mL. 6.69 mmol) was added dropwise. After 3 hours, the reaction was diluted with water and EtOAc, and the organic layer was washed with brine, dried (Na₂SO₄), filtered, and concentrated in vacuo to provide crude titled chloroacetamide which was used without further purification.

Step C: Preparation of (R)-4-(tert-butoxycarbonyl)-1-(3-chlorophenyl)-5-[S-(triphenylmethyl)thiomethyl]piperazin-2-one

To a solution of chloroacetamide from Step B (3.29 g crude, theoretically 4.95 mmol) in 53 mL of DMF at 0° C. was added cesium carbonate (4.84 g, 14.85 mmol). The solution was stirred for 48 hours, allowing it to warm to room temperature. The solution was poured into EtOAc, washed with water and brine, dried (Na₂SO₄), filtered, and concentrated in vacuo to provide the crude product as an oil. This material was purified by silica gel chromatography (20% EtOAc/hexane) to yield the product as a white solid.

Step D: Preparation of (R)-4-(tert-butoxycarbonyl)-1-(3-chlorophenyl)-5-(thiomethyl)piperazin-2-one

A solution of piperazinone from Step C (625 mg, 1.04 mmol) in degassed EtOAc (38 mL) and EtOH (12 mL) was warmed to 30° C. A solution of AgNO₃ (177 mg, 1.04 mmol) and pyridine (0.084 mL, 1.04 mmol) in 8 mL of EtOH was added, and the solution was heated to reflux. After 45 minutes, the reaction was concentrated in vacuo, then redissolved in 26 mL of degassed EtOAc. Through this solution was bubbled H₂S gas for 2.5 minutes, then activated charcoal was added after 4 minutes. The material was filtered through celite and rinsed with degassed EtOAc, concentrated in vacuo, then reconcentrated from degassed CH₂Cl₂ to provide the crude product which was used without further purification.

Step E: Preparation of (R)-4-(tert-butoxycarbonyl)-1-(3-chlorophenyl)-5-[(ethylthio)methyl]piperazin-2-one

A solution of the thiol from Step D (ca. 1.04 mmol) in 3 mL of THF was added via cannula to a suspension of NaH (51.4 mg, 60% disp. in mineral oil, 1.28 mmol) in 2 mL THF at 0° C. After 10 minutes, iodoethane was added (0.079 mL, 0.988 mmol), and-the solution was stirred for 1.5 hours. The reaction was poured into EtOAc, washed with sat. NaHCO₃ and brine, dried (Na₂SO₄), filtered, and concentrated in vacuo to provide the crude product. This material was purified by silica gel chromatography (1% MeOH/CH₂Cl₂) to yield the titled product.

Step F: Preparation of (R)-4-(tert-butoxycarbonyl)-1-(3-chlorophenyl)-5-[(ethanesulfonyl)methyl]piperazin-2-one

To a solution of the sulfide from Step E (217 mg, 0.563 mmol) in 3 mL of MeOH at 0° C. was added a solution of magnesium monoperoxyphthalate (835 mg, 1.69 mmol) in 2 mL MeOH. The reaction was stirred overnight, allowing it to warm to room temperature. The solution was cooled to 0° C., quenched by the addition of 4 mL 2N Na₂S203 soln., then concentrated in vacuo. The residue was partitioned between EtOAc and sat NaHCO₃ solution, and the organic layer was washed with brine, dried (Na₂SO₄), filtered, and concentrated in vacuo to provide the crude sulfone as a white waxy solid.

Step G: Preparation of (R)-1-(3-chlorophenyl)-4-[1-(4-cyanobenzyl)-5-imidazolylmethyl]-5-[2-(ethanesulfonyl)methyl]-2-piperazinone Dihydrochloride

To a solution of the Boc-protected piperazinone from Step F (224 mg, 0.538 mmol) in 5 mL of dichloromethane at 0° C. was added 2.5 mL of trifluoroacetic acid (TFA). After 45 minutes, the reaction was concentrated in vacuo, then azeotroped with benzene to remove the excess TFA. The residue was taken up in 4 mL of 1,2-dichloroethane and cooled to 0° C. To this solution was added 4 Å powdered molecular sieves (340 mg), followed by sodium triacetoxyborohydride (285 mg, 1.34 mmol) and several drops of triethylamine to achieve pH=6. The imidazole carboxaldehyde from Step E of Example 42 (125 mg, 0.592 mmol) was added, and the reaction was stirred at 0° C. After 2 days, the reaction was poured into EtOAc, washed with dilute aq. NaHCO₃, and brine, dried (Na₂SO₄), filtered, and concentrated in vacuo. The crude product was taken up in methanol and injected onto a preparative HPLC column and purified with a mixed gradient of 15%-50% acetonitrile/0.1% TFA; 85%-50% 0.1% aqueous TFA over 60 minutes. After concentration in vacuo, the resultant product was partitioned between dichloromethane and aq. NaHCO₃ soln., and the aqueous phase was extracted with CH₂Cl₂. The organic solution was washed with brine, dried (Na₂SO₄), filtered, and concentrated to dryness to provide the product free base, which was taken up in CH₂Cl₂ and treated with 2.1 equivalents of 1 M HCl/ether solution. After concentrated in vacuo, the product dihydrochloride was isolated as a white powder.

### Example 5

Preparation of (±)-19,20-Dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile Dihydrochloride

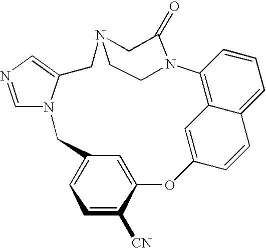

Step A: Preparation of N-(7-hydroxy-1-naphthyl)-2-[(2-(hydroxy)ethyl)amino]acetamide

To a solution of 8-amino-2-naphthol (15.00 g, 94.2 mmol) in 300 mL of isopropyl acetate and 250 mL of saturated NaHCO₃ solution at 0° C. was added chloroacetyl chloride (18.75 mL, 235 mmol). 30 minutes, the layers were separated, and the organic layer was filtered through a glass frit to remove insolubles. Ethanolamine was added (20.9 mL, 377 mmol), and the reaction was warmed to 50° C. for 2 hours, then cooled to room temperature. The solution was poured into EtOAc, washed with water and brine, dried (Na₂SO₄), filtered, and concentrated in vacuo. The titled product was obtained as a dark brown solid which was used in the next reaction without further purification.

Step B: Preparation of N-(7-hydroxy-1-naphthyl)-2-[(2-(hydroxy)ethyl)tert-butoxycarbonyl amino]acetamide

To a solution of the product from Step A (7.50 g, 28.8 mmol) in 100 mL of tetrahydrofuran at 0° C. was added di-tert-butyldicarbonate (6.29 g, 28.8 mmol). After 1.5 hours, the solution was concentrated in vacuo to provide the titled product as a dark brown foam which was used in the next reaction without further purification.

Step C: Preparation of 4-tert-butoxycarbonyl-1-(7-hydroxy-1-naphthyl)-2-piperazinone

To a solution of di-tert-butylazodicarboxylate (10.81 g, 43.2 mmol) in 60 mL of tetrahydrofuran at 0° C. was added tributylphosphine (10.76 mL, 43.2 mmol) dropwise. After 10 minutes, a solution of the crude product from Step B (ca. 28.8 mmol) in 30 mL of tetrahydrofuran was added dropwise, and the reaction was allowed to warm to room temperature. After two hours, HPLC analysis showed partial conversion. The solution was cooled to 0° C., and additional portions of tributylphosphine (3.0 mL, 18 mmol) and di-tert-butylazodicarboxylate (4.6 g, 18 mmol) were added. The reaction was warmed to room temperature, and stirred for 16 hours. The solution was concentrated in vacuo, and the resulting product was purified by silica gel chromatography (0-5% MeOH/CH₂Cl₂) to provide the titled product as a dark brown foam, contaminated with tributylphosphine oxide impurity. This material was used in the next reaction without further purification.

Step D: Preparation of 1-(7-benzyloxy-1-naphthyl)-4-tert-butoxycarbonyl-2-piperazinone

To a solution of the product from Step C (ca. 28.8 mmol) in 150 mL of acetone was added potassium carbonate (20.0 g, 145 mmol), followed by benzyl bromide (3.45 mL, 29 mmol). The reaction was heated to reflux, and stirred for 18 hours. After cooling to room temperature, the solution was concentrated in vacuo to a 50 mL volume, poured into EtOAc, washed with sat. aq. NaHCO₃ and brine, dried (Na₂SO₄), filtered, and concentrated in vacuo. The crude product mixture was purified by silica gel chromatography (40-50% EtOAc/hexane) to provide the titled compound as a pale brown foam.

Step E: Preparation of 1-(7-benzyloxy-1-naphthyl)-2-piperazinone Hydrochloride

Through a solution of the product from Step D (1.244 g, 2.88 mmol) in 50 mL of ethyl acetate at 0° C. was bubbled anhydrous HCl gas for 5 minutes. After 30 minutes, the solution was concentrated in vacuo to provide the titled salt as a brown powder (1.064 g) which was used in the next reaction without further purification.

Step F: Preparation of 1-(7-benzyloxy-1-naphthyl)-4-[1-(4-cyano-3-fluorobenzyl)-5-imidazolylmethyl]-2-piperazinone

To a solution of the crude amine hydrochloride from Step E (2.88 mmol) in 15 mL of 1,2-dichloroethane was added 4 Å powdered molecular sieves (2.0 g), followed by sodium triacetoxyborohydride (911 mg, 4.32 mmol). The aldehyde from Step G of Example 1 was added (659 mg, 2.88 mmol), and the reaction was stirred for 40 minutes. The reaction was poured into EtOAc, washed with sat. aq. NaHCO₃ and brine, dried (Na₂SO₄), filtered, and concentrated in vacuo. The titled product was obtained as a brown foam which was used in the next reaction without further purification.

Step G: Preparation of 1-(7-hydroxy-1-naphthyl)-4-[1-(4-cyano-3-fluorobenzyl)-5-imidazolylmethyl]-2-piperazinone Trifluoroacetate

To a solution of the benzyl ether from Step F (1.563 g, 2.85 mmol) in 25 mL of 1:1 MeOH/EtOAc was added trifluoroacetic acid (1.0 mL) and 10% palladium on carbon (900 mg). The solution was stirred under a balloon atmosphere of hydrogen at room temperature. After 8 hours, the solution was filtered through celite, and the filter pad was rinsed with 1:1 MeOH/THF. Concentration in vacuo provided the titled product as a white foam which was used in the next reaction without further purification.

Step H: Preparation of (±)-19,20-Dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile, Dihydrochloride

To a solution of the product from Step G (ca. 2.85 mmol) in 50 mL of DMSO was added cesium carbonate (2.815 g, 8.64 mmol). The reaction was warmed to 55° C. under argon for 45 minutes, then cooled to room temperature. The solution was poured into EtOAc and washed with water and brine, dried (Na₂SO₄), filtered, and concentrated in vacuo. The resulting product was purified by silica gel chromatography (5-8% MeOH/CH₂Cl₂) to provide the product as a pale yellow foam. A portion of this was taken up in CH₂Cl₂, treated with excess 1 M HCl/ether solution, and concentrated in vacuo to provide the titled product dihydrochloride as a pale yellow powder.

FAB mass spectrum m/e 436.3 (M+1).

Analysis calculated for C₂₆H₂₁N₅O₂.2.10HCl.1.10H₂O: C, 58.77; H, 4.80; N, 13.18; Found: C, 58.82; H, 4.79; N, 12.67.

### Example 6

(+)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile, Enantiomer A Dihydrochloride

A sample of free base of the compound described in Example 5, Step H (96 mg in 3 mL of MeOH) was resolved by preparative chiral HPLC at 310 nm using a Chiralcel OD 250×4.6 mm column, and eluting with a 80% ethanol/0.1% diethylamine-hexane at a flow rate of 1.0 mL/min. The faster eluting product was taken up in CH₂Cl₂, treated with excess 1 M HCl/ether solution, and concentrated in vacuo to provide the titled product dihydrochloride as a pale white powder. Assay for enantiomeric purity (retention time=8.04 min; Chiralcel OD 25×2 mm; 80-100% gradient: ethanol/0.1% diethylamine-hexane over 45 min; flow rate 8.0 mL/min; 310 nm) indicated 96.4% enantiomeric excess.

Analysis calculated for C₂₆H₂₁N₅O₂.2.15HCl.2.45H₂O: C, 55.97; H, 5.07; N, 12.55; Found: C, 56.00; H, 5.11; N, 12.34.

### Example 7

(−)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile, Enantiomer B Dihydrochloride

The titled product was produced under the same conditions described in Example 6. Assay of the slower-eluting product for enantiomeric purity (retention time=13.96 min; Chiralcel OD 25×2 mm; 80-100% gradient: ethanol/0.1% diethylamine-hexane over 45 min; flow rate 8.0 mL/min; 310 nm) indicated >99% enantiomeric excess.

Analysis calculated for C₂₆H₂₁N₅O₂.2.00HCl.2.30H₂O: C, 56.79; H, 5.06; N, 12.74; Found: C, 56.80; H, 5.38; N, 12.58.

### Example 8

1-(3-chlorophenyl)-4-[1-(4-cyano-3-methoxybenzyl)-5-imidazolylmethyl]-2-piperazinone Dihydrochloride

Step A: Preparation of Methyl 4-Amino-3-hydroxybenzoate

Through a solution of 4-amino-3-hydroxybenzoic acid (75 g, 0.49 mol) in 2.0 L of dry methanol at room temperature was bubbled anhydrous HCl gas until the solution was saturated. The solution was stirred for 48 hours, then concentrated in vacuo. The product was partitioned between EtOAc and saturated aq. NaHCO₃ solution, and the organic layer was washed with brine, dried (Na₂SO₄), and concentrated in vacuo to provide the titled compound.

Step B: Preparation of Methyl 3-Hydroxy-4-iodobenzoate

A cloudy, dark solution of the product from Step A (79 g, 0.47 mol), 3N HCl (750 mL), and THF (250 mL) was cooled to 0° C. A solution of NaNO₂ (35.9 g, 0.52 mol) in 115 mL of water was added over ca. 5 minutes, and the solution was stirred for another 25 minutes. A solution of potassium iodide (312 g, 1.88 mol) in 235 mL of water was added all at once, and the reaction was stirred for an additional 15 minutes. The mixture was poured into EtOAc, shaken, and the layers were separated. The organic phase was washed with water and brine, dried (Na₂SO₄), and concentrated in vacuo to provide the crude product (148 g). Purification by column chromatography through silica gel (0%-50% EtOAc/hexane) provided the titled product.

Step C: Preparation of Methyl 4-Cyano-3-hydroxybenzoate

A mixture of the iodide product from Step B (101 g, 0.36 mol) and zinc(II)cyanide (30 g, 0.25 mol) in 400 mL of dry DMF was degassed by bubbling argon through the solution for 20 minutes. Tetrakis(triphenylphosphine)palladium (8.5 g, 7.2 mmol) was added, and the solution was heated to 80° C. for 4 hours. The solution was cooled to room temperature, then stirred for an additional 36 hours. The reaction was poured into EtOAc/water, and the organic layer was washed with brine (4×), dried (Na₂SO₄), and concentrated in vacuo to provide the crude product. Purification by column chromatography through silica gel (30%-50% EtOAc/hexane) provided the titled product.

Step D: Preparation of Methyl 4-Cyano-3-methoxybenzoate

Sodium hydride (9 g, 0.24 mol as 60% wt. disp. mineral oil) was aded to a solution of the phenol from Step C (36.1 g, 204 mmol) in 400 mL of dry DMF at room temperature. Iodomethane was added (14 mL. 0.22 mol) was added, and the reaction was stirred for 2 hours. The mixture was poured into EtOAc/water, and the organic layer was washed with water and brine (4×), dried (Na₂SO₄), and concentrated in vacuo to provide the titled.

Step E: Preparation of 4-Cyano-3-methoxybenzyl Alcohol

To a solution of the ester from Step D (48.8 g, 255 mmol) in 400 mL of dry THF under argon at room temperature was added lithium borohydride (255 mL, 510 mmol, 2M THF) over 5 minutes. After 1.5 hours, the reaction was warmed to reflux for 0.5 hours, then cooled to room temperature. The solution was poured into EtOAc/1N HCl soln. [CAUTION], and the layers were separated. The organic layer was washed with water, sat Na₂CO₃ soln. and brine (4×), dried (Na₂SO₄), and concentrated in vacuo to provide the titled product.

Step F: Preparation of 4-Cyano-3-methoxybenzyl Bromide

A solution of the alcohol from Step E (35.5 g, 218 mmol) in 500 mL of dry THF was cooled to 0° C. Triphenylphosphine was added (85.7 g, 327 mmol), followed by carbontetrabromide (108.5 g, 327 mmol). The reaction was stirred at 0° C. for 30 minutes, then at room temperature for 21 hours. Silica gel was added (ca. 300 g), and the suspension was concentrated in vacuo. The resulting solid was loaded onto a silica gel chromatography column. Purification by flash chromatography (30%-50% EtOAc/hexane) provided the titled.

Step G: Preparation of 1-(4-cyano-3-methoxybenzyl)-5-(acetoxymethyl)-imidazole Hydrobromide

The titled product was prepared by reacting the bromide from Step F (21.7 g, 96 mmol) with the imidazole product from Step B of Example 8 (34.9 g, 91 mmol) using the procedure outlined in Step C of Example 8. The crude product was triturated with hexane to provide the titled product hydrobromide.

Step H: Preparation of 1-(4-cyano-3-methoxybenzyl)-5-(hydroxymethyl)-imidazole

The titled product was prepared by hydrolysis of the acetate from Step G (19.43 g, 68.1 mmol) using the procedure outlined in Step D of Example 1. The crude titled product was isolated both directly from extraction or through concentration of the aqueous extracts which provided solid material (ca. 100 g) which contained a significant quantity of the titled product, as judged by H NMR spectroscopy.

Step I: Preparation of 1-(4-cyano-3-methoxybenzyl)-5-imidazolecarboxaldehyde

The titled product was prepared by oxidizing the alcohol from Step H (11 g, 45 mmol) using the procedure outlined in Step E of Example 1. The titled aldehyde was isolated as a white powder which was sufficiently pure for use in the next step without further purification.

Step J: Preparation of 1-(3-chlorophenyl)-4-[1-(4-cyano-3-methoxybenzyl)-5-imidazolylmethyl-2-piperazinone Dihydrochloride

The titled product was prepared by reductive alkylation of the aldehyde from Step I (859 mg, 3.56 mmol) and the amine (hydrochloride) from Step E of Example 2 (800 mg, 3.24 mmol) using the procedure outlined in Step F of Example 2. Purification by flash column chromatography through silica gel (50%-75% acetone CH₂Cl₂) and conversion of the resulting white foam to its dihydrochloride salt provided the titled product as a white powder.

FAB ms (m+1) 437.

Anal. Calc. for C₂₃H₂₃ClN₅O₂.2.0HCl.0.35CH₂Cl₂: C, 51.97; H, 4.80; N, 12.98. Found: C, 52.11; H, 4.80; N, 12.21.

### Example 9

1-(3-trifluoromethoxyphenyl)-4-[1-(4-cyano-3-methoxybenzyl)-5-imidazolyl methyl]-2-piperazinone Dihydrochloride

1-(3-trifluoromethoxy-phenyl)-2-piperazinone hydrochloride was prepared from 3-trifluoromethoxyaniline using Steps A-E of Example 2. This amine (1.75 g, 5.93 mmol) was coupled to the aldehyde from Step I of Example 8 (1.57 g, 6.52 mmol) using the procedure outlined in Step F of Example 2. Purification by flash column chromatography through silica gel (60%-100% acetone CH₂Cl₂) and conversion of the resulting white foam to its dihydrochloride salt provided the titled product as a white powder.

FAB ms (m+1) 486.

Anal. Calc. for C₂₄H₂₃F₃N₅O₃.2.0HCl.0.60H₂O: C, 50.64; H, 4.46; N, 12.30. Found: C, 50.69; H, 4.52; N, 12.13.

### Example 10

4-{3-[4-(−2-Oxo-2-H-pyridin-1-yl)benzyl]-3-H-imidazol-4-ylmethyl]benzonitrile

Step 1: 4-Iodobenzyl Alcohol

Methyl 4-iodobenzoate (5 g, 19.07 mmol) was suspended in THF (100 mL). LiBH₄ (40 mmol) was added slowly, via syringe. Reaction mixture was heated to 60° for 4 days. 1N HCl was added slowly. Reaction mixture was stirred for ½ hour then was extracted 3 times with EtOAc. The organic layers were combined, washed with saturated NaHCO₃, brine, dried (MgSO₄), filtered and concentrated to give 4-iodobenzyl alcohol as a white solid.

¹H NMR (400 MHz, CDCl₃) δ 7.68 (d, 2H); 7.11 (d, 2H); 4.71 (d, 2H); 1.65 (t, 1H)

Step 2: 4-(−2-Oxo-2-H-pyridin-1-yl)benzyl alcohol 2-Hydroxypyridine (10.0 mmol; 956-mg), 4-iodobenzyl alcohol (17.09 mmol, 4.0 g), K₂CO₃ (11.0 mmol, 1.52 g), and copper (0.2 mmol, 15 mg) were mixed under argon and heated to 150° for 16 hours. The solid was partitioned between saturated NaHCO₃ and EtOAc. The layers were separated and the aqueous layer was back extracted twice with EtOAc. The organic layers were combined, washed with brine, dried over MgSO₄, filtered and concentrated to yield a yellow oil which was purified by flash chromatography (EtOAc) to give pure the title compound as a crystalline solid.

¹H NMR (400 MHz, CDCl₃) δ 7.50 (d, 2H); 7.43-7.41 (m, 4H); 6.68-6.65 (d, 1H): 6.27-6.23 (t, 1H); 4.75-4.75(d, 2H); 1.96-1.95 (bt, 1H).

Step 3: 4-(−2-Oxo-2-H-pyridin-1-yl)benzyl Bromide

A solution of NBS (1.59 g, 8.94 mmol) and CH₂Cl₂ was cooled to 0°. To this solution (under Ar) was added Me₂S (10.72 mmol, 0.78 mL) via syringe. This mixture was then cooled to −20° and added to a solution of the benzyl alcohol from Step 2 (1.2 g, 5.96 mmol) in CH₂Cl₂ via pipette. The reaction mixture was warmed to 0° and stirred for several hours. The residue was poured into ice water and extracted with CH₂Cl₂ (3×). The organic layers were combined, washed with brine, dried (MgSO₄), filtered and concentrated to give the title compound as a yellow solid, which will be used in the next step without further purification.

¹H NMR (400 MHz, CDCl₃) δ 7.53-7.51 (d, 2H); 7.37-7.31 (m, 4H); 6.67 (d, 1H); 6.25 (t, 1H); 4.52 (s, 2H).

Step 4: 4-(1-Trityl-1H-imidazol-4-ylmethyl)-benzonitrile

To a suspension of activated zinc dust (3.57 g, 54.98 mmol) in THF (50 mL) was added dibromoethane (0.315 mL, 3.60 mmol) and the reaction stirred under argon for 45 minutes, at 20° C. The suspension was cooled to 0° C. and α-bromo-p-tolunitrile (9.33 g, 47.6 mmol) in THF (100 mL) was added dropwise over a period of 10 minutes. The reaction was then allowed to stir at 20° C. for 6 hours and bis(triphenylphosphine)Nickel II chloride (2.40 g, 3.64 mmol) and 5-iodotrityl imidazole (15.95 g, 36.6 mmol) were added in one portion. The resulting mixture was stirred 16 hours at 20° C. and then quenched by addition of saturated NH₄Cl solution (100 mL) and the mixture stirred for 2 hours. Saturated aq. NaHCO₃ solution was added to give a pH of 8 and the solution was extracted with EtOAc (2×250 mL), dried (MgSO₄) and the solvent evaporated in vacuo. The residue was chromatographed (silica gel, 0-20% EtOAc in CH₂Cl₂) to afford the title compound as a white solid.

¹H NMR (CDCl₃, 400 Mz) δ (7.54 (2H, d, J=7.9 Hz), 7.38(1H, s), 7.36-7.29 (11H, m), 7.15-7.09(6H, m), 6.58(1H, s) and 3.93(2H, s) ppm.

Step 5: 4-{3-[4-(2-Oxo-2-H-pyridin-1-yl)benzyl]-3-H-imidazol-4-ylmethyl]benzonitrile

4-(−2-Oxo-2-H-pyridin-1-yl)benzyl bromide from Step 3 (1.7 mmol, 450 mg) and 4-(1-trityl-1H-imidazol-4-ylmethyl)-benzonitrile (1.7 mmol) were suspended in CH₃CN and heated to reflux for 3 hours. The reaction mixture was concentrated and the residue taken up in MeOH and refluxed for 2 hours. The MeOH was removed in-vacuo. The resulting oil was partitioned between EtOAc and saturated NaHCO₃. The aqueous layer was extracted twice with EtOAc. The organic layers were combined, washed with brine, dried (MgSO₄), filtered and concentrated to yield an oil which was purified by flash chromatography using 5% IPA/CHCl₃ saturated with NH₃ as an eluent. Pure fractions were collected and concentrated to give a white solid. The solids were washed with warm 50% EtOAc/Hexane and collected on a frit. The white solid was collected and dried under high vacuum at 50° for 12 hours to give the title compound.

¹H NMR (400 MHz, CDCl₃) δ 7.58-7.55 (m, 3H); 7.42-7.40 (m, 1H); 7.39 (d, 2H); 7.27 (s, 1H); 7.20 (d, 2H); 7.04 (d, 2H); 6.67 (d, 1H); 6.27 (t, 1H); 4.97 (s, 2H); 3.89 (s, 2H).

### Example 11

4-{1-[4-(5-Chloro-2-oxo-2H-pyridin-1-yl)-benzyl]-1H-imidazol-2-ylmethyl}-benzonitrile

Step 1: 5-Chloro-1-(4-hydroxymethyl-phenyl)-1H-pyridin-2-one

5-Chloro-2-pyridinol (0.61 g, 4.7 mmol), 4-iodobenzyl-alcohol (1.00 g, 4.27 mmol), Copper (0.27 g, 4.27 mmol) and K₂CO₃ (0.65 g, 4.70 mmol) were heated at 180° C. for 16 hrs. The brown reaction mixture was cooled, diluted with EtOAc and washed with saturated NaHCO₃. The aqueous layer was extracted with EtOAc (2×) and the combined organic extracts were washed with brine, dried (Na₂SO₄) and evaporated in vacuo. The residue was chromatographed (silica gel, EtOAc as eluent) to afford the title compound as a white solid.

¹H NMR (400 MHz, CD₃OD) δ 7.74 (d,J=2.7 Hz, 1H), 7.59 (dd, J=3.0 and 9.6 Hz, 1H), 7.51 (d, J=8.6 Hz, 2H), 7.37 (d, J=8.4 Hz, 2H), 6.61 (d, J=9.4 Hz, 1H) and 4.67(s,1H) ppm.

Step 2: 1-(4-Bromomethyl-phenyl)-5-chloro-1H-pyridin-2-one

To N-bromosuccinimide (0.166 g, 0.929 mmol) in CH₂Cl₂ (3 mL) at 0° C. was added dimethylsulfide (0.082 mL, 1.11 mmol). The resulting suspension was cooled to −20° C. and a solution of the alcohol from Step 1 (0.146 g, 0.62 mmol) in CH₂Cl₂ was added dropwise over 2 minutes. The reaction mixture was stirred at 0° C. for 6 hrs and then poured into water and extracted with CH₂Cl₂. The extracts were dried (Na₂SO₄) and evaporated in vacuo. The residue was chromatographed (silica gel, EtOAc: CH₂Cl₂ 1:1 as eluent) to afford the title compound as a white solid.

¹H NMR (400 MHz, CDCl₃) δ 7.54 (d,J=8.4 Hz, 2H), 7.40-7.32 (m, 4H), 6.63 (dd, J=9.7 and 0.7 Hz, 1H) and 4.51(s,2H) ppm.

Step 3: 4-{1-[4-(5-Chloro-2-oxo-2H-pyridin-1-yl)-benzyl]-1H-imidazol-2-ylmethyl}-benzonitrile

The bromide from Step 2 (0.154 g, 0.516 mmol) and 4-(1-trityl-1H-imidazol-4-ylmethyl)-benzonitrile (0.22 g, 0.516 mmol) prepared as described in Example 10, Step 4, in CH₃CN (2 mL) were heated at 55° C. After 18 hr methanol (3 mL) was added and the reaction was heated at reflux for 3 hrs, cooled and the solvent evaporated in vacuo. The residue was partitioned between sat. NaHCO₃ and CH₂Cl₂ and extracted with CH₂Cl₂. The organic extracts were dried (Na₂SO₄) and evaporated in vacuo. The residue was chromatographed (silica gel, MeOH: CH₂Cl₂ 5:95 as eluent) to afford the free base which was converted to the hydrochloride salt to afford the title compound as a white solid.

¹H NMR (400 MHz, CD₃OD) δ 9.03 (s, 1H), 7.80-7.55 (m, 4H), 7.55-7.20 (m, 7H) 6.64 (d, J=9.7 Hz, 1H), 5.45 (s,2H) and 4.18 (s,2H) ppm.

Analysis: % Calc for C₂₂H₁₇N₅O.1.00HCl, 0.55H₂O, 0.25 CH₃CN C, 61.39; H, 4.39; N, 16.31% Found C, 61.42; H, 4.61; N, 16.58.

### Example 12

4-[3-(2-Oxo-1-phenyl-1,2-dihydropyridin-4-ylmethyl)-3H-imidazol-4-ylmethyl]benzonitrile

Step 1: 4-Hydroxymethyl-1H-pyridin-2-one

2-Oxo-1,2-dihydropyridine-4-carboxylic acid methyl ester (1.8 g, 12.2 mmol), prepared as described in J. Org. Chem., 26, 428 (1961), was suspended in THF (100 ml). A small amount of DMF was added to help increase solubility. LiBH₄ (61 mmol) was added and the reaction was stirred for 18 hours at room temperature. MeOH and H₂O are added to quench the reaction. The reaction is then concentrated to yield a yellow oil. Flash chromatography (5% MeOH/CHCl₃ to 20% MeOH/CHCl₃) yielded 4-hydroxymethyl-1H-pyridin-2-one as a white solid.

¹H NMR (400 MHz, CD₃OD) δ 7.38-7.36 (1H,d); 6.56 (s, 1H); 6.37-6.36 (d, 1H); 4.50 s, 2H).

Step 2: 4-(tert-butyldimethylsilyloxymethyl)-1H-pyridin-2-one

4-Hydroxymethyl-1H-pyridin-2-one from Step 1 (1.3 g, 10.5 mmol) was dissolved in DMF. t-Butyl dimethylsilyl chloride (12.6 mmol, 1.9 g) and imidazole (12.6 mmol, 858 mg) were added and the reaction was stirred for 16 hours. The reaction mixture was diluted with EtOAc and washed with H₂O (2×) and brine. The organic layer was dried (MgSO₄), filtered and concentrated to yield a yellow oil. Flash chromatography (EtOAC) yielded 4-(tert-butyldimethylsilyloxy-methyl)-1H-pyridin-2-one as an off white solid.

¹H NMR (400 MHz, CDCl₃) δ 7.30-7.28 (d, 1H); 6.60 (s, 1H); 6.20-6.18 (d, 1H); 4.58 (s, 2H); 0.955 (s, 9H); 0.11 (s, 6H).

Step 3: 4-(tert-butyl-dimethyl-silanyloxymethyl)-1-phenyl-1H-pyridin-2-one

4-(Tert-butyldimethylsilyloxymethyl)-1H-pyridin-2-one from Step 2 (1.5 g, 6.3 mmol) was dissolved in iodobenzene (189 mmol, 21.12 mL) and treated with copper (6.3 mmol, 400 mg) and K₂CO₃ (6.93 mmol, 958 mg.). The brown slurry was heated to 180° for 16 hrs. The reaction mixture was cooled, diluted with CHCl₃ and washed with saturated NaHCO₃. The aqueous layer was back extracted with CHCl₃ (2×). The organic layers were combined, washed with brine, dried (MgSO₄), filtered and concentrated to yield a yellow oil. Flash Chromatography (20% EtOAc/Hexane) yielded 4-(tert-butyl-dimethyl-silanyloxymethyl)-1-phenyl-1H-pyridin-2-one as a white solid.

¹H NMR (400 MHz, CDCl₃) δ 7.49-7.47 (m, 2H); 7.43-7.39 (m, 3H); 7.29-7.28 (d, 2H); 6.65 (s, 1H); 6.19 (d, 2H); 4.59 (s, 2H); 0.97 (s, 9H); 0.14 (s, 6H).

Step 4: 4-Hydroxymethyl-1-phenyl-1H-pyridin-2-one

4-(Tert-butyl-dimethyl-silyloxymethyl)-1-phenyl-1H-pyridin-2-one from Step 3 (1.3 g) was dissolved in TBAF in 1M THF (15 mL). The clear reaction mixture was stirred for 16 hours. The reaction mixture was concentrated and purified on a column of silica eluting with 10% MeOH/EtOAc to yield 4-hydroxymethyl-1-phenyl-1H-pyridin-2-one as a tan solid.

¹H NMR (400 MHz, CDCl₃) δ 7.5-7.47 (m, 2H); 7.43 (d, 1H); 7.38-7.36 (m, 2H); 7.32-7.30 (d, 1H) 6.67 (s, 1H); 6.23 (d, 1H) 4.57 (d, 2H).

Step 5: 4-Bromomethyl-1-phenyl-1H-pyridin-2-one

4-Hydroxymethyl-1-phenyl-1H-pyridin-2-one from Step 4 (1.0 g, 5 mmol) was dissolved in CH₂Cl₂. CBr₄ (6 mmol, 2 g) was added and the reaction mixture was cooled to 0°. PPh₃ (6 mmol, 2.0 g) was added dropwise in CH₂Cl₂. The reaction mixture was stirred at 0° for 15 minutes and then warmed to room temperature. The reaction mixture was concentrated and purified on a column of silica eluting with (30% EtOAc /hexane to 50% EtOAc/hexane) to give 4-bromomethyl-1-phenyl-1H-pyridin-2-one (8,x=H) as a white solid.

¹H NMR (400 MHz, CDCl₃) δ 7.52-7.48 (m, 2H); 7.45-7.43 (d, 1H); 7.38-7.33 (m, 3H); 6.64 (s, 1H); 6.30-6.28 (d, 1H); 4.25 (d, 2H).

Step 6: 4-(1-Trityl-1H-imidazol-4-ylmethyl)-benzonitrile

To a suspension of activated zinc dust (3.57 g, 54.98 mmol) in THF (50 mL) was added dibromoethane (0.315 mL, 3.60 mmol) and the reaction stirred under argon for 45 minutes, at 20° C. The suspension was cooled to 0° C. and α-bromo-p-tolunitrile (9.33 g, 47.6 mmol) in THF (100 mL) was added dropwise over a period of 10 minutes. The reaction was then allowed to stir at 20° C. for 6 hours and bis(triphenylphosphine)Nickel II chloride (2.40 g, 3.64 mmol) and 5-iodotrityl imidazole (15.95 g, 36.6 mmol) were added in one portion. The resulting mixture was stirred 16 hours at 20° C. and then quenched by addition of saturated NH₄Cl solution (100 mL) and the mixture stirred for 2 hours. Saturated aq. NaHCO₃ solution was added to give a pH of 8 and the solution was extracted with EtOAc (2×250 mL), dried (MgSO₄) and the solvent evaporated in vacuo. The residue was chromatographed (silica gel, 0-20% EtOAc in CH₂Cl₂) to afford the title compound as a white solid.

¹H NMR (CDCl₃, 400 Mz) δ (7.54 (2H, d, J=7.9 Hz), 7.38(1H, s), 7.36-7.29 (11H, m), 7.15-7.09(6H, m), 6.58(1H, s) and 3.93(2H, s) ppm.

Step 7: 4-[3-(2-Oxo-1-phenyl-1,2-dihydropyridin-4-ylmethyl)-3H-imidazol-4-ylmethyl]benzonitrile, Hydrochloride

4-Bromomethyl-1-phenyl-1H-pyridin-2-one from Step 5 (1.1 g, 4.1 mmol) and 4-(1-trityl-1H-imidazol-4-ylmethyl)-benzonitrile from Step 6 (4.1 mmol, 1.7 g) were suspended in CH₃CN and heated to 80°. After 30 minutes the reaction became homogeneous. The reaction mixture was heated to 80° for 16 hours. The heterogeneous reaction mixture was concentrated, taken up in MeOH and refluxed for 1 hour. The reaction mixture was cooled, diluted with CHCl₃ and washed with saturated NaHCO₃. The aqueous layer was back extracted 4 times with CHCl₃. The organic layers were combined, washed with brine, dried (MgSO₄), filtered and concentrated to yield a yellow solid which was purified by flash chromatography (7% i-PrOH/CHCl₃ saturated with NH₃). Purest fractions were collected and concentrated to yield a white solid which was triturated with EtOAc. The solids were filtered, washed with EtOAc and dried under hi-vacuum for 16 hours to yield 4-[3-(2-Oxo-1-phenyl-1,2-dihydropyridin-4-ylmethyl)-3H-imidazol-4-ylmethyl]benzonitrile, hydrochloride as a white solid.

¹H NMR (400 MHz, CDCl₃) δ 7.61 (s, 1H); 7.58-757 (d, 2H) 7.52-7.49 (m, 2H); 7.46-7.44 (d, 1H); 7.34-7.32 (d, 2H); 7.26-7.25 (m, 2H); 6.97 (s, 1H); 6.20 (s, 1H); 5.77 (d, 1H); 4.77 (d, 2H); 3.96 (s, 2H).

### Example 13

Preparation of 4-imidazol-1-ylmethyl-(2-naphthalen-2-yloxy)-benzonitrile Hydrochloride

Step A: Preparation of 4-bromo-3-fluorobenzoic Acid

4-Bromo-3-fluorotoluene (40.0 g, 0.212 mol) was heated at 90° C in H₂O (200 mL) and pyridine (200 mL) with mechanical stirring under Ar. Potassium permanganate (KMnO₄) (67 g, 0.424 mol) was added portionwise over 3 h. After 4 h, an HPLC of a filtered sample indicated 50% conversion to the acid. An additional 30 g of KMnO₄ was added and heating continued overnight. HPLC indicated 81% conversion. Further KMnO₄ was added portionwise with reaction monitoring by HPLC until >95% conversion was obtained. The reaction mixture was filtered through Celite, the filter pad washed with H₂O, aq NaOH and EtOH. The filtrate was concentrated to a small volume, then partitioned between 3N NaOH solution and diethyl ether. The aqueous basic layer was separated, cooled in an ice-H₂O bath and acidified slowly with 6N HCl solution to precipitate the white solid product. This was collected by suction filtration and dried at 40° C. in a vacuum oven overnight to give the title compound. mp 190-192° C.

¹H NMR (CDCl₃) δ 7.83 (dd, 1H, J=2, 9 Hz), 7.78 (dd, 1H, J=2, 8 Hz), 7.67-7.71 (m, 1H).

Step B: Preparation of 4-bromo-3-fluorobenzyl Alcohol

4-Bromo-3-fluorobenzoic acid (40.8 g, 0.187 mol) was dissolved in THF (250 ml) with magnetic stirring under Ar in an ice-H₂O bath. The cloudy solution was treated dropwise with borane-THF complex (1 M) (374 mL, 0.374 mol) over a 1 h period maintaining the internal temperature at <10° C. The reaction mixture was left to warm to ambient temperature overnight, then cooled in an ice H₂O bath and treated dropwise with H₂O (150 mL). The THF was removed on a rotary evaporator, and the residue partitioned between EtOAc and H₂O. The aqueous layer was extracted with EtOAc (3×100 mL), the organic layers combined, washed with brine, and dried (Na₂SO₄), filtered, and concentrated to give the title compound as an oil which solidified on standing.

¹H NMR (CDCl₃) δ 7.52 (t, 1H, J=8 Hz), 7.16 (d, 1H, J=9 Hz), 7.02 (d, 1H, J=8 Hz), 4.67 (s, 2H), 1.47 (br s, 1H).

Step C: Preparation of 2-fluoro-4-hydroxymethylbenzonitrile

4-Bromo-3-fluorobenzyl alcohol (20 g, 0.097 mol) was dissolved in DMF (100 mL) and then placed under high vacuum for 15 min. The solution was then purged with Ar for 15 min. While purging continued, zinc cyanide (8 g, 0.068 mol) and the catalyst, Pd[(PPh₃)]₄, (5.63 g, 0.0049 mol) were added. The reaction mixture was heated at 95° C. under Ar for 18 h, then cooled to ambient temperature and added to H₂O. The mixture was extracted with EtOAc, then washed with 1M HCl, H₂O, brine, and dried (Na₂SO₄). Filtration and concentration to dryness gave the title compound as a white solid after chromatography (silica gel, hexane: EtOAc, 6.5:3.5.

¹H NMR (CDCl₃) δ 7.61 (t, 1H, J=8 Hz), 7.23-7.29 (m, 2H), 4.80 (d, 2H, J=6 Hz), 1.93 (t, 1H, J=6 Hz).

Step D: Preparation of 4-bromomethyl-2-fluoro-benzonitrile

N-Bromosuccinimide (6.6 g, 0.037 mol) was dissolved in CH₂Cl₂ (150 mL), cooled to 0° C. and treated with dimethylsulfide (3.27 mL, 0.0446 mol). The solution was cooled to −20° C. and then treated dropwise with a solution of 2-fluoro-4-hydroxymethylbenzonitrile (3.74 g, 0.0248 mol) in CH₂Cl₂ (30 mL). After the addition, the reaction mixture was stirred at 0° C. for 2 h then left to warm to ambient temperature overnight. The reaction mixture was added to ice/H₂O, extracted with EtOAc, the organic layer separated, washed with brine and dried (MgSO₄). Filtration and concentration to dryness gave the title compound which was purified chromatography (silica gel, 5-10% EtOAc/hexane.

¹H NMR (CDCl₃) δ 7.61 (dd, 1H, J=8, 8 Hz), 7.26-7.30 (m, 2H), 4.45 (s, 2H).

Step E: Preparation of 2-fluoro-4-imidazol-1-ylmethyl-benzonitrile

4-Bromomethyl-2-fluoro-benzonitrile (3.44 g, 16.0 mmol) and imidazole (5.47 g, 80.3 mmol) were dissolved in DMF (40 mL) and stirred at ambient temperature for 2 h. The DMF was removed in vacuo and the residue was partitioned between EtOAc (300 mL) and aqueous saturated NaHCO₃ solution. The organic layer was separated, washed with NaHCO₃ solution, H₂O, brine, and dried (MgSO₄). Filtration and concentration to dryness gave the title compound after chromatography (silica gel, 1-2% CH₃OH/CH₂Cl₂).

¹H NMR (CDCl₃) δ 7.62 (dd, 1H, J=8.5, 9.5 Hz), 7.57 (s, 1H), 7.16 (s, 1H), 7.00 (d, 1H, J=8.5 Hz), 6.94 (d, 1H, J=9.5 Hz), 6.91 (s, 1H), 5.21 (s, 2H).

Step F: Preparation of 2-(2-naphthyloxy)-4-imidazol-1-ylmethyl-benzonitrile Hydrochloride

2-Fluoro-4-imidazol-1-ylmethyl-benzonitrile (0.167 g, 0.830 mmol), 2-naphthol (0.143 g, 0.996 mmol) and cesium carbonate (0.54 g, 1.66 mmol) were dissolved in DMF (15 mL) and heated at 55° C. under Ar for 18 h. The reaction mixture was partitioned between EtOAc and 1N NaOH solution. The organic layer was separated, washed with 1N NaOH solution, H₂O, brine, and dried (MgSO₄). Filtration and concentration to dryness gave the title compound after chromatography (silica gel, 1% CH₃OH/CH₂Cl₂).

FAB mass spectrum (M+1) 326

Analysis calculated for C 21H N₃O.1.0HCl.0.75H₂O: C, 67.19; H, 4.70; N, 11.20; Found: C, 67.23; H, 4.89; N, 11.14.

### Example 14

Preparation of 2-(2-chloro-4-methoxyphenoxy)-4-imidazol-1-ylmethyl-benzonitrile Hydrochloride

Step A: Preparation of 2-(2-chloro-4-methoxyphenoxy)-4-imidazol-1-ylmethyl-benzonitrile Hydrochloride

2-Fluoro-4-imidazol-1-ylmethyl-benzonitrile, as described in Example 13, Step E, (0.118 g, 0.586 mmol), 2-chloro-4-methoxyphenol (0.112 g, 0.703 mmol), KF on alumina (40% by weight) (0.112 g, 0.703 mmol) and 18-crown-6 (0.11 g, 10% by weight of phenol) were dissolved in CH₃CN (5 mL) and heated at reflux under Ar for 18 h. The reaction mixture was filtered, dissolved in CH₃OH and purified by R^{P} HPLC on a Waters Prep Pak column eluting with a 0.1%TFA/H₂O: 0.1%TFA/CH₃CN gradient (95:5 to 5:95) to give the title compound after conversion to the hydrochloride salt.

FAB mass spectrum (M+1) 340

Analysis calculated for C₁₈H₁₄ClN₃O₂.1.0HCl.0.15 CH₂Cl₂: C, 56.04; H, 3.96; N, 10.80; Found: C, 56.25; H, 3.90; N, 10.42.

### Example 15

Preparation of 2-(2,4-dichloro-phenylsulfanyl)-4-[5-(2-morpholin-4-yl-ethyl)-imidazol-1-ylmethyl]-benzonitrile

Step A: Preparation of {2-[3-(4-cyano-3-fluoro-benzyl)-3H-imidazol-4-yl]-ethyl}-carbamic Acid tert-butyl Ester

To a solution of N^{Γ}-pivaloyloxymethyl-N^{α}-phthaloyl-histamine (J. C. Emmett, F. H. Holloway, and J. L. Turner, J. Chem. Soc., Perkin Trans. 1, 1341, (1979)) (4.59 g, 0.0124 mol) in acetonitrile (40 mL) was added 2-fluoro-4-imidazol-1-ylmethyl-benzonitrile (as described in Example 13, Step D) (2.8 g, 0.013 mol) and the mixture was heated to reflux for 18 hr. A white solid precipitate formed which after cooling to 0° C. was collected by filtration to obtain the quaternary salt. This intermediate was dissolved in EtOH (100 mL), hydrazine (1.46 mL, 0.046 mol) was added, and the mixture was heated at reflux for 4 hr. A white precipitate was observed and the reaction was cooled to 25° C. Dimethylphthalate (11.4 mL, 0.0699 mol) was added and the mixture was again refluxed for 18 hr. After cooling to 25° C. the precipitate was removed by filtration and washed with EtOAc. The filtrate was evaporated in vacuo and the residue was dissolved in THF (125 mL) and H₂O (25 mL). To this solution was added solid Na₂CO₃ (4.0 g, 0.0377 mol) and BOC₂O (4.47 g, 0.020 mol) and the reaction was stirred for 18 hr. The THF was removed in vacuo and the mixture was partitioned with EtOAc and saturated NaHCO₃. The EtOAc layer was washed with brine, dried with MgSO₄, and evaporated in vacuo to obtain the title product after chromatography (silica gel, CH₂Cl₂:MeOH:NH₄OH/97:3:0.3.

Step B: Preparation of 4-[5-(2-amino-ethyl)-imidazol-1-ylmethyl]-2-fluoro-benzonitrile Dihydrochloride

A solution of {2-[3-(4-cyano-3-fluoro-benzyl)-3H-imidazol-4-yl]-ethyl}-carbamic acid tert-butyl ester (1.0 g, 0.0029 mol) in EtOAc (30 mL) was cooled to −20° C. and saturated with HCl gas. The cooling bath was removed and the reaction was stirred for 2 hr. The solvent was removed in vacuo to obtain the title compound which was used without further purification.

Step C: Preparation of 2-fluoro-4-[5-(2-morpholin-4-yl-ethyl)-imidazol-1-ylmethyl]-benzonitrile

To a solution of 4-[5-(2-amino-ethyl)-imidazol-1-ylmethyl]-2-fluoro-benzonitrile dihydrochloride (0.92 g, 0.0029 mol) in acetonitrile (150 mL) and triethylamine (3.2 mL) was added 2-bromoethyl ether (0.839 mL, 0.0067 mol) and the mixture was refluxed for 48 hr. The solvents were removed in vacuo and the residue was dissolved in EtOAc which was washed twice with 1M HCl (100 mL). The HCl layers were combined and adjusted to pH=9 with solid Na₂CO₃ and extraxcted 3 times with EtOAc. The EtOAc layers were combined and dried with brine and MgSO₄. Removal of the EtOAc in vacuo yielded the title compound which was used as is in the next step.

Step D: Preparation of 2-(2,4-dichloro-phenylsulfanyl)-4-[5-(2-morpholin-4-yl-ethyl)-imidazol-1-ylmethyl]-benzonitrile

Following the procedure described in Example 14, Step A, the title compound was prepared using 2-fluoro-4-[5-(2-morpholin-4-yl-ethyl)-imidazol-1-ylmethyl]-benzonitrile (0.15 g, 0.477 mmol) and 2,4-dichlorothiophenol (0.086 g, 0.477 mmol).

FAB mass spectrum m/e 473 (M+1).

Analysis calculated for C₂₃H₂₂Cl₂N₄OS.0.85 TFA.0.3H₂O: C, 51.52; H, 4.11; N, 9.73. Found: C, 51.51; H, 4.29; N, 9.36.

Following the above methods, the following compound was prepared by utilizing 2,4-dichlorophenol in place of 2,4-dichlorothiophenol in Step D:

2-(2,4-dichloro-phenoxy)-4-[5-(2-morpholin-4-yl-ethyl)-imidazol-1-ylmethyl]-benzonitrile

FAB mass spectrum m/e 457 (M+1).

Analysis calculated for C₂₃H₂₂Cl₂N₄O₂.0.4H₂O: C, 59.34; H, 4.96; N, 12.04. Found: C, 59.32; H, 4.89; N, 11.75.

### Example 16

Preparation of 4-[hydroxy-(3-methyl-3H-imidazol-4-yl)-methyl]-2-(naphthalen-2-yloxy)-benzonitrile Hydrochloride

Step A: Preparation of 2-Fluoro-4-formylbenzonitrile

2-Fluoro-4-hydroxymethylbenzonitrile (Example 13, Step C) 10 g, 0.066 mol) and triethylamine (32.3 mL, 0.231 mol) were dissolved in CH₂Cl₂ (100 mL)-DMSO (20 mL) at <5° C. with stirring and treated dropwise with a solution of pyridine.SO₃ complex (31.5 g, 0.198 mol) in DMSO (70 mL) maintaining the reaction mixture temperature at <10° C. The reaction mixture was stirred at 5° C. for 1 hr after the addition, then at 20° C. for 1 hr, then partitioned between CH₂Cl₂ and H₂O. The organic layer was separated, washed well with H₂O, brine, and dried (Na₂SO₄). Filtration and concentration gave the title compound after purification by chromatography (silica gel, hexane: EtOAc, 3:1).

¹H NMR (CDCl₃) δ 10.06 (d, 1H, J=2 Hz), 7.86 (dd, 1H, J=5, 8 Hz), 7.798 (dd, 1H, J=1, 8 Hz), 7.728 (dd, 1H, J=1, 8 Hz).

Step B: Preparation of 2-fluoro-4-[hydroxy-(1-trityl-1H— imidazol-4-yl)-methyl]-benzonitrile

To a solution of 4-iodo-1-trityl-1H-imidazole (5.00 g, 11.5 mmol) in anhydrous CH₂Cl₂ (30 mL) was added a 3.0M solution of ethylmagnesium bromide (6.58 mL, 19.7 mmol) with stirring under Ar. After 3 h, the reaction mixture was cooled to −78° C. and a solution of 2-fluoro-4-formyl-benzonitrile (1.70 g, 11.5 mmol) dissolved in CH₂Cl₂ (20 mL) was added dropwise. The reaction was allowed to warm to RT over 2 h, quenched with saturated NH₄Cl solution, diluted with satd. NaHCO₃ solution to pH=8.5, and extracted with CH₂Cl₂ (3×). The combined organic layers were dried (MgSO₄), concentrated and purified using SiO₂ chromatography (0-1% MeOH/CH₂Cl₂) to yield the title compound.

Step C: Preparation of acetic acid (4-cyano-3-fluoro-phenyl)-(1-trityl-1H-imidazol-4-yl)-methyl Ester

2-Fluoro-4-[hydroxy-(1-trityl-1H-imidazol-4-yl)-methyl]-benzonitrile (4.05 g, 8.81 mmol), pyridine (2.14 mL, 26.4 mmol), and acetic anhydride (12.5 mL, 132 mmol) were stirred in anhydrous DMF (60 mL) for 3 h under Ar. The reaction was concentrated in vacuo, diluted with EtOAc (250 mL), washed with H₂O (2×), brine, dried (MgSO₄) and concentrated to give the title compound.

Step D: Preparation of acetic acid (4-cyano-3-fluoro-phenyl)-(3-methyl-3H-imidazol-4-yl)-methyl Ester

Acetic acid (4-cyano-3-fluoro-phenyl)-(1-trityl-1H-imidazol-4-yl)-methyl ester (4.60 g, 9.17 mmol) and dimethyl sulfate (0.83 mL, 8.81 mmol) were dissolved in EtOAc (20 mL) and heated at 60° C. overnight under Ar. The reaction was concentrated in vacuo, diluted with MeOH (30 mL), and refluxed for 1 h. Concentrated in vacuo and purified using SiO₂ chromatography (0.5-4% MeOH/CH₂Cl₂ with NH₄OH) to give the title compound.

Step E: Preparation of 2-fluoro-4-[hydroxy-(3-methyl-3H-imidazol-4-yl)-methyl]-benzonitrile

Acetic acid (4-cyano-3-fluoro-phenyl)-(3-methyl-3H-imidazol-4-yl)-methyl ester (1.26 g, 4.59 mmol) and NaOH (5.5 mL, 5.5 mmol) were dissolved in THF (15 mL) and H₂O (25 mL). After 1 h, the reaction was diluted with satd. NaHCO₃ solution, extracted with CH₂Cl₂ (3×), dried (MgSO₄) and concentrated to give the title compound.

Step F: Preparation of 4-[hydroxy-(3-methyl-3H-imidazol-4-yl)-methyl]-2-(naphthalen-2-yloxy)-benzonitrile Hydrochloride

2-Fluoro-4-[hydroxy-(3-methyl-3H-imidazol-4-yl)-methyl]-benzonitrile (0.099 g, 0.428 mmol), 2-naphthol (0.062 g, 0.4287 mmol) and Cs₂CO₃ (0.279 g, 0.856 mmol) were dissolved in anhydrous DMSO (5 mL) and heated at 80° C. under Ar for 1.5 h. The reaction was diluted with EtOAc, washed with satd. NaHCO₃ solution, water, and brine. The organic layer was dried (MgSO₄), concentrated and purified using SiO₂ chromatography (1-2.5% MeOH/CH₂Cl₂). The purified compound was dissolved in CH₂Cl₂ and treated with 1N HCl ethereal solution to give the title compound.

FAB MS (M+1)=365.

Analysis calculated for C₂₂H₁₇N₃O₂.1.00HCl.1.60H₂O: C, 62.81; H, 5.08; N, 9.99 Found: C, 62.81; H, 4.98; N, 10.20.

### Example 17

Preparation of 4-[1-hydroxy-1-(3-methyl-3H-imidazol-4-yl)-ethyl]-2-(naphthalen-2-yloxy)-benzonitrile

Step A: Preparation of 4-(3-methyl-3H-imidazole-4-carbonyl)-2-(naphthalen-2-yloxy)-benzonitrile

2-Fluoro-4-[hydroxy-(3-methyl-3H-imidazol-4-yl)-methyl]-benzonitrile (as described in Example 16, Step E) (0.172 g, 0.743 mmol), 2-naphthol (0.107 g, 0.743 mmol) and Cs₂CO₃ (0.727 g, 2.23 mmol) were dissolved in anhydrous DMF (5 mL) and heated at 60° C. under Ar for 2 days. The reaction was diluted with EtOAc, washed with satd. NaHCO₃ solution, water, and brine. The organic layer was dried (MgSO₄), concentrated and purified using SiO₂ chromatography (1-2% MeOH/CH₂Cl₂) to give the title compound.

Step B: Preparation of 4-[1-hydroxy-1-(3-methyl-3H-imidazol-4-yl)-ethyl]-2-(naphthalen-2-yloxy)-benzonitrile

4-(3-Methyl-3H-imidazole-4-carbonyl)-2-(naphthalen-2-yloxy)-benzonitrile (0.109 g, 0.308 mmol) was dissolved in anhydrous THF (5 mL) and a 3.0 M solution of MeMgBr (0.35 mL, 1.05 mmol) was added and stirred at RT. The reaction was quenched with NH₄Cl after 1 h, concentrated, diluted with EtOAc, washed with satd. NaHCO₃ solution, water, brine, dried (MgSO₄) and concentrated to give the title compound. FT/ICR MS (M+1)=370.

Analysis calculated for C₂₃H₁₉N₃O₂.0.40EtOAc.0.05H₂O: C, 72.85; H, 5.54; N, 10.36 Found: C, 72.87; H, 5.31; N, 10.29.

### Example 18

Preparation of 4-imidazol-1-ylmethyl-2-[2-(2-oxo-piperidin-1-yl)-phenoxy]-benzonitrile

Step A: Preparation of 4-Bromo-3-fluorobenzoic Acid

4-Bromo-3-fluorotoluene (40.0 g, 0.212 mol) was heated at 90° C. in H₂O (200 mL)-pyridine (200 mL) with mechanical stirring under Ar. Potassium permanganate (KMnO₄) (67 g, 0.424 mol) was added portionwise over 3 h. After 4 h, an HPLC of a filtered sample indicated 50% conversion to the acid. An additional 30 g of KMnO₄ was added and heating continued overnight. HPLC indicated 81% conversion. Further KMnO₄ was added portionwise with reaction monitoring by HPLC until >95% conversion was obtained. The reaction mixture was filtered through Celite, the filter pad washed with H₂O, aq NaOH and EtOH. The filtrate was concentrated to a small volume, then partitioned between 3N NaOH solution and diethyl ether. The aqueous basic layer was separated, cooled in an ice-H₂O bath and acidified slowly with 6N HCl solution to precipitate the white solid product. This was collected by suction filtration and dried at 40° C. in a vacuum oven overnight to give the title compound. mp 190-192° C.

¹H NMR (CDCl₃) δ 7.83 (dd, 1H, J=2, 9 Hz), 7.78 (dd, 1H, J=2, 8 Hz), 7.67-7.71 (m, 1H).

Step B: Preparation of 4-bromo-3-fluorobenzyl Alcohol

4-Bromo-3-fluorobenzoic acid, as described above, (40.8 g, 0.187 mol) was dissolved in THF (250 ml) with magnetic stirring under Ar in an ice-H₂O bath. The cloudy solution was treated dropwise with borane-THF complex (1 M) (374 mL, 0.374 mol) over a 1 h period maintaining the internal temperature at <10° C. The reaction mixture was left to warm to ambient temperature overnight, then cooled in an ice-H₂O bath and treated dropwise with H₂O (150 mL). The THF was removed on a rotary evaporator, and the residue partitioned between EtOAc and H₂O. The aqueous layer was extracted with EtOAc (3×100 mL), the organic layers combined, washed with brine, and dried (Na₂SO₄), filtered, and concentrated to give the title compound as an oil which solidified on standing.

¹H NMR (CDCl₃) δ 7.52 (t, 1H, J=8 Hz), 7.16 (d, 1H, J=9 Hz), 7.02 (d, 1H, J=8 Hz), 4.67 (s, 2H), 1.47 (br s, 1H).

Step C: Preparation of 2-fluoro-4-hydroxymethylbenzonitrile

4-Bromo-3-fluorobenzyl alcohol, as described in Step B above, (20 g, 0.097 mol) was dissolved in DMF (100 mL) then placed under high vacuum for 15 min. The solution was then purged with Ar for 15 min. While purging continued, zinc cyanide (8 g, 0.068 mol) and the catalyst, Pd[(PPh₂)14, (5.63 g, 0.0049 mol) were added. The reaction mixture was heated at 95° C. under Ar for 18 h, then cooled to ambient temperature and added to H₂O. The mixture was extracted with EtOAc, then washed with 1M HCl, H₂O, brine, and dried (Na₂SO₄). Filtration and concentration to dryness gave the title compound as a white solid after chromatography (silica gel, hexane: EtOAc, 6.5:3.5).

¹H NMR (CDCl₃) δ 7.61 (t, 1H, J=8 Hz), 7.23-7.29 (m, 2H), 4.80 (d, 2H, J=6 Hz), 1.93 (t, 1H, J=6 Hz).

Step D: Preparation of 4-Bromomethyl-2-fluoro-benzonitrile

N-Bromosuccinimide (6.6 g, 0.037 mol) was dissolved in CH₂Cl₂ (150 mL), cooled to 0° C. and treated with dimethylsulfide (3.27 mL, 0.0446 mol). The solution was cooled to −20° C. then treated dropwise with a solution of 2-fluoro-4-hydroxymethylbenzonitrile, as described in Step C above, (3.74 g, 0.0248 mol) in CH₂Cl₂ (30 mL). After the addition, the reaction mixture was stirred at 0° C. for 2 h then left to warm to ambient temperature overnight. The reaction mixture was added to ice/H₂O, extracted with EtOAc, the organic layer separated, washed with brine and dried (MgSO₄). Filtration and concentration to dryness gave the title compound which was purified after chromatography (silica gel, 5-10% EtOAc/hexane).

¹H NMR (CDCl₃) δ 7.61 (dd, 1H, J=8, 8 Hz), 7.26-7.30 (m, 2H), 4.45 (s, 2H).

Step E: Preparation of 2-fluoro-4-imidazol-1-ylmethyl-benzonitrile

4-Bromomethyl-2-fluoro-benzonitrile, as described in Step D above, (3.44 g, 16.0 mmol) and imidazole (5.47 g, 80.3 mmol) were dissolved in DMF (40 mL) and stirred at ambient temperature for 2 h. The DMF was removed in vacuo and the residue was partitioned between EtOAc (300 mL) and aqueous saturated NaHCO₃ solution. The organic layer was separated, washed with NaHCO₃ solution, H₂O, brine, and dried (MgSO₄). Filtration and concentration to dryness gave the title compound after chromatography (silica gel, 1-2% CH₃OH/CH₂Cl₂).

¹H NMR (CDCl₃) δ 7.62 (dd, 1H, J=8.5, 9.5 Hz), 7.57 (s, 1H), 7.16 (s, 1H), 7.00 (d, 1H, J=8.5 Hz), 6.94 (d, 1H, J=9.5 Hz), 6.91 (s, 1H), 5.21 (s, 2H).

Step F: Preparation of 2-(2-oxo-piperidin-1-yl)-phenol

To a solution of 2-aminophenol (1.09 g, 0.01 mol), Et₃N (4.46 mL, 0.032 mol) and 4-dimethylaminopyridine (0.122 g, 0.001 mol) in CHCl₃ (20 mL) in an ice-H₂O bath was added dropwise 5-bromovaleryl chloride (2.95 mL, 0.022 mol) with stirring. After 2 hr, the reaction mixture was washed with 1N HCl until the aqueous layer was acidic, then washed with H₂O, aqueous saturated NaHCO₃ solution, brine, and dried (Na₂SO₄). Filtration and concentration to dryness gave a yellow oil which solidifed on standing. This bisacylated product was dissolved in DMF (20 mL) and heated at 80° C. with cesium carbonate (4.89 g, 0.015 mol) for 3 hr, then partitioned between EtOAc and ice water. The aqueous layer was extracted with EtOAc (3×), the organics combined, washed with H₂O, aqueous saturated NaHCO₃ solution, brine, and dried (Na₂SO₄). Filtration and concentration to dryness gave a crude product which was treated with 1N NaOH solution (12 mL, 0.012 mol) in THF (20 mL)-H₂O (10 mL) with stirring at ambient temperature for 2 hr. The reaction mixture was neutralized with 1N HCl (12 mL, 0.012 mol), concentrated, and extracted with EtOAc (3×), the organics combined, washed with H₂O, aqueous saturated NaHCO₃ solution, brine, and dried (Na₂SO₄). Filtration and concentration to dryness gave the title compound.

Step G: Preparation of 4-imidazol-1-ylmethyl-2-[2-(2-oxo-piperidin-1-yl)-phenoxy]-benzonitrile

2-Fluoro-4-imidazol-1-ylmethyl-benzonitrile (as described in Step E above) (0.080 g, 0.4 mmol), 2-(2-oxo-piperidin-1-yl)-phenol (as described in Step F above) (0.091 g, 0.5 mmol) and cesium carbonate (0.261 g, 0.8 mmol) were combined in DMF (2.0 mL) and heated at 50° C. for 18 hr. The reaction mixture was partitioned between EtOAc and a minimum volume of H₂O. Additional product was salted out from the aqueous layer with solid NaCl, and extracted into EtOAc. The organic layers were combined, dried (Na₂SO₄), filtered and concentrated to give the title compound after R^{P} HPLC on a Waters Prep Pak column eluting with a 0.1%TFA/H₂O: 0.1%TFA/CH₃CN gradient followed by conversion to the free base.

FAB MS 373 (M+1).

Analysis calculated for C₂₂H₂₀N₄O₂.0.45H₂O: C, 69.43; H, 5.54; N, 14.72. Found: C, 69.41; H, 5.46; N, 14.67.

### Example 19

Preparation of 4-imidazol-1-ylmethyl-2-[2-(3-methyl-2-oxo-piperidin-1-yl)-phenoxy]-benzonitrile

Step A: Preparation of 2-(3-methyl-2-oxo-piperidin-1-yl)-phenol

Lithium diethylamide (2M solution in THF) (3.92 mL, 7.84 mmol) was added to a solution of 2-(2-oxo-piperidin-1-yl)-phenol (as described in Example 18, Step F) (0.50 g, 2.61 mmol) in THF (5 mL) at −78° C. with stirring under Ar. After 30 min, iodomethane (0.488 mL, 7.84 mmol) was added and the reaction left to come to room temperature overnight. The reaction was treated with H₂O, concentrated to remove the THF, then partitioned between diethyl ether and H₂O. The organic layer was separated, washed with brine, dried (Na₂SO₄), filtered, and concentrated to dryness to give the title compound after chromatography (silica gel, 1% CH₃OH, 29% EtOAc, 70% hexane).

¹H NMR (CDCl₃) δ 7.14-7.23 (m, 3H), 7.52 (dd, 1H, J=1.5, 8 Hz), 6.93-6.98 (m, 1H), 3.70-3.85 (m, 2H), 2.63-2.72 (m, 1H), 2.08-2.17 (m, 1H), 1.89-2.05 (m, 2H), 1.60-1.70 (m, 1H), 1.36 (d, 3H, J=7 Hz).

Step B: Preparation of 4-imidazol-1-ylmethyl-2-[2-(3-methyl-2-oxo-piperidin-1-yl)-phenoxy]-benzonitrile

Following the procedure outlined in Example 18, Step G, but substituting the phenol of Step A for the phenol used in Example 18, Step G, the title compound was prepared.

FAB MS 387(M+1).

Analysis calculated for C₂₅H₂₆N₄O₂.1.85HCl.0.35Et₂O: C, 61.07; H, 5.75; N, 11.66. Found: C, 60.98; H, 5.93; N, 11.68.

### Example 20

Preparation of 4-imidazol-1-ylmethyl-2-[2-(2-oxo-piperidin-1-yl)-phenoxy]-benzonitrile

Step A: Preparation of 4-Bromo-3-fluorobenzoic Acid

4-Bromo-3-fluorotoluene (40.0 g, 0.212 mol) was heated at 90° C in H₂O (200 mL)-pyridine (200 mL) with mechanical stirring under Ar. Potassium permanganate (KMnO₄) (67 g, 0.424 mol) was added portionwise over 3 h. After 4 h, an HPLC of a filtered sample indicated 50% conversion to the acid. An additional 30 g of KMnO₄ was added and heating continued overnight. HPLC indicated 81% conversion. Further KMnO₄ was added portionwise with reaction monitoring by HPLC until >95% conversion was obtained. The reaction mixture was filtered through Celite, the filter pad washed with H₂O, aq NaOH and EtOH. The filtrate was concentrated to a small volume, then partitioned between 3N NaOH solution and diethyl ether. The aqueous basic layer was separated, cooled in an ice-H₂O bath and acidified slowly with 6N HCl solution to precipitate the white solid product. This was collected by suction filtration and dried at 40° C. in a vacuum oven overnight to give the title compound. mp 190-192° C.

¹H NMR (CDCl₃) δ 7.83 (dd, 1H, J=2, 9 Hz), 7.78 (dd, 1H, J=2, 8 Hz), 7.67-7.71 (m, 1H).

Step B: Preparation of 4-bromo-3-fluorobenzyl Alcohol

4-Bromo-3-fluorobenzoic acid, as described above, (40.8 g, 0.187 mol) was dissolved in THF (250 ml) with magnetic stirring under Ar in an ice-H₂O bath. The cloudy solution was treated dropwise with borane-THF complex (1 M) (374 mL, 0.374 mol) over a 1 h period maintaining the internal temperature at <10C. The reaction mixture was left to warm to ambient temperature overnight, then cooled in an ice-H₂O bath and treated dropwise with H₂O (150 mL). The THF was removed on a rotary evaporator, and the residue partitioned between EtOAc and H₂O. The aqueous layer was extracted with EtOAc (3×100 mL), the organic layers combined, washed with brine, and dried (Na₂SO₄), filtered, and concentrated to give the title compound as an oil which solidified on standing.

¹H NMR (CDCl₃) δ 7.52 (t, 1H, J=8 Hz), 7.16 (d, 1H, J=9 Hz), 7.02 (d, 1H, J=8 Hz), 4.67 (s, 2H), 1.47 (br s, 1H).

Step C: Preparation of 2-fluoro-4-hydroxymethylbenzonitrile

4-Bromo-3-fluorobenzyl alcohol, as described in Step B above, (20 g, 0.097 mol) was dissolved in DMF (100 mL) then placed under high vacuum for 15 min. The solution was then purged with Ar for 15 min. While purging continued, zinc cyanide (8 g, 0.068 mol) and the catalyst, Pd[(PPh₂)]4, (5.63 g, 0.0049 mol) were added. The reaction mixture was heated at 95° C. under Ar for 18 h, then cooled to ambient temperature and added to H₂O. The mixture was extracted with EtOAc, then washed with 1M HCl, H₂O, brine, and dried (Na₂SO₄). Filtration and concentration to dryness gave the title compound as a white solid after chromatography (silica gel, hexane: EtOAc, 6.5:3.5).

¹H NMR (CDCl₃) δ 7.61 (t, 1H, J=8 Hz), 7.23-7.29 (m, 2H), 4.80 (d, 2H, J=6 Hz), 1.93 (t, 1H, J=6 Hz).

Step D: Preparation of 4-Bromomethyl-2-fluoro-benzonitrile

N-Bromosuccinimide (6.6 g, 0.037 mol) was dissolved in CH₂Cl₂ (150 mL), cooled to 0° C. and treated with dimethylsulfide (3.27 mL, 0.0446 mol). The solution was cooled to −20° C. then treated dropwise with a solution of 2-fluoro-4-hydroxymethylbenzonitrile, as described in Step C above, (3.74 g, 0.0248 mol) in CH₂Cl₂ (30 mL). After the addition, the reaction mixture was stirred at 0° C. for 2 h then left to warm to ambient temperature overnight. The reaction mixture was added to ice/H₂O, extracted with EtOAc, the organic layer separated, washed with brine and dried (MgSO₄). Filtration and concentration to dryness gave the title compound which was purified after chromatography (silica gel, 5-10% EtOAc/hexane).

¹H NMR (CDCl₃) δ 7.61 (dd, 1H, J=8, 8 Hz), 7.26-7.30 (m, 2H), 4.45 (s, 2H).

Step E: Preparation of 2-fluoro-4-imidazol-1-ylmethyl-benzonitrile

4-Bromomethyl-2-fluoro-benzonitrile, as described in Step D above, (3.44 g, 16.0 mmol) and imidazole (5.47 g, 80.3 mmol) were dissolved in DMF (40 mL) and stirred at ambient temperature for 2 h. The DMF was removed in vacuo and the residue was partitioned between EtOAc (300 mL) and aqueous saturated NaHCO₃ solution. The organic layer was separated, washed with NaHCO₃ solution, H₂O, brine, and dried (MgSO₄). Filtration and concentration to dryness gave the title compound after chromatography (silica gel, 1-2% CH₃OH/CH₂Cl₂).

¹H NMR (CDCl₃) δ 7.62 (dd, 1H, J=8.5, 9.5 Hz), 7.57 (s, 1H), 7.16 (s, 1H), 7.00 (d, 1H, J=8.5 Hz), 6.94 (d, 1H, J=9.5 Hz), 6.91 (s, 1H), 5.21 (s, 2H).

Step F: Preparation of 2-(2-oxo-piperidin-1-yl)-phenol

To a solution of 2-aminophenol (1.09 g, 0.01 mol), Et₃N (4.46 mL, 0.032 mol) and 4-dimethylaminopyridine (0.122 g, 0.001 mol) in CHCl₃ (20 mL) in an ice-H₂O bath was added dropwise 5-bromovaleryl chloride (2.95 mL, 0.022 mol) with stirring. After 2 hr, the reaction mixture was washed with 1N HCl until the aqueous layer was acidic, then washed with H₂O, aqueous saturated NaHCO₃ solution, brine, and dried (Na₂SO₄). Filtration and concentration to dryness gave a yellow oil which solidifed on standing. This bisacylated product was dissolved in DMF (20 mL) and heated at 80° C. with cesium carbonate (4.89 g, 0.015 mol) for 3 hr, then partitioned between EtOAc and ice water. The aqueous layer was extracted with EtOAc (3×), the organics combined, washed with H₂O, aqueous saturated NaHCO₃ solution, brine, and dried (Na₂SO₄). Filtration and concentration to dryness gave a crude product which was treated with 1N NaOH solution (12 mL, 0.012 mol) in THF (20 mL)-H₂O (10 mL) with stirring at ambient temperature for 2 hr. The reaction mixture was neutralized with 1N HCl (12 mL, 0.012 mol), concentrated, and extracted with EtOAc (3×), the organics combined, washed with H₂O, aqueous saturated NaHCO₃ solution, brine, and dried (Na₂SO₄). Filtration and concentration to dryness gave the title compound.

Step G: Preparation of 4-imidazol-1-ylmethyl-2-[2-(2-oxo-piperidin-1-yl)-phenoxy]-benzonitrile

2-Fluoro-4-imidazol-1-ylmethyl-benzonitrile (as described in Step E above) (0.080 g, 0.4 mmol), 2-(2-oxo-piperidin-1-yl)-phenol (as described in Step F above) (0.091 g, 0.5 mmol) and cesium carbonate (0.261 g, 0.8 mmol) were combined in DMF (2.0 mL) and heated at 50° C. for 18 hr. The reaction mixture was partitioned between EtOAc and a minimum volume of H₂O. Additional product was salted out from the aqueous layer with solid NaCl, and extracted into EtOAc. The organic layers were combined, dried (Na₂SO₄), filtered and concentrated to give the title compound after R^{P} HPLC on a Waters Prep Pak column eluting with a 0.1%TFA/H₂O: 0.1%TFA/CH₃CN gradient followed by conversion to the free base.

FAB MS 373 (M+1).

Analysis calculated for C₂₂H₂₀N₄O₂.0.45H₂O: C, 69.43; H, 5.54; N, 14.72. Found: C, 69.41; H, 5.46; N, 14.67.

### Example 20A

Preparation of 4-[5-(2-amino-ethyl)-2-methyl-imidazol-1-ylmethyl]-2-[3-(3-ethyl-1-methyl-2-oxo-azepan-3-yl)-phenoxy]-benzonitrile Dihydrochloride

Step A: Preparation of {2-[3-(4-cyano-3-fluoro-benzyl)-3H-imidazol-4-yl]-ethyl}-carbamic Acid tert-butyl Ester

To a solution of N^{Γ}-pivaloyloxymethyl-N^{α}-phthaloyl-histamine (J. C. Emmett, F. H. Holloway, and J. L. Turner, J. Chem. Soc., Perkin Trans. 1, 1341, (1979)) (4.59 g, 0.0124 mmol) in acetonitrile (40 mL) was added 2-fluoro-4-imidazol-1-ylmethyl-benzonitrile (as described in Example 20, Step E) (2.8 g, 0.013 mmol) and the mixture was heated to reflux for 18 hr. A white solid precipitate formed which after cooling to 0° C. was collected by filtration to obtain the quaternary salt. This intermediate was dissolved in EtOH (100 mL), hydrazine (1.46 mL, 0.046 mmol) was added, and the mixture was heated at reflux for 4 hr. A white precipitate was observed and the reaction was cooled to 25° C. Dimethylphthalate (11.4 mL, 0.0699 mmol) was added and the mixture was again refluxed for 18 hr. After cooling to 25° C. the precipitate was removed by filtration and washed with EtOAc. The filtrate was evaporated in vacuo and the residue was dissolved in THF (125 mL) and H₂O (25 mL). To this solution was added solid Na₂CO₃ (4.0 g, 0.0377 mmol) and BOC₂O (4.47 g, 0.020 mmol) and the reaction was stirred for 18 hr. The THF was removed in vacuo and the mixture was partitioned with EtOAc and saturated NaHCO₃. The EtOAc layer was washed with brine, dried with MgSO₄, and evaporated in vacuo to obtain the title product after chromatography (silica gel, CH₂Cl₂:MeOH:NH₄OH/97:3:0.3).

Step B: Preparation of [2-(3-{(4-cyano-3-[3-(3-ethyl-1-methyl-2-oxo-azepan-3-yl)-phenoxy]-benzyl}-2-methyl-3H-imidazol-4-yl)-ethyl]-carbamic Acid tert-butyl Ester

Following the procedure outlined in Example 19, but using {2-[3-(4-cyano-3-fluoro-benzyl)-3H-imidazol-4-yl]-ethyl}-carbamic acid tert-butyl ester, prepared as described in Step A (0.60 g, 1.67 mmol) and 3-ethyl-3-(3-hydroxy-phenyl)-1-methyl-azepan-2-one, commercially available from Maybridge (0.41 g, 1.67 mmol), the title compound was obtained after chromatography (CH₂Cl₂: CH₃OH: NH₄OH, 98:2:0.2).

Step C: Preparation of 4-[5-(2-amino-ethyl)-2-methyl-imidazol-1-ylmethyl]-2-[3-(3-ethyl-1-methyl-2-oxo-azepan-3-yl)-phenoxy]-benzonitrile Dihydrochloride

To a solution of [2-(3-{4-cyano-3-[3-(3-ethyl-1-methyl-2-oxo-azepan-3-yl)-phenoxy]-benzyl}-2-methyl-3H-imidazol-4-yl)-ethyl]-carbamic acid tert-butyl ester (as described in Step B above) (0.2 g, 0.41 mmol) in CH₂Cl₂ (6.0 mL) was added TFA (3.0 mL) and the solution was stirred for 0.5 hr. The solvents were removed in vacuo and the crude product was purified by preparative HPLC. Conversion to the HCl salt yielded the title compound.

FAB mass spectrum m/e 486 (m+1).

Analysis calculated for C₂₉H₃₅N₅O₂.2.2HCl: C, 61.55; H, 6.63; N, 12.38; Found: C, 61.56; H, 6.45; N, 11.83.

### Example 21

Preparation of 2-[3-(3-ethyl-1-methyl-2-oxo-azepan-3-yl)-phenoxy]-4-[2-methyl-5-(2-morpholin-4-yl-ethyl)-imidazol-1-ylmethyl]-benzonitrile

To a solution of 4-[5-(2-amino-ethyl)-2-methyl-imidazol-1-ylmethyl]-2-[3-(3-ethyl-1-methyl-2-oxo-azepan-3-yl)-phenoxy]-benzonitrile dihydrochloride (as described in Example 20D) (0.25 g, 0.456 mmol) in acetonitrile (35.0 mL) and triethylamine (1.8 mL) was added 2-bromoethyl ether (0.133 mL, 1.06 mmol) and the mixture was refluxed for 48 hr. The solvents were removed in vacuo to obtain the title compound after purification by preparative HPLC.

FAB mass spectrum m/e 556 (m+1).

Analysis calculated for C₃₃H₄₁N₅O₃.0.8H₂O: C, 69.51; H, 7.53; N, 12.28; Found: C, 69.51; H, 7.28; N, 12.13.

### Example 22

Preparation of 2-[3-(3-ethyl-1-methyl-2-oxo-azepan-3-yl)-phenoxy]-4-[hydroxy-(3-methyl-3-H-imidazol-4-yl)-methyl]-benzonitrile

Step A: Preparation of 2-Fluoro-4-formylbenzonitrile

2-Fluoro-4-hydroxymethylbenzonitrile (as described in Example 20, Step C) (10 g, 0.066 mol) and triethylamine (32.3 mL, 0.231 mol) were dissolved in CH₂Cl₂ (100 mL)-DMSO (20 mL) at <5° C. with stirring and treated dropwise with a solution of pyridine.SO₃ complex (31.5 g, 0.198 mol) in DMSO (70 mL) maintaining the reaction mixture temperature at <10° C. The reaction mixture was stirred at 5° C. for 1 hr after the addition, then at 20° C. for 1 hr, then partitioned between CH₂Cl₂ and H₂O. The organic layer was separated, washed well with H₂O, brine, and dried (Na₂SO₄). Filtration and concentration gave the title compound after purification by chromatography (silica gel, hexane: EtOAc, 3:1).

¹H NMR (CDCl₃) δ 10.06 (d, 1H, J=2 Hz), 7.86 (dd, 1H, J=5, 8 Hz), 7.798 (dd, 1H, J=1, 8 Hz), 7.728 (dd, 1H, J=1, 8 Hz).

Step B: Preparation of 2-fluoro-4-[hydroxy-(1-trityl-1H-imidazol-4-yl)-methyl]-benzonitrile

To a solution of 4-iodo-1-trityl-1H-imidazole (5.00 g, 11.5 mmol) in anhydrous CH₂Cl₂ (30 mL) was added a 3.0M solution of ethylmagnesium bromide (6.58 mL, 19.7 mmol) with stirring under Ar. After 3 h, the reaction mixture was cooled to −78° C. and a solution of 2-fluoro-4-formyl-benzonitrile (as described in Step A above) (1.70 g, 11.5 mmol) dissolved in CH₂Cl₂ (20 mL) was added dropwise. The reaction was allowed to warm to room temperature over 2 h, quenched with saturated NH₄Cl solution, diluted with satd. NaHCO₃ solution to pH=8.5, and extracted with CH₂Cl₂ (3×). The combined organic layers were dried (MgSO₄), concentrated and purified using SiO₂ chromatography (0-1% MeOH/CH₂Cl₂) to yield the title compound.

Step C: Preparation of acetic acid (4-cyano-3-fluoro-phenyl)-(1-trityl-1H-imidazol-4-yl)-methyl Ester

2-Fluoro-4-[hydroxy-(1-trityl-1H-imidazol-4-yl)-methyl]-benzonitrile (as described in Step B above) (4.05 g, 8.81 mmol), pyridine (2.14 mL, 26.4 mmol), and acetic anhydride (12.5 mL, 132 mmol) were stirred in anhydrous DMF (60 mL) for 3 h under Ar. The reaction was concentrated in vacuo, diluted with EtOAc (250 mL), washed with H₂O (2×), brine, dried (MgSO₄) and concentrated to give the title compound.

Step D: Preparation of acetic acid (4-cyano-3-fluoro-phenyl)-(3-methyl-3H-imidazol-4-yl)-methyl Ester

Acetic acid (4-cyano-3-fluoro-phenyl)-(1-trityl-1H-imidazol-4-yl)-methyl ester (as described in Step C above) (4.60 g, 9.17 mmol) and dimethyl sulfate (0.83 mL, 8.81 mmol) were dissolved in EtOAc (20 mL) and heated at 60° C. overnight under Ar. The reaction was concentrated in vacuo, diluted with MeOH (30 mL), and refluxed for 1 h. Concentrated in vacuo and purified using SiO₂ chromatography (0.5-4% MeOH/CH₂Cl₂ with NH₄OH) to give the title compound.

Step E: Preparation of 2-fluoro-4-[hydroxy-(3-methyl-3H-imidazol-4-yl)-methyl]-benzonitrile

Acetic acid (4-cyano-3-fluoro-phenyl)-(3-methyl-3H-imidazol-4-yl)-methyl ester (as described in Step C above) (1.26 g, 4.59 mmol) and NaOH (5.5 mL, 5.5 mmol) were dissolved in THF (15 mL) and H₂O (25 mL). After 1 h, the reaction was diluted with satd. NaHCO₃ solution, extracted with CH₂Cl₂ (3×), dried (MgSO₄) and concentrated to give the title compound.

Step F: Preparation of 2-[3-(3-ethyl-1-methyl-2-oxo-azepan-3-yl)-phenoxy]-4-[hydroxy-(3-methyl-3-H-imidazol-4-yl)-methyl]-benzonitrile

2-Fluoro-4-[hydroxy-(3-methyl-3H-imidazol-4-yl)-methyl]-benzonitrile (as described in Step E above) (0.162 g, 0.700 mmol), 3-ethyl-3-(3-hydroxy-phenyl)-1-methyl-azepan-2-one, commercially available from Maybridge (0.173 g, 0.700 mmol) and KF.Al₂_{O}₂₃ (0.208 g) and 18-Crown-6 (0.017 g, 0.064 mmol) were dissolved in anhydrous CH₃CN (7 mL) and refluxed under Ar for 24 h. The reaction was filtered, concentrated and purified using SiO₂ chromatography (1-3% MeOH/CH₂Cl₂).

FAB MS (M+1)=459.

### Example 22A

Preparation of 2-[3-(3-ethyl-1-methyl-2-oxo-azepan-3-yl)-benzyl]-4-(3-methyl-3H-imidazole-4-carbonyl)-benzonitrile Trifluoroacetate

Step A: Preparation of 2-fluoro-4-[amino-(3-methyl-3H-imidazol-4-yl)-methyl]-benzonitrile

2-Fluoro-4-[hydroxy-(3-methyl-3H-imidazol-4-yl)-methyl]-benzonitrile, prepared as described in Example 22, Step E (0.542 g, 2.31 mmol) was dissolved in SOCl₂ (15 mL) and stirred at room temperature for 2 h under Ar₂. The solution was concentrated in vacuo and azeotroped with CH₂Cl₂ (3×). The solid was dissolved in CHCl₃ (30 mL) and cooled to −78° C. NH₃ (g) was bubbled through the solution and stirred for 16 h while warming to room temperature under Ar. After concentration to dryness and chromatography (silica gel, 1-2% CH₃OH/CH₂Cl₂ with NH₃), the title compound was obtained.

Step B: Preparation of 2-[3-(3-ethyl-1-methyl-2-oxo-azepan-3-yl)-benzyl]-4-(3-methyl-3H-imidazole-4-carbonyl)-benzonitrile Trifluoroacetate

2-Fluoro-4-[amino-(3-methyl-3H-imidazol-4-yl)-methyl]-benzonitrile (as described in Step A above) (0.012 g, 0.052 mmol), 3-ethyl-3-(3-hydroxy-phenyl)-1-methyl-azepan-2-one, commercially avaiable from Maybridge (0.014 g, 0.056 mmol), KF.Al₂O₃ (0.020 g) and 18-Crown-6 (0.001 g) were dissolved in anhydrous CH₃CN (2 mL) and refluxed under Ar for 24 h. The reaction was filtered, concentrated and purified using R^{P} LC on a VYDAC column eluting with 0.1%TFA/H₂O: 0.1%TFA/CH₃CN to give the title compound.

FAB MS (M+1)=457.

### Example 23

Preparation of 2-[3-(3-ethyl-1-methyl-2-oxo-azepan-3-yl)-phenoxy]-4-[1-hydroxy-1-(3-methyl-3H-imidazol-4-yl)-ethyl]-benzonitrile

2-[3-(3-Ethyl-1-methyl-2-oxo-azepan-3-yl)-phenoxy]-4-(3-methyl-3H-imidazole-4-carbonyl)-benzonitrile (prepared as described in Example 22A) (0.216 g, 0.473 mmol) was dissolved in anhydrous THF (10 mL) and a 3.0 M solution of MeMgBr (1.10 mL, 3.30 mmol) was added and stirred at RT. The reaction was quenched with NH₄Cl after 1 h, concentrated, diluted with EtOAc, washed with satd. NaHCO₃ solution, water, brine, dried (MgSO₄), concentrated and purified using SiO₂ chromatography (1-3% MeOH/CH₂Cl₂ w/NH₄OH) to give the title compound. FT/ICR MS (M+1)=473.

### Example 24

Preparation of 2-[3-(3-ethyl-1-methyl-2-oxo-azepan-3-yl)-phenoxy]-4-[1-amino-1-(3-methyl-3H-imidazol-4-yl)-ethyl]-benzonitrile

2-[3-(3-ethyl-1-methyl-2-oxo-azepan-3-yl)-phenoxy]-4-[1-hydroxy-1-(3-methyl-3H-imidazol-4-yl)-ethyl]-benzonitrile (prepared as described in Example 23) (0.099 g, 0.209 mmol) was dissolved in SOCl₂ (5 mL) and stirred at RT for 2 h. The solution was concentrated in vacuo and azeotroped with anhydrous CH₂Cl₂ (3×). The solid was dissolved in CHCl₃ (5 mL) and cooled to −78° C. NH₃ (g) was bubbled through the solution and stirred for 2 h while warming to RT under Ar. The solution was concentrated in vacuo and purified using reverse phase chromatography (95/5-5/95H₂O/CH₃CN with 0.1% TFA, flow =65 mL/min). The compound was converted to its free base using saturated NaHCO₃ solution, extracted with CH₂Cl₂ (3×), dried (MgSO₄), filtered and treated with 1N HCl ethereal solution to give the title compound. FAB MS (M+1)=472.

Analysis calculated for C₂₈H₃₃N₅O₂0.35EtOAc: C, 70.28; H, 7.18; N, 13.94 Found: C, 70.37; H, 7.29; N, 13.88.

The four individual diastereomers of the title compound were prepared by the methods described in examples 22, 23 and 24 using chiral 3-ethyl-3-(3-hydroxy-phenyl)-1-methyl-azepan-2-one (the racemate was commercially obtained from Maybridge and the enantiomers separated on a chiral column) in the Example 22A, Step B reaction, and subsequently resovling the diastereomers by purification of the enantiomers at the amino group of the product from the eventual Example 24 reaction using chiral HPLC.

### Example 24A

Preparation of 1-(4-cyanobenzyl)-5-chloromethylimidazole HCl Salt

Step 1: Preparation of 4-Cyanobenzylamine

Method 1 (Hydrochloride salt): A 72 liter vessel was charged with 190 proof ethanol (14.4 L) followed by the addition of 4-cyanobenzylbromide (2.98 kg) and HMTA (2.18 kg) at ambient temperature. The mixture was heated to about 72-75° C. over about 60 min. On warming, the solution thickens and additional ethanol (1.0 liter) was added to facilitate stirring. The batch was aged at about 72-75° C. for about 30 min.

The mixture was allowed to cool to about 20° C. over about 60 min. and HCl gas (2.20 kg) was sparged into the slurry over about 4 hours during which time the temperature rose to about 65° C. The mixture was heated to about 70-72° C. and aged for about 1 hour. The slurry was cooled to about 30° C. and ethyl acetate (22.3 L) added over about 30 min. The slurry was cooled to about −5° C. over about 40 min and aged at about −3 to about −5° C. for about 30 min. The mixture was filtered and the crystalline solid was washed with chilled ethyl acetate (3×3 L). The solid was dried under a N₂ stream for about 1 hour before charging to a 50 liter vessel containing water (5.5 L). The pH was adjusted to about 10-10.5 with 50% NaOH (4.0 kg) maintaining the internal temperature below about 30° C. At about 25° C., methylene chloride (2.8 L) was added and stirring continued for about 15 min. The layers were allowed to settle and the lower organic layer was removed. The aqueous layer was extracted with methylene chloride (2×2.2 L). The combined organic layers were dried over potassium carbonate (650 g). The carbonate was removed via filtration and the filtrate concentrated in vacuo at about 25° C. to give a free base as a yellow oil.

The oil was transferred to a 50 liter vessel with the aid of ethanol (1.8 L). Ethyl acetate (4.1 L) was added at about 25° C. The solution was cooled to about 15° C. and HCl gas (600 g) was sparged in over about 3 hours, while keeping batch temperature below about 40° C. At about 20-25° C., ethyl acetate (5.8 L) was added to the slurry, followed by cooling to about −5° C. over about 1 hour. The slurry was aged at about −5° C. for about 1 hour and the solids isolated via filtration. The cake was washed with a chilled mixture of EtOAc/EtOH (9:1 v/v) (1×3.8 L), then the cake was washed with chilled EtOAc (2×3.8 L). The solids were dried in vacuo at about 25° C. to provide the above-titled compound.

¹H NMR (250 MHz, CDCl₃): δ 7.83-7.79 (d, 2H), 7.60-7.57 (d, 2H), 4.79 (s, 2H), 4.25 (s, 2H); ¹³C NMR (62.9 MHz, CDCl₃): δ 149.9, 139.8, 134.2, 131.2, 119.7, 113.4, 49.9, 49.5, 49.2, 48.8, 48.5, 48.2, 43.8.

Method 2 (phosphate salt): A slurry of HMTA in 2.5 L EtOH was added gradually over about 30 min to about 60 min to a stirred slurry of cyanobenzyl-bromide in 3.5 L EtOH and maintained at about 48-53° C. with heating & cooling in a 22L neck flask (small exotherm). Then the transfer of HMTA to the reaction mixture was completed with the use of 1.0 L EtOH. The reaction mixture was heated to about 68-73° C. and aged at about 68-73° C. for about 90 min. The reaction mixture was a slurry containing a granular precipitate which quickly settled when stirring stopped.

The mixture was cooled to a temperature of about 50° C. to about 55° C. Propionic acid was added to the mixture and the mixture was heated and maintained at a temperature of about 50° C. to about 55° C. Phosphoric acid was gradually added over about 5 min to about 10 min, maintaining the reaction mixture below about 65° C. to form a precipitate-containing mixture. Then the mixture was gradually warmed to about 65° C. to about 70° C. over about 30 min and aged at about 65° C. to about 70° C. for about 30 min. The mixture was then gradually cooled to about 20-25° C. over about 1 hour and aged at about 20-25° C. for about 1 hour.

The reaction slurry was then filtered. The filter cake was washed four times with EtOH, using the following sequence, 2.5 L each time. The filter cake was then washed with water five times, using 300 mL each time. Finally, the filter cake was washed twice with MeCN (1.0 L each time) and the above identified compound was obtained.

Step 2: Preparation of 1-(4-Cyanobenzyl)-2-Mercapto-5-Hydroxymethylimidazole

7% water in acetonitrile (50 mL) was added to a 250 mL roundbottom flask. Next, an amine phosphate salt (12.49 g), prepared as described in Step 1, was added to the flask. Next potassium thiocyanate (6.04 g) and dihydroxyacetone (5.61 g) was added. Lastly, propionic acid (10.0 mL) was added. Acetonitrile/water 93:7 (25 mL) was used to rinse down the sides of the flask. This mixture was then heated to 60° C., aged for about 30 minutes and seeded with 1% thioimidazole. The mixture was then aged for about 1.5 to about 2 hours at 60° C. Next, the mixture was heated to 70° C., and aged for 2 hours. The temperature of the mixture was then cooled to room temperature and was aged overnight. The thioimidazole product was obtained by vacuum filtration. The filter cake was washed four times acetonitrile (25 mL each time) until the filtrates became nearly colorless. Then the filter cake was washed three times with water (approximately 25-50 mL each time) and dried in vacuo to obtain the above-identified compound.

Step 3: Preparation of 1-(4-Cyanobenzyl)-5-Hydroxymethylimidazole

A 1L flask with cooling/heating jacket and glass stirrer (Lab-Max) was charged with water (200 mL) at 25° C. The thioimidazole (90.27 g), prepared as described in Step 2, was added, followed by acetic acid (120 mL) and water (50 mL) to form a pale pink slurry. The reaction was warmed to 40° C. over 10 minutes. Hydrogen peroxide (90.0 g) was added slowly over 2 hours by automatic pump maintaining a temperature of 35-45° C. The temperature was lowered to 25° C. and the solution aged for 1 hour.

The solution was cooled to 20° C. and quenched by slowly adding 20% aqueous Na₂SO₃ (25 mL) maintaining the temperature at less than 25° C. The solution was filtered through a bed of DARCO G-60 (9.0 g) over a bed of SolkaFlok (1.9 g) in a sintered glass funnel. The bed was washed with 25 mL of 10% acetic acid in water.

The combined filtrates were cooled to 15° C. and a 25% aqueous ammonia was added over a 30 minute period, maintaining the temperature below 25° C., to a pH of 9.3. The yellowish slurry was aged overnight at 23° C. (room temperature). The solids were isolated via vacuum filtration. The cake (100 mL wet volume) was washed with 2×250 mL 5% ammonia (25%) in water, followed by 100 mL of ethyl acetate. The wet cake was dried with vacuum/N₂ flow and the above-titled compound was obtained.

¹H NMR (250 MHz, CDCl₃): δ 7.84-7.72 (d, 2H), 7.31-7.28 (d, 2H), 6.85 (s, 1H), 5.34 (s, 2H), 5.14-5.11 (t, 1H), 4.30-4.28 (d, 2H), 3.35 (s, 1H).

Step 4: Preparation of 1-(4-cyanobenzyl)-5-chloromethylimidazole HCl Salt

Method 1: 1-(4-Cyanobenzyl)-5-hydroxymethylimidazole (1.0 kg), prepared as described above in Step 3, was slurried with DMF (4.8 L) at 22° C. and then cooled to −5° C. Thionyl chloride (390 mL) was added dropwise over 60 min during which time the reaction temperature rose to a maximum of 9° C. The solution became nearly homogeneous before the product began to precipitate from solution. The slurry was warmed to 26° C. and aged for 1 h.

The slurry was then cooled to 5° C. and 2-propanol (120 mL) was added dropwise, followed by the addition of ethyl acetate (4.8 L). The slurry was aged at 5° C. for 1 h before the solids were isolated and washed with chilled ethyl acetate (3×1 L). The product was dried in vacuo at 40° C. overnight to provide the above-titled compound.

¹H NMR (250 MHz DMSO-d₆): δ 9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d₆): δ_{c} 139.7, 137.7, 132.7, 130.1, 128.8, 120.7, 118.4, 111.2, 48.9, 33.1.

Method 2: To an ice cold solution of dry acetonitrile (3.2 L, 15 L/Kg hydroxymethylimidazole) was added 99% oxalyl chloride (101 mL, 1.15 mol, 1.15 equiv.), followed by dry DMF (178 mL, 2.30 mol, 2.30 equiv.), at which time vigorous evolution of gas was observed. After stirring for about 5 to 10 min following the addition of DMF, solid hydroxymethylimidazole (213 g, 1.00 mol), prepared as described above in Step 3, was added gradually. After the addition, the internal temperature was allowed to warm to a temperature of about 23° C. to about 25° C. and stirred for about 1 to 3 hours. The mixture was filtered, then washed with dry acetonitrile (400 mL displacement wash, 550 mL slurry wash, and a 400 mL displacement wash). The solid was maintained under a N₂ atmosphere during the filtration and washing to prevent hydrolysis of the chloride by adventitious H₂O. This yielded the crystalline form of the chloromethylimidazole hydrochloride.

¹H NMR (250 MHz DMSO-d₆): δ 9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d₆): δ_{c} 139.7, 137.7, 132.7, 130.1, 128.8, 120.7, 118.4, 111.2, 48.9, 33.1.

Method 3: To an ice cold solution of dry DMF (178 mL, 2.30 mol, 2.30 equiv.) in dry acetonitrile (2.56 L, 12 L/Kg Hydroxymethylimidazole) was added oxalyl chloride (101 mL, 1.15 mol, 1.15 equiv). The heterogeneous mixture in the reagent vessel was then transferred to a mixture of hydroxymethylimidazole (213 g, 1.00 mol), prepared as described in Step 3 above, in dry acetonitrile (1.7 L, 8 L/Kg hydroxymethylimidazole). Additional dry acetonitrile (1.1-2.3 L, 5-11 L/Kg hydroxymethylimidazole) was added to the remaining solid Vilsmeier reagent in the reagent vessel. This, now nearly homogenous, solution was transferred to the reaction vessel at Tᵢ≦+6° C. The reaction vessel temperature was warmed to a temperature of about 23° C. to about 25° C. and stirred for about 1 to 3 hours. The mixture was then cooled to 0° C. and aged 1 h. The solid was filtered and washed with dry, ice cold acetonitrile (400 mL displacement wash, 550 mL slurry wash, and a 400 mL displacement wash). The solid was maintained under a N₂ atmosphere during the filtration and washing to prevent hydrolysis of the chloride by adventitious H₂O. This yielded the crystalline form of the chloromethylimidazole hydrochloride.

### Example 24B

Preparation Of 1-(4′-Cyanobenzyl) imidazol-5-ylmethyl Piperazine

Step 1: Preparation of 1-(4′-Cyanobenzyl) imidazol-5-ylmethyl piperazine-4-carboxylic Acid Benzyl Ester

To an acetonitrile solution of 1-(4′-cyanobenzyl)-5-chloromethylimidazole (7.45 mmol), prepared as described in Example 24A, Step 4, and diisopropylethyl amine (22.4 mmol) was added 1-benzyl 1-piperazine carboxylate (10.4 mmol). This solution was stirred for 4.0 hours at 80° C. The product was isolated after silica column purification.

¹H-NMR (CDCl₃): δ 7.65 (d, 2H); 7.55 (s, 1H); 7.38 (m, 5H); 7.15 (d, 2H); 7.0 (s, 1H); 5.3 (s, 2H); 5.1 (s, 1H); 3.4 (m, 4H); 3.3 (s, 2H); 2.3 (m, 4H).

Step 2: Preparation of 1-(4′-Cyanobenzyl) imidazol-5-ylmethyl Piperazine

The product from Step 1 (6.17 mmol) was dissolved in absolute ethanol followed by the introduction of 10% Pd/C catalyst then hydrogen under atmospheric pressure. The catalyst was removed via filtration through filter-aid and the product was isolated by removing the solvent under reduced pressure.

¹H-NMR (CD₃OD): δ 7.8 (s, 1H); 7.75 (d, 2H); 7.3 (d, 2H); 6.9 (s, 1H); 5.45 (s, 2H); 3.3 (m, 4H); 2.6 (s, 2H); 2.3 (m, 4H).

### Example 24C

Preparation of 1-[1-(4′-Cyanobenzyl) imidazol-5-ylmethyl]piperazine-4-(DL-2-hydroxy-2-(o-methoxyphenyl)) Acetamide

Step 1: Preparation of 2-(DL-O-tert-butyl diphenylsilyl)-2-(2-methoxyphenyl)acetic Acid

The title product was obtained by treating DL-2-methoxymandelic acid (0.20 g, 1.1 mmol) with tert-butyldiphenyl silyl chloride (2.42 mmol) and imidazole (11 mmol) followed by selective hydrolysis as outlined in J. Chem. Soc., Perkin Trans 1,1985, 2361. For example, the silyl ester was selectively hydrolyzed by treatment with 10% aqueous K₂CO₃ followed by acidification (pH 3) with 1M KHSO₄.

¹H-NMR (CDCl₃): δ 6.8-7.7 (m, 14H); 5.4 (s, 1H); 3.65 (s, 3H); 1.05 (s, 9H).

Step 2: Preparation of 1-[1-(4′-Cyanobenzyl) imidazol-5-ylmethyl]piperazine-4-(DL-2-hydroxy-2-(o-methoxyphenyl)) Acetamide

A DMF solution of the product from Step 1 (0.346 g, 0.826 mmol), 1-(4′-cyanobenzyl) imidazol-5-ylmethyl piperazine (0.116 g, 0.413 mmol) (prepared as described in Example 24A, Step 2), HOBt (0.132 g, 0.87 mmol), EDC (0.173 g, 0.91 mmol) and NMM (1.24 mmol) was stirred for 18 hours at 25° C. The pure deprotected and protected products were obtained directly after preparative hplc separation and lyophilization.

FAB-MS: calc: 445.5, found: 446.2. ¹H-NMR (CD₃OD): δ 8.2 (s, 1H); 7.7 (d, 2H); 7.3-7.4 (m, 4H); 6.95-7.1 (m, 3H); 5.7 (s, 1H); 5.5 (s, 2H); 3.85 (s, 3H); 3.5 (m, 1H); 3.0-3.3 (m, 5H); 2.35 (m, 1H); 2.15 (m, 2H); 1.75 (m, 1H).

### Example 24D

Preparation of 1-[1-(4′-cyanobenzyl) imidazol-5-ylmethyl]-4-[1-(2,6-dimethylbenzyloxycarbonyl]piperazine Bis Trifloroacetate Salt

A solution of 250 mg (1.84 mmol) of 2,5-dimethylbenzyl alcohol and 409 mg (2.03 mmol) of p-nitrophenylchloroformate in 5 ml of 7:1 THF/acetonitrile under an argon atmosphere was treated with 164 ml (2.03 mmol) of pyridine, and the resulting suspension was stirred vigorously at room temp. for 18 h. The reaction was concentrated in vacuo to give a clear oil. The oil was dissolved in a minimum of chloroform and was chromatographed over silica gel with 9:1 hexanes/ethyl acetate as eluant. Product fractions were combined and concentrated in vacuo to give the carbonate intermediate as an off-white solid.

400 Mhz H¹ NMR(CDCl₃): δ 2.44 (d, 6H), 5.44 (s, 2H), 7.09 (d, 2H), 7.20 (t, 1H), 7.40 (d, 2H), 8.29 (d, 2H).

A solution of 219 mg (0.71 mmol) of the above prepared carbonate intermediate, 200 mg (0.71 mmol) of 1-(4′-Cyanobenzyl) imidazol-5-ylmethyl piperazine (prepared as described in Example 24B, Step 2) and 247 ml (1.42 mmol) of DIEA in 2 ml of methylene chloride was stirred at room temp. for 18 h. The reaction was concentrated in vacuo to a yellow oil. The oil was purified by reversed phase preparatory LC, and the pure fractions combined and concentrated to remove volatiles. Lyophilization of the aqueous residue provided the bis trifluoroacetic acid salt of the desired product as an amorphous fluffy white powder.

FAB MS: M⁺=444.2. 400 Mhz H¹ NMR(CDCl₃): δ 2.39 (s, 6H), 2.65 (br s, 4H), 3.58 (br s, 4H), 3.67 (s, 2H), 5.22 (s, 2H), 5.57 (s, 2H), 7.04 (d, 2H), 7.18 (t, 1H), 7.26 (d, 2H), 7.54 (s, 1H), 7.72 (d, 2H), 8.80 (s, 1H).

### Example 24E

Preparation of 1-[1-(4′-cyanobenzyl) imidazol-5-ylmethyl]-4-[1-(2-ethoxybenzyloxycarbonyl]piperazine

In a manner identical to that described above in Example 24D, from 160 mg (0.53 mmol) of (2-ethoxybenzyl)-(4-nitrophenyl) carbonate (prepared as described above in Example 24D from p-nitrophenylchloroformate and 2-ethoxybenzyl alcohol) and 150 mg (0.53 mmol) of 1-(4′-Cyanobenzyl) imidazol-5-ylmethyl piperazine (prepared as described above in Example 24B, Step 2) was obtained the bis trifluoroacetic acid salt of the title compound as an amorphous fluffy white powder.

High Res. FAB MS: M+theo.=460.2343, obs.=460.2364. 400 Mhz H¹ NMR(DMSO-d₆): δ 1.36 (t, 3H), 2.39 (br s, 4H), 3.21 (br s, 2H+4H), 3.56 (br s, 2H), 4.04 (q, 2H), 5.02 (s, 2H), 5.60 (s, 2H), 6.93 (t, 1H), 7.00 (d, 1H), 7.26 (t, 1H), 7.29 (d, 1H), 7.44 (d, 2H), 7.71 (s, 1H), 7.89 (d, 2H), 9.18 (s, 1H).

### Example 25

Preparation of [N-Ac-(4-trans-L-Hyp)]-Ala-Ser-Chg-Gln-Ser-Leu-Dox (SEQ.ID.NO.: 35)

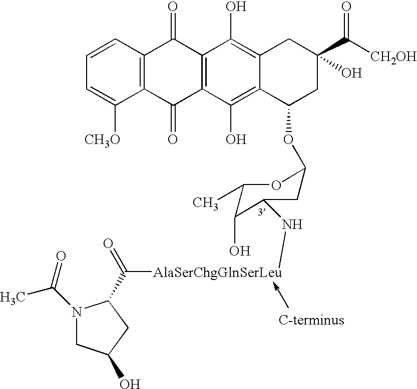

Step A: [N-Ac-(4-trans-L-Hyp(Bzl))]-Ala-Ser(Bzl)Chg-Gln-Ser(Bzl)Leu-PAM Resin (3-1).

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Acetic acid was used for the introduction of the N terminal acetyl group. Removal of the Boc group was performed using 50% TFA in methylene chloride and the TFA salt neutralized with diisopropylethylamine. At the completion of the synthesis the peptide resin was dried to yield Intermediate 3-1.

Step B: [N-Ac-(4-trans-L-Hyp)]-Ala-Ser-Chg-Gln-Ser-Leu-OH (3-2)

The protected peptide resin (3-1), 1.2 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield Intermediate 3-2.

Step C: [N-Ac-(4-trans-L-Hyp)-Ala-Ser-Chg-Gln-Ser-Leu-Dox

The above described intermediate (3-2), 1.157 g (1.45 mmol) was dissolved in DMSO (30 ml) and diluted with DMF (30 ml). To the solution was added doxorubicin hydrochloride, 516 mg (0.89 mmol) followed by 0.310 ml of diisopropylethylamine (1.78 mmol). The stirred solution was cooled (0° C.) and 0.276 ml of diphenylphosphoryl azide (1.28 mmol) added. After 30 minutes, an additional 0.276 ml (1.28 mmol) of DPPA was added and the pH adjusted to −7.5 (pH paper) with diisopropylethylamine (DIEA). The pH of the cooled reaction (0° C.) was maintained at −7.5 with DIEA for the next 3 hrs. and the reaction stirred at 0-4° C. overnight. After 18 hrs., the reaction (found to be complete by analytical HPLC, system A) was concentrated to an oil. Purification of the crude product was achieved by preparative HPLC, Buffer A=0.1% NH₄OAc-H₂O; B=CH₃CN. The crude product was dissolved in 400 ml of 100% A buffer, filtered and purified on a C-18 reverse phase HPLC radial compression column (Waters, Delta-Pak, 15 μM, 100 Å). A step gradient of 100% A to 60% A was used at a flow rate of 75 mmin (UV=214 nm). Homogeneous product fractions (evaluated by HPLC, system A) were pooled and freeze-dried. The product was dissolved in H₂O (300 ml), filtered and freeze-dried to provide the purified title compound.

Physical Properties

The physical/chemical properties of the product of Step C are shown below:

| | |
|---|---|
| Molecular Formula: | C₆₂H₈₅N₉O₂₃ |
| Molecular Weight: | 1323.6 |
| High Resolution ES Mass Spec: | 1341.7 (NH₄⁺) |
| HPLC: | System A |
| Column: | Vydac 15 cm #218TP5415, C18 |
| Eluant: | Gradient 95:5 (A:B) to 5:95 (A:B) over |
| | 45 min. A = 0.1% TFA/H₂O, |
| | B = 0.1% TFA/Acetonitrile |
| Flow: | 1.5 ml/min. |
| Wavelength: | 214 nm, 254 nm |
| Retention Time: | 18.2 min. |

Amino Acid Compositional Analysis¹:

| Theory | Found |
|---|---|
| Ala (1) | 1.00 |
| Ser (2) | 1.88 |
| Chg (1) | 0.91 |
| Gln² (1) | 1.00 (as Glu) |
| Hyp (1) | 0.80 |
| Leu (1) | 1.01 |
| Peptide Content: | 0.657 μmol/mg |

Note:
¹20 hr., 100° C., 6N HCl
²Gln converted to Glu

### Example 26

Preparation of [N-Glutaryl-(4-trans-L-Hyp)]-Ala-Ser-Chg-Gln-Ser-Leu-Dox (SEQ.ID.NO.: 60) (Compound B)

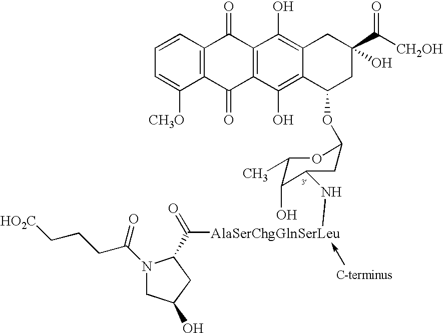

Step A: [N-Glutaryl(OFm)-(4-trans-L-Hyp)]-Ala-Ser-Chg-Gln-Ser-Leu-PAM Resin

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Fmoc-Ser(tBu), Fmoc-Gln(Trt), Fmoc-Chg, Fmoc-Ala, Boc-(4-trans-L-Hyp). Coupling was achieved using DCC and HOBT activation in methyl-2-pyrrolidinone. The intermediate mono fluorenylmethyl ester of glutaric acid [Glutaryl(OFm)]was used for the introduction of the N-terminal glutaryl group. Removal of the Fmoc group was performed using 20% piperidine. The acid sensitive protecting groups, Boc, Trt and tBu, were removed with 50% TFA in methylene chloride. Neutralization of the TFA salt was with diisopropylethylamine. At the completion of the synthesis, the peptide resin was dried to yield the title compound.

Step B: [N-Glutaryl(OFm)-(4-trans-L-Hyp)]-Ala-Ser-Chg-Gln-Ser-Leu-OH

The protected peptide resin from Step A, 1.2 g, was treated with HF (20 ml) for 1 hr at 0° C. in the presence of anisole (2 ml). After evaporation of the HF, the residue was washed with ether, filtered and extracted with DMF. The DMF filtrate (75 ml) was concentrated to dryness and triturated with H₂O. The insoluble product was filtered and dried to provide the title compound.

Step C: [N-Glutaryl(OFm)-(4-trans-L-Hyp)]-Ala-Ser-Chg-Gln-Ser-Leu-Dox

The above prepared intermediate from Step B, (1.33 g, 1.27 mmol) was dissolved in DMSO (6 ml) and DMF (69 ml). To the solution was added doxorubicin hydrochloride, 599 mg (1.03 mmol) followed by 376 μl of diisopropylethylamine (2.16 mmol). The stirred solution was cooled (0° C.) and 324 μl of diphenylphosphoryl azide (1.5 mmol) added. After 30 minutes, an additional 324 μl of DPPA was added and the pH adjusted to ˜7.5 (pH paper) with diisopropylethyl-amine (DIEA). The pH of the cooled reaction (0° C.) was maintained at ˜7.5 with DIEA for the next 3 hrs and the reaction stirred at 0-4° C. overnight. After 18 hrs., the reaction (found to be complete by analytical HPLC, system A) was concentrated to provide the title compound as an oil.

Step D: [N-Glutaryl-(4-trans-L-Hyp)]-Ala-Ser-Chg-Gln-Ser-Leu-Dox

The above product from Step C was dissolved in DMF (54 ml), cooled (0° C.) and 14 ml of piperidine added. The solution was concentrated to dryness and purified by preparative HPLC. (A=0.1% NH₄OAc-H₂O; B=CH₃CN.) The crude product was dissolved in 100 ml of 80% A buffer, filtered and purified on a C-18 reverse phase HPLC radial compression column (Waters, Delta-Pak, 15μ, 100 Å). A step gradient of 80% A to 67% A was used at a flow rate of 75 ml/min (uv=214 nm). Homogeneous product fractions (evaluated by HPLC, system A) were pooled and freeze-dried. The product was further purified using the above HPLC column. Buffer A=15% acetic acid-H₂O; B=15% acetic acid-methanol. The product was dissolved in 100 ml of 20% B/80% A buffer and purified. A step gradient of 20% B to 80% B was used at a flow rate of 75 ml/min (uv=260 nm). Homogeneous product fractions (evaluated by HPLC, system A) were pooled, concentrated and freeze-dried from H₂O to yield the purified title compound.

| | |
|---|---|
| High Resolution ES Mass Spec: | 1418.78 (Na⁺) |
| HPLC: | System A |
| Column: | Vydac 15 cm #218TP5415, C18 |
| Eluant: | Gradient 95:5 (A:B) to 5:95 (A:B) over |
| | 45 min. A = 0.1% TFA/H₂O, |
| | B = 0.1% TFA/Acetonitrile |
| Flow: | 1.5 ml/min. |
| Wavelength: | 214 nm, 254 nm |
| Retention Time: | 18.3 min. |

Amino Acid Compositional Analysis¹:

| Theory | FFound |
|---|---|
| Ala (1) | 0.99 |
| Ser (2) | 2.02 |
| Chg (1) | 1.00 |
| Gln² (1) | 1.01 (as Glu) |
| Hyp (1) | 0.99 |
| Leu (1) | 1.00 |
| Peptide Content: | 0.682 μmol/mg |

Note:
¹20 hr., 100° C., 6N HCl
²Gln converted to Glu

### Example 27

Preparation of (4-trans-L-Hyp)-Ala-Ser-Chg-Gln-Ser-Leu-Dox (SEQ.ID.NO.: 61)

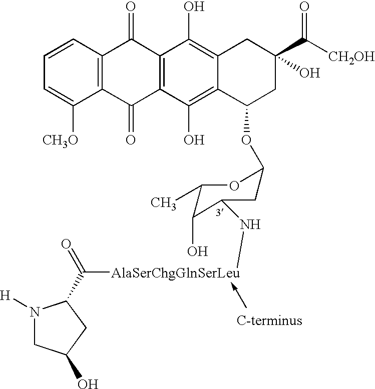

Step A: Fmoc-(4-trans-L-Hyp(Bzl))-Ala-Ser(Bzl)Chg-Gln-Ser(Bzl)Leu-PAM Resin

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Fmoc-OSu (succinamidyl ester of Fmoc) was used for the introduction of the N-terminal protecting group. Removal of the Boc group was performed using 50% TFA in methylene chloride and the TFA salt neutralized with diisopropylethylamine. At the completion of the synthesis the peptide resin was dried to yield the title intermediate.

Step B: Fmoc-(4-trans-L-Hyp)-Ala-Ser-Chg-Gln-Ser-Leu-OH

The protected peptide resin from Step A, 1.1 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield the title intermediate.

Step C: Fmoc-(4-trans-L-Hyp)-Ala-Ser-Chg-Gln-Ser-Leu-Dox The intermediate from Step B, 0.274 g, was dissolved in DMSO (10 ml) and diluted with DMF (10 ml). To the solution was added doxorubicin hydrochloride, 104 mg followed by 62 μL of diisopropylethylamine (DIEA). The stirred solution was cooled (0° C.) and 56 μL of diphenylphosphoryl azide added. After 30 minutes, an additional 56 μL of DPPA was added and the pH adjusted to ˜7.5 (pH paper) with DIEA. The pH of the cooled reaction (0° C.) was maintained at ˜7.5 with DIEA. After 4 hrs., the reaction (found to be complete by analytical HPLC, system A) was concentrated to an oil. HPLC conditions, system A.

Step D: (4-trans-L-Hyp)-Ala-Ser-Chg-Gln-Ser-Leu-Dox

The above product from Step C was dissolved in DMF (10 ml), cooled (0° C.) and 4 ml of piperidine added. The solution was concentrated to dryness and purified by preparative HPLC. (A=0.1% NH₄OAc-H₂O; B=CH₃CN.) The crude product was dissolved in 100 ml of 90% A buffer, filtered and purified on a C-18 reverse phase HPLC radial compression column (Waters, Delta-Pak, 15μ, 100 Å). A step gradient of 90% A to 65% A was used at a flow rate of 75 ml/min (uv=214 nm). Homogeneous product fractions (evaluated by HPLC, system A) were pooled and freeze-dried.

| | |
|---|---|
| Molecular Formula: | C₆₀H₈₃N₉O₂₂ |
| Molecular Weight: | 1281.56 |
| High Resolution ES Mass Spec: | 1282.59 (MH⁺) |
| HPLC: | System A |
| Column: | Vydac 15 cm #218TP5415, C18 |
| Eluant: | Gradient 95:5 (A:B) to 5:95 (A:B) over |
| | 45 min. A = 0.1% TFA/H₂O, |
| | B = 0.1% TFA/Acetonitrile |
| Flow: | 1.5 ml/min. |
| Wavelength: | 214 nm, 254 nm |
| Retention Time: | 17.6 min. |

Amino Acid Compositional Analysis¹:

| Theory | Found |
|---|---|
| Ala (1) | 1.00 |
| Ser (2) | 1.94 |
| Chg (1) | 0.94 |
| Gln² (1) | 1.05 (as Glu) |
| Hyp (1) | 0.96 |
| Leu (1) | 1.03 |
| Peptide Content: | 0.690 μmol/mg |

Note:
¹20 hr., 100° C., 6N HCl
²Gln converted to Glu

### Example 28

des-Acetylvinblastine-4-O—(N-Acetyl-4-trans-L-Hyp-Ser-Ser-Chg-Gln-Ser-Ser-Pro) ester (SEQ.ID.: 49)

Step A: Preparation of 4-des-Acetylvinblastine

A sample of 2.40 g (2.63 mmol) of vinblastine sulfate (Sigma V-1377) was dissolved under N₂ in 135 ml of absolute methanol and treated with 45 ml of anhydrous hydrazine, and the solution was stirred at 20-25° C. for 18 hr. The reaction was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂Cl₂ layers in turn was washed with 100 ml each of H₂O (2×) and saturated NaCl (1×). The combined organic layers were dried over anhydrous Na₂SO₄, and the solvent was removed at reduced pressure to yield the title compound as an off-white crystalline solid. This material was stored at −20° C. until use.

Step B: Preparation of 4-des-Acetylvinblastine 4-O-(Prolyl) Ester

A sample of 804 mg (1.047 mmol) of 4-des-acetylvinblastine, dissolved in 3 ml of CH₂Cl₂ and 18 ml of anhydrous pyridine under nitrogen, was treated with 1.39 g of Fmoc-proline acid chloride (Fmoc-Pro-Cl, Advanced Chemtech), and the mixture was stirred for 20 hr at 25° C. When analysis by HPLC revealed the presence of unreacted starting des-acetylvinblastine, another 0.50 g of Fmoc-Pro-Cl was added, with stirring another 20 hr to complete the reaction. Water (ca. 3 ml) was added to react with the excess acid chloride, and the solution was then evaporated to dryness and partitioned between 300 ml of EtOAc and 150 ml of saturated NaHCO₃, followed by washing twice with saturated NaCl. After drying (Na₂SO₄), the solvent was removed under reduced pressure to give an orange-brown residue, to which was added 30 ml of DMF and 14 ml of piperidine, and after 5 min the solution was evaporated under reduced pressure to give a orange-yellow semi-solid residue. After drying in vacuo for about 1 hr, approx. 200 ml of H₂O and 100 ml of ether was added to this material, followed by glacial HOAc dropwise with shaking and sonication until complete dissolution had occurred and the aqueous layer had attained a stable pH of 4.5-5.0 (moistened pH range 4-6 paper). The aqueous layer was then washed with 1 100-ml portion of ether, and each ether layer was washed in turn with 50 ml of H₂O. The combined aqueous layers were subjected to preparative HPLC in 2 portions on a Waters C4 Delta-Pak column 15 μM 300A (A=0.1% TFA/H₂O; B=0.1% TFA/CH₃CN), gradient elution 95-->70% A/70 min. Pooled fractions yielded, upon concentration and lyophilization, the title compound.

Step C: N-Acetyl-4-trans-L-Hyp-Ser-Ser-Chg-Gln-Ser-Ser-WANG Resin

Starting with 0.5 mmole (0.61 g) of Fmoc-Ser(t-Bu)-WANG resin loaded at 0.82 mmol/g, the protected peptide was synthesized on a ABI model 430A peptide synthesizer adapted for Fmoc/t-butyl-based synthesis. The protocol used a 2-fold excess (1.0 mmol) of each of the following protected amino acids: Fmoc-Ser(t-Bu)—OH, Fmoc-Gln-OH, Fmoc-Chg-OH, Fmoc-4-trans-L-Hyp-OH; and acetic acid (double coupling). During each coupling cycle Fmoc protection was removed using 20% piperidine in N-methyl-2-pyrrolidinone (NMP), followed by washing with NMP. Coupling was achieved using DCC and HOBt activation in NMP. At the completion of the synthesis, the peptide resin was dried to yield the title compound.

Step D: N-Acetyl-4-trans-L-Hyp-Ser-Ser-Chg-Gln-Ser-Ser-OH

One 0.5-mmol run of the above peptide-resin was suspended in 25 ml of TFA, followed by addition of 0.625 ml each of H₂O and triisopropylsilane, then stirring at 25° for 2.0 hr. The cleavage mixture was filtered, the solids were washed with TFA, the solvents were removed from the filtrate under reduced pressure, and the residue was triturated with ether to give a pale yellow solid, which was isolated by filtration and drying in vacuo to afford the title compound.

HPLC conditions, system A:

| Column . . . | Vydac 15 cm #218TP5415, C18 |
|---|---|
| Eluant . . . | Gradient (95% A --> 50% A) over 45 min. |
| | A = 0.1% TFA/H₂O, B = 0.1% |
| | TFA/acetonitrile |
| Flow . . . | 1.5 ml/min. |
| High Resolution ES/FT-MS: | 789.3 |

Step E: des-Acetylvinblastine-4-O—(N-Acetyl-4-trans-L-Hyp-Ser-Ser-Chg-Gln-Ser-Ser-Pro) Ester

Samples of 522 mg (0.66 mmol) of the peptide prepared as described in step D and 555 mg (ca. 0.6 mmol) of 4-des-Acetylvinblastine 4-O-(Prolyl) ester from Step B, prepared as above, were dissolved in 17 ml of DMF under N₂. Then 163 mg (1-13 mmol) of 1-hydroxy-7-azabenzotriazole (HOAt) was added, and the pH was adjusted to 6.5-7 (moistened 5-10 range pH paper) with 2,4,6-collidine, followed by cooling to 0° C. and addition of 155 mg (0.81 mmol) of 1-(3-dimethylaminopropyl)-3-ethylcarbodiimide hydrochloride (EDC). Stirring was continued at 0-5° C. until completion of the coupling as monitored by analytical HPLC (A=0.1% TFA/H₂O; B=0.1% TFA/CH₃CN), maintaining the pH at 6.5-7 by periodic addition of 2,4,6-collidine. After 12 hr the reaction was worked up by addition of ˜4 ml of H₂O and, after stirring 1 hr, concentrated to a small volume in vacuo and dissolution in ca. 150 ml of 5% HOAc. and preparative HPLC in two portions on a Waters C18 Delta-Pak column 15 μM 300A (A=0.1% TFA/H₂O; B=0.1% TFA/CH₃CN, gradient elution 95-->65% A/70 min). Homogeneous fractions containing the later-eluting product (evaluated by HPLC, system A, 95-->65% A/30 min) from both runs were pooled and concentrated to a volume of ˜50 ml and passed through approx. 40 ml of AG4X4 ion exchange resin (acetate cycle), followed by freeze-drying to give the title compound as a lyophilized powder.

High Resolution ES/FT-MS: 1637.0

### Example 29

des-Acetylvinblastine-4-O—(N-Acetyl-4-trans-L-Hyp-Ser-Ser-Chg-Gln-Ser-Ser-Pro) Ester Acetate (SEQ.ID.NO.: 49)

A sample of 4.50 g (3.7 mmol) of 4-O-(prolyl) desacetylvinblastine TFA salt, prepared as described in Example 28, Step B, was dissolved in 300 ml of DMF under N₂, and the solution was cooled to 0°. Then 1.72 g (10.5 mmol) of 3,4-dihydro-3-hydroxy-4-oxo-1,2,3-benzotriazine (ODHBT) was added, and the pH was adjusted to 7.0 (moistened 5-10 range pH paper) with N-methylmorpholine (NMM), followed by the addition of 4.95 g (5.23 mmol) of the N-acetyl-heptapeptide of Example 28, Step D, portionwise allowing complete dissolution between each addition. The pH was again adjusted to 7.0 with NMM, and 1.88 g (9.8 mmol) of 1-(3-dimethylaminopropyl)-3-ethylcarbodiimide hydrochloride (EDC) was added, followed by stirring of the solution at 0-5° C. until completion of the coupling as monitored by analytical HPLC (system A), maintaining the pH at ca. 7 by periodic addition of NMM. The analysis showed the major component at 26.3 min retention time preceded by a minor component (ca. 10%) at 26.1 min, identified as the D-Ser isomer of the title compound. After 20 hr the reaction was worked up by addition of 30 ml of H₂O and, after stirring 1 hr, concentrated to a small volume in vacuo and dissolution in ca. 500 ml of 20% HOAc. and preparative HPLC in 12 portions on a Waters C18 Delta-Pak column 15 mM 300A (A=0.1% TFA/H₂O; B=0.1% TFA/CH₃CN), gradient elution 85-->65% A/90 min) at a flow rate of 80 m/min.

Homogeneous fractions (evaluated by HPLC, system C) representing approx. one-fourth of the total run were pooled and concentrated to a volume of ˜150 ml and passed through approx. 200 ml of Bio-Rad AG4X4 ion exchange resin (acetate cycle), followed by freeze-drying of the eluant gave the acetate salt of the title compound as a lyophilized powder: retention time (system A) 26.7 min, 98.9% pure; high resolution ES/FT-MS m/e 1636.82; amino acid compositional analysis 20 hr, 100° C., 6N HCl (theory/found), Ser4/3.91 (corrected), Glu 1/0.92 (Gln converted to Glu), Chg 1/1.11, Hyp 1/1.07, Pro 1/0.99, peptide content 0.516 mmol/mg.

Further combination of homogeneous fractions and purification from side fractions, processing as above through approx. 500 ml of ion exchange resin, afforded an additional amounts of the title compound.

HPLC conditions, system A:

| | |
|---|---|
| Column . . . | Vydac 15 cm #218TP5415, C18 |
| Flow . . . | 1.5 ml/min. |
| Eluant . . . | Gradient (95% A --> 50% A) over 45 min. |
| | A = 0.1% TFA/H₂O, B = 0.1% |
| | TFA/acetonitrile |
| Wavelenth . . . | 214 nm, 280 nm |

HPLC conditions, system C:

| | |
|---|---|
| Column . . . | Vydac 15 cm #218TP5415, C18 |
| Flow . . . | 1.5 ml/min. |
| Eluant . . . | Gradient (85% A --> 65% A) over 30 min. |
| | A = 0.1% TFA/H₂O, B = 0.1% |
| | TFA/acetonitrile |
| Wavelenth . . . | 214 nm, 280 nm |

### Example 30

Preparation of 4-des-Acetylvinblastine-23-(4′-aminomethylbicyclo-[2.2.2]octane) methylamide (BDAM-(dAc)vinblastine)

Step A Preparation of 4-des-Acetylvinblastine-23-hydrazide

A sample of 3.99 g (4.38 mmol) of vinblastine sulfate (Sigma V-1377) was dissolved in 30.4 ml of 1:1 (v/v) absolute ethanol/anhydrous hydrazine, under N₂, and the solution was heated in an oil bath at 60-65° C. for 23 hr. Upon cooling, the solution was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂Cl₂ layers in turn was washed with 100 ml each of H₂O (2×) and saturated NaCl (1×). The combined organic layers were dried over anhydrous Na₂SO₄, and the solvent was removed in vacuo to yield, after drying 20 hr in vacuo, the title compound as a white crystalline solid. This material was dissolved in 82 ml of dry, degassed DMF for storage at −20° C. until use (conc. 36 mg/ml).

Step B Boc-4-aminomethylbicyclo-[2.2.2]octane Carboxylic Acid

A sample of 8.79 g (40.0 mmol) of 4-carboxybicyclo-[2.2.2]octanemethylamine hydrochloride salt suspended in 100 ml each of THF and H₂O was treated with 20.0 ml (14.6 g=3.3 equiv.) of TEA, followed by 11.8 g (47.9 mmol) of BOC-ON reagent. All went into solution, and after stirring 24 hr the solution was concentrated in vacuo to a volume of about 50 ml and partitioned between 100 ml of ether and 300 ml of H₂O. After addition of about 2 ml of TEA the aqueous layer was washed with ether (3×), each ether in turn washed with H₂O, and the combined aqueous layer was acidified with 5% KHSO₄ to give the title compound as a white solid, isolated by filtration and drying in vacuo.

Step C Boc-4-aminomethylbicyclo-[2.2.2]octane Carboxamide

A stirred solution under N₂ of 12.0 g (42.5 mmol) of the product from step B in 100 ml of DMF was treated with 8.0 g (49.3 mmol) of carbonyldiimidazole. After 30 min the DMF was evaporated in vacuo to afford 50-60 ml of a light brown paste, which was stirred and treated with 70 ml of conc. NH₄OH rapidly added. The initial solution turned to a white paste within 30 min, after which H₂O was added up to a total volume of 400 ml to complete precipitation of product, which was triturated and isolated by filtration and washing with H₂O, and dried in vacuo to yield the title compound as a white solid.

Step D Boc-4-aminomethylbicyclo-[2.2.2]octane Nitrile

A solution of 7.52 g (26.6 mmol) of the product from step C in 50 ml of CH₂Cl₂ and 80 ml of anhydrous pyridine was treated with 11.12 g of (methoxycarbonylsulfamoyl)-triethyl-ammonium hydroxide inner salt (Burgess reagent) in 1-g portions over 5 min. After stirring for 1.5 hr, TLC (90-10-1, CHCl₃—CH₃OH—H₂O) showed complete conversion to product, and the solution was evaporated to give a paste, to which H₂O was added, up to 400 ml, with trituration and stirring to afford, after standing 20 hr at 0° C., filtration and drying in vacuo, the title compound as a white solid.

Step E Boc-4-aminomethylbicyclo-[2.2.2]octane Methylamine

A solution of 6.75 g (25.5 mmol) of the product from step D in 200 ml of CH₃OH plus 4 ml of HOAc and 2 ml of H₂O was hydrogenated over 1.63 g of PtO₂ in a Parr shaker at 55 psi for 22 hr. The catalyst was removed by filtration through Celite, and the filtrate was concentrated in vacuo to an oily residue, which was flushed/evaporated with CH₃OH (1×) and CH₂Cl₂ (2×). Product began to crystallize toward the end of the evaporation, and ether (up to 300 ml) was added to complete the precipitation. The white solid was triturated and isolated by filtration and washing with ether to give, after drying in vacuo, the title compound as the acetate salt.

400 Mhz ¹H-NMR (CDCl₃): δ (ppm, TMS) 4.5 (1s, Boc-NH); 2.9 (2br d, —CH₂—NH-Boc); 2.45 (2br s, —CH₂—NH₂); 2.03 (3s, CH₃COOH); 1.45 (9s, Boc); 1.40 (12s, ring CH₂).

Step F Preparation of 4-des-Acetylvinblastine-23-(4′-aminomethylbicyclo-[2.2.2]octane) Methylamide (BDAM-(dAc)vinblastine)

A 30-ml aliquot of the above DMF solution of 4-des-acetylvinblastine-23-hydrazide (1.41 mmol), cooled to −15° C. under Argon, was converted to the azide in situ by acidification with 4M HCl in dioxane to pH<1.5 (moistened 0-2.5 range paper), followed by addition of 0.27 ml (1.3 equiv) of isoamyl nitrite and stirring for 1 hr at 10-15° C. The pH was brought to 7 by the addition of DIEA, and a slurry of 1.27 g (3.8 mmol) of the Boc diamine product from step E above in 20 ml of DMF was then added, and the reaction was allowed to warm slowly to 15-20° C. over 2 hr, at which point coupling was complete, as monitored by analytical HPLC (A=0.1% TFA/H₂O; B=0.1% TFA/CH₃CN). The solvent was removed in vacuo and the residue partitioned between EtOAc and 5% NaHCO₃, the organic layer washed with 5% NaCl, and the aqueous layers back-extracted with CH₂Cl₂ to assure removal of the intermediary Boc-BDAM-(dAc)vinblastine. The combined organic layers were dried over Na₂SO₄, the solvent was removed under reduced pressure, and the residue, after flush/evaporation twice from CH₂Cl₂, was dissolved in 30 ml of CH₂Cl₂ and treated with 30 ml of TFA for 30 min. The solvents were rapidly removed in vacuo, and the residue was dissolved in 300 ml of 10% HOAc for purification by preparative HPLC in 5 portions on a Waters C4 Delta-Pak column 15 μM 300A (A=0.1% TFA/H₂O; B=0.1% TFA/CH₃CN), gradient elution 95-->70% A/60 min, isocratic 70%/20 min. Homogeneous fractions (evaluated by HPLC, system A, 95-->50% A) from the five runs were pooled and concentrated in vacuo, followed by freeze-drying to give of the title compound as the lyophilized TFA salt.

HPLC conditions, system A:

| | |
|---|---|
| Column . . . | Vydac 15 cm #218TP5415, C18 |
| Eluant . . . | Gradient (A --> B) over 45 min. |
| | A = 0.1% TFA/H₂O, B = 0.1% |
| | TFA/acetonitrile |
| Flow . . . | 1.5 ml/min. |
| Retention time: | BDAM (dAc) vinblastine 23.5 min. |
| | (95% --> 50% A) 97% purity |
| High Resolution ES/FT-MS: | 905.63 |
| Compound content by elemental | N (calc) = 9.28 N (found) = 6.00 |
| analysis = 0.714 μmol/mg: | |

### Example 31

Preparation of 4-des-Acetylvinblastine-23-(N-Acetyl-Ser-Ser-Ser-Chg-Gln-Ser-Val-BDAM) Amide Acetate Salt (SEQ.ID.NO.: 45)

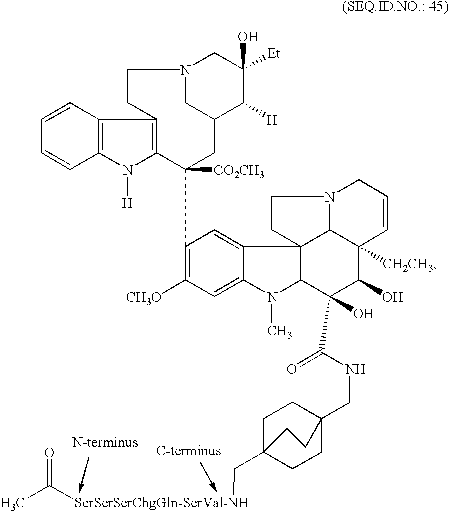

Step A: N-Acetyl-Ser-Ser-Ser-Chg-Gln-Ser-Val-PAM Resin (SEQ.ID.NO.: 45)

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH; and acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-Acetyl-Ser-Ser-Ser-Chg-Gln-Ser-Val-OH (SEQ.ID.NO.: 45)

Three 0.5-mmol runs of the above peptide-resin (3.5 g) were combined and treated with liquid HF (65 ml) for 1.5 hr at 0° C. in the presence of anisole (6 ml). After evaporation of the HF, the residue was washed with ether, filtered and leached with 150 ml of DMF in several portions, adding DIEA to pH ˜8, followed by removal of the DMF in vacuo to a volume of 100 ml. The concentration was determined as ca. 11.7 mg/ml (by weighing the dried resin before and after leaching. The sample purity was determined as 96% by HPLC. The solution was used directly for conjugation with BDAM-(dAc)vinblastine.

Step C: 4-Des-acetylvinblastine-23-(N-Acetyl-Ser-Ser-Ser-Chg-Gln-Ser-Val-BDAM) Amide Acetate Salt (SEQ.ID.NO.: 45)

To 58 ml (equivalent to 0.875 mmol of peptide) of the solution from step B was added 530 mg (0.520 mmol) of BDAM-(dAc)vinblastine, prepared as described in Example 30, Step F, under N₂, cooling to 0° C., and the pH was adjusted to −8 (moistened 5-10 range pH paper) with DIEA. Then 0.134 ml (0.62 mmol) of DPPA was added, followed by stirring at 0-5° C. until completion of the coupling as monitored by analytical HPLC (A=0.1% TFA/H₂O; B=0.1% TFA/CH₃CN), maintaining the pH at >7 by periodic addition of DIEA. After 24 hr, the reaction was worked up by addition of 10 ml of H₂O, stirring 1 hr and concentration to small volume in vacuo, then dissolution in ca. 100 ml of 10% HOAc/5% CH₃CN, adjustment of the pH to 5 with NH₄HCO₃, filtration to remove insolubles, and preparative HPLC in 3 portions on a Waters C4 Delta-Pak column 15 μM 300A (A=0.1% NH₄HCO₃/H₂O; B=CH₃CN), gradient elution 95-->40% A/70 min. Fractions from each run containing product were pooled, acidified to pH 3 with glacial HOAc, concentrated in vacuo to a volume of ˜50 ml, and purified by preparative HPLC on a Waters C18 Delta-Pak column 15 μM 300A (A=0.1% TFA/H₂O; B=0.1% TFA/CH₃CN), gradient elution 95-->70% A/60 min, isocratic 70%/20 min. Homogeneous fractions (evaluated by HPLC, system A, 95-->50% A) from all three runs were pooled and concentrated to a volume of ˜100 ml., diluted with 5% CH₃CN, and passed through AG4X4 ion exchange resin (acetate cycle), followed by freeze-drying to give the title compound as a lyophilized powder.

| HPLC conditions, system A: | |
|---|---|
| Column . . . | Vydac 15 cm #218TP5415, C18 |
| Eluant . . . | Gradient (A --> B) over 45 min. |
| | A = 0.1% TFA/H₂O, B = 0.1% |
| | TFA/acetonitrile |
| Flow . . . | 1.5 ml/min. |

Retention times: BDAM (dAc) vinblastine 23.5 min.

N-Acetyl-Ser-Ser-Ser-Chg-Gln-Ser-Val-OH 14.5 min.

4-Des-acetylvinblastine-23-(N-Acetyl-Ser-Ser-Ser-Chg-Gln-Ser-Val-BDAM) amide 29.5 min.

High Resolution ES/FT-MS: 1662.03

Amino Acid Compositional Analysis¹ (theory/found):

| | | | |
|---|---|---|---|
| ²Ser 4/3.6 | ³Glu 1/2.10 | ⁴Val 1/0.7 | Chg 1/0.95 |

| | | |
|---|---|---|
| Peptide content | 0.504 | μmol/mg |

Note:
¹20 hr, 100° C., 6N HCl
²Uncorrected
³Gln converted to Glu
⁴Incomplete hydrolysis

### Example 32

Preparation of 4-des-Acetylvinblastine-23-(N-methoxy-diethylene-oxyacetyl-4-trans-L-Hyp-Ser-Ser-Chg-Gln-Ser-Val-BDAM) Amide Acetate Salt (SEQ.ID.NO.: 46)

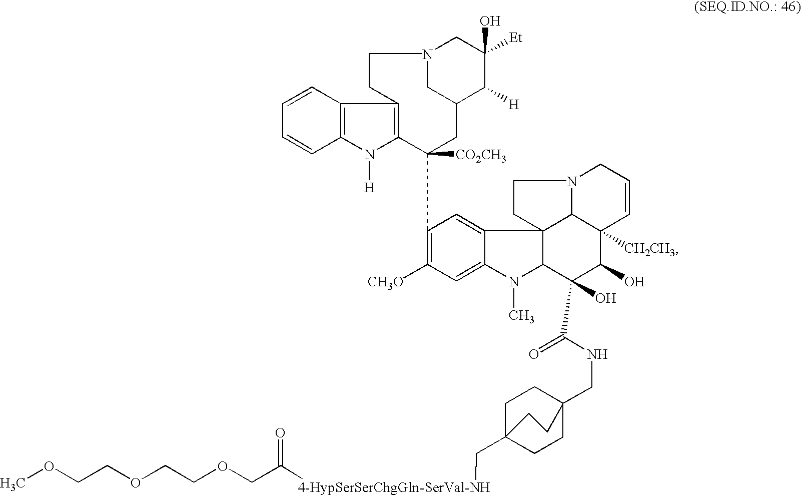

Step A: N-methoxydiethyleneoxyacetyl-4-trans-L-Hyp-Ser-Ser-Chg-Gln-Ser-Val-PAM Resin (SEQ.ID.NO.: 46)

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH, Boc-4-trans-Hyp(Bzl)-OH; and 2-[2-(2-methoxyethoxy)-ethoxy]acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-methoxydiethyleneoxyacetyl-4-trans-L-Hyp-Ser-Ser-Chg-Gln-Ser-Val-OH (SEQ.ID.NO.: 46)

Two 0.5-mmol runs of the above peptide-resin (2.4 g) were combined and treated with liquid HF (40 ml) for 1.5 hr at 0° C. in the presence of anisole (4 ml). After evaporation of the HF, the residue was washed with ether, filtered and leached with 150 ml of H₂O in several portions, followed by preparative HPLC on a Waters C18 Delta-Pak column 15 μM 100 Å (A=0.1% TFA/H₂O; B=0.1% TFA/CH₃CN), gradient elution 95-->70% A/70 min, and pooling of homogeneous fractions and freeze drying to give the title compound as lyophilized powder. The sample purity was determined as 99% by HPLC.

Step C: 4-des-Acetylvinblastine-23-(N-methoxydiethylene-oxyacetyl-4-trans-L-Hyp-Ser-Ser-Chg-Gln-Ser-Val-BDAM) Amide Acetate Salt (SEQ.ID.NO.: 46)

Samples of 440 mg (0.47 mmol) of the peptide from step B and 340 mg (0.33 mmol) of BDAM-(dAc)vinblastine, prepared as described in Example 30, Step F, were dissolved in 25 ml of DMF under N₂, cooling to 0° C. Then 85 mg (0.63 mmol) of 1-hydroxy-7-azabenzotriazole (HOAt) was added, and the pH was adjusted to 6.5-7 (moistened 5-10 range pH paper) with 2,4,6-collidine, followed by addition of 117 mg (0.61 mmol) of 1-(3-dimethylaminopropyl)-3-ethylcarbodiimide hydrochloride (EDC). Stirring was continued at 0-5° C. until completion of the coupling as monitored by analytical HPLC (A=0.1% TFA/H₂O; B=0.1% TFA/CH₃CN), maintaining the pH at 6.5-7 by periodic addition of 2,4,6-collidine. After 3 hr the reaction was worked up by addition of ˜10 ml of H₂O, stirring 1 hr and concentration to small volume in vacuo, then dissolution in ca. 70 ml of 5% HOAc. and preparative HPLC on a Waters C18 Delta-Pak column 15 μM 300A (A=0.1% TFA/H₂O; B=0.1% TFA/CH₃CN), gradient elution 95-->40% A/70 min). Homogeneous fractions (evaluated by HPLC, system A, 95-->50% A) from all three runs were pooled and concentrated to a volume of −50 ml and passed through AG4X4 ion exchange resin (acetate cycle), followed by freeze-drying to give the title compound as a lyophilized powder.

HPLC conditions, system A:

| | |
|---|---|
| Column . . . | Vydac 15 cm #218TP5415, |
| | C18 |
| Eluant . . . | Gradient (A --> B) over |
| | 45 min. A = 0.1% |
| | TFA/H₂O, B = 0.1% |
| | TFA/acetonitrile |
| Flow . . . | 1.5 ml/min. |
| Retention times: BDAM (dAc) vinblastine | 23.5 min. |
| N-methoxydiethyleneoxyacetyl-4-trans-L- | 16.2 min. |
| Hyp-Ser-Ser-Chg-Gln-Ser-Val-OH | |
| 4-des- Acetylvinblastine-23- | 29.6 min. |
| (N-methoxydiethyleneoxyacetyl-4-trans-L- | |
| Hyp-Ser-Ser-Chg-Gln-Ser-Val-BDAM) | |
| amide | |
| High Resolution ES/FT-MS: | 1805.95 |
| Amino Acid Compositional | ²Ser3/1.7 ³Glu 1/1.01 |
| Analysis¹ (theory/found): | ⁴Val 1/0.93 |
| | Chg 1/0.98 Hyp 1/1.01 |
| | Peptide content = |
| | 0.497 μmol/mg |

Note:
¹20 hr, 100° C., 6N HCl
²Uncorrected
³Gln converted to Glu
⁴Incomplete hydrolysis

### Example 33

Preparation of 4-des-Acetylvinblastine-23-(N-Acetyl-4-trans-L-Hyp-Ser-Ser-Chg-Gln-Ser-HCAP) Amide Acetate Salt (2-7) (SEQ.ID.NO.: 62)

Step A: N-Acetyl-4-trans-L-Hyp-Ser-Ser-Chg-Gln-OH (2-1) (SEQ.ID.NO. 63)

Starting with 0.5 mmole (0.80 g) of Fmoc-Gln(Trt)-Wang resin, the protected peptide was synthesized on a ABI model 430A peptide synthesizer. The protocol used a 4-fold excess (2.0 mmol) of each of the following protected amino acids: Fmoc-Ser(tBu)—OH, Fmoc-Chg-OH, Fmoc-4-trans-Hyp(tBu)—OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95%TFA:2.5% H₂O:2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid-aqueous acetonitrile solvent systems using 100-70%A, 60 min linear gradient. Fractions containing product of at least 99% (HPLC) purity were combined to give the title compound.

FABMS: 615.3

Peptide Content: 1.03 mmole/mg.

HPLC: 99% pure @214 nm, retention time=10.16 min, (Vydac C18, gradient of 95%A/B to 50%A/B over 30 min, A=0.1%TFA-H₂O,

B=0.1%TFA-CH₃CN)

In a similar manner the following compound was prepared:

N-hydroxyacetyl-Abu-Ser-Ser-Chg-Gln-Ser-OH (3-1) (SEQ.ID.NO. 64)

Step B: N-Boc-(1S,2R)-(+)-Norephedrine (2-2)

A solution of 1.51 g (10 mmol) of (1S,2R)-(+)-Norephedrine in a mixture of 1,4 dioxane (20 ml), water (10 ml) and 1N NaOH (10 ml) was stirred and cooled in an ice-water bath. Di-(t-butyl) dicarbonate (2.4 g, 11 mmol) was added in portions over approx. 20 min. The reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with 1N KHSO₄. The aqueous phase was extracted 2× with EtOAc. The combined extracts were washed with water, brine and were concentrated and dried to provide the desired product as a white crystalline solid (2-2). FABMS: 252

Step C: N-Boc-HCAP (2-3)

A solution of 2.38 g of N-Boc-(1S,2R)-(+)-Norephedrine (2-2) in 50 ml acetic acid/10 ml H₂O was hydrogenated at 60 psi on a Parr apparatus over 500 mg of Ir black catalyst for 24 hrs. The reaction was filtered through a Celite pad, and the filtrate concentrated in vacuo to give a tan foam (2-3). FABMS: 258.2

Step D: N-Benzyloxycarbonyl-Ser-N-t-Boc-HCAP Ester (2-4)

A solution of 1.95 g (6.6 mmol) of N-Z-Ser(tBu)—OH, 1.54 g (6.0 mmol) of N-Boc-HCAP (2-3), 1.26 g (6.6 mmol) of EDC, and 146 mg (1.2 mmol) of DMAP in 30 ml of anh. CH₂Cl₂ was treated and the resulting solution stirred at room temp. in an N₂ atmosphere for 12 h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product (2-4).

In a similar manner the following compound was prepared:

N-Benzyloxycarbonyl-Pro-N-t-Boc-HCAP ester (3-2) by coupling of N-Z-Pro-OH with N-Boc-HCAP alcohol (2-3)

Step E: H-Ser(tBu)—N-t-Boc-HCAP Ester (2-5)

A 2.0 g of (2-4) in a solution of 90 ml EtOH, 20 ml water, and 10 ml acetic acid was hydrogenated on a Parr apparatus at 50 psi over 200 mg of Pd(OH)₂ catalyst for 3 h. The reaction was filtered through a Celite pad, and the filtrate wasconcentrated to small volume in vacuo, then purified by preparatory HPLC on a Delta-Pak C18 column with 0.1% trifluoroacetic acid-aqueous acetonitrile solvent systems using 95-50%A, 60 min linear gradient. Fractions containing product of at least 99% (HPLC) purity were combined to give the intermediate (2-5). FABMS: 401.3

In a similar manner the following compound was prepared:

H-Pro-N-t-Boc-HCAP ester (3-3) by hydrogenation of N-Benzyloxycarbonyl-Pro-N-t-Boc-HCAP ester (3-2)

Step F: N-Acetyl-4-trans-L-Hyp-Ser-Ser-Chg-Gln-Ser-HCAP Amine (2-6) (SEQ.ID.NO. 62)

A solution of 614 mg (1.0 mmol) of N-Acetyl-4-trans-L Hyp-Ser-Ser-Chg-Gln-OH (2-1), 400 mg (1.0 mmol) of H-Ser(tBu)—N-t-Boc-HCAP ester (2-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 h. The solvent was removed in vacuo, the residue was washed with ether and dried. The crude product was treated with 95%TFA:5% H₂O (20 ml) for 2 hr at r.t. under argon. After evaporation of the TFA, the residue was purified by preparatory HPLC on a Delta-Pak C18 column with 0.1% trifluoroacetic acid-aqueous acetonitrile solvent systems using 95-50%A, 60 min linear gradient. Fractions containing product of at least 99% (HPLC) purity were combined to give the intermediate compound (2-6).

FABMS: 841.8

Peptide Content: 863.39 NMole/mg.

HPLC: 99% pure @214 nm, retention time=13.7 min, (Vydac C18, gradient of 95%A/B to 5%A/B over 30 min, A=0.1%TFA-H₂O, B=0.1%TFA-CH₃CN)

In a similar manner the following compound was prepared:

N-Hydroxyacetyl-Abu-Ser-Ser-Chg-Gln-Ser-Pro-HCAP amine (3-4) (SEQ.ID.NO.65) by coupling of N-Hydroxyacetyl-Abu-Ser-Ser-Chg-Gln-Ser-OH (3-1) with H-Pro-N-t-Boc-HCAP ester (3-3) followed by deprotection.

Step G: 4-des-Acetylvinblastine-23-(N-Ac-4-trans-L-Hyp-Ser-Ser-Chg-Gln-Ser-HCAP) Amide Acetate Salt (2-7)

A solution of 0.461 of 4-des-acetylvinblastine-23-hydrazide (0.6 mmol) in 10 ml DMF cooled to −15° C. under Argon, was converted to the azide in situ by acidification with 4M HCl in dioxane to pH<1.5 (moistened 0-2.5 range paper), followed by addition of 0.105 ml (1.3 equiv) of isoamyl nitrite and stirring for 1 hr at 10-15° C. The pH was brought to 7 by the addition of DIEA, and 555 mg (0.66 mmol) of amine derivative (2-6) from step F was then added, and the reaction was stirred at 0° C. for 24 hrs, and purified by preparatory HPLC on a 15 μM, 100A, Delta-Pak C18 column with 0.1% trifluoroacetic acid-aqueous acetonitrile solvent systems using 95-50%A, 60 min linear gradient. Homogeneous fractions were pooled and concentrated in vacuo, followed by freeze-drying to give the title compound as the TFA salt which was converted to the corresponding HOAc salt by AG 4×4 resin (100-200 mesh, free base form, BIO-RAD) (2-7).

ES⁺: 1576.7

Peptide Content: 461.81 NMole/mg.

Ser 3.04; Hyp 1.07; Chg 1.02; Glu 1.00

HPLC: 99% pure @214 nm, retention time=18.31 min, (Vydac C18, gradient of 95%A/B to 5%A/B over 30 min, A=0.1%TFA-H₂O, B=0.1%TFA-CH₃CN)

In a similar manner the following compound was prepared:

4-des-Acetylvinblastine-23-(N-hydroxyacetyl-Abu-Ser-Ser-Chg-Gln-Ser-Pro-HCAP) amide (3-5) (SEQ.ID.NO.: 64) by coupling 4-des-Acetylvinblastine-23-hydrazide (1-1) with OH-Acetyl-Abu-Ser-Ser-Chg-Gln-Ser-Pro-HCAP amine (3-4)

4des-Acetylvinblastine-23-(N-hydroxyl-Ac-Abu-Ser-Ser-Chg-Gln-Ser-HCAP) Amide Acetate Salt (3-5)

ES⁺: 1661.9

Peptide Content: 499.87 NMole/mg.

Ser 2.98; Abu 1.01; Chg 1.02; Glu 1.00; Pro 0.98

HPLC: 99% pure @214 nm, retention time=18.83 min, (Vydac C18, gradient of 95%A/B to 5%A/B over 30 min, A=0.1%T-FA-H₂O, B=0.1%TFA-CH₃CN)

### Example 34

Preparation of 4-des-Acetylvinblastine-23-(N-Acetyl-Ser-Chg-Gln-Ser-Ser-Pro-HCAP) Amide Acetate Salt (2A-7) (SEQ.ID.NO.: 66)

Step A: N-Acetyl-Ser-Chg-Gln-Ser-Ser-OH (2A-1) (SEQ.ID.NO.: 67)

Starting with 0.5 mmole (0.80 g) of Fmoc-Ser(tBu)-Wang resin, the protected peptide was synthesized on a ABI model 430A peptide synthesizer. The protocol used a 4-fold excess (2.0 mmol) of each of the following protected amino acids: Fmoc-Ser(tBu)—OH, Fmoc-Gln-OH, Fmoc-Chg-OH, Fmoc-Ser(tBu)—OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95%TFA:2.5% H2O:2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid-aqueous acetonitrile solvent systems using 100-70%A, 60 min linear gradient. Fractions containing product of at least 99% (HPLC) purity were combined to give the title compound.

FABMS: 589.5

Peptide Content: 1.01 NMole/mg.

HPLC: 99% pure @214 nm, retention time=10.7 min, (Vydac C18, gradient of 95%A/B to 50%A/B over 30 min, A=0.1%TFA-H₂O, B=0.1%TFA-CH₃CN)

Step B: N-Boc-(1S,2R)-(+)-Norephedrine (2A-2)

A solution of 1.51 g (10 mmol) of (1S,2R)-(+)-Norephedrine in a mixture of 1,4 dioxane (20 ml), water (10 ml) and 1N NaOH (10 ml) is stirred and cooled in an ice-water bath. Di-(t-butyl) dicarbonate (2.4 g, 11 mmol) was added in portions over approx. 20 min. IThe reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with 1N KHSO₄. The aqueous phase was extracted 2× with EtOAc. The combined extracts were washed with water, brine and were concentrated and dried to provide the desired product as a white crystalline solid. FABMS: 252

Step C: N-Boc-HCAP (2A-3)

A solution of 2.38 g of N-Boc-(1S,2R)-(+)-Norephedrine (2A-2) in 50 ml acetic acid/10 ml H₂O was hydrogenated at 60 psi on a Parr apparatus over 500 mg of Ir black catalyst for 24 hrs. The reaction was filtered through a Celite pad, and the filtrate concentrated in vacuo to give a tan foam. FABMS: 258.2

Step D: N-Benzyloxycarbonyl-Pro-N-t-Boc-HCAP Ester (2A-4)

A solution of 1.62 g (6.6 mmol) of N-Z-Pro-OH, 1.54 g (6.0 mmol) of N-Boc-HCAP (2A-3), 1.26 g (6.6 mmol) of EDC, and 146 mg (1.2 mmol) of DMAP in 30 ml of anh. CH₂Cl₂ was treated and the resulting solution stirred at room temp. in an N₂ atmosphere for 12 h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product.

Step E: H-Pro-N-t-Boc-HCAP Ester (2A-5)

A 2.0 g of (2A-4) in a solution of 90 ml EtOH, 20 ml water, and 10 ml acetic acid was hydrogenated on a Parr apparatus at 50 psi over 200 mg of Pd(OH)₂ catalyst for 3 h. The reaction was filtered through a Celite pad, and the filtrate was concentrated to small volume in vacuo, then purified by preparatory HPLC on a Delta-Pak C18 column with 0.1% trifluoroacetic acid-aqueous acetonitrile solvent systems using 95-50%A, 60 min linear gradient. Fractions containing product of at least 99% (HPLC) purity were combined to give the title compound (2A-5). FABMS: 356.3

Step F: N-Acetyl-Ser-Chg-Gln-Ser-Ser-Pro-HCAP Amine (2A-6) (SEQ.ID.NO.: 65)

A solution of 589 mg (1.0 mmol) of N-Acetyl-Ser-Chg-Gln-Ser-Ser-OH (2-1), 356 mg (1.0 mmol) of H-Pro-N-t-Boc-HCAP ester (2A-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 h. The solvent was removed in vacuo, the residue was washed with ether and dried. The crude product was treated with 95%TFA:5% H₂O (20 ml) for 2 hr at r.t. under argon. After evaporation of the TFA, the residue was purified by preparatory HPLC on a Delta-Pak C18 column with 0.1% trifluoroacetic acid-aqueous acetonitrile solvent systems using 95-50%A, 60 min linear gradient. Fractions containing product of at least 99% (HPLC) purity were combined to give the title compound (2-6).

FABMS: 825.5

Peptide Content: 893.6 NMole/mg.

HPLC: 99% pure @214 nm, retention time=15.2 min, (Vydac C18, gradient of 95%A/B to 5%A/B over 30 min, A=0.1%TFA-H₂O, B=0.1%TFA-CH₃CN)

Step G: 4-des-Acetylvinblastine-23-(N-Ac-Ser-Chg-Gln-Ser-Ser-Pro-HCAP) Amide Acetate Salt (2A-7) (SEQ.ID.NO.: 66)

A solution of 0.461 of 4-des-acetylvinblastine-23-hydrazide (0.6 mmol) in 10 ml DMF cooled to −15° C. under Argon, was converted to the azide in situ by acidification with 4M HCl in dioxane to pH<1.5 (moistened 0-2.5 range paper), followed by addition of 0.105 ml (1.3 equiv) of isoamyl nitrite and stirring for 1 hr at 10-15° C. The pH was brought to 7 by the addition of DIEA, and 545 mg (0.66 mmol) of amine derivative (2A-6) from step F was then added, and the reaction was stirred at 0° C. for 24 hrs, and purified by preparatory HPLC on a 15 μM, 100A, Delta-Pak C18 column with 0.1% trifluoroacetic acid-aqueous acetonitrile solvent systems using 95-50%A, 60 min linear gradient. Homogeneous fractions were pooled and concentrated in vacuo, followed by freeze-drying to give the title compound as the TFA salt which was converted to title compound by AG 4×4 resin (100-200 mesh, free base form, BIO-RAD) (2A-7)

ES⁺: 1560.9

Peptide Content: 586.8 NMole/mg.

Ser 3.04; Chg 1.01; Glu 1.00; Pro 0.97

HPLC: 99% pure @214 nm, retention time=13.4 min, (Vydac C18, gradient of 95%A/B to 5%A/B over 30 min, A=0.1%TFA-H₂O, B=0.1%TFA-CH₃CN)

Biological Assays.

The ability of the compounds useful in the methods of the present invention of the present invention to inhibit prenyl protein transferases can be demonstrated using the following assays.

### Example 35

In vitro Inhibition of Ras Farnesyl Transferase

Transferase Assays. Isoprenyl-protein transferase activity assays are carried out at 30° C. unless noted otherwise. A typical reaction contains (in a final volume of 50 μL): [³H]farnesyl diphosphate, Ras protein, 50 mM HEPES, pH 7.5, 5 mM MgCl₂, 5 mM dithiothreitol, 10 μM ZnCl₂, 0.1% polyethyleneglycol (PEG) (15,000-20,000 mw) and isoprenyl-protein transferase. The FPTase employed in the assay is prepared by recombinant expression as described in Omer, C. A., Kral, A. M., Diehl, R. E., Prendergast, G. C., Powers, S., Allen, C. M., Gibbs, J. B. and Kohl, N. E. (1993) Biochemistry 32:5167-5176. After thermally pre-equilibrating the assay mixture in the absence of enzyme, reactions are initiated by the addition of isoprenyl-protein transferase and stopped at timed intervals (typically 15 min) by the addition of 1 M HCl in ethanol (1 mL). The quenched reactions are allowed to stand for 15 m (to complete the precipitation process). After adding 2 mL of 100% ethanol, the reactions are vacuum-filtered through Whatman GF/C filters. Filters are washed four times with 2 mL aliquots of 100% ethanol, mixed with scintillation fluid (10 mL) and then counted in a Beckman LS3801 scintillation counter.

For inhibition studies, assays are run as described above, except test compounds or compositions are prepared as concentrated solutions in 100% dimethyl sulfoxide and then diluted 20-fold into the enzyme assay mixture. Substrate concentrations for inhibitor IC₅₀ determinations are as follows: FTase, 650 nM Ras-CVLS (SEQ.ID.NO.: 1), 100 nM farnesyl diphosphate.

The compounds of the instant invention described in the above Examples 1-24E are tested for inhibitory activity against human FPTase by the assay described above.

### Example 36

Modified in vitro GGTase Inhibition Assay

The modified geranylgeranyl-protein transferase inhibition assay is carried out at room temperature. A typical reaction contains (in a final volume of 50 μL): [³H]geranylgeranyl diphosphate, biotinylated Ras peptide, 50 mM HEPES, pH 7.5, a modulating anion (for example 10 mM glycerophosphate or 5 mM ATP), 5 mM MgCl₂, 10 μM ZnCl₂, 0.1% PEG (15,000-20,000 mw), 2 mM dithiothreitol, and geranylgeranylprotein transferase type I (GGTase). The GGTase-type I enzyme employed in the assay is prepared as described in U.S. Pat. No. 5,470,832, incorporated by reference. The Ras peptide is derived from the K4B-Ras protein and has the following sequence: biotinyl-GKKKKKKSKTKCVIM (single amino acid code) (SEQ.ID.NO.:68). Reactions are initiated by the addition of GGTase and stopped at timed intervals (typically 15 min) by the addition of 200 μL of a 3 mg/mL suspension of streptavidin SPA beads (Scintilla-tion Proximity Assay beads, Amersham) in 0.2 M sodium phosphate, pH 4, containing 50 mM EDTA, and 0.5% BSA. The quenched reactions are allowed to stand for 2 hours before analysis on a Packard TopCount scintillation counter.

For inhibition studies, assays are run as described above, except test compounds or compositions are prepared as concentrated solutions in 100% dimethyl sulfoxide and then diluted 25-fold into the enzyme assay mixture. IC₅₀ values are determined with Ras peptide near KM concentrations. Enzyme and substrate concentrations for inhibitor IC₅₀ determinations are as follows: 75 pM GGTase-1,1.6 μM Ras peptide, 100 nM geranylgeranyl diphosphate.

The compounds of the instant invention described in the above Examples 1-24E are tested for inhibitory activity against human GGTase type I by the assay described above.

### Example 37

Cell-Based in vitro Ras Farnesylation Assay

The cell line used in this assay is a v-ras line derived from either Rat1 or NIH₃T3 cells, which expressed viral Ha-ras p²1. The assay is performed essentially as described in DeClue, J. E. et al., Cancer Research 51:712-717, (1991). Cells in 10 cm dishes at 50-75% confluency are treated with the test compound or composition (final concentration of solvent, methanol or dimethyl sulfoxide, is 0.1%). After 4 hours at 37° C., the cells are labeled in 3 ml methionine-free DMEM supple-mented with 10% regular DMEM, 2% fetal bovine serum and 400 μCi[3⁵S]methionine (1000 Ci/mmol). After an additional 20 hours, the cells are lysed in 1 ml lysis buffer (1% NP40/20 mM HEPES, pH 7.5/5 mM MgCl₂/1 mM DTT/10 mg/ml aprotinen/2 mg/ml leupeptin/2 mg/ml antipain/0.5 mM PMSF) and the lysates cleared by centrifugation at 100,000× g for 45 min. Aliquots of lysates containing equal numbers of acid-precipitable counts are bought to 1 ml with IP buffer (lysis buffer lacking DTT) and immunoprecipitated with the ras-specific monoclonal antibody Y13-259 (Furth, M. E. et al., J. Virol. 43:294-304, (1982)). Following a 2 hour antibody incubation at 4° C., 200 μl of a 25% suspension of protein A-Sepharose coated with rabbit anti rat IgG is added for 45 min. The immunoprecipitates are washed four times with IP buffer (20 nM HEPES, pH 7.5/1 mM EDTA/1% Triton X-100.0.5% deoxycholate/0.1%/SDS/0.1 M NaCl) boiled in SDS-PAGE sample buffer and loaded on 13% acrylamide gels. When the dye front reached the bottom, the gel is fixed, soaked in Enlightening, dried and autoradio-graphed. The intensities of the bands corresponding to farnesylated and nonfarnesylated ras proteins are compared to determine the percent inhibition of farnesyl transfer to protein.

### Example 38

Cell-Based in vitro Growth Inhibition Assay

To determine the biological consequences of FPTase inhibition, the effect of the instant compositions and the compounds useful in the instant invention on the anchorage-independent growth of Rat1 cells transformed with either a v-ras, v-raf, or v-mos oncogene is tested. Cells transformed by v-Raf and v-Mos maybe included in the analysis to evaluate the specificity of instant compounds for Ras-induced cell transformation.

Rat 1 cells transformed with either v-ras, v-raf, or v-mos are seeded at a density of 1×10⁴ cells per plate (35 mm in diameter) in a 0.3% top agarose layer in medium A (Dulbecco's modified Eagle's medium supplemented with 10% fetal bovine serum) over a bottom agarose layer (0.6%). Both layers contain 0.1% methanol or an appropriate concentration of the test compound or composition (dissolved in methanol at 1000 times the final concentration used in the assay). The cells are fed twice weekly with 0.5 ml of medium A containing 0.1% methanol or the concentration of the instant compound. Photomicrographs are taken 16 days after the cultures are seeded and comparisons are made.

### Example 39

Construction of SEAP Reporter Plasmid pDSE100

The SEAP reporter plasmid, PDSE10O was constructed by ligating a restriction fragment containing the SEAP coding sequence into the plasmid pCMV-RE-AKI. The SEAP gene is derived from the plasmid pSEAP2-Basic (Clontech, Palo Alto, Calif.). The plasmid pCMV-RE-AKI contains 5 sequential copies of the ‘dyad symmetry response element’ cloned upstream of a ‘CAT-TATA’ sequence derived from the cytomegalovirus immediate early promoter. The plasmid also contains a bovine growth hormone poly-A sequence.

The plasmid, pDSE100 was constructed as follows. A restriction fragment encoding the SEAP coding sequence was cut out of the plasmid pSEAP2-Basic using the restriction enzymes EcoRI and HpaI. The ends of the linear DNA fragments were filled in with the Klenow fragment of E. coli DNA Polymerase I. The ‘blunt ended’ DNA containing the SEAP gene was isolated by electrophoresing the digest in an agarose gel and cutting out the 1694 base pair fragment. The vector plasmid pCMV-RE-AKI was linearized with the restriction enzyme Bgl-II and the ends filled in with Klenow DNA Polymerase I. The SEAP DNA fragment was blunt end ligated into the pCMV-RE-AKI vector and the ligation products were transformed into DH5-alpha E. coli cells (Gibco-BRL). Transformants were screened for the proper insert and then mapped for restriction fragment orientation. Properly oriented recombinant constructs were sequenced across the cloning junctions to verify the correct sequence. The resulting plasmid contains the SEAP coding sequence downstream of the DSE and CAT-TATA promoter elements and upstream of the BGH poly-A sequence.

Alternative Construction of SEAP Reporter Plasmid, pDSE101

The SEAP repotrer plasmid, pDSE11 is also constructed by ligating a restriction fragment containing the SEAP coding sequence into the plasmid pCMV-RE-AKI. The SEAP gene is derived from plasmid pGEM7zf(−)/SEAP.

The plasmid pDSE101 was constructed as follows: A restriction fragment containing part of the SEAP gene coding sequence was cut out of the plasmid pGEM7zf(−)/SEAP using the restriction enzymes Apa I and KpnI. The ends of the linear DNA fragments were chewed back with the Klenow fragment of E. coli DNA Polymerase I. The “blunt ended” DNA containing the truncated SEAP gene was isolated by electrophoresing the digest in an agarose gel and cutting out the 1910 base pair fragment. This 1910 base pair fragment was ligated into the plasmid pCMV-RE-AKI which had been cut with Bgl-II and filled in with E. coli Klenow fragment DNA polymerase. Recombinant plasmids were screened for insert orientation and sequenced through the ligated junctions. The plasmid pCMV-RE-AKI is derived from plasmid pCMVIE-AKI-DHFR (Whang, Y., Silberklang, M., Morgan, A., Munshi, S., Lenny, A. B., Ellis, R. W., and Kieff, E. (1987) J. Virol., 61, 1796-1807) by removing an EcoRI fragment containing the DHFR and Neomycin markers. Five copies of the fos promoter serum response element were inserted as described previously (Jones, R. E., Defeo-Jones, D., McAvoy, E. M., Vuocolo, G. A., Wegrzyn, R. J., Haskell, K. M. and Oliff, A. (1991) Oncogene, 6, 745-751) to create plasmid pCMV-RE-AKI.

The plasmid pGEM7zf(−)/SEAP was constructed as follows. The SEAP gene was PCRed, in two segments from a human placenta cDNA library (Clontech) using the following oligos.

| | |
|---|---|
| Sense strand N-terminal SEAP: | |
| 5′GAGAGGGAATTCGGGCCCTTCCTGCAT | (SEQ.ID.NO.:69) |
| GCTGCTGCTGCTGCTGCTGCTGGGC 3′ | |
| Antisense strand N-terminal SEAP: | |
| 5′GAGAGAGCTCGAGGTTAACCCGGGT | (SEQ.ID.NO.:70) |
| GCGCGGCGTCGGTGGT 3′ | |
| Sense strand C-terminal SEAP: | |
| 5′GAGAGAGTCTAGAGTTAACCCGTGGTCC | (SEQ.ID.NO.:71) |
| CCGCGTTGCTTCCT 3′ | |
| Antisense strand C-terminal SEAP: | |
| 5′GAAGAGGAAGCTTGGTACCGCCACTG | (SEQ.ID.NO.:72) |
| GGCTGTAGGTGGTGGCT 3′ | |

The N-terminal oligos (SEQ.ID.NO.: 4 and SEQ.ID.NO.: 5) were used to generate a 1560 bp N-terminal PCR product that contained EcoRI and HpaI restriction sites at the ends. The Antisense N-terminal oligo (SEQ.ID.NO.: 4) introduces an internal translation STOP codon within the SEAP gene along with the HpaI site. The C-terminal oligos (SEQ.ID.NO.: 5 and SEQ.ID.NO.: 6) were used to amplify a 412 bp C-terminal PCR product containing HpaI and HindIII restriction sites. The sense strand C-terminal oligo (SEQ.ID.NO.: 5) introduces the internal STOP codon as well as the HpaI site. Next, the N-terminal amplicon was digested with EcoRI and HpaI while the C-terminal amplicon was digested with HpaI and HindIII. The two fragments comprising each end of the SEAP gene were isolated by electrophoresing the digest in an agarose gel and isolating the 1560 and 412 base pair fragments. These two fragments were then co-ligated into the vector pGEM7zf(−) (Promega) which had been restriction digested with EcoRI and HindIII and isolated on an agarose gel. The resulting clone, pGEM7zf(−)/SEAP contains the coding sequence for the SEAP gene from amino acids.

Construction of a Constitutively Expressing SEAP Plasmid pCMV-SEAP

An expression plasmid constitutively expressing the SEAP protein was created by placing the sequence encoding a truncated SEAP gene downstream of the cytomegalovirus (CMV) IE-1 promoter. The expression plasmid also includes the CMV intron A region 5′ to the SEAP gene as well as the 3′ untranslated region of the bovine growth hormone gene 3′ to the SEAP gene.

The plasmid pCMVIE-AKI-DHFR (Whang et al, 1987) containing the CMV immediate early promoter was cut with EcoRI generating two fragments. The vector fragment was isolated by agarose electrophoresis and religated. The resulting plasmid is named pCMV-AKI. Next, the cytomegalovirus intron A nucleotide sequence was inserted downstream of the CMV IE1 promter in pCMV-AKI. The intron A sequence was isolated from a genomic clone bank and sub-cloned into pBR³²² to generate plasmid p16T-286. The intron A sequence was mutated at nucleotide 1856 (nucleotide numbering as in Chapman, B. S., Thayer, R. M., Vincent, K. A. and Haigwood, N. L., Nuc.Acids Res. 19, 3979-3986) to remove a SacI restriction site using site directed mutagenesis. The mutated intron A sequence was PCRed from the plasmid p16T-287 using the following oligos.

| | |
|---|---|
| Sense strand: | |
| 5′ GGCAGAGCTCGTTTAGTGAACCGTCAG 3′ | (SEQ.ID.NO.:73) |
| Antisense strand: | |
| 5′ GAGAGATCTCAAGGACGGTGACTGCAG 3′ | (SEQ.ID.NO.:74) |

These two oligos generate a 991 base pair fragment with a SacI site incorporated by the sense oligo and a Bgl-II fragment incorporated by the antisense oligo. The PCR fragment is trimmed with SacI and Bgl-II and isolated on an agarose gel. The vector pCMV-AKI is cut with SacI and Bgl-II and the larger vector fragment isolated by agarose gel electrophoresis. The two gel isolated fragments are ligated at their respective SacI and Bgl-II sites to create plasmid pCMV-AKI-InA.

The DNA sequence encoding the truncated SEAP gene is inserted into the pCMV-AKI-InA plasmid at the Bgl-II site of the vector. The SEAP gene is cut out of plasmid pGEM7zf(−)/SEAP (described above) using EcoRI and HindIII. The fragment is filled in with Klenow DNA polymerase and the 1970 base pair fragment isolated from the vector fragment by agarose gel electrophoresis. The pCMV-AKI-InA vector is prepared by digesting with Bgl-II and filling in the ends with Klenow DNA polymerase. The final construct is generated by blunt end ligating the SEAP fragment into the pCMV-AKI-InA vector. Transformants were screened for the proper insert and then mapped for restriction fragment orientation. Properly oriented recombinant constructs were sequenced across the cloning junctions to verify the correct sequence. The resulting plasmid, named pCMV-SEAP, contains a modified SEAP sequence downstream of the cytomegalovirus immediately early promoter IE-1 and intron A sequence and upstream of the bovine growth hormone poly-A sequence. The plasmid expresses SEAP in a constitutive manner when transfected into mammalian cells.

Cloning of a Myristylated Viral-H-Ras Expression Plasmid

A DNA fragment containing viral-H-ras can be PCRed from plasmid “H-1” (Ellis R. et al. J. Virol. 36, 408, 1980) or “HB-1” (deposited in the ATCC under Budapest Treaty on Aug. 27, 1997, and designated ATCC 209,218) using the following oligos.

| | | |
|---|---|---|
| Sense strand: | 5′TCTCCTCGAGGCCACCATGGGGAGTAGCAAGAGCAAGCCTAA | (SEQ.ID.NO.:75) |
| | GGACCCCAGCCAGCGCCGGATGACAGAATACAAGCTTGTGGTGG 3′. | |
| Antisense: | 5′CACATCTAGATCAGGACAGCACAGACTTGCAGC 3′. | (SEQ.ID.NO.:76) |

A sequence encoding the first 15 aminoacids of the v-src gene, containing a myristylation site, is incorporated into the sense strand oligo. The sense strand oligo also optimizes the ‘Kozak’ translation initiation sequence immediately 5′ to the ATG start site. To prevent prenylation at the viral-ras C-terminus, cysteine 186 would be mutated to a serine by substituting a G residue for a C residue in the C-terminal antisense oligo. The PCR primer oligos introduce an XhoI site at the 5′ end and a XbaI site at the 3′end. The XhoI-XbaI fragment can be ligated into the mammalian expression plasmid pCI (Promega) cut with XhoI and XbaI. This results in a plasmid in which the recombinant myr-viral-H-ras gene is constitutively transcribed from the CMV promoter of the pCI vector.

Cloning of a Viral-H-Ras-CVLL Expression Plasmid

A viral-H-ras clone with a C-terminal sequence encoding the amino acids CVLL can be cloned from the plasmid “H-1” (Ellis R. et al. J. Virol. 36, 408, 1980) or “HB-1” (deposited in the ATCC under Budapest Treaty on Aug. 27, 1997, and designated ATCC 209,218) by PCR using the following oligos.

| | | |
|---|---|---|
| Sense strand: | 5′TCTCCTCGAGGCCACCATGACAGAATACAAGCTTGTGGTGG-3′ | (SEQ.ID.NO.:77) |
| Antisense strand: | 5′CACTCTAGACTGGTGTCAGAGCAGCACACACTTGCAGC-3′ | (SEQ.ID.NO.:78) |

The sense strand oligo optimizes the ‘Kozak’ sequence and adds an XhoI site. The antisense strand mutates serine 189 to leucine and adds an XbaI site. The PCR fragment can be trimmed with XhoI and XbaI and ligated into the XhoI-XbaI cut vector pCI (Promega). This results in a plasmid in which the mutated viral-H-ras-CVLL gene is constitutively transcribed from the CMV promoter of the pCI vector.

Cloning of c-H-ras-Leu61 Expression Plasmid

The human c-H-ras gene can be PCRed from a human cerebral cortex cDNA library (Clontech) using the following oligonucleotide primers.

| | | |
|---|---|---|
| Sense strand: | 5′-GAGAGAATTCGCCACCATGACGGAATATAAGCTGGTGG-3′ | (SEQ.ID.NO.:79) |
| Antisense strand: | 5′-GAGAGTCGACGCGTCAGGAGAGCACACACTTGC-3′ | (SEQ.ID.NO.:80) |

The primers will amplify a c-H-ras encoding DNA fragment with the primers contributing an optimized ‘Kozak’ translation start sequence, an EcoRI site at the N-terminus and a Sal I stite at the C-terminal end. After trimming the ends of the PCR product with EcoRI and Sal I, the c-H-ras fragment can be hgated ligated into an EcoRI-Sal I cut mutagenesis vector pAlter-1 (Promega). Mutation of glutamine-61 to a leucine can be accomplished using the manufacturer's protocols and the following oligonucleotide:

5′-CCGCCGGCCTGGAGGAGTACAG-3′ (SEQ.ID.NO.: 81)

After selection and sequencing for the correct nucleotide substitution, the mutated c-H-ras-Leu61 can be excised from the pAlter-1 vector, using EcoRI and Sal I, and be directly ligated into the vector pCI (Promega) which has been digested with EcoRI and Sal I. The new recombinant plasmid will constitutively transcribe c-H-ras-Leu61 from the CMV promoter of the pCI vector.

Cloning of a c-N-Ras-Val-12 Expression Plasmid

The human c-N-ras gene can be PCRed from a human cerebral cortex cDNA library (Clontech) using the following oligonucleotide primers.

| | | |
|---|---|---|
| Sense strand: | 5′-GAGAGAATTCGCCACCATGACTGAGTACAAACTGGTGG-3′ | (SEQ.ID.NO.:82) |
| Antisense strand: | 5′-GAGAGTCGACTTGTTACATCACCACACATGGC-3′ | (SEQ.ID.NO.:83) |

The primers will amplify a c-N-ras encoding DNA fragment with the primers contributing an optimized ‘Kozak’ translation start sequence, an EcoRI site at the N-terminus and a Sal I stite at the C-terminal end. After trimming the ends of the PCR product with EcoRI and Sal I, the c-N-ras fragment can be ligated into an EcoRI-Sal I cut mutagenesis vector pAlter-1 (Promega). Mutation of glycine-12 to a valine can be accomplished using the manufacturer's protocols and the following oligonucleotide:

5′-GTTGGAGCAGTTGGTGTTGGG-3′ (SEQ.ID.NO.: 84)

After selection and sequencing for the correct nucleotide substitution, the mutated c-N-ras-Val-12 can be excised from the pAlter-1 vector, using EcoRI and Sal I, and be directly ligated into the vector pCI (Promega) which has been digested with EcoRI and Sal I. The new recombinant plasmid will constitutively transcribe c-N-ras-Val-12 from the CMV promoter of the pCI vector.

Cloning of a c-K-Ras-Val-12 Expression Plasmid

The human c-K-ras gene can be PCRed from a human cerebral cortex cDNA library (Clontech) using the following oligonucleotide primers.

| | | |
|---|---|---|
| Sense strand: | 5′-GAGAGGTACCGCCACCATGACTGAATATAAACTTGTGG-3′ | (SEQ.ID.NO.:85) |
| Antisense strand: | 5′-CTCTGTCGACGTATTTACATAATTACACACTTTGTC-3′ | (SEQ.ID.NO.:86) |

The primers will amplify a c-K-ras encoding DNA fragment with the primers contributing an optimized ‘Kozak’ translation start sequence, a KpnI site at the N-terminus and a Sal I stite at the C-terminal end. After trimming the ends of the PCR product with Kpn I and Sal I, the c-K-ras fragment can be ligated into a KpnI-Sal I cut mutagenesis vector pAlter-1 (Promega). Mutation of cysteine-12 to a valine can be accomplished using the manufacturer's protocols and the following oligonucleotide:

5′-GTAGTTGGAGCTGTTGGCGTAGGC-3′ (SEQ.ID.NO.:87)

After selection and sequencing for the correct nucleotide substitution, the mutated c-K-ras-Val-12 can be excised from the pAlter-1 vector, using KpnI and Sal I, and be directly ligated into the vector pCI (Promega) which has been digested with KpnI and Sal I. The new recombinant plasmid will constitutively transcribe c-K-ras-Val-12 from the CMV promoter of the pCI vector.

SEAP Assay

Human C33A cells (human epitheial carcenoma—ATTC collection) are seeded in 10 cm tissue culture plates in DMEM+10% fetal calf serum+1× Pen/Strep+1× glutamine+1× NEAA. Cells are grown at 37° C. in a 5% CO₂ atmosphere until they reach 50-80% of confluency.

The transient transfection is performed by the CaPO₄ method (Sambrook et al., 1989). Thus, expression plasmids for H-ras, N-ras, K-ras, Myr-ras or H-ras-CVLL are co-precipitated with the DSE-SEAP reporter construct. For 10 cm plates 600 μl of CaCl₂-DNA solution is added dropwise while vortexing to 600 μl of 2× HBS buffer to give 1.2 ml of precipitate solution (see recipes below). This is allowed to sit at room temperature for 20 to 30 minutes. While the precipitate is forming, the media on the C33A cells is replaced with DMEM (minus phenol red; Gibco cat. # 31053-028)+0.5% charcoal stripped calf serum+1× (Pen/Strep, Glutamine and nonessential aminoacids). The CaPO₄-DNA precipitate is added dropwise to the cells and the plate rocked gently to distribute. DNA uptake is allowed to proceed for 5-6 hrs at 37° C. under a 5% CO₂ atmosphere.

Following the DNA incubation period, the cells are washed with PBS and trypsinized with 1 ml of 0.05% trypsin. The 1 ml of trypsinized cells is diluted into 10 ml of phenol red free DMEM+0.2% charcoal stripped calf serum+1× (Pen/Strep, Glutamine and NEAA). Transfected cells are plated in a 96 well microtiter plate (100 μl/well) to which drug, diluted in media, has already been added in a volume of 100 μl. The final volume per well is 200 μl with each drug concentration repeated in triplicate over a range of half-log steps.

Incubation of cells and test compounds or compositions is for 36 hrs at 37° C. under CO₂. At the end of the incubation period, cells are examined microscopically for evidence of cell distress. Next, 100 μl of media containing the secreted alkaline phosphatase is removed from each well and transferred to a microtube array for heat treatment at 65° C. for 1 hr to inactivate endogenous alkaline phosphatases (but not the heat stable secreted phosphatase).

The heat treated media is assayed for alkaline phosphatase by a luminescence assay using the luminescence reagent CSPD® (Tropix, Bedford, Mass.). A volume of 50 μl media is combined with 200 μl of CSPD cocktail and incubated for 60 minutes at room temperature. Luminesence is monitored using an ML2200 microplate luminometer (Dynatech). Luminescence reflects the level of activation of the fos reporter construct stimulated by the transiently expressed protein.

| DNA-CaPO₄ precipitate for 10 cm. plate of cells | | |
|---|---|---|
| Ras expression plasmid (1 μg/μl) | 10 | μl |
| DSE-SEAP Plasmid (1 μg/μl) | 2 | μl |
| Sheared Calf Thymus DNA (1 μg/μl) | 8 | μl |
| 2M CaCl₂ | 74 | μl |
| dH₂O | 506 | μl |
| 2X HBS Buffer | | |
| 280 mM NaCl | | |
| 10 mM KCl | | |
| 1.5 mM Na₂HPO₄ 2H₂O | | |
| 12 mM dextrose | | |
| 50 mM HEPES | | |
| Final pH = 7.05 | | |
| Luminesence Buffer (26 ml) | | |
| Assay Buffer | 20 | ml |
| Emerald Reagent ™ (Tropix) | 2.5 | ml |
| 100 mM homoarginine | 2.5 | ml |
| CSPD Reagent ® (Tropix) | 1.0 | ml |
| Assay Buffer | | |
| Add 0.05M Na₂CO₃ to 0.05M | | |
| NaHCO₃ to obtain pH 9.5. | | |
| Make 1 mM in MgCl₂ | | |

### Example 40

The processing assays employed in this example and in Example 41 are modifications of that described by DeClue et al [Cancer Research 51, 712-717, 1991].

K4B-Ras Processing Inhibition Assay

PSN-1 (human pancreatic carcinoma) are used for analysis of protein processing. Subconfluent cells in 100 mm dishes are fed with 3.5 ml of media (methionine-free RPMI supplemented with 2% fetal bovine serum or cysteine-free/methionine-free DMEM supplemented with 0.035 ml of 200 mM glutamine (Gibco), 2% fetal bovine serum, respectively) containing the desired concentration of farnesyl-protein transferase inhibitor, HMG-CoA reductase inhibitor, instant combination composition or solvent alone. Test compounds or compositions are prepared as 1000× concentrated solutions in DMSO to yield a final solvent concentration of 0.1%. Following incubation at 37° C. for two hours 204 μCi/ml [³⁵S]Pro-Mix (Amersham, cell labeling grade) is added.

After introducing the label amino acid mixture, the cells are incubated at 37° C. for an additional period of time (typically 6 to 24 hours). The media is then removed and the cells are washed once with cold PBS. The cells are scraped into 1 ml of cold PBS, collected by centrifugation (10,000× g for 10 sec at room temperature), and lysed by vortexing in 1 ml of lysis buffer (1% Nonidet P-40, 20 mM HEPES, pH 7.5, 150 mM NaCl, 1 mM EDTA, 0.5% deoxycholate, 0.1% SDS, 1 mM DTT, 10 μg/ml AEBSF, 10 μg/ml aprotinin, 2 μg/ml leupeptin and 2 μg/ml antipain). The lysate is then centrifuged at 15,000× g for 10 min at 4° C. and the supernatant saved.

For immunoprecipitation of Ki4B-Ras, samples of lysate supernatant containing equal amounts of protein are utilized. Protein concentration is determined by the bradford method utilizing bovine serum albumin as a standard. The appropriate volume of lysate is brought to 1 ml with lysis buffer lacking DTT and 8 μg of the pan Ras monoclonal antibody, Y13-259, added. The protein/antibody mixture is incubated on ice at 4° C. for 24 hours. The immune complex is collected on pansorbin (Calbiochem) coated with rabbit antiserum to rat IgG (Cappel) by tumbling at 4° C. for 45 minutes. The pellet is washed 3 times with 1 ml of lysis buffer lacking DTT and protease inhibitors and resuspended in 100 μl elution buffer (10 mM Tris pH 7.4, 1% SDS). The Ras is eluted from the beads by heating at 95° C. for 5 minutes, after which the beads are pelleted by brief centrifugation (15,000× g for 30 sec. at room temperature).

The supernatant is added to 1 ml of Dilution Buffer 0.1% Triton X-100, 5 mM EDTA, 50 mM NaCl, 10 mM Tris pH 7.4) with 2 μg Kirsten-ras specific monoclonal antibody, c-K-ras Ab-1 (Calbiochem). The second protein/antibody mixture is incubated on ice at 4° C. for 1-2 hours. The immune complex is collected on pansorbin (Calbiochem) coated with rabbit antiserum to rat IgG (Cappel) by tumbling at 4° C. for 45 minutes. The pellet is washed 3 times with 1 ml of lysis buffer lacking DTT and protease inhibitors and resuspended in Laemmli sample buffer. The Ras is eluted from the beads by heating at 95° C. for 5 minutes, after which the beads are pelleted by brief centrifugation. The supernatant is subjected to SDS-PAGE on a 12% acrylamide gel (bis-acrylamide:acrylamide, 1:100), and the Ras visualized by fluorography.

hDJ Processing Inhibition Assay

PSN-1 cells are seeded in 24-well assay plates. For each compoundor composition to be tested, the cells are treated with a minimum of seven concentrations in half-log steps. The final solvent (DMSO) concentration is 0.1%. A vehicle-only control is included on each assay plate. The cells are treated for 24 hours at 37° C./5% CO₂.

The growth media is then aspirated and the samples are washed with PBS. The cells are lysed with SDS-PAGE sample buffer containing 5% 2-mercaptoethanol and heated to 95° C. for 5 minutes. After cooling on ice for 10 minutes, a mixture of nucleases is added to reduce viscosity of the samples.

The plates are incubated on ice for another 10 minutes. The samples are loaded onto pre-cast 8% acrylamide gels and electrophoresed at 15 mA/gel for 3-4 hours. The samples are then transferred from the gels to PVDF membranes by Western blotting.

The membranes are blocked for at least 1 hour in buffer containing 2% nonfat dry milk. The membranes are then treated with a monoclonal antibody to HDJ-2 (Neomarkers Cat. # MS-225), washed, and treated with an alkaline phosphatase-conjugated secondary antibody. The membranes are then treated with a fluorescent detection reagent and scanned on a phosphorimager.

For each sample, the percent of total signal corresponding to the unprenylated species of HDJ (the slower-migrating species) is calculated by densitometry. Dose-response curves and IC₅₀ values are generated using 4-parameter curve fits in SigmaPlot software.

### Example 41

K4B-Ras Processing Inhibition Assay

PSN-1 (human pancreatic carcinoma) cells are used for analysis of protein processing. Subconfluent cells in 150 mm dishes are fed with 20 ml of media (RPMI supplemented with 15% fetal bovine serum) containing the desired concentration of test composition, compound, lovastatin or solvent alone. Cells treated with lovastatin (5-10 μM), a compound that blocks Ras processing in cells by inhibiting a rate-limiting step in the isoprenoid biosynthetic pathway, serve as a positive control. Test compounds and compositions are prepared as 1000× concentrated solutions in DMSO to yield a final solvent concentration of 0.1%.

The cells are incubated at 37° C. for 24 hours, the media is then removed and the cells are washed twice with cold PBS. The cells are scraped into 2 ml of cold PBS, collected by centrifugation (10,000× g for 5 min at 4° C.) and frozen at −70° C. Cells are lysed by thawing and addition of lysis buffer (50 mM HEPES, pH 7.2, 50 mM NaCl, 1% CHAPS, 0.7 μg/ml aprotinin, 0.7 μg/ml leupeptin 300 μg/ml pefabloc, and 0.3 mM EDTA). The lysate is then centrifuged at 100,000× g for 60 min at 4° C. and the supernatant saved. The supernatant may be subjected to SDS-PAGE, HPLC analysis, and/or chemical cleavage techniques.

The lysate is applied to a HiTrap-SP (Pharmacia Biotech) column in buffer A (50 mM HEPES pH 7.2) and resolved by gradient in buffer A plus 1 M NaCl. Peak fractions containing Ki4B-Ras are pooled, diluted with an equal volume of water and immunoprecipitated with the pan Ras monoclonal antibody, Y13-259 linked to agarose or Kirsten-ras specific monoclonal antibody, c-K-ras Ab-1 (Calbiochem). The protein/antibody mixture is incubated at 4° C. for 12 hours. The immune complex is washed 3 times with PBS, followed by 3 times with water. The Ras is eluted from the beads by either high pH conditions (pH>10) or by heating at 95° C. for 5 minutes, after which the beads are pelleted by brief centrifugation. The supernatant may be subjected to SDS-PAGE, HPLC analysis, and/or chemical cleavage techniques.

### Example 42

Rap1 Processing Inhibition Assay

Protocol A:

Cells are labeled, incubated and lysed as described in Example 41.

For immunoprecipitation of Rap1, samples of lysate supernatant containing equal amounts of protein are utilized. Protein concentration is determined by the bradford method utilizing bovine serum albumin as a standard. The appropriate volume of lysate is brought to 1 ml with lysis buffer lacking DTT and 2 μg of the Rap1 antibody, Rap1/Krev1 (121) (Santa Cruz Biotech), is added. The protein/antibody mixture is incubated on ice at 4° C. for 1 hour. The immune complex is collected on pansorbin (Calbiochem) by tumbling at 4° C. for 45 minutes. The pellet is washed 3 times with 1 ml of lysis buffer lacking DTT and protease inhibitors and resuspended in 100 ml elution buffer (10 mM Tris pH 7.4, 1% SDS). The Rapi is eluted from the beads by heating at 95° C. for 5 minutes, after which the beads are pelleted by brief centrifugation (15,000× g for 30 sec. at room temperature).

The supernatant is added to 1 ml of Dilution Buffer (0.1% Triton X-100, 5 mM EDTA, 50 mM NaCl, 10 mM Tris pH 7.4) with 2 mg Rap1 antibody, Rap1/Krev1 (121) (Santa Cruz Biotech). The second protein/antibody mixture is incubated on ice at 4° C. for 1-2 hours. The immune complex is collected on pansorbin (Calbiochem) by tumbling at 4° C. for 45 minutes. The pellet is washed 3 times with 1 ml of lysis buffer lacking DTT and protease inhibitors and resuspended in Laemmli sample buffer. The Rap1 is eluted from the beads by heating at 95° C. for 5 minutes, after which the beads are pelleted by brief centrifugation. The supernatant is subjected to SDS-PAGE on a 12% acrylamide gel (bis-acrylamide:acrylamide, 1:100), and the Rap1 visualized by fluorography.

Protocol B:

PSN-1 cells are passaged every 3-4 days in 10 cm plates, splitting near-confluent plates 1:20 and 1:40. The day before the assay is set up, 5×10⁶ cells are plated on 15 cm plates to ensure the same stage of confluency in each assay. The media for these cells is RPM11640 (Gibco), with 15% fetal bovine serum and 1× Pen/Strep antibiotic mix.

The day of the assay, cells are collected from the 15 cm plates by trypsinization and diluted to 400,000 cells/ml in media. 0.5 ml of these diluted cells are added to each well of 24-well plates, for a final cell number of 200,000 per well. The cells are then grown at 37° C. overnight.

The compounds or compositionsto be assayed are diluted in DMSO in 1/2-log dilutions. The range of final concentrations to be assayed is generally 0.1-100 μM. Four concentrations per compound is typical. The compounds are diluted so that each concentration is 1000× of the final concentration (i.e., for a 10 μM data point, a 10 mM stock of the compound is needed).

2 μL of each 1000× compound stock is diluted into 1 ml media to produce a 2× stock of compound. A vehicle control solution (2 μL DMSO to 1 ml media), is utilized. 0.5 ml of the 2× stocks of compound are added to the cells.

After 24 hours, the media is aspirated from the assayplates. Each well is rinsed with 1 ml PBS, and the PBS is aspirated. 180 μL SDS-PAGE sample buffer (Novex) containing 5% 2-mercaptoethanol is added to each well. The plates are heated to 100° C. for 5 minutes using a heat block containing an adapter for assay plates. The plates are placed on ice. After 10 minutes, 20 μL of an RNAse/DNase mix is added per well. This mix is 1 mg/ml DNaseI (Worthington Enzymes), 0.25 mg/ml Rnase A (Worthington Enzymes), 0.5M Tris-HCl pH 8.0 and 50 mM MgCl₂. The plate is left on ice for 10 minutes. Samples are then either loaded on the gel, or stored at −70° C. until use.

Each assay plate (usually 3 compounds, each in 4-point titrations, plus controls) requires one 15-well 14% Novex gel. 25 μl of each sample is loaded onto the gel. The gel is run at 15 mA for about 3.5 hours. It is important to run the gel far enough so that there will be adequate separation between 21 kd (Rap1) and 29 kd (Rab6).

The gels are then transferred to Novex pre-cut PVDF membranes for 1.5 hours at 30V (constant voltage). Immediately after transferring, the membranes are blocked overnight in 20 ml Western blocking buffer (2% nonfat dry milk in Western wash buffer (PBS+0.1% Tween-20). If blocked over the weekend, 0.02% sodium azide is added. The membranes are blocked at 4° C. with slow rocking.

The blocking solution is discarded and 20 ml fresh blocking solution containing the anti Rap1a antibody (Santa Cruz Biochemical SC1482) at 1:1000 (diluted in Western blocking buffer) and the anti Rab6 antibody (Santa Cruz Biochemical SC310) at 1:5000 (diluted in Western blocking buffer) are added. The membranes are incubated at room temperature for 1 hour with mild rocking. The blocking solution is then discarded and the membrane is washed 3 times with Western wash buffer for 15 minutes per wash. 20 ml blocking solution containing 1:1000 (diluted in Western blocking buffer) each of two alkaline phosphatase conjugated antibodies (Alkaline phosphatase conjugated Anti-goat IgG and Alkaline phosphatase conjugated anti-rabbit IgG [Santa Cruz Biochemical]) is then added. The membrane is incubated for one hour and washed 3× as above.

About 2 ml per gel of the Amersham ECF detection reagent is placed on an overhead transparency (ECF) and the PVDF membranes are placed face-down onto the detection reagent. This is incubated for one minute, then the membrane is placed onto a fresh transparency sheet.

The developed transparency sheet is scanned on a phosphorimager and the Rap1a Minimum Inhibitory Concentration is determined from the lowest concentration of compound that produces a detectable Rap1a Western signal. The Rap1a antibody used recognizes only unprenylated/unprocessed Rap1a, so that the precence of a detectable Rap1a Western signal is indicative of inhibition of Rap1a prenylation.

The ability of the PSA conjugate compounds useful in the methods of the present invention of the present invention to be selectively cleaved by enzymatically active PSA and the selective cytotoxicity of those conjugate compounds can be demonstrated using the following assays.

Protocol C:

This protocol allows the determination of an EC₅₀ for inhibition of processing of Rap1a. The assay is run as described in Protocol B with the following modifications. 20 Al of sample is run on pre-cast 10-20% gradient acrylamide mini gels (Novex Inc.) at 15 mA/gel for 2.5-3 hours. Prenylated and unprenylated forms of Rap1a are detected by blotting with a polyclonal antibody (Rap1/Krev-1 Ab#121; Santa Cruz Research Products #sc-65), followed by an alkaline phosphatase-conjugated anti-rabbit IgG antibody. The percentage of unprenylated Rap1a relative to the total amount of Rap1a is determined by peak integration using Imagequant® software (Molecular Dynamics). Unprenylated Rap1a is distinguished from prenylated protein by virtue of the greater apparent molecular weight of the prenylated protein. Dose-response curves and EC₅₀ values are generated using 4-parameter curve fits in SigmaPlot software.

### Example 43

Assessment of the Recognition of Oligopeptide-Cytotoxic Drug Conjugates by Free PSA

The conjugates prepared as described in Examples 28-34 are individually dissolved in PSA digestion buffer (50 mM tris(hydroxymethyl)-aminomethane pH 70.4, 140 mM NaCl) and the solution added to PSA at a molar ration of 100 to 1. Alternatively, the PSA digestion buffer utilized is 50 mM tris(hydroxymethyl)-aminomethane pH 7.4, 140 mM NaCl. The reaction is quenched after various reaction times by the addition of trifluoroacetic acid (TFA) to a final 1% (volume/volume). Alternatively the reaction is quenched with 10 mM ZnCl₂. The quenched reaction is analyzed by HPLC on a reversed-phase C18 column using an aqueous 0.1%TFA/acetonitrile gradient. The amount of time (in minutes) required for 50% cleavage of the noted oligopeptide-cytotoxic agent conjugates with enzymatically active free PSA were then calculated.

### Example 44

In vitro Assay of Cytotoxicity of Peptidyl Derivatives of Doxorubicin:

The cytotoxicities of the cleaveable oligopeptide-doxorubicin conjugates, prepared as described in Examples 25-27, against a line of cells which is known to be killed by unmodified doxorubicin are assessed with an Alamar Blue assay. Specifically, cell cultures of LNCap prostate tumor cells (which express enzymatically active PSA) or DuPRO cells in 96 well plates are diluted with medium (Dulbecco's Minimum Essential Medium-α [MEM-α]) containing various concentrations of a given conjugate (final plate well volume of 200 μl). The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures.

### Example 45

In vitro Assay of Cytotoxicity of Peptidyl Derivatives of Vinca Drugs

The cytotoxicities of the cleaveable oligopeptide-vinca drug conjugates, prepared as described in Examples 28-34, against a line of cells which is known to be killed by unmodified vinca drug was assessed with an Alamar Blue assay. Specifically, cell cultures of LNCap prostate tumor cells, Colo320DM cells (designated C320) or T47D cells in 96 well plates are diluted with medium containing various concentrations of a given conjugate (final plate well volume of 200 μl). The Colo320DM cells, which do not express free PSA, are used as a control cell line to determine non-mechanism based toxicity. The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures and an EC₅₀ was determined.

### Example 46

In vivo Efficacy of Peptidyl-Cytotoxic Agent Conjugates

LNCaP.FGC or DuPRO-1 cells are trypsinized, resuspended in the growth medium and centifuged for 6 mins. at 200× g. The cells are resuspended in serum-free α-MEM and counted. The appropriate volume of this solution containing the desired number of cells is then transferred to a conical centrifuge tube, centrifuged as before and resuspended in the appropriate volume of a cold 1:1 mixture of α-MEM-Matrigel. The suspension is kept on ice until the animals are inoculated.

Harlan Sprague Dawley male nude mice (10-12 weeks old) are restrained without anesthesia and are inoculated with 0.5 mL of cell suspension on the left flank by subcutaneous injection using a 22G needle. Mice are either given approximately 5×10⁵ DuPRO cells or 1.5×10⁷ LNCaP.FGC cells.

Following inoculation with the tumor cells the mice are treated under one of two protocols:

Protocol A:

One day after cell inoculation the animals are dosed with a 0.1-0.5 mL volume of test conjugate, vinca drug or vehicle control (sterile water). Dosages of the conjugate and vinca drug are initially the maximum non-lethal amount, but may be subsequently titrated lower. Identical doses are administered at 24 hour intervals for 5 days. After 10 days, blood samples are removed from the mice and the serum level of PSA is determined. Similar serum PSA levels are determined at 5-10 day intervals. At the end of 5.5 weeks the mice are sacrificed and weights of any tumors present are measured and serum PSA again determined. The animals' weights are determined at the beginning and end of the assay.

Protocol B:

Ten days after cell inoculation, blood samples are removed from the animals and serum levels of PSA are determined. Animals are then grouped according to their PSA serum levels. At 14-15 days after cell inoculation, the animals are dosed with a 0.1-0.5 mL volume of test conjugate, vinca drug or vehicle control (sterile water). Dosages of the conjugate and vinca drug are initially the maximum non-lethal amount, but may be subsequently titrated lower. Identical doses are administered at 24 hour intervals for 5 days. Serum PSA levels are determined at 5-10 day intervals. At the end of 5.5 weeks the mice are sacrificed, weights of any tumors present are measured and serum PSA again determined. The animals' weights are determined at the beginning and end of the assay.

### Example 47

In vivo Efficacy of Administration of a Combination of a PSA Conjugate and a Prenyl Protein Transferase Inhibitor

Male nude mice (4 groups of 15) were injected subcutaneously with 1.5×10⁷ LNCaP.FGC cells (available from the American Type Culture Collection, ATCC No. CRL-1740; see also J. S. Horoszewicz et al. Cancer Res., 43:1809-1818 (1983)) in 80% Matrigel.

Compound A, prepared as described in Example 2 (1.8 g) was dissolved in 4.4 mL 50% aqueous DMSO; filtered through a Millipore Steriflip™ filter unit (0.22 μm membrane) and stored at room temperature. ALZET® micro-osmotic pumps (model 1007D, mean pumping rate 0.5 μl/hr, mean fill volume 98.1 μL) were filled with either the solution of Compound A or vehicle (50% aqueous DMSO), placed in warm isotonic saline and kept in a 37° C. waterbath until used.

On the forth day after the injection of the LNCaP.FGC cells, the mice were anesthestized and the pumps implanted subdermally as follows:

Group A: Pump containing Compound A solution

Group B: Pump containing vehicle

Group C: Pump containing Compound A solution

Group D: Pump containing vehicle

48 Hours after the implantation of the osmotic pumps, three mice from each group were bled from the tail vein to assess serum levels of Compound A. After the levels of Compound A were assessed, 0.20 mL of a solution of Compound B, prepared as described in Example 26 (37.1 mg dissolved in 34.1 mL D5W+80 μL 7.5% sodium bicarbonate) was administered to Groups A and B. Vehicle (0.20 mL) was administered to Groups C and D.

Four additional doses (one/day) of Compound B solution or vehicle were administered to the respective Groups over four days. The mice were then maintained for 22 days.

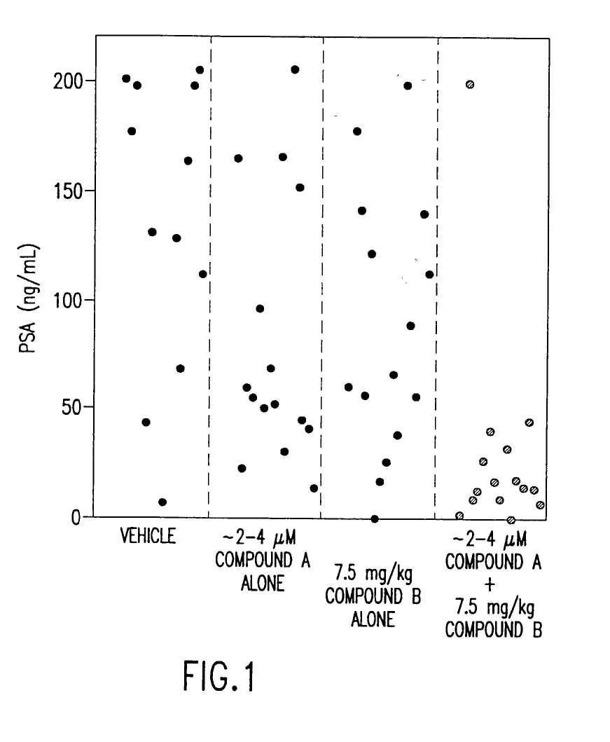

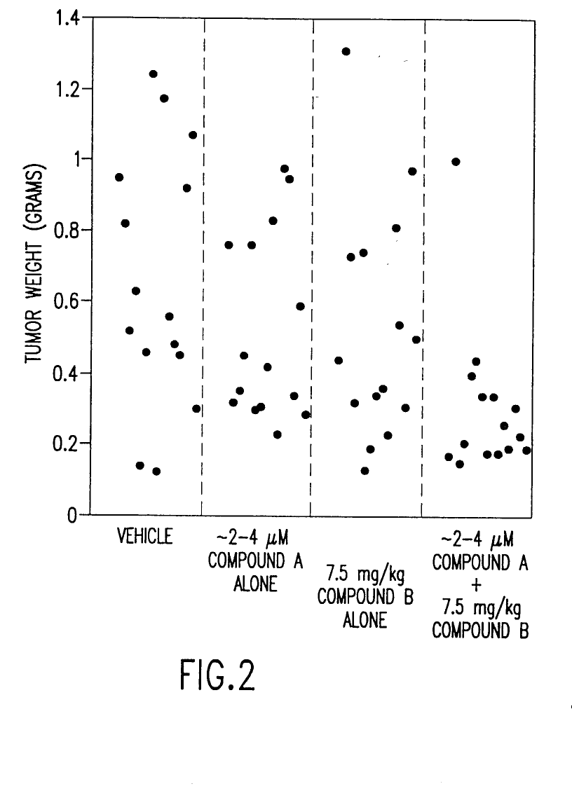

At the end of 22 days after the last injection of Compound B solution or vehicle the mice were bled from the tail vein and the plasma PSA level was measured using a Tandem®-E PSA-ImmunoEnzyMetri Assay kit (Hybritech). The mice were then sacrificed, weighed, tumors excised and weighed. The results are shown in FIGS. 1 and 2.

### Example 48

In vitro Determination of Proteolytic Cleavage of Conjugates by Endogenous Non-PSA Proteases

Step A: Preparation of Proteolytic Tissue Extracts

All procedures are carried out at 4° C. Appropriate animals are sacrificed and the relevant tissues are isolated and stored in liquid nitrogen. The frozen tissue is pulverized using a mortar and pestle and the pulverized tissue is transfered to a Potter-Elvejeh homogenizer and 2 volumes of Buffer A (50 mM Tris containing 1.15% KCl, pH 7.5) are added. The tissue is then disrupted with 20 strokes using first a lose fitting and then a tight fitting pestle. The homogenate is centrifuged at 10,000× g in a swinging bucket rotor (HB4-5), the pellet is discarded and the re-supernatant centrifuged at 100,000× g (Ti 70). The supernatant (cytosol)is saved.

The pellet is resuspended in Buffer B (10 mM EDTA containing 1.15% KCl, pH 7.5) using the same volume used in step as used above with Buffer A. The suspension is homogenized in a dounce homogenizer and the solution centrifuged at 100,000× g. The supernatant is discarded and the pellet resuspended in Buffer C(10 mM potassium phosphate buffer containing 0.25 M sucrose, pH 7.4), using ½ the volume used above, and homogenized with a dounce homogenizer.

Protein content of the two solutions (cytosol and membrane) is determine using the Bradford assay. Assay aliquots are then removed and frozen in liquid N₂. The aliquots are stored at −70° C.

Step B: Proteolytic Cleavage Assay

For each time point, 20 microgram of peptide-vinca drug conjugate and 150 micrograms of tissue protein, prepared as described in Step A and as determined by Bradford in reaction buffer are placed in solution of final volume. of 200 microliters in buffer (50 mM TRIS, 140 mM NaCl, pH 7.2). Assay reactions are run for 0, 30, 60, 120, and 180 minutes and are then quenched with 9 microliters of 0.1 M ZnCl₂ and immediately placed in boiling water for 90 seconds. Reaction products are analyzed by HPLC using a VYDAC C18 15 cm column in water/acetonitrile (5% to 50% acetonitrile over 30 minutes).

What is claimed is:

1. A method for achieving a therapeutic effect in a mammal in need thereof which comprises administering to said mammal amounts of at least one inhibitor of prenyl-protein transferase and at least one PSA conjugate.

2. The method according to claim 1 wherein an amount of a prenyl-protein transferase inhibitor and an amount of an PSA conjugate are administered consecutively.

3. The method according to claim 1 wherein an amount of a prenyl-protein transferase inhibitor and an amount of an PSA conjugate are administered simultaneously.

4. The method according to claim 1 wherein the therapeutic effect is treatment of cancer.

5. The method according to claim 4 wherein the therapeutic effect is selected from inhibition of cancerous tumor growth and regression of cancerous tumors.

6. The method according to claim 4 wherein the cancer is a cancer related to cells that express enzymatically active PSA.

7. The method according to claim 4 wherein the cancer is prostate cancer.

8. The method according to claim 1 wherein the PSA conjugate is selected from:
a) a compound represented by the formula IX: 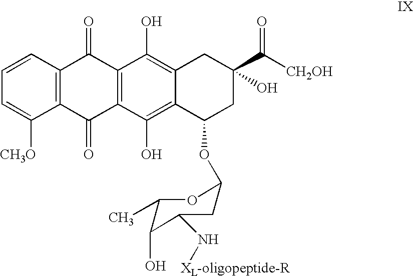
wherein:
oligopeptide is an oligopeptide which is specifically recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine;
R is hydrogen or —(C═O)R¹; and
R¹ is C₁-C₆-alkyl or aryl,
or the pharmaceutically acceptable salt thereof;
b) a compound represented by the formula X: 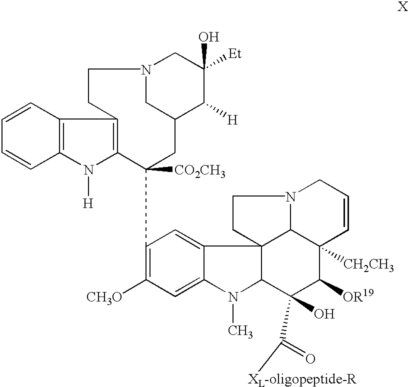
wherein:
oligopeptide is an oligopeptide which is specifically recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine; or
X_{L} is —NH—(CH₂)ₙ—NH—
R is hydrogen or —(C═O)R¹;
R¹ is C₁-C₆-alkyl or aryl;
R¹⁹ is hydrogen or acetyl; and
n is 1, 2, 3, 4 or 5,
or the pharmaceutically acceptable salt thereof;
c) a compound represented by the formula XI: 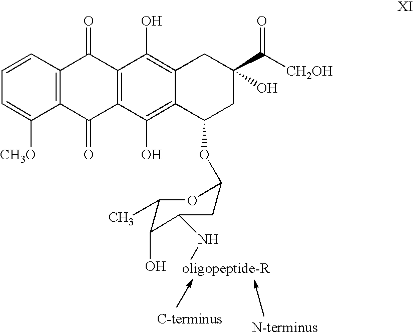
wherein:
oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula: 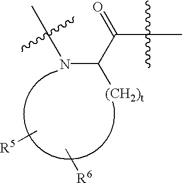
and wherein
the C-terminus carbonyl is covalently bound to the amine of doxorubicin;
R is selected from
a) hydrogen,
b) —(C═O)R^{1a}, 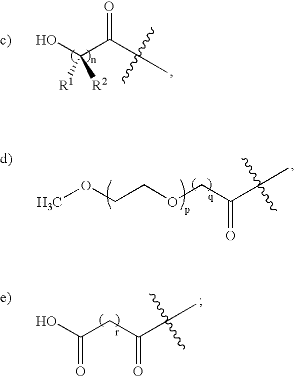
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R⁵ is selected from HO— and C₁-C₆ alkoxy;
R⁶ is selected from hydrogen, halogen, C₁-C₆ alkyl, HO— and C₁-C₆ alkoxy; and
n is 1, 2, 3 or 4;
p is zero or an integer between 1 and 100;
q is 0 or 1, provided that if p is zero, q is 1;
r is an integer between 1 and 10; and
t is 3 or 4;
or a pharmaceutically acceptable salt thereof;
d) a compound represented by the formula X: 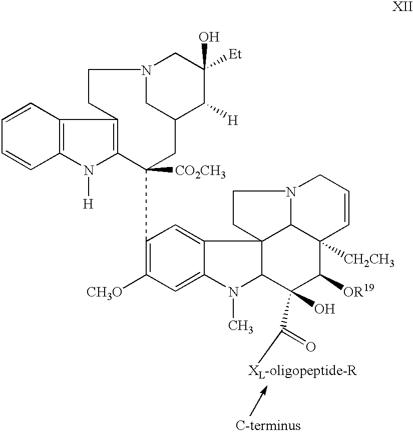
wherein:
oligopeptide is an oligopeptide which is specifically recognized by the free prostate specific antigen (PSA) and is capable of being proteolytically cleaved by the enzymatic activity of the free prostate specific antigen, and the oligopeptide comprises a cyclic amino acid of the formula: 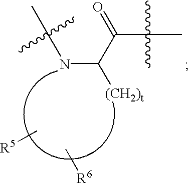
XL is —NH—(CH₂)ᵤ—NH—
R is selected from
a) hydrogen,
b) —(C═O)R^{1a}, 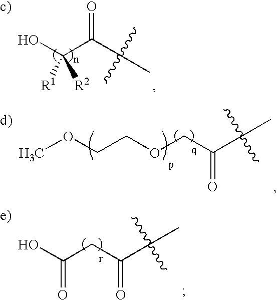
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2 or 3;
t is 3 or 4;
u is 1, 2, 3, 4 or 5,
or the pharmaceutically acceptable salt thereof;
e) a compound represented by the formula XI: 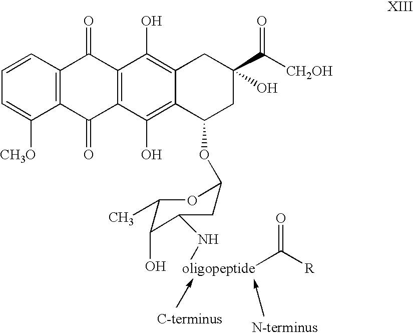
wherein:
oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;
R is selected from 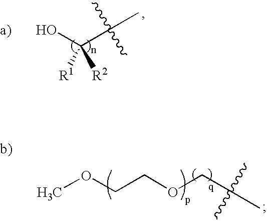
R¹ and R² are independently selected from: hydrogen, OH, C₁-C₆ alkyl, C₁-C₆ alkoxy, C₁-C₆ aralkyl and aryl;
n is 1, 2, 3 or 4;
p is zero or an integer between 1 and 100;
q is 0 or 1, provided that if p is zero, q is 1;
or the pharmaceutically acceptable salt thereof;
f) a compound represented by the formula XIV: 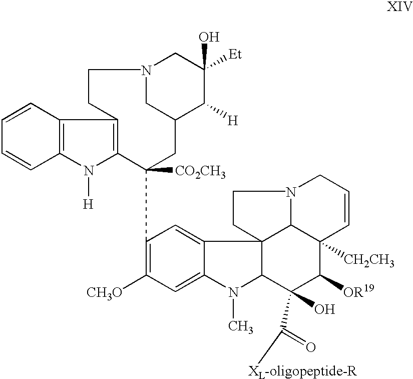
wherein:
oligopeptide is an oligopeptide which is specifically recognized by the free prostate specific antigen (PSA) and is capable of being proteolytically cleaved by the enzymatic activity of the free prostate specific antigen;
X_{L} is —NH—(CH₂)ᵣ—NH—
R is selected from 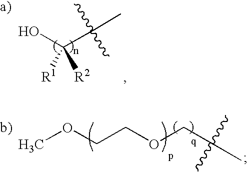
R¹ and R² are independently selected from: hydrogen, OH, C₁-C₆ alkyl, C₁-C₆ alkoxy, C₁-C₆ aralkyl and aryl;
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2, 3, 4 or 5;
or the pharmaceutically acceptable salt thereof;
g) a compound represented by the formula XV: 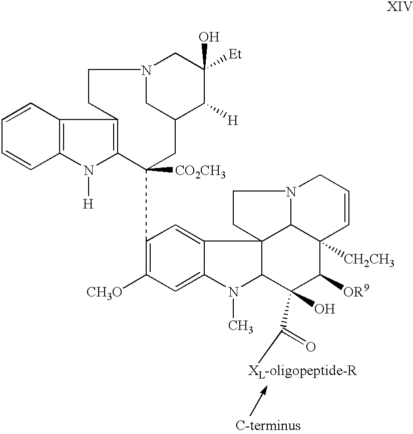
wherein:
oligopeptide is an oligopeptide which is specifically recognized by the free prostate specific antigen (PSA) and is capable of being proteolytically cleaved by the enzymatic activity of the free prostate specific antigen,
X_{L} is —NH—(CH₂)ᵤ—W—(CH₂)ᵤ—NH—
R is selected from
a) hydrogen,
b) (C═O)R^{1a}, 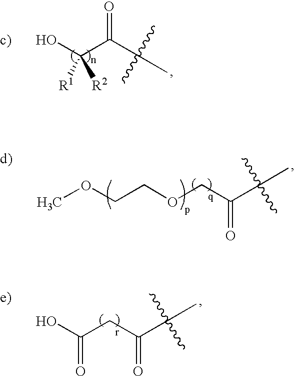
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3;
or the pharmaceutically acceptable salt thereof; and
h) a compound represented by the formula XVI: 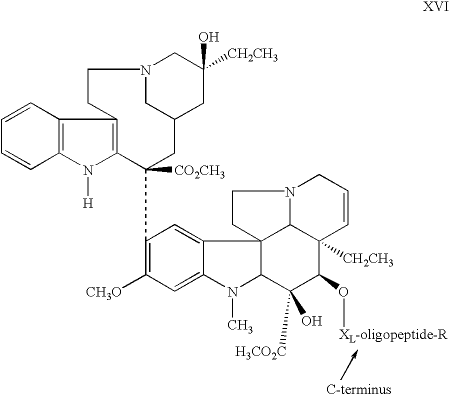
wherein:
oligopeptide is an oligopeptide which is specifically recognized by the free prostate specific antigen (PSA) and is capable of being proteolytically cleaved by the enzymatic activity of the free prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R is selected from
a) hydrogen,
b) —(C═O)R^{1a}, 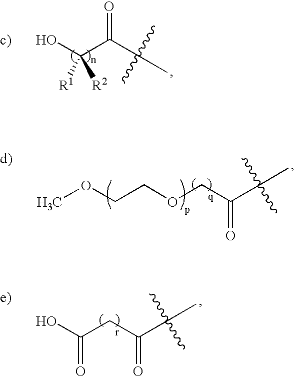
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from a branched or straight chain C₁-C₆-alkyl, cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3,
or the pharmaceutically acceptable salt or optical isomer thereof.

9. The method according to claim 8 wherein the PSA conjugate is selected from:
i)

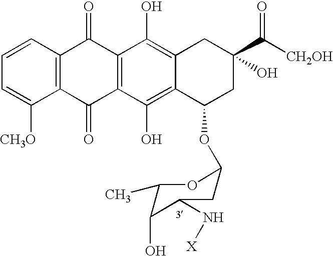

| | |
|---|---|
| wherein X is: | |
| AsnLysIleSerTyrGlnSer- | (SEQ.ID.NO.: 14), |
| AsnLysIleSerTyrGlnSerSer- | (SEQ.ID.NO.: 15), |
| AsnLysIleSerTyrGlnSerSerSer- | (SEQ.ID.NO.: 16), |
| AsnLysIleSerTyrGlnSerSerSerThr- | (SEQ.ID.NO.: 17), |
| AsnLysIleSerTyrGlnSerSerSerThrGlu- | (SEQ.ID.NO.: 18), |
| AlaAsnLysIleSerTyrGlnSerSerSerThrGlu- | (SEQ.ID.NO.: 19), |
| Ac-AlaAsnLysIleSerTyrGlnSerSerSerThr- | (SEQ.ID.NO.: 20), |
| Ac-AlaAsnLysIleSerTyrGlnSerSerSerThrLeu- | (SEQ.ID.NO.: 21), |
| Ac-AlaAsnLysAlaSerTyrGlnSerAlaSerThrLeu- | (SEQ.ID.NO.: 22), |
| Ac-AlaAsnLysAlaSerlyrGlnSerAlaSerLeu- | (SEQ.ID.NO.: 23), |
| Ac-AlaAsnLysAlaSerTyrGlnSerSerSerLeu- | (SEQ.ID.NO.: 24), |
| Ac-AlaAsnLysAlaSerTyrGlnSerSerLeu- | (SEQ.ID.NO.: 25), |
| Ac-SerTyrGlnSerSerSerLeu- | (SEQ.ID.NO.: 26), |
| Ac-hArgTyrGlnSerSerSerLeu- | (SEQ.ID.NO.: 27). |
| Ac-LysTyrGlnSerSerSerLeu- | (SEQ.ID.NO.: 28), |
| Ac-LysTyrGlnSerSerNle- | (SEQ.ID.NO.: 29), |
| ii) | |
| 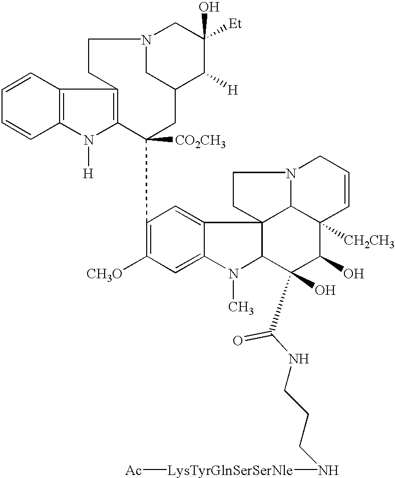 | (SEQ.ID.NO.: 30), |
| 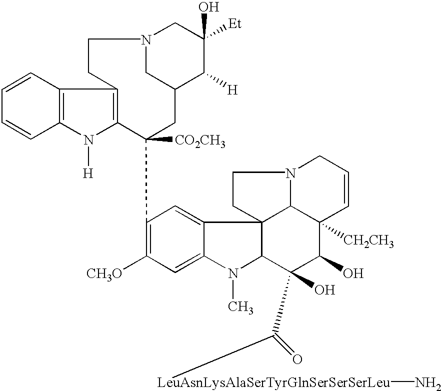 | (SEQ.ID.NO.: 31), |
| iii) | |
| 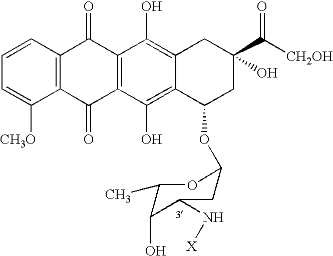 | |
| wherein X is: | |
| 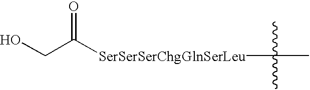 | (SEQ.ID.NO.: 32), |
| 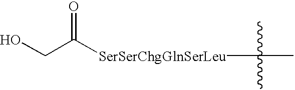 | (SEQ.ID.NO.: 33), |
| 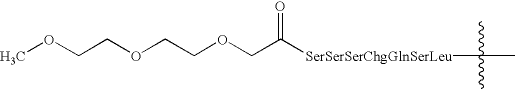 | (SEQ.ID.NO.: 34), |
| iv) | |
| 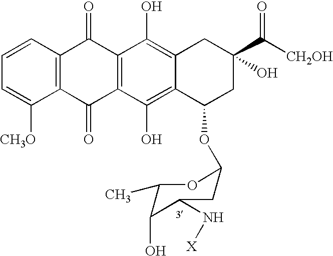 | |
| wherein X is: | |
| 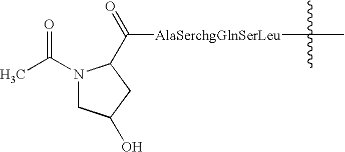 | (SEQ.ID.NO. 35,) |
| 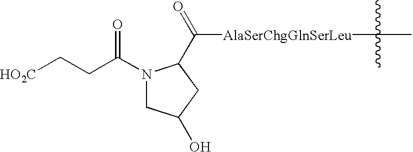 | (SEQ.ID.NO. 36,) |
| 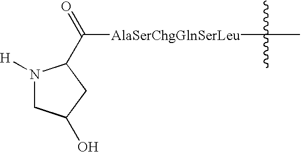 | (SEQ.ID.NO.: 24), |
| 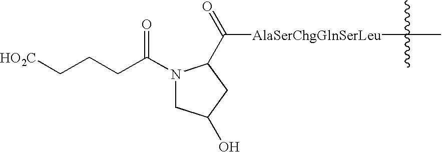 | (SEQ.ID.NO.: 25), |
| 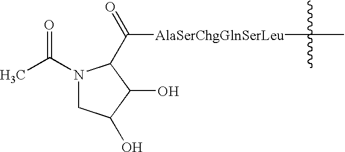 | (SEQ.ID.NO.: 26), |
| 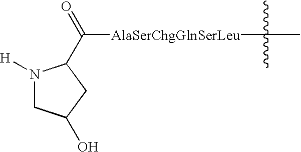 | (SEQ.ID.NO.: 37), |
| 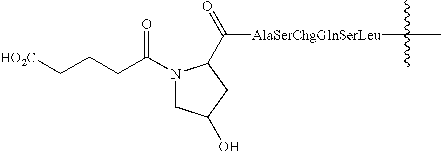 | (SEQ.ID.NO.: 38), |
| 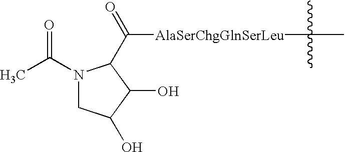 | (SEQ.ID.NO.: 39), |
| 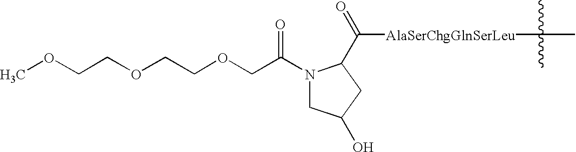 | (SEQ.ID.NO.: 40), |
| 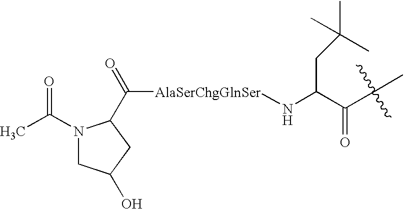 | (SEQ.ID.NO.: 41), |
| v) | |
| 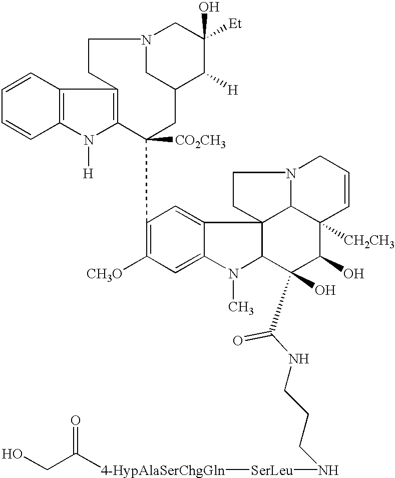 | (SEQ.ID.NO.: 42), |
| 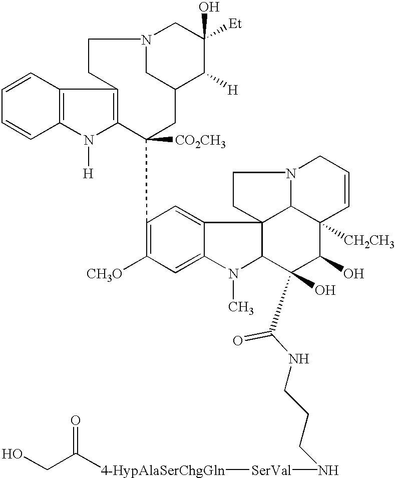 | (SEQ.ID.NO.: 43), |
| 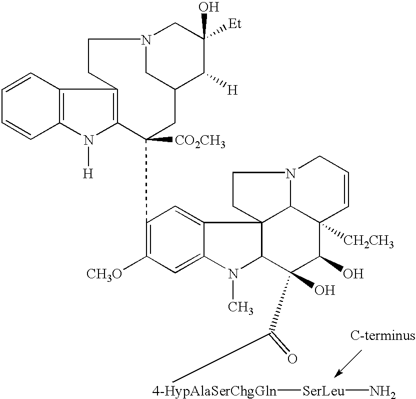 | (SEQ.ID.NO.: 44), |
| vi) | |
| 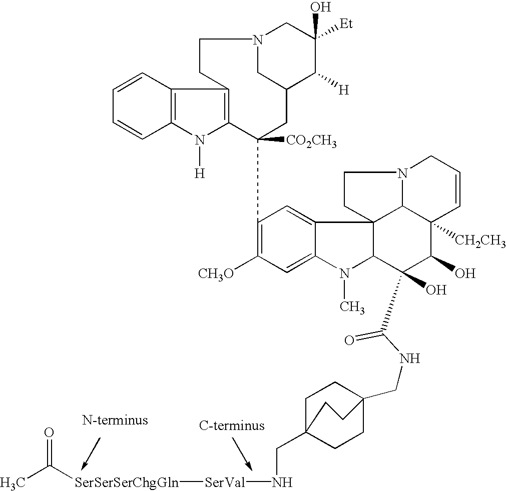 | (SEQ.ID.NO.: 45), |
| 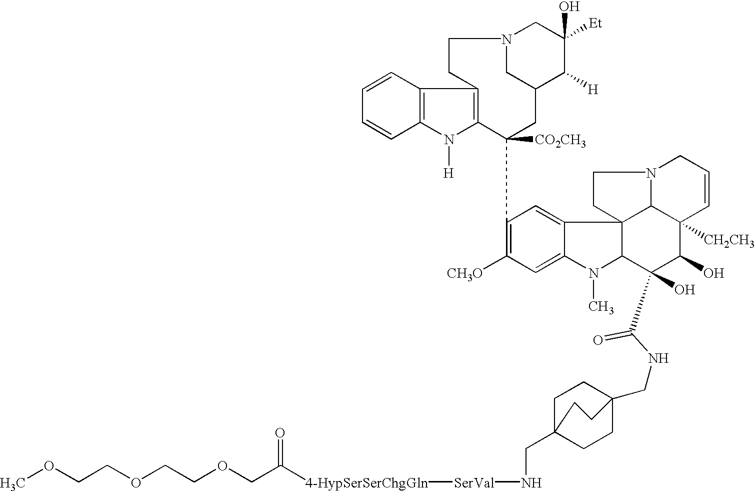 | (SEQ.ID.NO.: 46), |
| 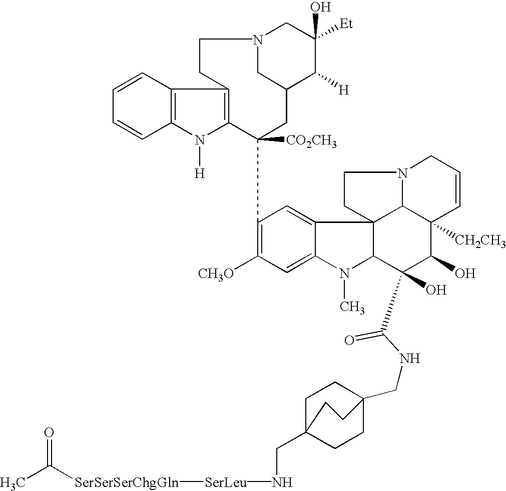 | (SEQ.ID.NO.: 47), |
| 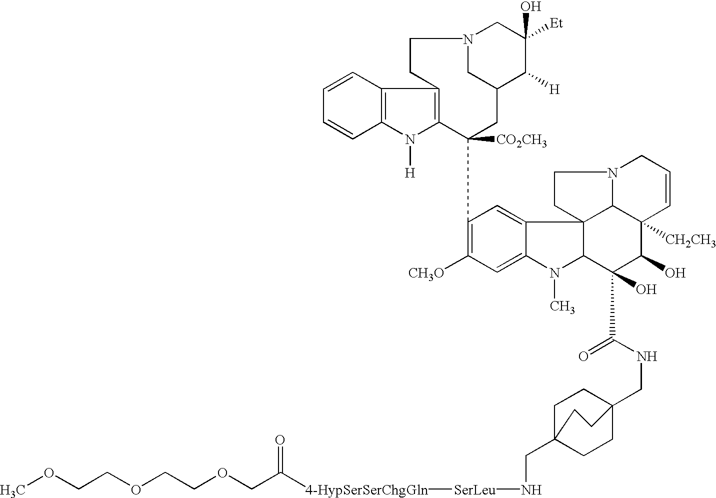 | (SEQ.ID.NO.: 48), |
| vii) | |
| 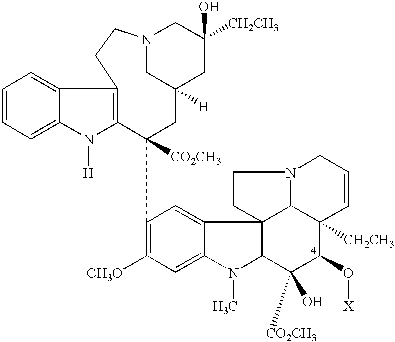 | |
| wherein X is | |
| 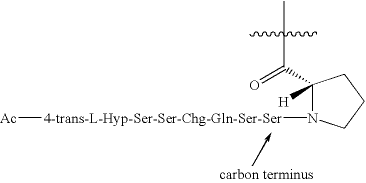 | (SEQ.ID.NO.: 49) |
| 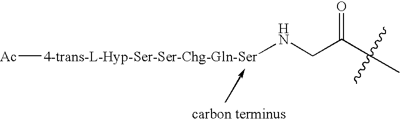 | (SEQ.ID.NO.: 50) |
| 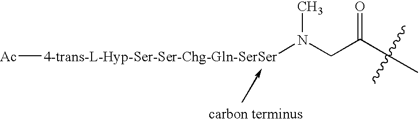 | (SEQ.ID.NO.: 51) |
| 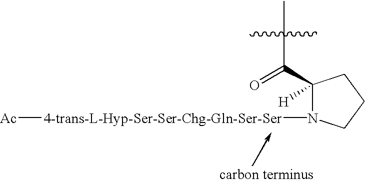 | (SEQ.ID.NO.: 52) |
| 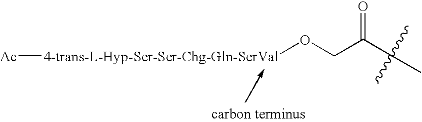 | (SEQ.ID.NO.: 53) |
| 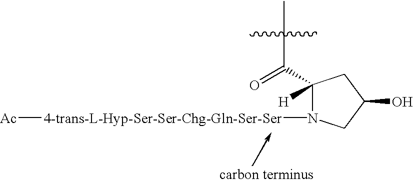 | (SEQ.ID.NO.: 54) |
| 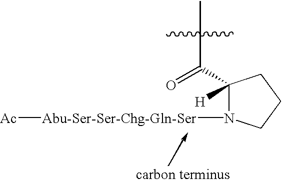 | (SEQ.ID.NO.: 55) |
| 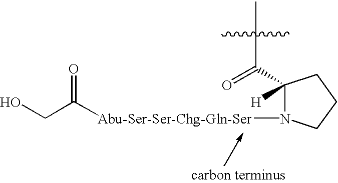 | (SEQ.ID.NO.: 56) |
| 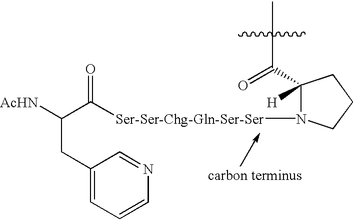 | (SEQ.ID.NO.: 57) |
| 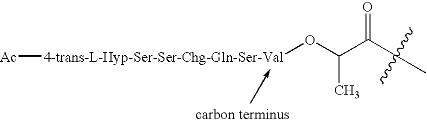 | (SEQ.ID.NO.: 58) |
| 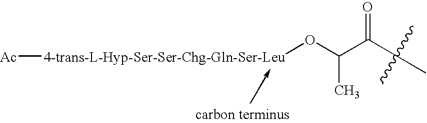 | (SEQ.ID.NO.: 59) |

or a pharmaceutically acceptable salt or optical isomer thereof.

10. The method according to claim 1 wherein the inhibitor of prenyl-protein transferase is a selective inhibitor of farnesyl-protein transferase.

11. The method according to claim 1 wherein the inhibitor of prenyl-protein transferase is a dual inhibitor of farnesyl-protein transferase and geranylgeranyl-protein transferase type I.

12. The method according to claim 11 wherein the dual inhibitor of farnesyl-protein transferase and geranylgeranyl-protein transferase type I is a Class II inhibitor.

13. The method according to claim 11 wherein the dual inhibitor of farnesyl-protein transferase and geranylgeranyl-protein transferase type I is a Class III inhibitor.

14. The method according to claim 1 wherein the inhibitor of prenyl-protein transferase is selected from:
2(S)-Butyl-1-(2,3-diaminoprop-1-yl)-1-(1-naphthoyl)piperazine;
1-(3-Amino-2-(2-naphthylmethylamino)prop-1-yl)-2(S)-butyl-4-(1-naphthoyl)piperazine;
2(S)-Butyl-1-{5-[1-(2-naphthylmethyl)]-4,5-dihydroimidazol}methyl-4-(1-naphthoyl)piperazine;
1-[5-(1-Benzylimidazol)methyl]-2(S)-butyl-4-(1-naphthoyl)piperazine;
1-{(5-[1-(4-nitrobenzyl)]imidazolylmethyl}-2(S)-butyl-4-(1-naphthoyl)piperazine;
1-(3-Acetamidomethylthio-2(R)-aminoprop-1-yl)-2(S)-butyl-4-(1-naphthoyl)piperazine;
2(S)-Butyl-1-[2-(1-imidazolyl)ethyl]sulfonyl-4-(1-naphthoyl)piperazine;
2(R)-Butyl-1-imidazolyl-4-methyl-4-(1-naphthoyl)piperazine; 2(S)-Butyl-4-(1-naphthoyl)-1-(3-pyridylmethyl)piperazine;
1-2(S)-butyl-(2(R)-(4-nitrobenzyl)amino-3-hydroxypropyl)-4-(1-naphthoyl)piperazine;
1-(2(R)-Amino-3-hydroxyheptadecyl)-2(S)-butyl-4-(1-naphthoyl)-piperazine;
2(S)-Benzyl-1-imidazolyl-4-methyl-4-(1-naphthoyl)piperazine;
1-(2(R)-Amino-3-(3-benzylthio)propyl)-2(S)-butyl-4-(1-naphthoyl)piperazine;
1-(2(R)-Amino-3-[3-(4-nitrobenzylthio)propyl])-2(S)-butyl-4-(1-naphthoyl)piperazine;
2(S)-Butyl-1-[(4-imidazolyl)ethyl]-4-(1-naphthoyl)piperazine;
2(S)-Butyl-1-[(4-imidazolyl)methyl]-4-(1-naphthoyl)piperazine;
2(S)-Butyl-1-[(1-naphth-2-ylmethyl)-1H-imidazol-5-yl)acetyl]-4-(1-naphthoyl)piperazine;
2(S)-Butyl-1-[(1-naphth-2-ylmethyl)-1H-imidazol-5-yl)ethyl]-4-(1-naphthoyl)piperazine;
1-(2(R)-Amino-3-hydroypropyl)-2(S)-butyl-4-(1-naphthoyl)piperazine;
1-(2(R)-Amino-4-hydroxybutyl)-2(S)-butyl-4-(1-naphthoyl)piperazine;
1-(2-Amino-3-(2-benzyloxyphenyl)propyl)-2(S)-butyl-4-(1-naphthoyl)piperazine;
1-(2-Amino-3-(2-hydroxyphenyl)propyl)-2(S)-butyl-4-(1-naphthoyl)piperazine;
1-[3-(4-imidazolyl)propyl]-2(S)-butyl-4-(1-naphthoyl)-piperazine;
2(S)-n-Butyl-4-(2,3-dimethylphenyl)-1-(4-imidazolylmethyl)-piperazin-5-one;
2(S)-n-Butyl-1-[1-(4-cyanobenzyl)imidazol-5-ylmethyl]-4-(2,3-dimethylphenyl)piperazin-5-one;
1-[1-(4-Cyanobenzyl)imidazol-5-ylmethyl]-4-(2,3-dimethylphenyl)-2(S)-(2-methoxyethyl)piperazin-5-one;
2(S)-n-Butyl-4-(1-naphthoyl)-1-[1-(1-naphthylmethyl)imidazol-5-ylmethyl]-piperazine;
2(S)-n-Butyl-4-(1-naphthoyl)-1-[1-(2-naphthylmethyl)imidazol-5-ylmethyl]-piperazine;
2(S)-n-Butyl-1-[1-(4-cyanobenzyl)imidazol-5-ylmethyl]-4-(1-naphthoyl)piperazine;
2(S)-n-Butyl-1-[1-(4-methoxybenzyl)imidazol-5-ylmethyl]-4-(1-naphthoyl)piperazine;
2(S)-n-Butyl-1-[1-(3-methyl-2-butenyl)imidazol-5-ylmethyl-4-(1-naphthoyl)piperazine;
2(S)-n-Butyl-1-[1-(4-fluorobenzyl)imidazol-5-ylmethyl]-4-(1-naphthoyl)piperazine;
2(S)-n-Butyl-1-[1-(4-chlorobenzyl)imidazol-5-ylmethyl]-4-(1-naphthoyl)piperazine;
1-[1-(4-Bromobenzyl)imidazol-5-ylmethyl]-2(S)-n-butyl-4-(1-naphthoyl)piperazine;
2(S)-n-Butyl-4-(1-naphthoyl)-1-[1-(4-trifluoromethylbenzyl)imidazol-5-ylmethyl]-piperazine;
2(S)-n-Butyl-1-[1-(4-methylbenzyl)imidazol-5-ylmethyl]-4-(1-naphthoyl)-piperazine;
2(S)-n-Butyl-1-[1-(3-methylbenzyl)imidazol-5-ylmethyl]-4-(1-naphthoyl)-piperazine;
1-[1-(4-Phenylbenzyl)imidazol-5-ylmethyl]-2(S)-n-butyl-4-(1-naphthoyl)-piperazine;
2(S)-n-Butyl-4-(1-naphthoyl)-1-[1-(2-phenylethyl)imidazol-5-ylmethyl]-piperazine;
2(S)-n-Butyl-4-(1-naphthoyl)-1-[1-(4-trifluoromethoxy)imidazol-5-ylmethyl]piperazine;
1-1 [1-(4-cyanobenzyl)-1H-imidazol-5-yl]acetyl]-2(S)-n-butyl-4-(1-naphthoyl)piperazine;
(S)-1-(3-Chlorophenyl)-4-[1-(4-cyanobenzyl)-5-imidazolylmethyl]-5-[2-(methanesulfonyl)ethyl]-2-piperazinone;
(S)-1-(3-Chlorophenyl)-4-[1-(4-cyanobenzyl)-5-imidazolylmethyl]-5-[2-(ethanesulfonyl)ethyl]-2-piperazinone;
(R)-1-(3-Chlorophenyl)-4-[1-(4-cyanobenzyl)-5-imidazolylmethyl]-5-[2-(ethanesulfonyl)methyl]-2-piperazinone;
(S)-1-(3-Chlorophenyl)-4-[1-(4-cyanobenzyl)-5-imidazolylmethyl]-5-[N-ethyl-2-acetamido]-2-piperazinone;
(±)-5-(2-Butynyl)-1-(3-chlorophenyl)-4-[1-(4-cyanobenzyl)-5-imidazolylmethyl]-2-piperazinone;
1-(3-Chlorophenyl)-4-[1-(4-cyanobenzyl)-5-imidazolylmethyl]-2-piperazinone;
5(S)-Butyl-4-[1-(4-cyanobenzyl-2-methyl)-5-imidazolylmethyl]-1-(2,3-dimethylphenyl)-piperazin-2-one;
4-[1-(2-(4-Cyanophenyl)-2-propyl)-5-imidazolylmethyl]-1-(3-chlorophenyl)-5(S)-(2-methylsulfonylethyl)piperazin-2-one;
5(S)-n-Butyl-4-[1-(4-cyanobenzyl)-5-imidazolylmethyl]—(2-methylphenyl)piperazin-2-one;
4-[1-(4-Cyanobenzyl)-5-imidazolylmethyl]-5(S)-(2-fluoroethyl)-1-(3-chlorophenyl)piperazin-2-one;
4-[3-(4-Cyanobenzyl)pyridin-4-yl]-1-(3-chlorophenyl)-5(S)-(2-methylsulfonylethyl)-piperazin-2-one;
4-[5-(4-Cyanobenzyl)-1-imidazolylethyl]-1-(3-chlorophenyl)piperazin-2-one;
4-{3-[4-(−2-Oxo-2-H-pyridin-1-yl)benzyl-3-H-imidazol-4-ylmethyl]benzonitrile;
4-{3-[4-3-Methyl-2-oxo-2-H-pyridin-1-yl)benzyl]-3-H-imidazol-4-ylmethyl]benzonitrile;
4-{3-[4-(−2-Oxo-piperidin-1-yl)benzyl]-3-H-imidazol-4-ylmethyl]benzonitrile;
4-{3-[3-Methyl-4-(2-oxopiperidin-1-yl)-benzyl]-3-H-imidizol-4-ylmethyl}-benzonitrile;
(4-{3-[4-(2-Oxo-pyrrolidin-1-yl)-benzyl]-3H-imidizol-4-ylmethyl}-benzonitrile;
4-13-[4-(3-Methyl-2-oxo-2-H-pyrazin-1-yl)-benzyl-3-H-imidizol-4-ylmethyl}-benzonitrile;
4-{3-[2-Methoxy-4-(2-oxo-2-H-pyridin-1-yl)-benzyl]-3-H-imidizol-4-ylmethyl}-benzonitrile;
4-{1-[4-(5-Chloro-2-oxo-2H-pyridin-1-yl)-benzyl]-1H-pyrrol-2-ylmethyl}-benzonitrile;
4-[1-(2-Oxo-2H-[1,2′]bipyridinyl-5′-ylmethyl)-1H-pyrrol-2-ylmethyl]-benzonitrile;
4-[1-(5-Chloro-2-oxo-2H-[1,2′]bipyridinyl-5′-ylmethyl)-1H-pyrrol-2-ylmethyl]-benzonitrile;
4-[3-(2-Oxo-1-phenyl-1,2-dihydropyridin-4-ylmethyl)-3H-imidazol-4-ylmethyl]benzonitrile;
4-{3-[1-(3-Chloro-phenyl)-2-oxo-1,2-dihydropyridin-4-ylmethyl]-3H-imidazol-4-ylmethyl}benzonitrile;
19,20-Dihydro-19-oxo-5H,17H-18,21-ethano-6,10:12,16-dimetheno-22H-imidazo[3,4-h][1,8,11,14]oxatriazacycloeicosine-9-carbonitrile;
19-Chloro-22,23-dihydro-22-oxo-5H-21,24-ethano-6,10-metheno-25H-dibenzo[b,e]imidazo[4,3-l][1,4,7,10,13]dioxatriazacyclononadecine-9-carbonitrile;
22,23-Dihydro-22-oxo-5H-21,24-ethano-6,10-metheno-25H-dibenzo[b,e]imidazo[4,3-l][1,4,7,10,13]dioxatriazacyclononadecine-9-carbonitrile;
20-Chloro-23,24-dihydro-23-oxo-5H-22′,25-ethano-6,10:12,16-dimetheno-12H,26H-benzo[b]imidazo[4,3-i][1,17,4,7,10]dioxatriazacyclohemicosine-9-carbonitrile;
(S)-20-Chloro-23,24-dihydro-27-[2-(methylsulfonyl)ethyl]-23-oxo-5H-22,25-ethano-6,10:12,16-dimetheno-12H,26H-benzo[b]imidazo[4,3-i][1,17,4,7,10]dioxatriazacyclohemicosine-9-carbonitrile;
(±)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile;
(+)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile;
(−)-19,20-Dihydro-19-oxo-5H-18,21-ethano-12,14-etheno-6,10-metheno-22H-benzo[d]imidazo[4,3-k][1,6,9,12]oxatriazacyclooctadecine-9-carbonitrile;
5H,17H,20H-18,21-Ethano-6,10:12,16-dimetheno-22H-imidazo[3,4-h][1,8,11,14]oxatriazacycloeicosin-20-one;
(±)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile;
(+) or (−)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile; (Enantiomer A)
(−) or (+)-19,20-Dihydro-3-methyl-19-oxo-5H-18,21-ethano-12,14-etheno-6,10-metheno-22H-benzo[d]imidazo[4,3-k][1,6,9,12]oxatriazacyclooctadecine-9-carbonitrile; (Enantiomer B)
(±)-19,20-Dihydro-19,22-dioxo-5H-18,21-ethano-12,14-etheno-6,10-metheno-22H-benzo[d]imidazo[4,3-k][1,6,9,12]oxatriazacyclooctadecine-9-carbonitrile; 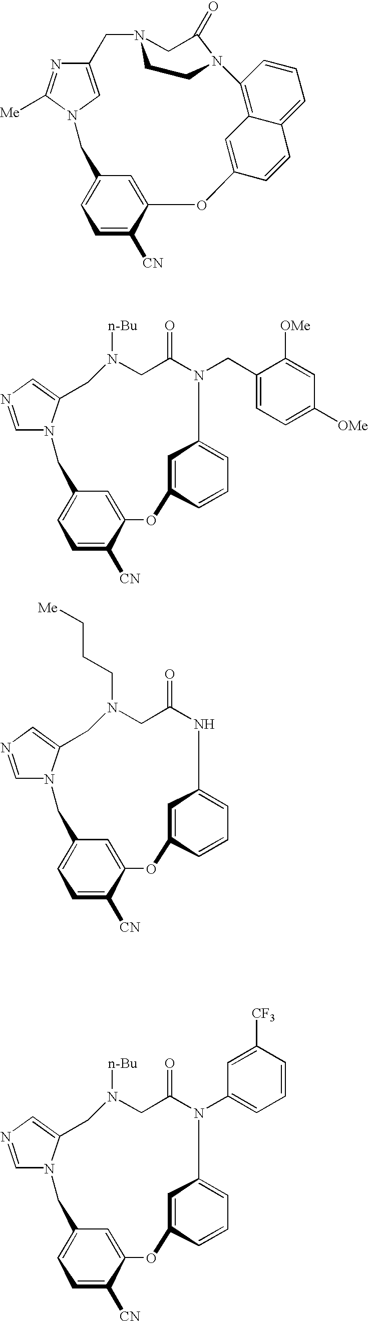
18,19-dihydro-19-oxo-5H, 17H-6, 10:12, 16-dimetheno-1H-imidazo[4,3-c][1,11,4]dioxaazacyclononadecine-9-carbonitrile;
17,18-dihydro-18-oxo-5H-6,10:12,16-dimetheno-12H,20H-imidazo[4,3-c][1,11,4]dioxaazacyclooctadecine-9-carbonitrile;
(±)-17,18,19,20-tetrahydro-19-phenyl-5H-6,10:12,16-dimetheno-21H-imidazo[3,4-h][1,8,11]oxadiazacyclononadecine-9-carbonitrile;
21,22-dihydro-5H-6,10:12,16-dimetheno-23H-benzo[g]imidazo[4,3-1][1,8,11]oxadiazacyclononadecine-9-carbonitrile;
22,23-dihydro-23-oxo-5H,21H-6,10:12,16-dimetheno-24H-benzo g]imidazo[4,3-m][1,8,12]oxadiazaeicosine-9-carbonitrile;
22,23-dihydro-5H,21H-6,10:12,16-dimetheno-24H-benzo[g]imidazo[4,3-m][1,8,11]oxadiazaeicosine-9-carbonitrile;
1-(3-trifluoromethoxyphenyl)-4-[1-(4-cyano-3-methoxybenzyl)-5-imidazolyl methyl]-2-piperazinone;
or a pharmaceutically acceptable salt, stereoisomer or optical isomer thereof.
Specific examples of a farnesyl-protein transferase inhibitor are 1-(3-Chlorophenyl)-4-[1-(4-cyanobenzyl)-5-imidazolylmethyl]-2-piperazinone;
(R)-1-(3-Chlorophenyl)-4-[1-(4-cyanobenzyl)-5-imidazolylmethyl]-5-[2-(ethanesulfonyl)methyl]-2-piperazinone;
4-[1-(5-Chloro-2-oxo-2H-[1,2′]bipyridinyl-5′-ylmethyl)-1H-pyrrol-2-ylmethyl]-benzonitrile; and
1-[N-(1-(4-cyanobenzyl)-5-imidazolylmethyl)-N-(4-cyanobenzyl)amino]-4-(phenoxy)benzene;
(±)-19,20-Dihydro-19-oxo-5H-18,21-ethano-12, 14-etheno-6,10-metheno-22H-benzo[d]imidazo[4,3-k][1,6,9,12]oxatriaza-cyclooctadecine-9-carbonitrile;
1-(3-trifluoromethoxyphenyl)-4-[1-(4-cyano-3-methoxybenzyl)-5-imidazolyl methyl]-2-piperazinone;
3-(biphenyl-4-ylmethoxy)-4-imidazol-1-ylmethyl-benzonitrile;
3-(biphenyl-4-yl-2-ethoxy)-4-imidazol-1-ylmethylbenzonitrile;
3-(biphenyl-3-ylmethoxy)-4-imidazol-1-ylmethyl-benzonitrile;
2-(biphenyl-4-ylmethoxy)-4-imidazol-1-ylmethyl-benzonitrile;
2-(biphenyl-4-yl-2-ethoxy)-4-imidazol-1-ylmethyl-benzonitrile;
1-tert-butoxycarbonyl-4-(3-chlorophenyl)-2(S)-[2-(2-cyano-5-imidazol-1-ylmethyl-phenoxy)ethyl]piperazine;
2-(3-chlorophenoxy)-4-imidazol-1-ylmethyl-benzonitrile;
2-(4-chlorophenyl-2-ethoxy)-4-imidazol-1-ylmethyl-benzonitrile;
2-(3-chlorophenyl-2-ethoxy)-4-imidazol-1-ylmethyl-benzonitrile;
2-(2-chlorophenyl-2-ethoxy)-4-imidazol-1-ylmethyl-benzonitrile;
2-(phenyl-2-ethoxy)-4-imidazol-1-ylmethyl-benzonitrile;
2-(3-chlorobenzyloxy)-4-imidazol-1-ylmethyl-benzonitrile;
2-(4-chlorobenzyloxy)-4-imidazol-1-ylmethyl-benzonitrile;
2-(2,4-dichlorobenzyloxy)-4-imidazol-1-ylmethyl-benzonitrile;
2-(benzyloxy)-4-imidazol-1-ylmethyl-benzonitrile;
2-(biphenyl-2-ylmethoxy)-4-imidazol-1-ylmethyl-benzonitrile;
2-(phenyl-4-butoxy)-4-imidazol-1-ylmethyl-benzonitrile;
2-(phenyl-3-propoxy)-4-imidazol-1-ylmethyl-benzonitrile;
2-(biphenyl-4-yl-2-ethoxy)-4-(1,2,4-triazol-1-yl)methyl-benzonitrile;
2-(biphenyl-4-yl-2-ethoxy)-4-(2-methyl-imidazol-1-yl)methyl-benzonitrile;
2-(biphenyl-4-yl-2-ethoxy)-4-benzimidazol-1-yl)methyl-benzonitrile;
4-imidazol-1-ylmethyl-2-(naphthalen-2-yloxy)-benzonitrile;
2-(3-cyanophenoxy)-4-imidazol-1-ylmethyl-benzonitrile;
2-(3-bromophenoxy)-4-imidazol-1-ylmethyl-benzonitrile;
2-(biphen-3-yloxy)-4-imidazol-1-ylmethyl-benzonitrile;
2-(biphen-4-yloxy)-4-imidazol-1-ylmethyl-benzonitrile;
2-(3-acetylphenoxy)-4-imidazol-1-ylmethyl-benzonitrile;
2-(2-acetylphenoxy)-4-imidazol-1-ylmethyl-benzonitrile;
2-(3-trifluoromethylphenoxy)-4-imidazol-1-ylmethyl-benzonitrile;
2-(3-methylphenoxy)-4-imidazol-1-ylmethyl-benzonitrile;
2-(2-methylphenoxy)-4-imidazol-1-ylmethyl-benzonitrile;
2-(4-methylphenoxy)-4-imidazol-1-ylmethyl-benzonitrile;
2-(3-methoxyphenoxy)-4-imidazol-1-ylmethyl-benzonitrile;
2-(2-methoxyphenoxy)-4-imidazol-1-ylmethyl-benzonitrile;
2-(4-methoxyphenoxy)-4-imidazol-1-ylmethyl-benzonitrile;
2-(3,5-dimethylphenoxy)-4-imidazol-1-ylmethyl-benzonitrile;
2-(3,4-dimethylphenoxy)-4-imidazol-1-ylmethyl-benzonitrile;
2-(3,5-dimethoxyphenoxy)-4-imidazol-1-ylmethyl-benzonitrile;
2-(1-naphthyloxy)-4-imidazol-1-ylmethyl-benzonitrile;
2-(2,4-dichlorophenoxy)-4-imidazol-1-ylmethyl-benzonitrile;
2-(3-fluorophenoxy)-4-imidazol-1-ylmethyl-benzonitrile;
2-(3-t-butylphenoxy)-4-imidazol-1-ylmethyl-benzonitrile;
2-[3-(N,N-diethylamino)phenoxy]-4-imidazol-1-ylmethyl-benzonitrile;
2-(3-n-propylphenoxy)-4-imidazol-1-ylmethyl-benzonitrile;
2-(2,3-dimethoxyphenoxy)-4-imidazol-1-ylmethyl-benzonitrile;
2-(2,3-dimethylphenoxy)-4-imidazol-1-ylmethyl-benzonitrile;
2-(3,4-dimethoxyphenoxy)-4-imidazol-1-ylmethyl-benzonitrile;
2-(2,5-dimethoxyphenoxy)-4-imidazol-1-ylmethyl-benzonitrile;
2-(3,4-dichlorophenoxy)-4-imidazol-1-ylmethyl-benzonitrile;
2-(2,4-dimethylphenoxy)-4-imidazol-1-ylmethyl-benzonitrile;
2-(4-chloro-2-methylphenoxy)-4-imidazol-1-ylmethyl-benzonitrile;
2-(5-chloro-2-methylphenoxy)-4-imidazol-1-ylmethyl-benzonitrile;
2-(2-chloro-4,5-dimethylphenoxy)-4-imidazol-1-ylmethyl-benzonitrile;
2-(5-hydroxymethyl-2-methoxyphenoxy)-4-imidazol-1-ylmethyl-benzonitrile;
4-imidazol-1-ylmethyl-2-(3-phenylamino-phenoxy)-benzonitrile;
4-imidazol-1-ylmethyl-2-[3-(2-methylphenylamino)-phenoxy]-benzonitrile;
4-imidazol-1-ylmethyl-2-(3-phenoxy-phenoxy)-benzonitrile;
2-(2-benzoyl-phenoxy)-4-imidazol-1-ylmethyl-benzonitrile;
1-(5-chloro-2-methoxy-phenyl)-3-[3-(2-cyano-5-imidazol-1-ylmethyl-phenoxy)-phenyl]-urea;
1-(2,5-dimethoxy-phenyl)-3-[3-(2-cyano-5-imidazol-1-ylmethyl-phenoxy)-phenyl]-urea;
2-(3-benzyloxy-phenoxy)-4-imidazol-1-ylmethyl-benzonitrile;
2-(4-benzyloxy-phenoxy)-4-imidazol-1-ylmethyl-benzonitrile;
2-(2-benzyl-phenoxy)-4-imidazol-1-ylmethyl-benzonitrile;
2-(3-ethynyl-phenoxy)-4-imidazol-1-ylmethyl-benzonitrile;
2-(4-acetyl-3-methyl-phenoxy)-4-imidazol-1-ylmethyl-benzonitrile;
4-imidazol-1-ylmethyl-2-(1H-indazol-6-yloxy)-benzonitrile;
4-imidazol-1-ylmethyl-2-(5,6,7,8-tetrahydro-naphthalen-1-yloxy)-benzonitrile;
4-imidazol-1-ylmethyl-2-(8-oxo-5,6,7,8-tetrahydro-naphthalen-1-yloxy)-benzonitrile;
4-imidazol-1-ylmethyl-2-(1H-indol-7-yloxy)-benzonitrile;
4-imidazol-1-ylmethyl-2-(3-oxo-indan-4-yloxy)-benzonitrile;
4-imidazol-1-ylmethyl-2-(1H-indol-4-yloxy)-benzonitrile;
2-[3-(2-hydroxy-ethoxy)-phenoxy]-4-imidazol-1-ylmethyl-benzonitrile;
4-imidazol-1-ylmethyl-2-(4-imidazol-1-yl-phenoxy)-benzonitrile;
4-(2-cyano-5-imidazol-1-ylmethyl-phenoxy)-biphenyl-4-carbonitrile;
N-[3-(2-cyano-5-imidazol-1-ylmethyl-phenoxy)-phenyl]-acetamide;
4-imidazol-1-ylmethyl-2-(9-oxo-9H-fluoren-4-yloxy)-benzonitrile;
3-(2-cyano-5-imidazol-1-ylmethyl-phenoxy)-Nphenyl-benzamide;
3-(2-cyano-5-imidazol-1-ylmethyl-phenoxy)-N-ethyl-N-phenyl-benzamide;
3-(2-cyano-5-imidazol-1-ylmethyl-phenoxy)-N-cyclopropylmethyl-N-phenyl-benzamide;
2-(5-chloro-pyridin-3-yloxy)-4-imidazol-1-ylmethyl-benzonitrile;
N-[3-(2-cyano-5-imidazol-1-ylmethyl-phenoxy)-phenyl]-benzenesulfonamide;
4-imidazol-1-ylmethyl-2-(indan-5-yloxy)-benzonitrile;
3-(9H-carbazol-2-yloxy)-4-imidazol-1-ylmethyl-benzonitrile;
4-imidazol-1-ylmethyl-2-(5,6,7,8-tetrahydro-naphthalen-2-yloxy)-benzonitrile;
4-imidazol-1-ylmethyl-2-(2-methoxy-4-propenyl-phenoxy)-benzonitrile;
4-imidazol-1-ylmethyl-2-[4-(3-oxo-butyl)-phenoxy]-benzonitrile;
2-(3-chlorophenoxy)-5-imidazol-1-ylmethyl-benzonitrile;
2-(4-chlorophenoxy)-4-imidazol-1-ylmethyl-benzonitrile;
2-(3,5-dichlorophenoxy)-4-imidazol-1-ylmethyl-benzonitrile;
2-(pyridin-3-yloxy)-4-imidazol-1-ylmethyl-benzonitrile;
2-(2-chlorophenoxy)-4-imidazol-1-ylmethyl-benzonitrile;
2-(3-chlorophenoxy)-5-(4-phenyl-imidazol-1-ylmethyl)-benzonitrile;
2-(biphen-2-yloxy)-4-imidazol-1-ylmethyl-benzonitrile;
2-(phenoxy)-4-imidazol-1-ylmethyl-benzonitrile;
2-(2-chloro-4-methoxyphenoxy)-4-imidazol-1-ylmethyl-benzonitrile;
2-(2-chlorophenylsulfanyl)-4-imidazol-1-ylmethyl-benzonitrile;
4-imidazol-1-ylmethyl-2-(naphthalen-2-ylsulfanyl)-benzonitrile;
2-(2,4-dichlorophenylsulfanyl)-4-imidazol-1-ylmethyl-benzonitrile;
2-(2,4-dichloro-benzenesulfinyl)-4-imidazol-1-ylmethyl-benzonitrile;
2-(2,4-dichloro-benzenesulfonyl)-4-imidazol-1-ylmethyl-benzonitrile;
2-(2-methyl-pyridin-3-yloxy)-4-imidazol-1-ylmethyl-benzonitrile;
2-(2,4-dimethyl-pyridin-3-yloxy)-4-imidazol-1-ylmethyl-benzonitrile;
2-(4-chloro-2-methoxyphenoxy)-4-imidazol-1-ylmethyl-benzonitrile;
2-(2-chlorophenoxy)-4-(5-methyl-imidazol-1-ylmethyl)-benzonitrile;
2-(2-chlorophenoxy)-4-(4-methyl-imidazol-1-ylmethyl)-benzonitrile;
2-(3-chloro-5-trifluoromethyl-pyridin-2-yloxy)-4-imidazol-1-ylmethyl-benzonitrile;
2-(2,4-dichlorophenoxy)-4-(2-methyl-imidazol-1-ylmethyl)-benzonitrile;
N-[3-(2-cyano-5-imidazol-1-ylmethyl-phenoxy)-phenyl]-benzamide;
2-[3-(2-cyano-5-imidazol-1-ylmethyl-phenoxy)-phenyl]-N-phenyl-acetamide;
4-imidazol-1-ylmethyl-2-(quinolin-6-yloxy)-benzonitrile;
4-imidazol-1-ylmethyl-2-(2-oxo-1,2-dihydro-quinolin-6-yloxy)-benzonitrile;
N-[3-(2-cyano-5-imidazol-1-ylmethyl-phenoxy)-phenyl]-2-phenyl-acetamide;
5-(2-cyano-5-imidazol-1-ylmethyl-phenoxy)-N-cyclohexyl-nicotinamide;
N-(3-chloro-phenyl)-5-(2-cyano-5-imidazol-1-ylmethyl-phenoxy)-nicotinamide;
2-(2,3-dimethoxyphenoxy)-4-(2,4-dimethyl-imidazol-1-ylmethyl)-benzonitrile;
4-(2-methyl-imidazol-1-ylmethyl)-2-(naphthalen-2-yloxy)-benzonitrile;
4-(1-imidazol-1-yl-1-methyl-ethyl)-2-(naphthalen-2-yloxy)-benzonitrile;
1-[4-iodo-3-(naphthalen-2-yloxy)-benzyl]-1H-imidazole; acetic acid 3-[3-(2-chloro-phenoxy)-4-cyano-benzyl]-3H-imidazol-4-ylmethyl ester;
2-(2-chloro-phenoxy)-4-(5-hydroxymethyl-imidazol-1-ylmethyl)-benzonitrile;
4-(5-aminomethyl-imidazol-1-ylmethyl)-2-(2-chloro-phenoxy)-benzonitrile;
N-{3-[4-cyano-3-(2,3-dimethoxy-phenoxy)-benzyl]-3H-imidazol-4-ylmethyl}-2-cyclohexyl-acetamide;
2-(3-chloro-phenoxy)-4-[(4-chloro-phenyl)-imidazol-1-yl-methyl]-benzonitrile;
2-(3-chloro-phenoxy)-4-[1-(4-chloro-phenyl)-2-hydroxy-1-imidazol-1-yl-ethyl]-benzonitrile;
2-(3-chloro-phenoxy)-4-[(4-chloro-phenyl)-hydroxy-(3H-imidazol-4-yl)-methyl]-benzonitrile;
2-(2,4-dichloro-phenylsulfanyl)-4-[5-(2-morpholin-4-yl-ethyl)-imidazol-1-ylmethyl]-benzonitrile;
2-(2,4-dichloro-phenoxy)-4-[5-(2-morpholin-4-yl-ethyl)-imidazol-1-ylmethyl]-benzonitrile;
4-[hydroxy-(3-methyl-3H-imidazol-4-yl)-methyl]-2-(naphthalen-2-yloxy)-benzonitrile;
4-[amino-(3-methyl-3H-imidazol-4-yl)-methyl]-2-(naphthalen-2-yloxy)-benzonitrile;
4-[1-hydroxy-1-(3-methyl-3H-imidazol-4-yl)-ethyl]-2-(naphthalen-2-yloxy)-benzonitrile;
4-[1-amino-1-(3-methyl-3H-imidazol-4-yl)-ethyl]-2-(naphthalen-2-yloxy)-benzonitrile hydrochloride;
3-{2-cyano-5-[1-amino-1-(3-methyl-3H-imidazol-4-yl)-ethyl]-phenoxy}-N-ethyl-N-phenyl-benzamide;
3-{2-cyano-5-[1-hydroxy-1-(3-methyl-3H-imidazol-4-yl)-ethyl]-phenoxy}-N-ethyl-N-phenyl-benzamide;
4-[1-hydroxy-1-(3-methyl-3H-imidazol-4-yl)-ethyl]-2-(3-phenylamino-phenoxy)-benzonitrile;
4-[1-hydroxy-1-(3-methyl-3H-imidazol-4-yl)-ethyl]-2-(3-phenoxy-phenoxy)-benzonitrile;
2-(3-benzoyl-phenoxy)-4-[1-hydroxy-1-(3-methyl-3H-imidazol-4-yl)-ethyl]-benzonitrile;
2-(3-tert-butyl-phenoxy)-4-[1-hydroxy-1-(3-methyl-3H-imidazol-4-yl)-ethyl]-benzonitrile;
2-(3-diethylamino-phenoxy)-4-[1-hydroxy-1-(3-methyl-3H-imidazol-4-yl)-ethyl]-benzonitrile;
2-(5-chloro-2-oxo-2H-[1,2′]bipyridinyl-5′-ylmethoxy)-4-imidazol-1-ylmethyl-benzonitrile;
4-Imidazol-1-ylmethyl-2-[2-(2-oxo-2H-pyridin-1-yl)-phenoxy]-benzonitrile;
4-Imidazol-1-ylmethyl-2-[3-(2-oxo-2H— pyridin-1-yl)-phenoxy]-benzonitrile;
4-Imidazol-1-ylmethyl-2-[4-(2-oxo-2H— pyridin-1-yl)-phenoxy]-benzonitrile;
4-imidazol-1-ylmethyl-2-[3-(2-oxo-piperidin-1-yl)-phenoxy]-benzonitrile;
4-imidazol-1-ylmethyl-2-[4-(2-oxo-piperidin-1-yl)-phenoxy]-benzonitrile;
4-imidazol-1-ylmethyl-2-[2-(3-methyl-2-oxo-piperidin-1-yl)-phenoxy]-benzonitrile;
4-imidazol-1-ylmethyl-2-(3-morpholin-4-yl-phenoxy)-benzonitrile;
4-imidazol-1-ylmethyl-2-(3-piperidin-1-ylmethyl-phenoxy)-benzonitrile;
2-[2-(3,3-dimethyl-2-oxo-piperidin-1-yl)-phenoxy]-4-imidazol-1-ylmethyl-benzonitrile;
2-[3-(3-ethyl-1-methyl-2-oxo-azepan-3-yl)-phenoxy]-4-imidazol-1-ylmethyl-benzonitrile;
2-[3-(3-ethyl-1-methyl-2-oxo-azepan-3-yl)-phenoxy]-4-(2-methyl-imidazol-1-yl)methyl-benzonitrile;
2-[3-(3-ethyl-1-methyl-2-oxo-azepan-3-yl)-phenoxy]-4-(5-methyl-imidazol-1-yl)methyl-benzonitrile;
2-[3-(3-ethyl-1-methyl-2-oxo-azepan-3-yl)-phenoxy]-4-(2,5-dimethyl-imidazol-1-yl)methyl-benzonitrile;
2-[3-(3-ethyl-1-methyl-2-oxo-azepan-3-yl)-phenoxy]-4-[1,2,4]triazol-4-ylmethyl-benzonitrile;
2-[3-(3-ethyl-1-methyl-2-oxo-azepan-3-yl)-phenoxy]-4-[1,2,4]triazol-1-ylmethyl-benzonitrile;
4-imidazol-1-ylmethyl-2-[3-(1-methyl-2-oxo-azepan-3-yl)-phenoxy]-benzonitrile;
4-imidazol-1-ylmethyl-2-[3-(1-methyl-2-oxo-azocan-3-yl)-phenoxy]-benzonitrile;
4-imidazol-1-ylmethyl-2-[3-(1-methyl-2-oxo-piperidin-3-yl)-phenoxy]-benzonitrile;
4-imidazol-1-ylmethyl-2-[3-(3-ethyl-1-methyl-2-oxo-piperidin-3-yl)-phenoxy]-benzonitrile;
4-imidazol-1-ylmethyl-2-[3-(2-oxo-azepan-3-yl)-phenoxy]-benzonitrile;
2-[3-(3-hydroxymethyl-1-methyl-2-oxo-azepan-3-yl)-phenoxy]-4-imidazol-1-ylmethyl-benzonitrile;
2-[3-(3-cyclopropylmethyl-1-methyl-2-oxo-azepan-3-yl)-phenoxy]-4-imidazol-1-ylmethyl-benzonitrile;
2-[4-bromo-3-(3-cyclopropylmethyl-1-methyl-2-oxo-azepan-3-yl)-phenoxy]-4-imidazol-1-ylmethyl-benzonitrile;
2-[3-(3-methoxymethyl-1-methyl-2-oxo-azepan-3-yl)-phenoxy]-4-imidazol-1-ylmethyl-benzonitrile;
2-[3-(3-ethyl-2-oxo-azepan-3-yl)-phenoxy]-4-imidazol-1-ylmethyl-benzonitrile;
2-[3-(3-ethyl-azepan-3-yl)-phenoxy]-4-imidazol-1-ylmethyl-benzonitrile;
2-[3-(1-acetyl-3-ethyl-azepan-3-yl)-phenoxy]-4-imidazol-1-ylmethyl-benzonitrile;
3-[3-(2-cyano-5-imidazol-1-ylmethyl-phenoxy)-phenyl]-3-ethyl-azepane-1-carboxylic acid-tert-butyl ester;
4-[5-(2-amino-ethyl)-2-methyl-imidazol-1-ylmethyl]-2-[3-(3-ethyl-1-methyl-2-oxo-azepan-3-yl)-phenoxy]-benzonitrile;
2-[3-(3-ethyl-1-methyl-2-oxo-azepan-3-yl)-phenoxy]-4-[2-methyl-5-(2-morpholin-4-yl-ethyl)-imidazol-1-ylmethyl]-benzonitrile;
N-[2-(3-{4-cyano-3-[3-(3-ethyl-1-methyl-2-oxo-azepan-3-yl)-phenoxy]-benzyl}-2-methyl-3H-imidazol-4-yl)-ethyl]-acetamide;
3-ethyl-3-[3-(3-imidazol-1-ylmethyl-phenoxy)-phenyl]-1-methyl-azepan-2-one;
2-[3-(3-ethyl-1-methyl-2-oxo-azepan-3-yl)-phenoxy]-4-(3-methyl-3-H-imidazol-4-ylmethyl)-benzonitrile;
2-[3-(3-ethyl-1-methyl-2-oxo-azepan-3-yl)-phenoxy]-4-(3H-imidazol-4-ylmethyl)-benzonitrile;
2-[3-(3-ethyl-1-methyl-2-oxo-azepan-3-yl)-phenoxy]-4-[hydroxy-(3-methyl-3-H-imidazol-4-yl)-methyl]-benzonitrile;
4-[amino-(3-methyl-3-H-imidazol-4-yl)-methyl]-2-[3-(3-ethyl-1-methyl-2-oxo-azepan-3-yl)-phenoxy]-benzonitrile;
2-[3-(3-ethyl-1-methyl-2-oxo-azepan-3-yl)-benzyl]-4-(3-methyl-3H-imidazole-4-carbonyl)-benzonitrile;
2-[3-(3-ethyl-1-methyl-2-oxo-azepan-3-yl)-phenoxy]-4-(hydroxy-pyridin-3-yl-methyl)-benzonitrile;
2-[3-(3-ethyl-1-methyl-2-oxo-azepan-3-yl)-phenoxy]-4-pyridin-3-ylmethyl-benzonitrile;
2-[3-(3-ethyl-1-methyl-2-oxo-azepan-3-yl)-phenoxy]-4-pyridin-2-ylmethyl-benzonitrile;
2-[3-(3-ethyl-1-methyl-2-oxo-azepan-3-yl)-phenoxy]-4-[1-hydroxy-1-(3-methyl-3H-imidazol-4-yl)-ethyl]-benzonitrile;
2-[3-(3-ethyl-1-methyl-2-oxo-azepan-3-yl)-phenoxy]-4-[1-amino-1-(3-methyl-3H-imidazol-4-yl)-ethyl]-benzonitrile;
1-[1-(4′-Cyanobenzyl) imidazol-5-ylmethyl]-4-[1-phenyl-1-cyclopentylcarbonyl]piperazine;
1-[1-(4′-Cyanobenzyl) imidazol-5-ylmethyl]-4-[Cyclohexylphenylacetyl]piperazine;
1-[1-(4′-Cyanobenzyl) imidazol-5-ylmethyl]-4-[1-(3-methoxyphenyl)-1-cyclopentylcarbonyl]piperazine;
1-[1-(4′-Cyanobenzyl) imidazol-5-ylmethyl]-4-[1-(3-phenoxyphenyl)-1-cyclopentylcarbonyl]piperazine;
1-[1-(4′-Cyano-3-fluorobenzyl) imidazol-5-ylmethyl]-4-[1-(3-hydroxyphenyl)-1-cyclohexylcarbonyl]piperazine;
1-[1-(4′-Cyanobenzyl) imidazol-5-ylmethyl]piperazine-4-carboxylic acid-(2,6-dimethoxy)benzyl ester;
1-[1-(4′-Cyanobenzyl) imidazol-5-ylmethyl]piperazine-4-(DL-2-hydroxy-2-(o-methoxyphenyl)) acetamide;
1-[1-(4′-Cyanobenzyl) imidazol-5-ylmethyl]-4-[1-(2,6-dimethylbenzyloxycarbonyl]piperazine;
1-[1-(4′-Cyanobenzyl) imidazol-5-ylmethyl]-4-[1-(2-methoxyphenyl)-1-cyclopentylcarbonyl]piperazine;
(+/−) 1-[1-(4′-Cyanobenzyl) imidazol-5-ylmethyl]-4-[1-(bicyclo[3.1.0]hex-3-yl)-1-(3-methoxyphenyl)-carbonyl]piperazine;
(R/S) 2 [4-((Phenyl)methyloxycarbonyl-1-piperazine)]-2-[1-(4′-cyanobenzyl)-2-methyl-5-imidazol]acetonitrile;
1-[1-(4′-methylbenzyl) imidazol-5-ylmethyl]-4-[1-(2,6-dimethylbenzyloxycarbonyl]piperazine;
1-[1-(4′-Cyanobenzyl) imidazol-5-ylmethyl]piperazine-4-carboxylic acid-(4-nitro)phenyl ester;
1-[1-(4-Cyanobenzyl) imidazol-5-ylmethyl]-4-[3-(4-fluorophenyl)-3-(tricyclo[3.3.1.1^{3 7}]dec-2-yl)-propionyl]piperazine;
2-(1-(4′-cyanobenzyl)imidazol-5-yl-2-[4-(phenylmethyloxy carbonyl)piperazin-1-yl]acetamide;
1-[1-(4′-cyanobenzyl) imidazol-5-ylmethyl]-4-[1-(2-methoxy-5-chlorobenzyloxycarbonyl]piperazine;
1-[1-(4′-cyanobenzyl) imidazol-5-ylmethyl]-4-[1-(pentafluororobenzyloxycarbonyl]piperazine;
1-[1-(4′-cyanobenzyl) imidazol-5-ylmethyl]-4-[1-(2-ethoxybenzyloxycarbonyl]piperazine;
1-[1-(4′-cyanobenzyl) imidazol-5-ylmethyl]-4-{1-[(2-methoxypyridin-3-yl)methyloxycarbonyl]}piperazine;
1-[1-(4′-cyanobenzyl) imidazol-5-ylmethyl]-4-[1-(2-trifluoromethoxybenzyloxycarbonyl]piperazine;
1-[1-(4′-cyanobenzyl) imidazol-5-ylmethyl]-4-[1-(2,3-methylenedioxybenzyloxycarbonyl]piperazine;
1-[1-(4′-Cyanobenzyl) imidazol-5-ylmethyl]piperazine-4-carboxylic acid benzyl ester;
1-[1-(4′-Cyanobenzyl) imidazol-5-ylmethyl]-piperazine-3-carboxylic acid-4-carboxylic acid benzyl ester;
1-[1-(4′-Cyanobenzyl) imidazol-5-ylmethyl]-3-methyl carboxy-piperazine-4-carboxylic acid benzyl ester;
1-[1-(4′-Cyanobenzyl) imidazol-5-ylmethyl]piperazine-4-(N-3-isopropenyl-1,1-dimethylbenzyl)carboxamide;
1-[(1-(4′-cyanobenzyl) imidazol-5-ylmethyl]-4-phenylmethanesulfonyl-(cis)-2,6-dimethylpiperazine;
2-((4′-cyanobenzyl)-5-imidazolyl))-2-[(4′-phenylmethyloxycarbonyl) piperazin-1′-yl]acetonitrile;
1-[1-(4′-Cyanobenzyl) imidazol-5-ylmethyl]-4-(2-tert-butyl-3-phenyl)propionyl piperazine;
1-[1-(4′-Cyanobenzyl) imidazol-5-ylmethyl]-4-[1-(4-methoxyphenyl)-1-cyclohexyl]carbonyl piperazine;
1-[1-1-(4′-Cyanobenzyl) imidazol-5-ylmethyl]-4-{1-[(2-ethoxypyridin-3-yl)methyloxycarbonyl]piperazine;
1-[1-(4′-Cyanobenzyl) imidazol-5-ylmethyl]-4-[1-(2-methanesulfonylbenzyloxycarbonyl) piperazine;
1-[1-(4′-Cyanobenzyl) imidazol-5-yl)-2-(ethoxybenzyl)]piperazine-4-carbamide;
[1-((1(4′-Cyanobenzyl)-2-methyl)imidazol-5-yl)-4-(benzyloxycarbonyl)]piperazine;
1-[1-(4′-Cyanobenzyl) imidazol-5-ylmethyl]piperazine-4-(N-3-methylbenzyl)carboxamide;
1-[1-(4′-Cyanobenzyl) imidazol-5-ylmethyl]piperazine-4-(N-2-chlorobenzyl)carboxamide;
1-[1-(4′-Cyanobenzyl)imidazol-5-yl)-(2-methoxybenzyl)]piperazine-4-carboxamide;
1-[1-(4-Cyanobenzyl)imidazol-5-yl)-(3-methoxy-6-chlorobenzyl)]piperazine-4-carboxamide;
1-[1-(4-Cyanobenzyl)imidazol-5-yl)-(2-methyl-5-chlorobenzyl)]piperazine-4-carboxamide;
1-[1-(4′-Cyanobenzyl)imidazol-5-yl)-(3-phenylpropyl)]piperazine-4-carboxamide;
1-[1-(4-Cyanobenzyl)imidazol-5-yl)-(2,5-dimethylbenzyl)]piperazine-4-carbamide;
1-[1-(4′-Cyanobenzyl)imidazole-5-ylmethyl]-4-benzyloxycarbonyl)-(trans)-2,5-dimethylpiperazine;
1-[(4-cyanophenyl)methylimidazol-5-ylmethyl]piperazine-4-2,4-dimethylbenzyloxycarbonyl;
1-[(4-cyanophenyl)methylimidazol-5-ylmethyl]piperazine-4-(2-methylbenzyloxycarbonyl);
1-[(4-cyanophenyl)methylimidazol-5-ylmethyl]piperazine-4-(4′-acetamidobenzyloxycarbonyl);
1-[(4-cyanophenyl)methylimidazol-5-ylmethyl]piperazine-4-[(3′-methylbenzyloxycarbonyl);
1-[(4-cyanophenyl)methylimidazol-5-ylmethyl]piperazine-4-(2′-methoxybenzyloxycarbonyl);
1-[(4-cyanophenyl)methylimidazol-5-ylmethyl]piperazine-4-(3′-methoxybenzyloxycarbonyl);
1-[(4-cyanophenyl)methylimidazol-5-ylmethyl]piperazine-4-(1-oxypyridine-3-methyloxycarbonyl);
1-[(4-cyanophenyl)methylimidazol-5-ylmethyl]piperazine-4-(3-pyridinemethyloxycarbonyl);
1-[(4-cyanophenyl)methylimidazol-5-ylmethyl]piperazine-4-(4′-pyridinemethyloxycarbonyl);
1-[(4-cyanophenyl)methylimidazol-5-ylmethyl]piperazine-4-(2′,5′-dimethylbenzyloxycarbonyl);
1-[(4-cyanophenyl)methylimidazol-5-ylmethyl]piperazine-4-[(1,3-benzodioxolan-5-methyl)oxycarbonyl];
or a pharmaceutically acceptable salt or optical isomer thereof.

15. The method according to claim 14 wherein the prenyl-protein transferase inhibitor is selected from:
1-(3-Chlorophenyl)-4-[1-(4-cyanobenzyl)-5-imidazolylmethyl]-2-piperazinone;
(R)-1-(3-Chlorophenyl)-4-[1-(4-cyanobenzyl)-5-imidazolylmethyl]-5-[2-(ethanesulfonyl)methyl]-2-piperazinone;
4-[1-(5-Chloro-2-oxo-2H-[1,2′]bipyridinyl-5′-ylmethyl)-1H-pyrrol-2-ylmethyl]-benzonitrile and
1-[N-(1-(4-cyanobenzyl)-5-imidazolylmethyl)-N-(4-cyanobenzyl)amino]-4-(phenoxy)benzene
or a pharmaceutically acceptable salt or optical isomer thereof.

16. A pharmaceutical composition for achieving a therapeutic effect in a mammal in need thereof which comprises amounts of at least one inhibitor of prenyl-protein transferase and at least one PSA conjugate.

17. The pharmaceutical composition according to claim 16 comprising an amount of a prenyl-protein transferase inhibitor and an amount of a PSA conjugate.

18. The pharmaceutical composition according to claim 16 wherein the therapeutic effect is treatment of cancer.

19. The pharmaceutical composition according to claim 16 wherein the therapeutic effect is selected from inhibition of cancerous tumor growth and the regression of cancerous tumors.

20. The method according to claim 14 wherein the cancer is a cancer related to cells that express enzymatically active PSA.

21. The method according to claim 20 wherein the cancer is prostate cancer.

22. A method of preparing a pharmaceutical composition for achieving a therapeutic effect in a mammal in need thereof which comprises mixing amounts of at least one inhibitor of prenyl-protein transferase and at least one PSA conjugate.

23. The method of preparing a pharmaceutical composition according to claim 22 comprising mixing an amount of a prenyl-protein transferase inhibitor and an amount of an PSA conjugate.

24. A method of treating cancer in a mammal in need thereof which comprises administering to said mammal amounts of at least one inhibitor of prenyl-protein transferase and at least one PSA conjugate and applying to the mammal radiation therapy.

25. The method according to claim 24 wherein an amount of a prenyl-protein transferase inhibitor and an amount of a PSA conjugate are administered simultaneously.

26. The method according to claim 24 wherein an amount of a prenyl-protein transferase inhibitor and an amount of an PSA conjugate are administered consecutively.


SEQUENCE LISTING (87 sequences)

SEQ ID NO: 1 - protein, 4 aa, Artificial Sequence
  C-terminus of human protein
CVIM

SEQ ID NO: 2 - protein, 4 aa, Artificial Sequence
  C-terminus of human protein
CVLL

SEQ ID NO: 3 - protein, 4 aa, Artificial Sequence
  C-terminus of human protein
CVVM

SEQ ID NO: 4 - protein, 4 aa, Artificial Sequence
  C-terminus of human protein
CIIM

SEQ ID NO: 5 - protein, 4 aa, Artificial Sequence
  C-terminus of human protein
CLLL

SEQ ID NO: 6 - protein, 4 aa, Artificial Sequence
  C-terminus of human protein
CQLL

SEQ ID NO: 7 - protein, 4 aa, Artificial Sequence
  completely synthetic sequence
CSIM

SEQ ID NO: 8 - protein, 4 aa, Artificial Sequence
  completely synthetic sequence
CAIM

SEQ ID NO: 9 - protein, 4 aa, Artificial Sequence
  C-terminus of human protein
CKVL

SEQ ID NO: 10 - protein, 4 aa, Artificial Sequence
  C-terminus of human protein
CLIM

SEQ ID NO: 11 - protein, 4 aa, Artificial Sequence
  C-terminus of human protein
CVLS

SEQ ID NO: 12 - protein, 4 aa, Artificial Sequence
  C-terminus of human protein
CVIL

SEQ ID NO: 13 - protein, 4 aa, Artificial Sequence
  C-terminus of human protein
CNIQ

SEQ ID NO: 14 - protein, 7 aa, Artificial Sequence
  completely synthetic amino acid sequence
NKISYQS

SEQ ID NO: 15 - protein, 8 aa, Artificial Sequence
  completely synthetic amino acid sequence
NKISYQSS

SEQ ID NO: 16 - protein, 9 aa, Artificial Sequence
  completely synthetic amino acid sequence
NKISYQSSS

SEQ ID NO: 17 - protein, 10 aa, Artificial Sequence
  completely synthetic amino acid sequence
NKISYQSSST

SEQ ID NO: 18 - protein, 11 aa, Artificial Sequence
  completely synthetic amino acid sequence
NKISYQSSSTE

SEQ ID NO: 19 - protein, 12 aa, Artificial Sequence
  completely synthetic amino acid sequence
ANKISYQSSSTE

SEQ ID NO: 20 - protein, 11 aa, Artificial Sequence
  completely synthetic amino acid sequence
ANKISYQSSST

SEQ ID NO: 21 - protein, 12 aa, Artificial Sequence
  completely synthetic amino acid sequence
ANKISYQSSSTL

SEQ ID NO: 22 - protein, 12 aa, Artificial Sequence
  completely synthetic amino acid sequence
ANKASYQSASTL

SEQ ID NO: 23 - protein, 11 aa, Artificial Sequence
  completely synthetic amino acid sequence
ANKASYQSASL

SEQ ID NO: 24 - protein, 11 aa, Artificial Sequence
  completely synthetic amino acid sequence
ANKASYQSSSL

SEQ ID NO: 25 - protein, 10 aa, Artificial Sequence
  completely synthetic amino acid sequence
ANKASYQSSL

SEQ ID NO: 26 - protein, 7 aa, Artificial Sequence
  completely synthetic amino acid sequence
SYQSSSL

SEQ ID NO: 27 - protein, 7 aa, Artificial Sequence
  completely synthetic amino acid sequence
RYQSSSL

SEQ ID NO: 28 - protein, 7 aa, Artificial Sequence
  completely synthetic amino acid sequence
KYQSSSL

SEQ ID NO: 29 - protein, 6 aa, Artificial Sequence
  completely synthetic amino acid sequence
KYQSSL

SEQ ID NO: 30 - protein, 7 aa, Artificial Sequence
  completely synthetic amino acid sequence
KYQSSSL

SEQ ID NO: 31 - protein, 11 aa, Artificial Sequence
  completely synthetic amino acid sequence
LNKASYQSSSL

SEQ ID NO: 32 - protein, 7 aa, Artificial Sequence
  completely synthetic amino acid sequence
SSSXQSL

SEQ ID NO: 33 - protein, 6 aa, Artificial Sequence
  completely synthetic amino acid sequence
SSXQSL

SEQ ID NO: 34 - protein, 7 aa, Artificial Sequence
  completely synthetic amino acid sequence
SSSXQSL

SEQ ID NO: 35 - protein, 7 aa, Artificial Sequence
  completely synthetic amino acid sequence
PASXQSL

SEQ ID NO: 36 - protein, 7 aa, Artificial Sequence
  completely synthetic amino acid sequence
PASXQSL

SEQ ID NO: 37 - protein, 7 aa, Artificial Sequence
  completely synthetic amino acid sequence
PASXQSL

SEQ ID NO: 38 - protein, 7 aa, Artificial Sequence
  completely synthetic amino acid sequence
PASXQSL

SEQ ID NO: 39 - protein, 7 aa, Artificial Sequence
  completely synthetic amino acid sequence
PASXQSL

SEQ ID NO: 40 - protein, 7 aa, Artificial Sequence
  completely synthetic amino acid sequence
PASXQSL

SEQ ID NO: 41 - protein, 7 aa, Artificial Sequence
  completely synthetic amino acid sequence
PASXQSX

SEQ ID NO: 42 - protein, 7 aa, Artificial Sequence
  completely synthetic amino acid sequence
PASXQSL

SEQ ID NO: 43 - protein, 7 aa, Artificial Sequence
  completely synthetic amino acid sequence
PASXQSV

SEQ ID NO: 44 - protein, 7 aa, Artificial Sequence
  completely synthetic amino acid sequence
PASXQSL

SEQ ID NO: 45 - protein, 7 aa, Artificial Sequence
  completely synthetic amino acid sequence
SSSXQSV

SEQ ID NO: 46 - protein, 7 aa, Artificial Sequence
  completely synthetic amino acid sequence
PSSXQSV

SEQ ID NO: 47 - protein, 7 aa, Artificial Sequence
  completely synthetic amino acid sequence
SSSXQSL

SEQ ID NO: 48 - protein, 7 aa, Artificial Sequence
  completely synthetic amino acid sequence
PSSXQSL

SEQ ID NO: 49 - protein, 8 aa, Artificial Sequence
  completely synthetic amino acid sequence
PSSXQSSP

SEQ ID NO: 50 - protein, 7 aa, Artificial Sequence
  completely synthetic amino acid sequence
PSSXQSG

SEQ ID NO: 51 - protein, 8 aa, Artificial Sequence
  completely synthetic amino acid sequence
PSSXQSSG

SEQ ID NO: 52 - protein, 8 aa, Artificial Sequence
  completely synthetic amino acid sequence
PSSXQSSP

SEQ ID NO: 53 - protein, 7 aa, Artificial Sequence
  completely synthetic amino acid sequence
PSSXQSV

SEQ ID NO: 54 - protein, 8 aa, Artificial Sequence
  completely synthetic amino acid sequence
PSSXQSSP

SEQ ID NO: 55 - protein, 7 aa, Artificial Sequence
  completely synthetic amino acid sequence
ASSXQSP

SEQ ID NO: 56 - protein, 7 aa, Artificial Sequence
  completely synthetic amino acid sequence
ASSXQSP

SEQ ID NO: 57 - protein, 8 aa, Artificial Sequence
  completely synthetic amino acid sequence
XSSXQSSP

SEQ ID NO: 58 - protein, 7 aa, Artificial Sequence
  completely synthetic amino acid sequence
PSSXQSV

SEQ ID NO: 59 - protein, 7 aa, Artificial Sequence
  completely synthetic amino acid sequence
PSSXQSL

SEQ ID NO: 60 - protein, 7 aa, Artificial Sequence
  completely synthetic amino acid sequence
PASXQSL

SEQ ID NO: 61 - protein, 7 aa, Artificial Sequence
  completely synthetic amino acid sequence
PASXQSL

SEQ ID NO: 62 - protein, 6 aa, Artificial Sequence
  completely synthetic amino acid sequence
PSSXQS

SEQ ID NO: 63 - protein, 5 aa, Artificial Sequence
  completely synthetic amino acid sequence
PSSXQ

SEQ ID NO: 64 - protein, 6 aa, Artificial Sequence
  completely synthetic amino acid sequence
XSSXQS

SEQ ID NO: 65 - protein, 7 aa, Artificial Sequence
  completely synthetic amino acid sequence
XSSXQSP

SEQ ID NO: 66 - protein, 6 aa, Artificial Sequence
  completely synthetic amino acid sequence
XXQSSP

SEQ ID NO: 67 - protein, 5 aa, Artificial Sequence
  completely synthetic amino acid sequence
XXQSS

SEQ ID NO: 68 - protein, 15 aa, Artificial Sequence
  completely synthetic amino acid sequence
GKKKKKKSKTKCVIM

SEQ ID NO: 69 - DNA, 52 bp, Artificial Sequence
  completely synthetic DNA sequence
gagagggaattcgggcccttcctgcatgctgctgctgctgctgctgctgggc

SEQ ID NO: 70 - DNA, 41 bp, Artificial Sequence
  completely synthetic DNA sequence
gagagagctcgaggttaacccgggtgcgcggcgtcggtggt

SEQ ID NO: 71 - DNA, 42 bp, Artificial Sequence
  completely synthetic DNA sequence
gagagagtctagagttaacccgtggtccccgcgttgcttcct

SEQ ID NO: 72 - DNA, 43 bp, Artificial Sequence
  completely synthetic DNA sequence
gaagaggaagcttggtaccgccactgggctgtaggtggtggct

SEQ ID NO: 73 - DNA, 27 bp, Artificial Sequence
  completely synthetic DNA sequence
ggcagagctcgtttagtgaaccgtcag

SEQ ID NO: 74 - DNA, 27 bp, Artificial Sequence
  completely synthetic DNA sequence
gagagatctcaaggacggtgactgcag

SEQ ID NO: 75 - DNA, 86 bp, Artificial Sequence
  completely synthetic DNA sequence
tctcctcgaggccaccatggggagtagcaagagcaagcctaaggaccccagccagcgccggatgacagaatacaagcttgtggtgg

SEQ ID NO: 76 - DNA, 33 bp, Artificial Sequence
  completely synthetic DNA sequence
cacatctagatcaggacagcacagacttgcagc

SEQ ID NO: 77 - DNA, 41 bp, Artificial Sequence
  completely synthetic DNA sequence
tctcctcgaggccaccatgacagaatacaagcttgtggtgg

SEQ ID NO: 78 - DNA, 38 bp, Artificial Sequence
  completely synthetic DNA sequence
cactctagactggtgtcagagcagcacacacttgcagc

SEQ ID NO: 79 - DNA, 38 bp, Artificial Sequence
  completely synthetic DNA sequence
gagagaattcgccaccatgacggaatataagctggtgg

SEQ ID NO: 80 - DNA, 33 bp, Artificial Sequence
  completely synthetic DNA sequence
gagagtcgacgcgtcaggagagcacacacttgc

SEQ ID NO: 81 - DNA, 22 bp, Artificial Sequence
  completely synthetic DNA sequence
ccgccggcctggaggagtacag

SEQ ID NO: 82 - DNA, 38 bp, Artificial Sequence
  completely synthetic DNA sequence
gagagaattcgccaccatgactgagtacaaactggtgg

SEQ ID NO: 83 - DNA, 32 bp, Artificial Sequence
  completely synthetic DNA sequence
gagagtcgacttgttacatcaccacacatggc

SEQ ID NO: 84 - DNA, 21 bp, Artificial Sequence
  completely synthetic DNA sequence
gttggagcagttggtgttggg

SEQ ID NO: 85 - DNA, 38 bp, Artificial Sequence
  completely synthetic DNA sequence
gagaggtaccgccaccatgactgaatataaacttgtgg

SEQ ID NO: 86 - DNA, 36 bp, Artificial Sequence
  completely synthetic DNA sequence
ctctgtcgacgtatttacataattacacactttgtc

SEQ ID NO: 87 - DNA, 24 bp, Artificial Sequence
  completely synthetic DNA sequence
gtagttggagctgttggcgtaggc